(19) United States
(12) Patent Application Publication
(10) Pub. No.: US 2011/0097339 A1
(43) Pub. Date: Apr. 28, 2011

(54) Compositions monovalent for CD28 binding and methods of use

(75) Inventors: Steven Holmes, Cambridge (GB); Carrie Enever, Cambridge (GB); Steven Grant, Cambridge (GB); David Sansom, Birmingham (GB)

(73) Assignee: Domantis Limited, Cambridge (GB)

(21) Appl. No.: 12/456,703

(22) Filed: Jun. 18, 2009

Related U.S. Application Data
(60) Provisional application No. 61/007,553, filed on Jul. 18, 2008.

(51) Int. Cl. A61K 39/395; C07K 16/28; C07H 21/00; C12N 15/63; C12N 5/10; C12P 21/02; A61P 11/06; A61P 37/08; A61P 35/00; A61P 31/00

(52) U.S. Cl. 424/158.1; 530/389.2; 530/387.3; 530/391.1; 536/23.4; 536/23.53; 435/320.1; 435/325; 435/69.6

(57) ABSTRACT

The disclosure relates to a monovalent polypeptide domain which specifically binds CD28, as well as to an antagonist of CD28, where the antagonist comprises a monovalent polypeptide domain which specifically binds CD28. This disclosure encompasses monovalent polypeptide domains comprising an antibody single variable domain that monovalently binds CD28. An antibody single variable domain that is monovalent for binding of CD28 can inhibit CD28 activity. In one aspect, a monovalent anti-CD28 antibody single variable domain consists of or comprises an antibody single variable domain that specifically binds and antagonizes the activity of CD28, in an aspect, without substantially agonizing CD28 activity. In another aspect, the monovalent anti-CD28 antibody single variable domain is a human antibody single variable domain. The disclosure further encompasses methods of antagonizing CD80 and/or CD86 interactions with CD28 in an individual and methods of treating diseases or disorders involving CD80 and/or CD86 interactions with CD28, the methods involving administering a monovalent anti-CD28 antibody single variable domain to the individual.

This application claims the benefit of U.S. provisional application 61/007,553, filed Jul. 18, 2008, which entire contents of which are incorporated by reference herein.

### BACKGROUND

Antigen-nonspecific intercellular interactions between T-lymphocytes and antigen-presenting cells (APCs) generate T cell costimulatory signals that generate T cell responses to antigen (Jenkins and Johnson (1993) Curr. Opin. Immunol. 5:361 367). Costimulatory signals determine the magnitude of a T cell response to antigen, and whether this response activates or inactivates subsequent responses to antigen (Mueller et al. (1989) Annu. Rev. Immunol. 7: 445 480). T cell activation in the absence of costimulation results in an aborted or anergic T cell response (Schwartz, R. H. (1992) Cell 71:1065 1068). One key costimulatory signal is provided by interaction of the T cell surface receptor CD28 with B7 related molecules on antigen presenting cells (e.g., also known as B7-1 and B7-2, or CD80 and CD86, respectively) (P. Linsley and J. Ledbetter (1993) Annu. Rev. Immunol. 11:191 212).

The interaction of CD28 with B7-1 (CD80) and B7-2 (CD86) costimulatory molecules provide a major signaling pathway for augmenting and sustaining T-cell responses. CD28 on the surface of T cells can receive a costimulatory signal delivered by a ligand on B cells or other APCs. Ligands for CD28 include members of the B7 family of B lymphocyte activation antigens, such as B7-1 and/or B7-2 (Freedman, A. S. et al. (1987) J. Immunol. 137, 3260-3267; Freeman, G. J. et al. (1989) J. Immunol. 143, 2714-2722; Freeman, G. J. et al. (1991) J. Exp. Med. 174, 625-631; Freeman, G. J. et al. (1993) Science 262, 909-911; Azuma, M. et al. (1993) Nature 366, 76-79; Freeman, G. J. et al. (1993) J. Exp. Med. 178, 2185-2192). B7-1 and B7-2 are also ligands for another molecule, CTLA4, present on the surface of activated T cells.

CD28 is constitutively expressed on the surface of T cells, virtually all human CD4+ T cells, 50% of human CD8+ T cells, some natural killer cells and all murine T cells. CD28 is a type I transmembrane glycoprotein and is a member of the Immunoglobulin family by virtue of its single Ig variable-like extracellular domain which has a MYPPPY motif required for binding CD80 and 86, (Peach et al. 1994, J. Exp. Med. 180:2049-2058). CD28 has a cysteine residue located after the Ig variable-like domain, which is involved in its homodimerization.

CD28 transmits a signal that synergizes with the T cell receptor (TCR) signal to promote the activation of naïve T cells (Lanzavecchia et al. (1999) Cell 96:1-4). CD28 signaling regulates the threshold for T-cell activation and significantly reduces the number of TCR engagements needed for effective T cell activation (Viola et al. 1996. Science 273:104-6). CD28 co-stimulation results in enhanced T cell proliferation, production of multiple cytokines, and cytokine receptors, increased expression of proteins involved in cell cycle progression, sustaining T cell survival, and sustained CD40 Ligand (CD40 L) expression on T cells (Sharpe et al. Fundamental Immunology, W.E. Paul Ed. Fifth Edition, Page 396).

CD28 signals have a critical role in regulating CD4 and CD8 T cell differentiation. CD28 also optimizes the responses of previously activated T cells, promoting IL-2 production, and T-cell survival. IL-4 production by naïve T cells is highly dependent on B7-1/B7-2 co-stimulation. Interruption of the CD28/B7 pathway during activation of naïve T cells impairs T-cell proliferation and differentiation, while interruption of the CD28/B7 pathway in previously activated T cells diminishes T-cell expansion but not effector cytokine production (Sharpe et al. Fundamental Immunology, W.E. Paul Ed. Fifth Edition, Page 393-404).

T helper cell-dependent antibody responses use the B7-CD28 pathway to provide co-stimulatory signals essential for cognate T cell B cell interactions required for Immunoglobulin class switching and germinal center formation. In CD28 knock out mice, potentially reactive B cells accumulate within lymphoid follicles after antigenic stimulation, but are not able to proliferate or undergo somatic mutation, (Ferguson et al. J. Immunol. (1996) 156:4576-4581).

B7-1/B7-2 co-stimulation of inhibitory signals occur when B7-1/B7-2 bind a second, higher affinity receptor on T cells, CTLA-4 (CD152), (Brunet et al. Nature (1987)328:267-270, Linsley et al. (1991) J. Exp. Med. 174:561-569). The outcome of an immune response involves a balance between CD28 mediated T cell activation and CTLA-4 mediated T cell inhibition. Inhibition of CD28 mediated T cell activation could inhibit undesired T cell responses occurring during autoimmunity, transplant rejection, or allergic responses. For example, inhibiting CD28 mediated T cell activation could delay graft rejection, prevent acute allograft rejection, induce donor specific tolerance, and prevent development and interrupt the progression of chronic allograft rejection, as well as prevent graft versus host disease (GVH), i.e., when transplanted, T cells mount a vigorous immune response against host tissue alloantigens, (Salama et al. (2001) J. Clin. Invest. 108:943-48). Not only would inhibiting CD28 mediated T cell activation dampen the immune response through negating activation signaling through CD28, it may also increase the amount binding of CD86 and CD80 to CTLA-4, thereby further hastening inhibition of the T cell response.

Thus, inhibiting CD28 mediated T cell activation could be used to prevent induction of autoimmunity and moderate the progression and/or severity of established autoimmune diseases, including models of collagen induced arthritis, autoimmune thyroiditis, autoimmune uveitis, myasthenia gravis and lupus, (Saloman et al. Ann. Rev. Immunol. (2001)) 19:225-252). Inhibiting CD28 mediated T cell activation could be used to prevent graft rejection and/or graft versus host disease, and/or moderate the progression and/or severity of transplantation related diseases, including models. Inhibiting and/or reducing inflammation mediated at least in part through CD28 signaling is also desirable. What is needed is a way to inhibit CD28-mediated T cell activation, without stimulation of CD28 signaling pathways. The disclosure set forth herein meets and addresses this need.

### SUMMARY

Disclosed herein are monovalent binders of CD28, including antibody single variable domains that monovalently bind CD28. In one aspect, the monovalent binders of CD28, including antibody single variable domains that monovalently bind CD28, antagonize an activity of CD28. Because of the clear importance of CD28 in the regulation of the T cell response as well as in the production of antibodies, the CD28/B7 (CD80 and CD86) interaction and pathways present important targets for the development of therapeutic approaches for the treatment of diseases and disorders that involve inappropriate and/or undesirable cellular immune responses, and/or excessive antibody responses, such as transplantation rejection, inflammation and autoimmunity. With respect to inhibiting CD28-mediated T cell activation, an antagonist of CD28 that specifically binds CD28 in a monovalent fashion, has unique advantages in antagonizing CD28 mediated T cell responses, e.g., T cell activation and/or T cell proliferation, over an anti-CD28 dimeric antibody which clusters the cell surface CD28 molecules, promoting T cell stimulation. Thus, an antagonist antibody single variable domain which is monovalent for binding of CD28 can inhibit CD28 activity, dampening an immune response, while avoiding potential undesirable stimulatory affects that can occur with antibodies capable of divalent or multivalent binding of cell surface CD28. A monovalent antibody single variable domain which specifically binds CD28 can also be applied to any of a number of uses for which a standard divalent antibody is used, e.g., in vivo imaging and diagnosis.

Described herein are CD28 antagonists, which comprise or consist of a monovalent binder of CD28, including an antibody single variable domain, which inhibits CD28 mediated signaling. In one aspect, the inhibited CD28 mediated signaling results from reduced binding to CD28 by CD80 and/or CD86. The amount of inhibition or reduction in CD28 mediated signaling as a result of the presence of monovalent antagonists to CD28, including CD28 specific antibody single variable domains described herein, can be measured based on receptor binding assays well known in the art.

The antagonist monovalent binders of CD28 described herein, do not bind the common motif, MYPPPY found on the extracellular portion on CTLA4 and CD28. Thus, the antagonist monovalent binders of CD28 described herein, including antibody single variable domains, do not cross-react with CTLA4, allowing CD80 and CD86 to be available for binding to CTLA4, further contributing to inhibition of the T cell response. As described above, CTLA4 is a known inhibitor of T cell responsiveness.

In one aspect, the CD28 antagonist described herein, which comprises or consists of a monovalent binder of CD28, including a single domain antibody, that specifically binds and antagonizes an activity of CD28, without substantially agonizing CD28 activity. Administering a human or a humanized monovalent binder of CD28, such as an antibody single variable domain, is particularly useful for the treatment or prevention of disease, especially a disease where it is desirable to avoid the generation of an alloreactive immune response and/or to suppress a T cell mediated immune response. In one aspect, the antibody single variable domain is essentially a human antibody single variable domain that monovalently binds CD28, which, in an exemplary embodiment, binds and antagonizes CD28 signaling without substantially agonizing CD28 activity.

In another embodiment, the monovalent domain which specifically binds CD28, competes for binding to CD28 with DOM21-4, DOM21-18 and/or DOM21-28, and/or competes for binding with one or more of DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24, DOM21-25, DOM21-26, DOM21-27, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-58, DOM21-59, DOM21-60, DOM21-61, DOM21-62, DOM21-63, DOM21-64, DOM21-65, DOM21-66, DOM21-67, and/or DOM21-68.

In another embodiment, the monovalent domain which specifically binds CD28 and/or antagonizes CD28 mediated signaling without substantially agonizing CD28 activity, competes for binding to CD28 with DOM21-4, DOM21-18 and/or DOM21-28, and/or competes for binding with one or more of DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24, DOM21-25, DOM21-26, DOM21-27, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-58, DOM21-59, DOM21-60, DOM21-61, DOM21-62, DOM21-63, DOM21-64, DOM21-65, DOM21-66, DOM21-67, and/or DOM21-68.

In an aspect, the monovalent domain which specifically binds CD28, and/or antagonizes CD28 mediated signaling, and/or antagonizes CD28 mediated signaling without substantially agonizing CD28 activity, and/or competes for binding to CD28 with one or more of the DOM21 peptides selected from the following: DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-27, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-58, DOM21-59, DOM21-60, DOM21-61, DOM21-62, DOM21-63, DOM21-64, DOM21-65, DOM21-66, DOM21-67, and/or DOM21-68, comprises a sequence which is at least 70% identical, or at least 75% identical to a sequence selected from the group consisting of the amino acid sequence of DOM21-4, DOM21-18 and/or DOM21-28, and/or is at least 70% identical, or at least 75% identical to one or more of the DOM21 peptides selected from the following: DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24, DOM21-25, DOM21-26, DOM21-27, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-58, DOM21-59, DOM21-60, DOM21-61, DOM21-62, DOM21-63, DOM21-64, DOM21-65, DOM21-66, DOM21-67, and/or DOM21-68.

In an aspect, the monovalent domain which specifically binds CD28, and/or antagonizes CD28 mediated signaling, and/or antagonizes CD28 mediated signaling without substantially agonizing CD28 activity, and/or competes for binding to CD28 with one or more of the DOM21 peptides described above, and/or comprises a sequence which is at least 70% identical or at least 75% identical to the amino acid sequence of one or more of the DOM21 peptides described above, is an antibody single variable domain.

In another aspect, the antibody single variable domain and/or monovalent domain which specifically binds CD28, and/or antagonizes CD28 mediated signaling, and/or antagonizes CD28 mediated signaling without substantially agonizing CD28 activity, and/or competes for binding to CD28 with one or more of the DOM21 peptides described above, and/or comprises a sequence which is at least 70% identical to or at least 75% identical to one or more of the DOM21 peptides described above, inhibits binding to CD28 by CD80 and/or CD86, and in one aspect more strongly inhibits the binding to CD28 by CD86 relative to the binding to CD28 by CD80. In an aspect, the antibody single variable domain inhibits the binding to CD28 by CD80 and/or CD86 with an IC₅₀ in the range of 50 pM up to and including 3.0 pM, ranging from about 5 nM, about 4 nM, about 3 nM, about 2.5 nM, about 2.0 nM, about 1.9 nM, about 1.8 nM, about 1.7 nM, about 1.6 nM, about 1.5 nM, about 1.4 nM, about 1.3 nM, about 1.2, nM, about 1.1 nM, about 1.0 nM, about 0.9 nM, about 0.8 nM, about 0.7 nM, about 0.6 nM, about 0.5 nM, about 0.4 nM, about 0.3 nM, about 0.2 nM, to about 0.1 nM. In an aspect, the antagonist, monovalent domain and or single variable domain which specifically binds CD28, does not cross react with CTLA4.

In one embodiment the CD28 antagonist and/or antibody single variable domain has a CDR1 sequence that is at least 50% identical to the CDR1 sequence of the group consisting of DOM21-4, DOM21-18 and/or DOM21-28. In one embodiment the CD28 antagonist and/or antibody single variable domain has a CDR2 sequence that is at least 50% identical to the CDR2 sequence of the group consisting of DOM21-4, DOM21-18 and/or DOM21-28. In one embodiment the CD28 antagonist and/or antibody single variable domain has a CDR3 sequence that is at least 50% identical to the CDR3 sequence selected from the group consisting of DOM21-4, DOM21-18 and/or DOM21-28. In one embodiment the CD28 antagonist and/or antibody single variable domain has a CDR1 sequence that is at least 50% identical to the CDR1 sequence selected from the group consisting of DOM21-4, DOM21-18 and/or DOM21-28, and a CDR2 sequence that is at least 50% identical to the CDR2 sequence selected from the group consisting of DOM21-4, DOM21-18 and/or DOM21-28. In one embodiment the CD28 antagonist and/or antibody single variable domain has a CDR2 sequence that is at least 50% identical to the CDR2 sequence selected from the group consisting of DOM21-4, DOM21-18 and/or DOM21-28, and a CDR3 sequence that is at least 50% identical to the CDR3 sequence selected from the group consisting of DOM21-4, DOM21-18 and/or DOM21-28. In one embodiment the CD28 antagonist and/or antibody single variable domain has a CDR1 sequence that is at least 50% identical to the CDR1 sequence selected from the group consisting of DOM21-4, DOM21-18 and/or DOM21-28, and a CDR3 sequence that is at least 50% identical to the CDR3 sequence selected from the group consisting of DOM21-4, DOM21-18 and/or DOM21-28. In one embodiment the CD28 antagonist and/or antibody single variable domain has a CDR1 sequence that is at least 50% identical to the CDR1 sequence selected from the group consisting of DOM21-4, DOM21-18 and/or DOM21-28, and a CDR2 sequence that is at least 50% identical to the CDR2 sequence selected from the group consisting of DOM21-4, DOM21-18 and/or DOM21-28, and a CDR3 sequence that is at least 50% identical to the CDR3 sequence of selected from the group consisting of DOM21-4, DOM21-18 and/or DOM21-28.

In one embodiment the antagonist comprises an antibody single variable domain selected from the group consisting of: DOM21-4, DOM21-18 and DOM21-28, or an antibody single variable which is at least 50% identical to the amino acid sequence of an antibody single variable domain selected from the group consisting of: DOM21-4, DOM21-18 and DOM21-28. In one embodiment the CD28 antagonist and/or antibody single variable domain binds to the same epitope of CD28 bound by a antibody single variable domain selected from the group consisting of: DOM21-4, DOM21-18 and DOM21-28. In one embodiment the CD28 antagonist and/or antibody single variable domain binds to the same epitope of CD28 bound by an antibody single variable domain encoded by a nucleic acid molecule comprising a sequence selected from the group consisting of SEQ ID NOs: 1-57. In one embodiment the CD28 antagonist and/or antibody single variable domain comprises one or more of CDR1, CDR2, and CDR3 from the group consisting of: DOM21-4, DOM21-18 and DOM21-28. In one embodiment the CD28 antagonist and/or antibody single variable domain comprises one or more of CDR1, CDR2, and CDR3 of an antibody single variable domain polypeptide encoded by a nucleic acid sequence comprising a sequence selected from the group consisting of SEQ ID NOs:1-57. In one embodiment the CD28 antagonist and/or antibody single variable domain comprises two or more of CDR1, CDR2, or CDR3 of DOM21-4, DOM21-18 and/or DOM21-28. In one embodiment the CD28 antagonist and/or antibody single variable domain comprises two or more of CDR1, CDR2, and CDR3 of an antibody single variable domain polypeptide encoded by a nucleic acid comprising a sequence selected from the group consisting of SEQ ID NOs: 1-57.

In one embodiment the CD28 antagonist and/or antibody single variable domain further comprises an additional antibody single variable domain or ligand binding site which specifically binds an antigen other than CD28. In one embodiment, the antigen other than CD28 is a T cell surface antigen, e.g., a T cell receptor. In one aspect, the T cell receptor binds an antigen including a transplantation antigen and an autoantigen. The autoantigen bound by the T cell receptor in T cells mediating an autoimmune disease includes but is not limited to the diseases of systemic lupus erythematosis, multiple sclerosis, rheumatoid arthritis, diabetes, diabetes, psoriasis, scleroderma, atherosclerosis, inflammatory bowel disease, Crohn's disease, and ulcerative colitis. The T cell receptor can recognize the antigen in the context of class II MHC. The transplantation antigen is bound by the T cell receptor in T cells mediating a transplantation based disease selected from the group consisting of: allograft rejection, xenograft transplant rejection, and graft vs. host disease.

In one embodiment, the antigen other than CD28 is serum albumin, e.g., human serum albumin. In one embodiment, the antagonist of CD28 further comprises PEG. PEG can be linked to said antibody single variable domain, e.g. through a cysteine or a lysine residue.

One embodiment is an isolated composition comprising the CD28 antagonist and/or the antibody single variable domain, and a carrier. In one aspect, the composition is a pharmaceutical composition, and the carrier is a pharmaceutical carrier. In another aspect, composition comprises a therapeutically-effective amount of said antagonist and/or antibody single variable domain.

Disclosed herein is a method of treating or preventing an autoimmune disease in an individual, the method comprising administering the antagonist or composition thereof, of any one of the previous claims to said individual. The autoimmune disease includes, but are preferably not limited to, systemic lupus erythematosis, multiple sclerosis, rheumatoid arthritis, diabetes, diabetes, psoriasis, scleroderma, atherosclerosis, inflammatory bowel disease, Crohn's disease, and ulcerative colitis. Disclosed herein is a method of treating or preventing a transplantation disease in an individual, the method comprising administering the CD28 antagonist or composition thereof, described herein. The transplantation disease includes allograft rejection, xenograft transplant rejection, and graft vs. host disease.

In one embodiment, the human antibody single variable domain dissociates from human CD28 with a K_{d} in the range of 50 nM to 1 pM, inclusive, as measured by surface plasmon resonance. For example, the K_{d} for human CD28 can be 25 nM to 20 pM, 10 nM to 20 pM, 5 nm to 20 pM, 1 nM to 20 pM, 0.5 nM to 20 pM, 0.1 nM to 20 pM, 0.1 nM to 50 nM, 75 pM to 20 pM, 50 pM to 20 pM, or even 20 pM to 1 pM.

In another embodiment, the antibody single variable domain inhibits the binding of CD28 to CD80. In another embodiment, the antibody single variable domain inhibits the binding of CD28 to CD86. In another embodiment, the antibody single variable domain preferably inhibits the binding of CD28 to CD86 relative to its inhibition of the binding of CD28 to CD80.

In another embodiment, the binding of the antibody single variable domain to CD28 does not substantially agonize CD28 activity.

In another embodiment, the human antibody single variable domain inhibits the binding of CD80 to CD28, and does not substantially agonize signaling by CD28. In another embodiment, the human antibody single variable domain inhibits the binding of CD86 to CD28, and does not substantially agonize signaling by CD28.

In another embodiment, the human antibody single variable domain comprises an antibody single variable domain that binds CD28.

In another embodiment, the human antibody single variable domain is PEG-linked. In one embodiment, the PEG is covalently linked to the human antibody single variable domain. In one preferred embodiment, the PEG-linked human antibody single variable domain has a hydrodynamic size of at least 24 kD. In another preferred embodiment, the PEG is linked to the antibody single variable domain at a cysteine or lysine residue. In another preferred embodiment, the total PEG size is from 20 to 60 kD, inclusive. In another preferred embodiment, the PEG-linked human antibody single variable domain has a hydrodynamic size of at least 200 kD.

In one embodiment, the antibody single variable domain has an increased in vivo t-α or t-βhalf-life relative to the same antibody single variable domain composition lacking polyethylene glycol.

In another embodiment, the tα-half-life of the antibody single variable domain composition is increased by 10% or more when compared to an unmodified protein assayed under otherwise identical conditions. In another embodiment, the tα-half-life of the antibody single variable domain composition is increased by 50% or more. In another embodiment, the tα-half-life of the antibody single variable domain composition is increased by 2× or more. In another embodiment, the tα-half-life of the antibody single variable domain composition is increased by 5× or more, e.g., 10×, 15×, 20×, 25×, 30×, 40×, or more. In another embodiment, the tα-half-life of the antibody single variable domain composition is increased by 50× or more.

In another embodiment, the PEG-linked antibody single variable domain has a tα half-life of 0.25 to 6 hours, inclusive. In another embodiment, the tα half-life is in the range of 30 minutes to 12 hours, inclusive. In another embodiment, the tα-half-life of the antibody single variable domain composition is in the range of 1 to 6 hours.

In another embodiment, the tβ-half-life of the antibody single variable domain composition is increased by 10% or more when compared to an unmodified protein assayed under otherwise identical conditions. In another embodiment, the tβ-half-life of the antibody single variable domain composition is increased by 50% or more. In another embodiment, the tβ-half-life of the antibody single variable domain composition is increased by 2× or more. In another embodiment, the tβ-half-life of the antibody single variable domain composition is increased by 5× or more, e.g., 10×, 15×, 20×, 25×, 30×, 40×, or more. In another embodiment, the tβ-half-life of the antibody single variable domain composition is increased by 50× or more.

In another embodiment, the antibody single variable domain composition has a tβ half-life in the range of 1 hour to 1 month. In one embodiment, the antibody single variable domain composition has a tβ half-life in the range of 12 hours to 4 weeks. In a preferred embodiment, the antibody single variable domain composition has a tβ half-life of two weeks. In another embodiment, the tβ-half-life is in the range of 12 to 48 hours, inclusive. In another embodiment, the tβ-half-life is in the range of 12 to 26 hours, inclusive. In another embodiment the tβ-half life of is at least 2 weeks. In a further embodiment, the antibody single variable domain composition has a tβ half-life in a range wherein the low end of the range is at least 12, 13, 14, 15, 20, 24, 36, 48, or 72 hours and the upper end of the range is up to 24 hours, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, and up to 31 days. In one embodiment an antibody single variable domain composition has a tβ half-life in the range of at least 12 hours up to 24 hours, 14 days, 28 days, 4 weeks, and up to one month.

In addition to, or alternative to the above criteria, a dAb containing composition is provided comprising an antibody single variable domain having an AUC value (area under the curve) in the range of 1 mg.min/ml or more. In one embodiment, the lower end of the range is 5, 10, 15, 20, 30, 100, 200, or 300 mg.min/ml. In addition, or alternatively, a ligand or composition has an AUC in the range of up to 600 mg.min/ml. In one embodiment, the upper end of the range is 500, 400, 300, 200, 150, 100, 75, or 50 mg.min/ml. Advantageously a ligand will have an AUC in the range selected from the group consisting of the following: 15 to 150 mg.min/ml, 15 to 100 mg.min/ml, 15 to 75 mg.min/ml, and 15 to 50 mg.min/ml.

In another embodiment, the antibody single variable domains described herein can be linked to human serum albumin (HSA), which also has the effect of increasing the in vivo half-life of the molecule. The human serum albumin coding sequences can be obtained by PCR using primers derived from the cDNA sequence available at GenBank Accession No. NM000477. Such coding sequences can be fused to the coding sequence for a monovalent anti-CD28 antibody single variable domain as described herein, and the fusion can be expressed by one of skill in the art.

In another embodiment, the tα-half-life of the HSA-linked human antibody single variable domain composition is increased by 10% or more. In another embodiment, the tα-half-life of the HSA-linked human antibody single variable domain composition is in the range of 0.25 hours to 6 hours. In another embodiment, the tβ-half-life of the HSA-linked human antibody single variable domain composition is increased by 10% or more. In another embodiment, the tβ-half-life of the HSA-linked human antibody single variable domain composition is in the range of 12 to 48 hours.

In another embodiment, the human antibody single variable domain which specifically binds CD28 comprises an amino acid sequence encoded by a polynucleotide having a sequence selected from the group consisting of SEQ ID NO:1-57.

In another embodiment, the human antibody single variable domain inhibits binding of CD28 to CD80 with an IC₅₀ in the range of 1 pM to 1.5 μM, inclusive; IC₅₀ for inhibition of CD28 binding to CD80. The IC₅₀ can be in the range of 1 pM to 1 μM, 1 pM to 900 nM, 1 pM to 800 nM, 1 pM to 700 nM, 1 pM to 600 nM, 1 pM to 500 nM, 1 pM to 400 nM, 1 pM to 300 nM, 1 pM to 200 nM, 1 pM to 100 nM, or 1 pM to 50 nM. Further acceptable or preferred ranges include, for example, 50 pM to 1 μM, 100 pM to 500 nM, 125 pM to 250 nM, 150 pM to 200 nM, 150 pM to 100 nM, and 200 pM to 50 nM.

In another embodiment, the human antibody single variable domain inhibits binding of CD28 to CD86 with an IC₅₀ in the range of 1 pM to 1.5 μM, inclusive; IC₅₀ for inhibition of CD28 binding to CD86. The IC₅₀ can be in the range of 1 pM to 1 μM, 1 pM to 900 nM, 1 pM to 800 nM, 1 pM to 700 nM, 1 pM to 600 nM, 1 pM to 500 nM, 1 pM to 400 nM, 1 pM to 300 nM, 1 pM to 200 nM, 1 pM to 100 nM, or 1 pM to 50 nM. Further acceptable or preferred ranges include, for example, 50 pM to 1 μM, 100 pM to 500 nM, 125 pM to 250 nM, 150 pM to 200 nM, 150 pM to 100 nM, and 200 pM to 50 nM.

In another embodiment, the antibody single variable domain is fused to a second antibody single variable domain which binds a ligand other than CD28. In another embodiment, the human antibody single variable domain is free of an Fc domain. The limits of an Fc domain are set out in Kabat et al. (1991, Sequences of Immunological Interest, 5^{th} ed. U.S. Dept. Health & Human Services, Washington, D.C.; incorporated herein by reference). In the alternative, an Fc domain consists of the CH2-CH₃ regions, optionally including a hinge region linked to the CH2.

In another embodiment is provided a human antibody single variable domain which has an amino acid sequence at least 85% identical to an amino acid sequence encoded by a nucleic acid molecule having a sequence selected from the group consisting of SEQ ID NOS:1-57, which antibody single variable domain specifically and monovalently binds CD28.

In another embodiment, an antigen-binding polypeptide is provided for, the polypeptide comprising an antibody single variable domain which specifically and monovalently binds CD28. Another embodiment provides for a polypeptide comprising a moiety which specifically binds CD28, which moiety consists of an immunoglobulin single variable domain, preferably an antibody single variable domain.

In one embodiment, the polypeptide consists of a human antibody single variable domain, preferably an antibody single variable domain.

In another embodiment, the polypeptide inhibits the binding of CD28 to CD80. In another embodiment, the polypeptide inhibits the binding of CD28 to CD86.

In another embodiment, the polypeptide inhibits the binding of CD28 to CD80 and has an IC₅₀ in the range of 1 pM to 1.5 μM, inclusive. For example, the IC₅₀ can be in the range of 1 pM to 1 μM, 1 pM to 900 nM, 1 pM to 800 nM, 1 pM to 700 nM, 1 pM to 600 nM, 1 pM to 500 nM, 1 pM to 400 nM, 1 pM to 300 nM, 1 pM to 200 nM, 1 pM to 100 nM, or 1 pM to 50 nM. Further acceptable or preferred ranges include, for example, 50 pM to 1 μM, 100 pM to 500 nM, 125 pM to 250 nM, 150 pM to 200 nM, 150 pM to 100 nM, and 200 pM to 50 nM.

In another embodiment, the polypeptide inhibits the binding of CD28 to CD86 and has an IC₅₀ in the range of 1 pM to 1.5 μM, inclusive. For example, the IC₅₀ can be in the range of 1 pM to 1 μM, 1 pM to 900 nM, 1 pM to 800 nM, 1 pM to 700 nM, 1 pM to 600 nM, 1 pM to 500 nM, 1 pM to 400 nM, 1 pM to 300 nM, 1 pM to 200 nM, 1 pM to 100 nM, or 1 pM to 50 nM. Further acceptable or preferred ranges include, for example, 50 pM to 1 μM, 100 pM to 500 nM, 125 pM to 250 nM, 150 pM to 200 nM, 150 pM to 100 nM, and 200 pM to 50 nM.

In another embodiment, the binding of the polypeptide to CD28 does not substantially agonize CD28 activity.

In another embodiment, the antibody single variable domain is a human antibody single variable domain, preferably a human antibody single variable domain.

In another embodiment, the antibody single variable domain is a V_{H} or a V_{L} domain.

In one embodiment, the polypeptide is PEG-linked. In one embodiment, the PEG is covalently linked. In one preferred embodiment, the PEG-linked antigen-binding polypeptide has a hydrodynamic size of at least 24 kD. In another preferred embodiment, the PEG is linked to the antigen-binding polypeptide at a cysteine or lysine residue. In another preferred embodiment, the total PEG size is from 20 to 60 kD, inclusive. In another preferred embodiment, the PEG-linked antigen-binding polypeptide has a hydrodynamic size of at least 200 kD.

In another embodiment, the PEG-linked polypeptide has an increased in vivo half-life relative to the same polypeptide composition lacking linked polyethylene glycol. In another embodiment, the tα-half-life of the polypeptide composition is increased by 10% or more. In another embodiment, the tα-half-life of the polypeptide composition is increased by 50% or more. In another embodiment, the tα-half-life of the polypeptide composition is increased by 2× or more. In another embodiment, the tα-half-life of the polypeptide composition is increased by 5× or more, e.g., 10×, 15×, 20×, 25×, 30×, 40×, or more. In another embodiment, the tα-half-life of the polypeptide composition is increased by 50× or more.

In another embodiment, the PEG-linked antibody single variable domain has a tα half-life of 0.25 to 6 hours, inclusive. In another embodiment, the tα half-life is in the range of 30 minutes to 12 hours, inclusive. In another embodiment, the tα-half-life of the polypeptide composition is in the range of 1 to 6 hours.

In another embodiment, the tβ-half-life of the polypeptide composition is increased by 10% or more. In another embodiment, the tβ-half-life of the polypeptide composition is increased by 50% or more. In another embodiment, the tβ-half-life of the polypeptide composition is increased by 2× or more. In another embodiment, the tβ-half-life of the polypeptide composition is increased by 5× or more, e.g., 10×, 15×, 20×, 25×, 30×, 40×, or more. In another embodiment, the tβ-half-life of the polypeptide composition is increased by 50× or more.

In another embodiment, the antibody single variable domain composition has a tβ half-life of 1 to 170 hours, inclusive. In another embodiment, the tβ-half-life is in the range of 12 to 48 hours, inclusive. In another embodiment, the tβ-half-life is in the range of 12 to 26 hours, inclusive. In a further embodiment, the antibody single variable domain composition has a tβ half-life in a range wherein the low end of the range is at least 12, 13, 14, 15, 20, 24, 36, 48, or 72 hours and the upper end of the range is up to 24 hours, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, and up to 31 days. In one embodiment an antibody single variable domain composition has a tβ half-life in the range of at least 12 hours up to 24 hours, 14 days, 28 days, 4 weeks, and up to one month.

In another embodiment, the composition has an AUC value (area under the curve) in the range of 1 mg.min/ml or more. In one embodiment, the lower end of the range is 5, 10, 15, 20, 30, 100, 200, or 300 mg.min/ml. In addition, or alternatively, a ligand or composition has an AUC in the range of up to 600 mg.min/ml. In one embodiment, the upper end of the range is 500, 400, 300, 200, 150, 100, 75, or 50 mg.min/ml. Advantageously a ligand will have an AUC in the range selected from the group consisting of the following: 15 to 150 mg.min/ml, 15 to 100 mg.min/ml, 15 to 75 mg.min/ml, and 15 to 50 mg.min/ml.

In another embodiment, the antibody single variable domain is linked to human serum albumin (HSA). In another embodiment, the antibody single variable domain has an increased in vivo half-life relative to the same polypeptide composition lacking linked HSA. In another embodiment, the antibody single variable domain has a tα-half-life that is increased by 10% or more relative to a molecule lacking linked HSA. In another embodiment, the tα-half-life of the polypeptide composition is in the range of 0.25 minutes to 6 hours. In another embodiment, the tβ-half-life of the polypeptide composition is increased by 10% or more. In another embodiment, the tβ-half-life is in the range of 12 to 48 hours. In one embodiment, the antibody single variable domain linked to HSA has a tβ-half-life of at least 50% of the tβ-half-life of HSA alone in a mammal; preferably a primate; preferably a human. In a further embodiment, the antibody single variable domain linked to HSA has a tβ-half-life of at least 60%, 70%, 80%, 90% and up to 100% or more of the tβ-half-life of HSA alone in a mammal, such as a primate or human.

In another embodiment, the antigen-binding polypeptide comprises an amino acid sequence encoded by a nucleic acid molecule having a sequence selected from the group consisting of SEQ ID NOs:1-57.

In another embodiment, an antibody single variable domain is provided for which has an amino acid sequence at least 50% identical to an amino acid sequence encoded by a polynucleotide having a sequence selected from the group consisting of SEQ ID NOs:1-57, which polypeptide specifically and monovalently binds CD28.

In yet another aspect, an antibody single variable domain is provided for which has an amino acid sequence at least 85% identical to an amino acid sequence encoded by a polynucleotide having a sequence selected from the group consisting of SEQ ID NOs:1-57, which polypeptide specifically and monovalently binds CD28.

In one embodiment, the an antibody single variable domain antagonizes the binding of CD80 to CD28. In one embodiment, the an antibody single variable domain antagonizes the binding of CD86 to CD28.

In another embodiment, the an antibody single variable domain inhibits the binding of CD80 to CD28 and has an IC₅₀ in the range of 1 pM to 1.5 μM, inclusive. For example, the IC₅₀ can be in the range of 1 pM to 1 μM, 1 pM to 900 nM, 1 pM to 800 nM, 1 pM to 700 nM, 1 pM to 600 nM, 1 pM to 500 nM, 1 pM to 400 nM, 1 pM to 300 nM, 1 pM to 200 nM, 1 pM to 100 nM, or 1 pM to 50 nM. Further acceptable or preferred ranges include, for example, 50 pM to 1 μM, 100 pM to 500 nM, 125 pM to 250 nM, 150 pM to 200 nM, 150 pM to 100 nM, and 200 pM to 50 nM.

In another embodiment, the an antibody single variable domain inhibits the binding of CD86 to CD28 and has an IC₅₀ in the range of 1 pM to 1.5 μM, inclusive. For example, the IC₅₀ can be in the range of 1 pM to 1 μM, 1 pM to 900 nM, 1 pM to 800 nM, 1 pM to 700 nM, 1 pM to 600 nM, 1 pM to 500 nM, 1 pM to 400 nM, 1 pM to 300 nM, 1 pM to 200 nM, 1 pM to 100 nM, or 1 pM to 50 nM. Further acceptable or preferred ranges include, for example, 50 pM to 1 μM, 100 pM to 500 nM, 125 pM to 250 nM, 150 pM to 200 nM, 150 pM to 100 nM, and 200 pM to 50 nM.

In another embodiment, the an antibody single variable domain inhibits the interaction of CD80 with CD28, but does not substantially agonize intracellular signaling by CD28. In another embodiment, the antigen-binding polypeptide further comprises a second antibody single variable domain which binds a ligand other than CD28.

In one embodiment is provided a CD28 antagonist comprising an antibody single variable domain which specifically and monovalently binds CD28 and which comprises one or more framework regions comprising an amino acid sequence that is the same as the amino acid sequence of a corresponding framework region encoded by a human germline antibody gene segment, or the amino acid sequence of one or more of said framework regions collectively comprises up to 5 amino acid differences relative to the amino acid sequence of said corresponding framework region encoded by a human germline antibody gene segment.

In one embodiment, the amino acid sequences of FW1, FW2, FW3, and FW4 of the anti-CD28 antibody single variable domain or dAb are the same as the amino acid sequences of corresponding framework regions encoded by a human germline antibody gene segment, or the amino acid sequences of FW1, FW2, FW3, and FW4 collectively contain up to 10 amino acid differences relative to the amino acid sequences of corresponding framework regions encoded by said human germline antibody gene segment. In a further embodiment, the amino acid sequences of FW1, FW2, and FW3 of the anti-CD28 variable domain or dAb are the same as the amino acid sequences of corresponding framework regions encoded by human germline antibody gene segments.

In another embodiment the amino acid sequences of FW1, FW2, FW3, and FW4 collectively preferably contain one or more of the amino acid residues at positions 11, 37, 44, 45, 47, 83, 84, 103, 104 and 108 according to the Kabat numbering are chosen from L and V for position 11, V and F at position 37, G at position 44, L at position 45, W and Y at position 47, R and K at position 83, A, T and D at position 84, W at position 103, G at position 104, and L, M and T at position 108.

In a further embodiment of the foregoing, the human germline antibody gene segment can be selected from the group consisting of DP47, DP45, DP48, and DPK9.

A method of antagonizing the binding of CD80 to CD28 in an individual is provided for, the method comprising administering a monovalent anti-CD28 antibody single variable domain as described herein to the individual, wherein the polypeptide antagonizes the binding of CD80 to CD28 in the individual.

A method of antagonizing the binding of CD86 to CD28 in an individual comprises administering a monovalent anti-CD28 antibody single variable domain as described herein to the individual, wherein the polypeptide antagonizes the binding of CD86 to CD28 in the individual.

A method of antagonizing an activity of CD28 in an individual comprises administering a monovalent anti-CD28 antibody single variable domain as described herein to the individual, wherein the polypeptide antagonizes an activity of CD28.

A composition comprising an extended release formulation comprises an immunoglobulin single variable domain, including, but not limited to, a polypeptide comprising a an antibody single variable domain that binds CD28. In one embodiment, the immunoglobulin single variable domain is a non-human mammalian antibody single variable domain. In another embodiment, the immunoglobulin single variable domain is a human antibody single variable domain.

A method of treating or preventing a disease or disorder mediated by CD28 in an individual in need of such treatment comprises administering to the individual a therapeutically effective amount of a composition comprising a monovalent anti-CD28 single human immunoglobulin variable domain, including a composition comprising an antibody single variable domain that binds CD28, whereby reducing the degree of immune system activation would reduce or prevent symptoms. An area of particular interest is the prevention of organ transplant rejection (e.g., allograft/xenograft rejection). In one embodiment, the disease or disorder is an autoimmune disease or disorder.

A method of treating or preventing a symptom of systemic lupus erythematosus (SLE) in an individual comprises administering a monovalent anti-CD28 antibody single variable domain to said individual in an amount effective to treat or prevent a symptom of systemic lupus erythematosis (SLE). Another method is also provided herein for reducing or alleviating a symptom of a disease such as multiple sclerosis, rheumatoid arthritis, allograft rejection, xenograft rejection, and Type I diabetes, and inflammatory bowel disease (IBD), comprising administering a monovalent anti-CD28 antibody single variable domain to an individual having or suspected of having such a disease, in an amount effective to treat or prevent the disease or a symptom of the disease.

An antibody single variable domain that is monovalent for binding to CD28 comprises a universal antibody framework.

In one embodiment, the universal antibody framework comprises a VH framework selected from the group consisting of DP47, DP45, and DP38, and/or the VL framework is DPK9.

In another embodiment, the antibody single variable domain comprises a generic ligand binding site. In another embodiment, the generic ligand binding site binds a generic ligand selected from the group consisting of protein A, protein L, and protein G.

In another embodiment, the antibody single variable domain comprises a variable domain having one or more framework regions comprising an amino acid sequence that is the same as the amino acid sequence of a corresponding framework region encoded by a human germline antibody gene segment, or the amino acid sequences of one or more of the framework regions collectively comprises up to 5 amino acid differences relative to the amino acid sequence of the corresponding framework region encoded by a human germline antibody gene segment.

In another embodiment, the antibody single variable domain comprises a variable domain, wherein the amino acid sequences of FW1, FW2, FW3, and FW4 are the same as the amino acid sequences of corresponding framework regions encoded by a human germline antibody gene segment, or the antibody sequences of FW1, FW2, FW3, and FW4 collectively contain up to 10 amino acid differences relative to the amino acid sequences of corresponding framework regions encoded by the human germline antibody gene segment.

In another embodiment, the antibody single variable domain comprises an antibody variable domain comprising FW1, FW2, and FW3 regions, and the amino acid sequence of said FW1, FW2, and FW3 are the same as the amino acid sequences of corresponding framework regions encoded by human germline antibody gene segments. In another embodiment, the human germline antibody gene segment is selected from the group consisting of DP47, DP45, DP48, and DPK9.

An antibody single variable domain polypeptide that binds to CD28 has an amino acid sequence that is identical to the amino acid sequence selected from the group consisting of SEQ ID NOs:1-57, or differs from the selected amino acid sequence at no more than 25 amino acid positions and has a sequence that is at least 50% identical to the selected sequence. In one embodiment, the antibody single variable domain polypeptide differs from the selected amino acid sequence at 25 or fewer amino acid positions, 20 or fewer amino acid positions, 15 or fewer amino acid positions, 10 or fewer amino acid positions, 5 or fewer amino acid positions, 2 or fewer amino acid positions, or as few as one amino acid position. In a further embodiment, the antibody single variable domain polypeptide is at least 50% identical to the selected sequence, for example, at least 50% identical, at least 55% identical, at least 60% identical, at least 65% identical, at least 70% identical, at least 80% identical, at least 85% identical, at least 90% identical, at least 95% identical, and up to and including 96%, 97%, 98%, or 99% identical. In another embodiment, one or more of the CDR regions of an antibody single variable domain polypeptide is at least 50% identical to the selected sequence, for example, at least 50% identical, at least 55% identical, at least 60% identical, at least 65% identical, at least 70% identical, at least 80% identical, at least 85% identical, at least 90% identical, at least 95% identical, and up to and including 96%, 97%, 98%, or 99% identical.

The invention includes an antibody single variable domain polypeptide that binds to CD28, wherein the polypeptide has an amino acid sequence that is identical to the amino acid sequence of DOM21-4, or differs from the amino acid sequence of DOM2′-4 at no more than 25 amino acid positions and has a CDR1 sequence that is at least 50% identical to the CDR1 sequence of DOM21-4, or has a CDR2 sequence that is at least 50% identical to the CDR2 sequence of DOM21-4, or has a CDR3 sequence that is at least 50% identical to the CDR3 sequence of DOM21-4.

The invention also includes an antibody single variable domain polypeptide that binds CD28, wherein the dAb has an amino acid sequence that is identical to the amino acid sequence of DOM21-4, or differs from the amino acid sequence of DOM21-4 at no more than 25 amino acid positions and has a CDR1 sequence that is at least 50% identical to the CDR1 sequence of DOM21-4 and has a CDR2 sequence that is at least 50% identical to the CDR2 sequence of DOM21-4.

The invention also includes an antibody single variable domain polypeptide that binds CD28, wherein the dAb has an amino acid sequence that is identical to the amino acid sequence of DOM21-4, or differs from the amino acid sequence of DOM21-4 at no more than 25 amino acid positions and has a CDR2 sequence that is at least 50% identical to the CDR2 sequence of DOM21-4 and has a CDR3 sequence that is at least 50% identical to the CDR3 sequence of DOM21-4.

The invention also includes an antibody single variable domain polypeptide that binds CD28, wherein the dAb has an amino acid sequence that is identical to the amino acid sequence of DOM21-4, or differs from the amino acid sequence of DOM21-4 at no more than 25 amino acid positions and has a CDR1 sequence that is at least 50% identical to the CDR1 sequence of DOM21-4 and has a CDR3 sequence that is at least 50% identical to the CDR3 sequence of DOM21-4.

The invention also includes an antibody single variable domain polypeptide that binds CD28, wherein the dAb has an amino acid sequence that is identical to the amino acid sequence of DOM21-4, or differs from the amino acid sequence of DOM21-4 at no more than 25 amino acid positions and has a CDR1 sequence that is at least 50% identical to the CDR1 sequence of DOM21-4 and has a CDR2 sequence that is at least 50% identical to the CDR2 sequence of DOM21-4 and has a CDR3 sequence that is at least 50% identical to the CDR3 sequence of DOM21-4.

In one embodiment, the antibody single variable domain polypeptide that binds to CD28, if not identical in sequence to that of DOM21-4, differs form the amino acid sequence of DOM21-4 at 25 or fewer amino acid positions, 20 or fewer amino acid positions, 15 or fewer amino acid positions, 10 or fewer amino acid positions, 5 or fewer amino acid positions, 2 or fewer amino acid positions, or as few as one amino acid position.

The invention also includes a CD28 antagonist having a CDR1 sequence that is at least 50% identical to the CDR1 sequence of DOM21-4.

The invention also includes a CD28 antagonist having a CDR2 sequence that is at least 50% identical to the CDR2 sequence of DOM21-4.

The invention also includes a CD28 antagonist having a CDR3 sequence that is at least 50% identical to the CDR3 sequence of DOM21-4.

The invention also includes a CD28 antagonist having a CDR1 sequence that is at least 50% identical to the CDR1 sequence of DOM21-4 and a CDR2 sequence that is at least 50% identical to the CDR2 sequence of DOM21-4.

The invention also includes a CD28 antagonist having a CDR2 sequence that is at least 50% identical to the CDR2 sequence of DOM21-4 and a CDR3 sequence that is at least 50% identical to the CDR3 sequence of DOM21-4.

The invention also includes a CD28 antagonist having a CDR1 sequence that is at least 50% identical to the CDR1 sequence of DOM21-4 and a CDR3 sequence that is at least 50% identical to the CDR3 sequence of DOM21-4.

The invention also includes a CD28 antagonist having a CDR1 sequence that is at least 50% identical to the CDR1 sequence of DOM21-4 and a CDR2 sequence that is at least 50% identical to the CDR2 sequence of DOM21-4 and a CDR3 sequence that is at least 50% identical to the CDR3 sequence of DOM21-4.

The invention includes an antibody single variable domain polypeptide that binds to CD28, wherein the polypeptide has an amino acid sequence that is identical to the amino acid sequence of DOM21-18, or differs from the amino acid sequence of DOM21-18 at no more than 25 amino acid positions and has a sequence that is at least 80% identical to the sequence of DOM21-18. In one embodiment, the antibody single variable domain polypeptide differs form the amino acid sequence of DOM21-18 at 25 or fewer amino acid positions, 20 or fewer amino acid positions, 15 or fewer amino acid positions, 10 or fewer amino acid positions, 5 or fewer amino acid positions, 2 or fewer amino acid positions, or as few as one amino acid position. In a further embodiment, the antibody single variable domain polypeptide is at least 80% identical to the sequence of DOM21-18, for example, at least 85% identical, at least 90% identical, at least 95% identical, and up to and including 96%, 97%, 98%, or 99% identical.

The invention includes an antibody single variable domain polypeptide that binds to CD28, wherein the polypeptide has an amino acid sequence that is identical to the amino acid sequence of DOM21-4, or differs from the amino acid sequence of DOM21-18 at no more than 25 amino acid positions and has a CDR1 sequence that is at least 50% identical to the CDR1 sequence of DOM21-18, or has a CDR2 sequence that is at least 50% identical to the CDR2 sequence of DOM21-18, or has a CDR3 sequence that is at least 50% identical to the CDR3 sequence of DOM21-18.

The invention also includes an antibody single variable domain polypeptide that binds CD28, wherein the dAb has an amino acid sequence that is identical to the amino acid sequence of DOM21-18, or differs from the amino acid sequence of DOM21-18 at no more than 25 amino acid positions and has a CDR1 sequence that is at least 50% identical to the CDR1 sequence of DOM21-18 and has a CDR2 sequence that is at least 50% identical to the CDR2 sequence of DOM21-18.

The invention also includes an antibody single variable domain polypeptide that binds CD28, wherein the dAb has an amino acid sequence that is identical to the amino acid sequence of DOM21-18, or differs from the amino acid sequence of DOM21-18 at no more than 25 amino acid positions and has a CDR2 sequence that is at least 50% identical to the CDR2 sequence of DOM21-18 and has a CDR3 sequence that is at least 50% identical to the CDR3 sequence of DOM21-18.

The invention also includes an antibody single variable domain polypeptide that binds CD28, wherein the dAb has an amino acid sequence that is identical to the amino acid sequence of DOM21-18, or differs from the amino acid sequence of DOM21-18 at no more than 25 amino acid positions and has a CDR1 sequence that is at least 50% identical to the CDR1 sequence of DOM21-18 and has a CDR3 sequence that is at least 50% identical to the CDR3 sequence of DOM21-18.

The invention also includes an antibody single variable domain polypeptide that binds CD28, wherein the dAb has an amino acid sequence that is identical to the amino acid sequence of DOM21-18, or differs from the amino acid sequence of DOM21-18 at no more than 25 amino acid positions and has a CDR1 sequence that is at least 50% identical to the CDR1 sequence of DOM21-18 and has a CDR2 sequence that is at least 50% identical to the CDR2 sequence of DOM21-18 and has a CDR3 sequence that is at least 50% identical to the CDR3 sequence of DOM21-18.

In one embodiment, the antibody single variable domain polypeptide that binds to CD28, if not identical in sequence to that of DOM21-18, differs form the amino acid sequence of DOM21-18 at 25 or fewer amino acid positions, 20 or fewer amino acid positions, 15 or fewer amino acid positions, 10 or fewer amino acid positions, 5 or fewer amino acid positions, 2 or fewer amino acid positions, or as few as one amino acid position.

The invention also includes a CD28 antagonist having a CDR1 sequence that is at least 50% identical to the CDR1 sequence of DOM21-18.

The invention also includes a CD28 antagonist having a CDR2 sequence that is at least 50% identical to the CDR2 sequence of DOM21-18.

The invention also includes a CD28 antagonist having a CDR3 sequence that is at least 50% identical to the CDR3 sequence of DOM21-18.

The invention also includes a CD28 antagonist having a CDR1 sequence that is at least 50% identical to the CDR1 sequence of DOM21-18 and a CDR2 sequence that is at least 50% identical to the CDR2 sequence of DOM21-18.

The invention also includes a CD28 antagonist having a CDR2 sequence that is at least 50% identical to the CDR2 sequence of DOM21-18 and a CDR3 sequence that is at least 50% identical to the CDR3 sequence of DOM21-18.

The invention also includes a CD28 antagonist having a CDR1 sequence that is at least 50% identical to the CDR1 sequence of DOM21-18 and a CDR3 sequence that is at least 50% identical to the CDR3 sequence of DOM21-18.

The invention also includes a CD28 antagonist having a CDR1 sequence that is at least 50% identical to the CDR1 sequence of DOM21-18 and a CDR2 sequence that is at least 50% identical to the CDR2 sequence of DOM21-18 and a CDR3 sequence that is at least 50% identical to the CDR3 sequence of DOM21-18.

The invention includes an antibody single variable domain polypeptide that binds to CD28, wherein the polypeptide has an amino acid sequence that is identical to the amino acid sequence of DOM21-28, or differs from the amino acid sequence of DOM21-28 at no more than 25 amino acid positions and has a sequence that is at least 80% identical to the sequence of DOM21-28. In one embodiment, the antibody single variable domain polypeptide differs form the amino acid sequence of DOM21-28 at 25 or fewer amino acid positions, 20 or fewer amino acid positions, 15 or fewer amino acid positions, 10 or fewer amino acid positions, 5 or fewer amino acid positions, 2 or fewer amino acid positions, or as few as one amino acid position. In a further embodiment, the antibody single variable domain polypeptide is at least 80% identical to the sequence of DOM21-28, for example, at least 85% identical, at least 90% identical, at least 95% identical, and up to and including 96%, 97%, 98%, or 99% identical.

The invention includes an antibody single variable domain polypeptide that binds to CD28, wherein the polypeptide has an amino acid sequence that is identical to the amino acid sequence of DOM21-28, or differs from the amino acid sequence of DOM21-28 at no more than 25 amino acid positions and has a CDR1 sequence that is at least 50% identical to the CDR1 sequence of DOM21-28, or has a CDR2 sequence that is at least 50% identical to the CDR2 sequence of DOM21-28, or has a CDR3 sequence that is at least 50% identical to the CDR3 sequence of DOM21-28.

The invention also includes an antibody single variable domain polypeptide that binds CD28, wherein the dAb has an amino acid sequence that is identical to the amino acid sequence of DOM21-28, or differs from the amino acid sequence of DOM21-28 at no more than 25 amino acid positions and has a CDR1 sequence that is at least 50% identical to the CDR1 sequence of DOM21-28 and has a CDR2 sequence that is at least 50% identical to the CDR2 sequence of DOM21-28.

The invention also includes an antibody single variable domain polypeptide that binds CD28, wherein the dAb has an amino acid sequence that is identical to the amino acid sequence of DOM21-28, or differs from the amino acid sequence of DOM21-28 at no more than 25 amino acid positions and has a CDR2 sequence that is at least 50% identical to the CDR2 sequence of DOM21-28 and has a CDR3 sequence that is at least 50% identical to the CDR3 sequence of DOM21-28.

The invention also includes an antibody single variable domain polypeptide that binds CD28, wherein the dAb has an amino acid sequence that is identical to the amino acid sequence of DOM21-28, or differs from the amino acid sequence of DOM21-28 at no more than 25 amino acid positions and has a CDR1 sequence that is at least 50% identical to the CDR1 sequence of DOM21-28 and has a CDR3 sequence that is at least 50% identical to the CDR3 sequence of DOM21-28.

The invention also includes an antibody single variable domain polypeptide that binds CD28, wherein the dAb has an amino acid sequence that is identical to the amino acid sequence of DOM21-28, or differs from the amino acid sequence of DOM21-28 at no more than 25 amino acid positions and has a CDR1 sequence that is at least 50% identical to the CDR1 sequence of DOM21-28 and has a CDR2 sequence that is at least 50% identical to the CDR2 sequence of DOM21-28 and has a CDR3 sequence that is at least 50% identical to the CDR3 sequence of DOM21-28.

In one embodiment, the antibody single variable domain polypeptide that binds to CD28, if not identical in sequence to that of DOM21-28, differs form the amino acid sequence of DOM21-28 at 25 or fewer amino acid positions, 20 or fewer amino acid positions, 15 or fewer amino acid positions, 10 or fewer amino acid positions, 5 or fewer amino acid positions, 2 or fewer amino acid positions, or as few as one amino acid position.

The invention also includes a CD28 antagonist having a CDR1 sequence that is at least 50% identical to the CDR1 sequence of DOM21-28.

The invention also includes a CD28 antagonist having a CDR2 sequence that is at least 50% identical to the CDR2 sequence of DOM21-28.

The invention also includes a CD28 antagonist having a CDR3 sequence that is at least 50% identical to the CDR3 sequence of DOM21-28.

The invention also includes a CD28 antagonist having a CDR1 sequence that is at least 50% identical to the CDR1 sequence of DOM21-28 and a CDR2 sequence that is at least 50% identical to the CDR2 sequence of DOM21-28.

The invention also includes a CD28 antagonist having a CDR2 sequence that is at least 50% identical to the CDR2 sequence of DOM21-28 and a CDR3 sequence that is at least 50% identical to the CDR3 sequence of DOM21-28.

The invention also includes a CD28 antagonist having a CDR1 sequence that is at least 50% identical to the CDR1 sequence of DOM21-28 and a CDR3 sequence that is at least 50% identical to the CDR3 sequence of DOM21-28.

The invention also includes a CD28 antagonist having a CDR1 sequence that is at least 50% identical to the CDR1 sequence of DOM21-28 and a CDR2 sequence that is at least 50% identical to the CDR2 sequence of DOM21-28 and a CDR3 sequence that is at least 50% identical to the CDR3 sequence of DOM21-28.

Disclosed herein is an antagonist of CD28 which either contains or consists of a monovalent polypeptide domain which specifically binds CD28 and which competes for binding to CD28 with an antibody single variable domain selected from the group consisting of DOM21-4, DOM21-18, DOM21-28, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-27, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-58, DOM21-59, DOM21-60, DOM21-61, DOM21-62, DOM21-63, DOM21-64, DOM21-65, DOM21-66, DOM21-67, and DOM21-68.

Disclosed herein is an antagonist of CD28 which either contains or consists of a monovalent polypeptide domain which specifically binds CD28 and which comprises an amino sequence which is at least 70% identical to an amino acid sequence of an antibody single variable domain selected from the group consisting of: DOM21-4, DOM21-18, DOM21-28, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-27, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-58, DOM21-59, DOM21-60, DOM21-61, DOM21-62, DOM21-63, DOM21-64, DOM21-65, DOM21-66, DOM21-67, and DOM21-68.

In one aspect of the CD28 antagonist, the monovalent polypeptide domain which specifically binds CD28 has a CDR1 amino acid sequence that is at least 50% identical to the amino acid sequence of a CDR1 of a single variable domain selected from the group consisting of the V_{L} antibody single variable domains of: DOM21-18, DOM21-27, DOM21-28 and DOM21-58. The CDR1 amino acid sequence can contain or consist of any of the following amino acid sequences and/or motifs: RASQ, RASQX₁I, wherein X₁ is any amino acid or X₁ is an amino acid selected from the group consisting of Y, S and N; RASQX₁IG, wherein X₁ is any amino acid or X₁ is an amino acid selected from the group consisting of Y, S and N; RASQX₁IGX₂X₃L, wherein X₁ is any amino acid or X₁ is an amino acid selected from the group consisting of Y, S and N, and/or where X₂ is any amino acid or is an amino acid selected from the group consisting of: T, H and D.

In one aspect of the CD28 antagonist, the monovalent polypeptide domain which specifically binds CD28 has a CDR1 amino acid sequence that is at least 50% identical to the CDR1 amino acid sequence of a V_{H} antibody single variable domain selected from the group consisting of: DOM21-4, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-59, DOM21-60, DOM21-61, DOM21-62, DOM21-63, DOM21-64, DOM21-65, DOM21-66, DOM21-67, and DOM21-68.

In one aspect of the CD28 antagonist, the monovalent polypeptide domain which specifically binds CD28 has a CDR2 amino acid sequence that is at least 50% identical to the amino acid sequence of a CDR2 of a V_{L} single variable domain selected from the group consisting of: DOM21-18, DOM21-27, DOM21-28 and DOM21-58. The CDR2 can contain or consist of any of the following amino acid sequences and/or motifs: XₐSX_{b}LQS wherein Xₐ is any amino acid and/or X_{b} is any amino acid, and/or Xₐ is an amino acid selected from the group consisting of: A and I, and/or wherein X_{b} is an amino acid selected from the group consisting of: I, L and R; QASLLQS, RASILQS, WASLLQS and RISRLQS.

In one aspect of the CD28 antagonist, the monovalent polypeptide domain which specifically binds CD28 has a CDR2 amino acid sequence that is at least 50% identical to the CDR2 amino acid sequence of a V_{H} single variable domain selected from the group consisting of: DOM21-4, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-59, DOM21-60, DOM21-61, DOM21-62, DOM21-63, DOM21-64, DOM21-65, DOM21-66, DOM21-67, and DOM21-68. The CDR2 can contain or consist of any of the following amino acid sequences and/or motifs: YYADSVKG, TYYAD, TYY, TYYA, TYYADS, TYYADSV, TYYADSVK and TYYADSVKG, wherein T is at Kabat position 57, and wherein YY is at Kabat position 58 and 59, respectively; and/or a T at Kabat position 57, and/or a Y at Kabat position 58, and/or a Y at Kabat position 59.

In one aspect of the CD28 antagonist, the monovalent polypeptide domain which specifically binds CD28 has a CDR3 amino acid sequence that is at least 50% identical to the amino acid sequence of a CDR3 of a V_{L} single variable domain selected from the group consisting of: DOM21-18, DOM21-27, DOM21-28 and DOM21-58. The CDR3 can contain or consist of any of the following amino acid sequences and/or motifs: QQX_{c}X_{d}XₑX_{f}PX_{g}T, wherein each of X_{c}, X_{d}, Xₑ, X_{f} and X_{g} is any amino acid, and/or wherein X_{c} is selected from the group consisting of L, T, G and F, and/or X_{d} is selected from the group consisting of: A, T, M and G, and/or Xₑ is selected from the group consisting of L and T, and/or X_{f} is selected from the group consisting of: R, Q, T and Y, and X_{g} is selected from the group consisting of M, F and T.

In one aspect of the CD28 antagonist, the monovalent polypeptide domain which specifically binds CD28 has a CDR1 amino acid sequence selected from the group consisting of: RASQYIGTSLN, RASQSIGTGLR, RASQSISHSLV and RASQNIGDRLH, a CDR2 amino acid sequence selected from the group consisting of: QASLLQS, RASILQS, WASLLQS and RISRLQS, and a CDR3 amino acid sequence selected from the group consisting of: QQLALRPMT, QQTTLQPFT, QQGMTTPFT and QQFGLYPTT. In one aspect of the CD28 antagonist, the monovalent polypeptide domain which specifically binds CD28 has a CDR1 amino acid sequence of RASQYIGTSLN, a CDR2 amino acid sequence of QASLLQS, and a CDR3 amino acid sequence of QQLALRPMT. In one aspect of the CD28 antagonist, the monovalent polypeptide domain which specifically binds CD28 has a has a CDR1 amino acid sequence of RASQSIGTGLR, a CDR2 amino acid sequence of RASILQS, and a CDR3 amino acid sequence of QQTTLQPFT. In one aspect of the CD28 antagonist, the monovalent polypeptide domain which specifically binds CD28 has a CDR1 amino acid sequence of RASQSISHSLV, a CDR2 amino acid sequence of WASLLQS, and a CDR3 amino acid sequence of QQGMTTPFT. In one aspect of the CD28 antagonist, the monovalent polypeptide domain which specifically binds CD28 has a CDR1 amino acid sequence of RASQNIGDRLH, a CDR2 amino acid sequence of RISRLQS, and a CDR3 amino acid sequence of QQFGLYPTT.

In one aspect of the CD28 antagonist, the monovalent polypeptide domain which specifically binds CD28 has a CDR3 amino acid sequence that is at least 50% identical to the CDR3 amino acid sequence of a V_{H} single variable domain selected from the group consisting of DOM21-4, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-59, DOM21-60, DOM21-61, DOM21-62, DOM21-63, DOM21-64, DOM21-65, DOM21-66, DOM21-67, and DOM21-68. The CDR3 can contain or consist of any of the following amino acid sequences and/or motifs: FDY, wherein D is at Kabat position 101 and/or F at Kabat position immediately preceding Kabat position 101, and/or a D at Kabat position 101, and/or a Y at Kabat position 102; and/or first residue of said CDR amino acid sequence is selected from the group consisting of K, E and Q. In one aspect of the CD28 antagonist, the monovalent polypeptide domain which specifically binds CD28 has a CDR1 amino acid sequence of RYHMA, a CDR2 amino acid sequence of VIDSLGLQTYYADSVKG, and a CDR3 amino acid sequence of EYGGAFDY.

In one aspect of the CD28 antagonist, the monovalent polypeptide domain which specifically binds CD28 has a VL FR1 amino acid sequence that can contain or consist of any of the following amino acid sequences and/or motifs: DIQMTQSPSSLSASVGDRVTITC, and/or has a VL FR2 amino acid sequence comprising WYQQKPGKAPXₕLLXᵢY, wherein each of X_{h and X}ᵢ is any amino acid and/or where Xₕ is selected from the group consisting of: K and M, and/or wherein Xᵢ is selected from the group consisting of T and I; and/or a VL FR3 amino acid sequence comprising GVPSRFSGSGSGTDFTLTISSLQPEDFATYYC, and/or a VL FR4 amino acid sequence comprising FGQGTKVEIKR.

In one aspect of the CD28 antagonist, the monovalent polypeptide domain which specifically binds CD28 has a VH FR1 amino acid sequence that can contain or consist of any of the following amino acid sequences and/or motifs: QLL at Kabat positions 3, 4 and 5, respectively, according to the Kabat numbering system, and/or FTFXⱼ, located at the carboxyl terminus of said FR1, where Xⱼ is selected from the group consisting of: E, I, G, T, S, D, R, P, N, A and K, and/or VXLLE, EVXLLE, EVXLLES, wherein optionally X is Q; LLES, LLESG, LLESGG, EVQLLESGGGLVQPGGSLRLSCAASGFTF, EVQLLESGGGLVQPGGSLRLSCAASGFTFS, wherein E is at Kabat position 6 and/or a L at Kabat position 5, and/or Q at position 3; and/or has a VH FR1 with a L at position 5; and/or has a FR2 with a V or an A at position 37 and/or a G at position 44, and/or a W at position 47, and/or GLEW at positions 44-47, and/or VXXXXXXGLEW or AXXXXXXGLEW, wherein X is any amino acid and A is at Kabat position 37, and/or: VRQAPGKGLEW, AVRQAPGKGLEW and/or AVRQAPGKGLEWVS; and/or a FR3 with a S at position 74, and/or TLY at positions 77-79, and/or a T at position 77, and/or a L at position 78, and/or a Y at position 79, and/or an A at position 84, and/or SKNTLY, wherein said S is at position 74, and/or SKNTLYXXXXXXXA, wherein said S is at position 74, and X is any amino acid, and/or SKNTLYLQMNSLRAEDTAVYYCA wherein said S is at position 74 and/or RFTISRDNSKNTLYLQMNSLRAEDTAVYYCA, wherein said S is at position 74, and/or a FR4 with the amino acid L at position 108 and/or a FR4 that contains TLV, GLTV, LTVS, QGLTV, LTVSS, and WGQGTLVTVSS, wherein L at position 108.

In one embodiment, the monovalent polypeptide domain which specifically binds CD28 is fused to a heterologous protein or molecule.

In one embodiment, a CD28 antagonist has a CDR1 sequence that is at least 50% identical to the CDR1 sequence of the amino acid sequence selected from the group consisting of SEQ ID NOs:1-57. In one embodiment, a CD28 antagonist has a CDR1 sequence that is at least 50% identical to the CDR2 sequence of the amino acid sequence selected from the group consisting of SEQ ID NOs:1-57. In one embodiment, a CD28 antagonist has a CDR1 sequence that is at least 50% identical to the CDR3 sequence of the amino acid sequence selected from the group consisting of SEQ ID NOs:1-57.

In one embodiment the CD28 antagonist inhibits the binding of CD80 to CD28, and/or inhibits the binding of CD80 to CD28, and/or inhibits an activity of CD28.

Also included is a dual specific ligand comprising a first immunoglobulin single variable domain having a binding specificity to a first antigen and a second single variable domain having a binding activity to a second antigen, wherein the first antigen is CD28 and binding of the second single variable domain to the second antigen acts to increase the half-life of the ligand in vivo. In one embodiment, the dual specific ligand is a four chain IgG antibody, wherein at least one chain comprises an antibody single variable domain which specifically binds CD28.

In one embodiment, at least one chain of the four chain IgG comprises an antibody single variable domain which specifically binds CD28 and at least one chain comprises a single domain such as an antibody single variable domain which independently specifically binds a second molecule, such as a T cell receptor. In another embodiment, at least one chain of the four chain IgG comprises an antibody single variable domain which specifically binds CD28 and at least a pair of chains such as a heavy and light chain pair which cooperatively specifically binds a second antigen, such as a T cell receptor. The binding of the T cell receptor can be to the T cell receptor itself or to an epitope formed by the interaction of a T cell receptor and the antigen presented by the T cell receptor.

Also included is a dual specific ligand comprising an anti-human CD28 antibody single variable domain and an anti-SA antibody single variable domain.

In one embodiment, the dAbs or antibody single variable domains are camelid V_{HH} domains.

In one embodiment of the dual specific ligand, either (i) the first and second antibody single variable domains are heavy chain variable domains; or (ii) the first and the second antibody variable domains are light chain variable domains.

In one embodiment, the ligand is provided as an IgG immunoglobulin comprising four heavy chain single variable domains or four light chain single variable domains. The heavy chain can comprise camelid V_{HH} domains.

In a further embodiment of the dual specific ligand, the first and second domains bind independently, such that the dual specific ligand may simultaneously bind both the first and second antigens.

In one embodiment of the dual specific ligand, the first single variable domain has a dissociation constant (K_{d}) of 1×10⁻⁷ M or less for human CD28, and a K_{off} rate constant of 1×10⁻² s⁻¹ or less, as determined by surface plasmon resonance.

In one embodiment of the dual specific ligand, the second single variable domain is specific for serum albumin (SA) and has a dissociation constant (K_{d}) of 1 nM to 500 μm for SA, as determined by surface plasmon resonance.

In a further embodiment, the second domain binds SA in a standard ligand binding assay with an IC₅₀ of 1 nM to 500 pM. The second single variable domain may be specific for SA, and comprise the amino acid sequence of MSA-16 (SEQ ID NO:323) or a sequence that is at least 80% identical thereto. Alternatively, the second single variable domain may be specific for SA, and comprise the amino acid sequence of MSA-26 (SEQ ID NO: 325) or a sequence that is at last 80% identical thereto.

In one embodiment of the dual specific ligand, the anti-CD28 variable domain or dAb comprises a universal framework. The anti-CD28 variable domain or dAb may also comprise a V_{H} framework selected from the group consisting of DP47, DP45 and DP38; or a V_{L} framework which is DPK9. In a further embodiment, the dual specific ligand or dAb can comprise a binding site for a generic ligand.

In one embodiment, the generic ligand binding site is selected from the group consisting of protein A, protein L and protein G binding site.

In one embodiment of the dual specific ligand or monovalent polypeptide that binds CD28, the anti-CD28 variable domain or dAb comprises one or more framework regions comprising an amino acid sequence that is the same as the amino acid sequence of a corresponding framework region encoded by a human germline antibody gene segment, or the amino acid sequence of one or more of said framework regions collectively comprises up to 5 amino acid differences relative to the amino acid sequence of said corresponding framework region encoded by a human germline antibody gene segment.

In one embodiment, the amino acid sequences of FW1, FW2, FW3, and FW4 of the anti-CD28 variable domain or dAb are the same as the amino acid sequences of corresponding framework regions encoded by a human germline antibody gene segment, or the amino acid sequences of FW1, FW2, FW3, and FW4 collectively contain up to 10 amino acid differences relative to the amino acid sequences of corresponding framework regions encoded by said human germline antibody gene segment.

In one embodiment, the amino acid sequences of said FW1, FW2, and FW3 of the anti-CD28 variable domain or dAb are the same as the amino acid sequences of corresponding framework regions encoded by human germline antibody gene segments. The human germline antibody gene segments may be selected from the group consisting of DP47, DP45, DP48, and DPK9. In one embodiment, the FW2 sequence encoded by a germline gene segment. In a further embodiment, the germline gene segment is selected from the group consisting of DP47 and DPK9.

Also included is a method for producing a dual specific ligand as described herein, comprising a first immunoglobulin single variable domain having a binding specificity for CD28 and a second single immunoglobulin single variable domain having a binding specificity for a protein which increases the half-life of the ligand in vivo, the method comprising the steps of: selecting a first variable domain by its ability to bind CD28; selecting a second variable domain by its ability to bind to said protein; combining the variable domains; and selecting the ligand by its ability to bind to CD28 and said protein.

In one embodiment, the first variable domain is selected for binding to CD28 in absence of a complementary variable domain.

Also included is nucleic acid encoding a dual specific ligand described herein. The nucleic acid may comprise the nucleic acid sequence of MSA-16 or a sequence that is at least 80% identical thereto, or alternatively may comprise, the nucleic acid sequence of MSA-26 or a sequence that is at least 70% identical thereto. The nucleic acid may be incorporated into a vector, which may be incorporated into a host cell.

Also included is a pharmaceutical composition comprising a dual specific ligand as described herein and a pharmaceutically acceptable excipient, carrier or diluent.

Also included is a dAb monomer specific for CD28, which monomer has a dissociation constant (k_{d}) of 1×10⁻⁸ M or less for human CD28, and a K_{off} rate constant of 1×10⁻³ s⁻¹ or less, as determined by surface plasmon resonance.

In one embodiment, the dAb monomer specific for CD28 has a dissociation constant (k_{d}) of 1×10⁻⁷ M or less, as determined by surface plasmon resonance.

In one embodiment, the dAb monomer has binding specificity to CD28 with a dissociation constant (K_{d}) of 1×10⁻⁸ M or less, as determined by surface plasmon resonance.

In one embodiment, the dAb monomer has binding specificity to CD28 with a dissociation constant (k_{d}) of 50 nM to 20 pM, as determined by surface plasmon resonance.

In one embodiment, the monomer inhibits binding of CD80 to CD28 with an IC₅₀ of 50 nM or less. In one embodiment, the monomer inhibits binding of CD86 to CD28 with an IC₅₀ of 50 nM or less.

In a further embodiment, the dAb monomer has binding specificity to CD28 with a K_{off} rate constant of 1×10⁻³ s⁻¹ or less, 1×10⁻⁴ s⁻¹ or less, 1×10⁻⁵ s⁻¹ or less, or 1×10⁻⁶ s⁻¹ or less, as determined by surface plasmon resonance.

In one embodiment, the dAb monomer neutralizes CD28 in a standard assay with an ND50 of 50 nM or less.

Also included is a dual specific ligand comprising first and second heavy chain single variable domains, or first and second light chain single variable domains, wherein the first variable domain is an anti-CD28 dAb monomer.

In one embodiment, the second variable domain has binding specificity for an antigen other than CD28.

Also included is a dual specific ligand comprising a first immunoglobulin single variable domain having a binding specificity to a first antigen and a second single variable domain having a binding activity to a second antigen, wherein the first antigen is CD28 and the second single variable domain is an Antigen Presenting Cell surface antigen or a T cell surface antigen. The Antigen Presenting Cell surface antigen can be selected from one of the group consisting of dendritic cell surface antigens, activated macrophage surface antigens, activated B cell surface antigens, co-stimulatory signal pathway surface antigens, and MHC antigens.

In one embodiment, the MHC antigen is a MHC class II antigen, and the class II antigen can be the alpha and/or beta chain.

The Antigen Presenting Cell surface antigen or a T cell surface antigen may be selected from the group consisting of CD40, CD40L, Inducible costimulatory molecule (ICOS), CD27, CD30, OX40, CD45, CD69, CD3, CD70, Inducible costimulatory molecule ligand (ICOSL), OX40L, CD80, CD86, HVEM (Herpes Virus Entry Mediator), and LIGHT, including one of CD40L, Inducible costimulatory molecule (ICOS), CD27, CD30, OX40, CD45, CD69, or CD3.

An exemplary surface antigen is a B7 gene surface antigen such as CD86 or CD80.

In one aspect, the invention provides a domain antibody that binds to CD28, the antibody having at least three characteristics selected from the group consisting of: prevents CD80 and CD86 binding to CD28; does not cross-react with CTLA4; has a to half-life of about 15 seconds to about 12 hours; and has a tβ half-life of about 12 hours to about 96 hours.

In one embodiment, an antagonist of CD28 as described herein has a serum tβ half-life in the range of about 12 hours to about 31 days. In a further embodiment, an antagonist of CD28 has a serum tβ half-life in the range of about 12 hours to about 28 days. In a still further embodiment, an antagonist of CD28 has a serum tβ half-life in the range of about 12 hours to about 14 days.

In one aspect, the invention provides an immunoglobulin single variable domain that specifically binds to CD28, and that modulates, inhibits, prevents or blocks the interaction between a target on an antigen presenting cell (APC) and a target on a T-cell, as well as that modulates, inhibits, prevents or blocks an interaction between the APC and the T-cell.

The immunoglobulin single variable domain can be directed against and/or that can specifically bind to a target on a T-cell. In one embodiment, the immunoglobulin single variable domain inhibits and/or blocks T-cell activation. In one embodiment, the immunoglobulin single variable domain inhibits and/or blocks cytokine production. In a further embodiment, the immunoglobulin single variable domain decreases T-cell survival. In a still further embodiment, the immunoglobulin single variable domain decreases the differentiation of naive T-cells into activated cytokine secreting T-cells.

In one embodiment, the binding target of the immunoglobulin single variable domain belongs to the B7:CD28 superfamily.

In one embodiment, the immunoglobulin single variable domain modulates, inhibits, prevents and/or blocks the interaction of B7-1 (CD80) with CD28.

In a further embodiment, the immunoglobulin single variable domain modulates, inhibits, prevents and/or blocks the interaction of B7-1 with CD28, while the interaction of B7-1 with CTLA4 is not modulated, inhibited or prevented.

In one embodiment, the immunoglobulin single variable domain modulates, inhibits, prevents and/or blocks the interaction of B7-2 (CD86) with CD28.

In one embodiment, the immunoglobulin single variable domain modulates, inhibits, prevents and/or blocks the interaction of B7-2 with CD28, while the interaction of B7-2 with CTLA4 is not modulated, inhibited or prevented.

In a further embodiment, the immunoglobulin single variable domain modulates, inhibits, prevents and/or blocks the interaction of B7-1 with CD28 and the interaction of B7-2 with CD28.

In one embodiment, the immunoglobulin single variable domain modulates, inhibits, prevents and/or blocks the interaction of B7-1 with CD28, while the interaction of B7-2 with CD28 is not modulated, inhibited or prevented.

In one embodiment, the immunoglobulin single variable domain modulates, inhibits, prevents and/or blocks the interaction of B7-2 with CD28, while the interaction of B7-1 with CD28 is not modulated, inhibited or prevented.

In one embodiment, the immunoglobulin single variable domain can specifically bind to an APC target or a T-cell target with a dissociation constant (K D) of 10⁻⁵ to 10⁻¹² moles/litre or less, and preferably 10⁻⁷ to 10⁻¹² moles/litre or less and more preferably 10⁻⁸ to 10⁻¹² moles/litre.

In a further embodiment, the immunoglobulin single variable domain can specifically bind to an APC target or a T-cell target with a rate of association (kon-rate) of between 10² M⁻¹S⁻¹ to about 10⁷ M⁻¹ S⁻¹, preferably between 10³ M⁻¹S⁻¹ and 10⁷ M⁻¹S⁻¹, more preferably between 10⁴ M⁻¹S⁻¹ and 10⁷ M⁻¹S⁻¹, such as between 10⁵ M⁻¹S⁻¹ and 10⁷ M⁻¹S⁻¹.

In one embodiment, the immunoglobulin single variable domain can specifically bind to an APC target or a T-cell target with a rate of dissociation (koff rate) between 1 s⁻¹ and 10⁻⁶ s⁻¹ preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

In one embodiment, the immunoglobulin single variable domain can specifically bind to an APC target or a T-cell target with an affinity less than 500 nM, preferably less than 200 nM, more preferably less than 10 nM, such as less than 500 pM.

In one embodiment, the immunoglobulin single variable domain is a naturally occurring Immunoglobulin single variable domain (from any suitable species) or a synthetic or semi-synthetic Immunoglobulin single variable domain.

In one embodiment, the immunoglobulin single variable domain is a V_{HH} sequence, a partially humanized V_{HH} sequence, a fully humanized V_{HH} sequence, a camelized heavy chain variable domain or an Immunoglobulin single variable domain that has been obtained by techniques such as affinity maturation.

In one embodiment, the immunoglobulin single variable domain i) has at least 80% amino acid identity with at least one of the amino acid sequences of SEQ ID NOs: 58-114, in which for the purposes of determining the degree of amino acid identity, the amino acid residues that form the CDR sequences are disregarded; and in which preferably one or more of the amino acid residues at positions one or more of the amino acid residues at positions 11, 37, 44, 45, 47, 83, 84, 103, 104 and 108 according to the Kabat numbering are chosen from L and V for position 11, V and F at position 37, G at position 44, L at position 45, W and Y at position 47, R and K at position 83, A, T and D at position 84, W at position 103, G at position 104, and L, M and T at position 108.

In one embodiment, the immunoglobulin single variable domain i) has at least 80% amino acid identity with at least one of the amino acid sequences of SEQ ID NOs: SEQ ID NOs: 58-114, in which for the purposes of determining the degree of amino acid identity, the amino acid residues that form the CDR sequences are disregarded; and in which preferably one or more of the amino acid residues at positions one or more of the amino acid, residues at positions 11, 37, 44, 45, 47, 83, 84, 103, 104 and 108 according to the Kabat numbering are chosen from L and V for position 11, V and F at position 37, G at position 44, L at position 45, W and Y at position 47, R and K at position 83, A, T and D at position 84, W at position 103, G at position 104, and L, M and T at position 108.

In one embodiment of a immunoglobulin single variable domain as described herein, or an amino acid sequence as described herein, it comprises one or more, or two or more, or three or more stretches of amino acid residues selected from the group consisting of: a) the amino acid sequences encoding a CDR1 region of SEQ ID NOs: 58-114; b) amino acid sequences that have at least 80% amino acid identity with at least one of the amino acid sequences encoding a CDR1 region of SEQ ID NOs: 58-114; c) amino acid sequences that have 3, 2, or 1 amino acid difference with at least one of the amino acid sequences encoding a CDR1 region of SEQ ID NOs: 58-114; d) the amino acid sequences encoding a CDR2 region of SEQ ID NOs: 58-114; e) amino acid sequences that have at least 80% amino acid identity with at least one of the amino acid sequences encoding a CDR2 region of SEQ ID NOs: 58-114; amino acid sequences that have 3, 2, or 1 amino acid difference with at least one of the amino acid sequences encoding a CDR2 region of SEQ ID NOs: 58-114; g) the amino acid sequences encoding a CDR3 region of SEQ ID NOs: 58-114; h) amino acid sequences that have at least 80% amino acid identity with at least one of the amino acid sequences encoding a CDR3 region of SEQ ID NOs: 58-114; i) amino acid sequences that have 3, 2, or 1 amino acid difference with at least one of the amino acid sequences encoding a CDR3 region of SEQ ID NOs: 58-114; or any suitable combination thereof.

In one embodiment of an immunoglobulin variable domain described herein, one or more of the CDR sequences have at least 70% amino acid identity, preferably at least 80% amino acid identity, more preferably at least 90% amino acid identity, such as 95% amino acid identity or more or even essentially 100% amino acid identity with one or more of the CDR sequences of at least one of the amino acid sequences of SEQ ID NOs: 58-114.

In one embodiment, a immunoglobulin single variable domain is chosen from the group consisting of SEQ ID NOs: 58-114, or from the group consisting of from amino acid sequences that have more than 80%, preferably more than 90%, more preferably more than 95%, such as 99% or more sequence identity with at least one of the amino acid sequences of SEQ ED NOs: 58-114.

In one embodiment, the immunoglobulin single variable domain is chosen from the group consisting of SEQ ID NOs: 58-114.

The invention also provides an amino acid sequence that modulates, inhibits, prevents or blocks the interaction between a target on an antigen presenting cell (APC) and a target on a T-cell.

In one embodiment, the amino acid sequence is directed against and/or that can specifically bind to a target on an APC.

In one embodiment, the amino acid sequence is directed against and/or that can specifically bind to a target on a T-cell.

In one embodiment, the amino acid sequence inhibits and/or blocks T-cell activation.

In one embodiment, the amino acid sequence promotes and/or increases T-cell activation.

In one embodiment, the amino acid sequence inhibits and/or blocks cytokine production.

In one embodiment, the amino acid sequence increases cytokine production.

In one embodiment, the amino acid sequence increases T-cell survival.

In one embodiment, the amino acid sequence decreases T-cell survival.

In one embodiment, the amino acid sequence increases the differentiation of naive T-cells into activated cytokine secreting T-cells.

In one embodiment, the amino acid sequence decreases the differentiation of naive T-cells into activated cytokine secreting T-cells.

In one embodiment, the target on a T-cell or APC belongs to the B7:CD28 superfamily, and in a further embodiment the target is CD28.

In one embodiment, the amino acid sequence modulates, inhibits, prevents and/or blocks the interaction of B7-1 with CD28.

In one embodiment, the amino acid sequence modulates, inhibits, prevents and/or blocks the interaction of B7-1 with CD28 and the interaction of B7-1 with CTLA4.

In one embodiment, the amino acid sequence modulates, inhibits, prevents and/or blocks the interaction of B7-1 with CD28, while the interaction of B7-1 with CTLA4 is not modulated, inhibited or prevented.

In one embodiment, the amino acid sequence modulates, inhibits, prevents and/or blocks the interaction of B7-2 with CD28.

In one embodiment, the amino acid sequence modulates, inhibits, prevents and/or blocks the interaction of B7-2 with CD28 and the interaction of B7-1 with CTLA4.

In one embodiment, the amino acid sequence modulates, inhibits, prevents and/or blocks the interaction of B7-2 with CD28, while the interaction of B7-2 with CTLA4 is not modulated, inhibited or prevented.

In one embodiment, the amino acid sequence modulates, inhibits, prevents and/or blocks the interaction of B7-1 with CD28 and the interaction of B7-2 with CD28.

In one embodiment, the amino acid sequence modulates, inhibits, prevents and/or blocks the interaction of B7-1 with CD28, while the interaction of B7-2 with CD28 is not modulated, inhibited or prevented.

In one embodiment, the amino acid sequence modulates, inhibits, prevents and/or blocks the interaction of B7-2 with CD28, while the interaction of B7-1 with CD28 is not modulated, inhibited or prevented.

The invention also provides an amino acid sequence that comprises one or more stretches of amino acid residues chosen from the group consisting of: a) the amino acid sequences encoding a CDR1 region of SEQ ID NOs: 58-114; b) amino acid sequences that have at least 80% amino acid identity with at least one of the amino acid sequences encoding a CDR1 region of SEQ ID NOs: 58-114; c) amino acid sequences that have a 3, 2, or 1 amino acid difference with at least one of the amino acid sequences encoding a CDR1 region of SEQ ID NOs: 58-114; d) the amino acid sequences encoding a CDR2 region of SEQ ID NOs: 58-114; e) amino acid sequences that have at least 80% amino acid identity with at least one of the amino acid sequences encoding a CDR2 region of SEQ ID NOs: 58-114; f) amino acid sequences that have 3, 2, or 1 amino acid difference with at least one of the amino acid sequences encoding a CDR2 region of SEQ ID NOs: 58-114; g) the amino acid sequences encoding a CDR3 region of SEQ ID NOs: 58-114; h) amino acid sequences that have at least 80% amino acid identity with at least one of the amino acid sequences encoding a CDR3 region of SEQ ID NOs: 58-114; i) amino acid sequences that have 3, 2, or 1 amino acid difference with at least one of the amino acid sequences of encoding a CDR3 region of SEQ ID NOs: 58-114; or any suitable combination thereof.

In one embodiment, at least one of said stretches of amino acid residues of the amino acid sequence forms part of the antigen binding site for binding against CD28.

In one embodiment, the amino acid sequence comprises two or more stretches of amino acid residues chosen from the group consisting of: a) the amino acid sequences encoding a CDR1 region of SEQ ID NOs: 58-114; b) amino acid sequences that have at least 80% amino acid identity with at least one of the amino acid sequences encoding a CDR1 region of SEQ ID NOs: 58-114; c) amino acid sequences that have 3, 2, or 1 amino acid difference with at least one of the amino acid sequences encoding a CDR1 region of SEQ ID NOs: 58-114; d) the amino acid sequences encoding a CDR2 region of SEQ ID NOs: 58-114; e) amino acid sequences that have at least 80% amino acid identity with at least one of the amino acid sequences encoding a CDR2 region of SEQ ID NOs: 58-114; f) amino acid sequences that have 3, 2, or 1 amino acid difference with at least one of the amino acid sequences of encoding a CDR2 region of SEQ ID NOs: 58-114; g) the amino acid sequences encoding a CDR3 region of SEQ ID NOs: 58-114; h) amino acid sequences that have at least 80% amino acid identity with at least one of the amino acid sequences encoding a CDR3 region of SEQ ID NOs: 58-114; i) amino acid sequences that have 3, 2, or 1 amino acid difference with at least one of the amino acid sequences encoding a CDR3 region of SEQ ID NOs: 58-114; such that (i) when the first stretch of amino acid residues corresponds to one of the amino acid sequences according to a), b) or c), the second stretch of amino acid residues corresponds to one of the amino acid sequences according to d), e), g), h) or i). In one embodiment, at least two stretches of amino acid residues of the amino acid sequence forms part of the antigen binding site for binding against CD28.

In one embodiment, the amino acid sequence comprises three or more stretches of amino acid residues, in which the first stretch of amino acid residues is chosen from the group consisting of: a) the amino acid sequences encoding a CDR1 region of SEQ ID NOs: 58-114; b) amino acid sequences that have at least 80% amino acid identity with at least one of the amino acid sequences encoding a CDR1 region of SEQ ID NOs: 58-114; c) amino acid sequences that have 3, 2, or 1 amino acid difference with at least one of the amino acid sequences encoding a CDR1 region of SEQ ID NOs: 58-114; the second stretch of amino acid residues is chosen from the group consisting of: d) the amino acid sequences encoding a CDR2 region of SEQ ID NOs: 58-114; e) amino acid sequences that have at least 80% amino acid identity with at least one of the amino acid sequences encoding a CDR2 region of SEQ ID NOs: 58-114; f) amino acid sequences that have 3, 2, or 1 amino acid difference with at least one of the amino acid sequences encoding a CDR2 region of SEQ ID NOs: 58-114; and the third stretch of amino acid residues is chosen from the group consisting of: g) the amino acid sequences encoding a CDR3 region of SEQ ID NOs: 58-114; h) amino acid sequences that have at least 80% amino acid identity with at least one of the amino acid sequences encoding a CDR3 region of SEQ ID NOs: 58-114; i) amino acid sequences that have 3, 2, or 1 amino acid difference with at least one of the amino acid sequences encoding a CDR3 region of SEQ ID NOs: 58-114.

In one embodiment, at least three stretches of amino acid residues of the amino acid sequence forms part of the antigen binding site for binding against CD28.

In one embodiment, the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NOs: 58-114.

The invention further relates to an amino acid sequence that essentially consists of 4 framework regions (FR1 to FR4, respectively) and 3 complementarity determining regions (CDR1 to CDR3, respectively), in which: CDR1 is chosen from the group consisting of: a) the amino acid sequences encoding a CDR3 region of SEQ ID NOs: 58-114; b) amino acid sequences that have at least 80% amino acid identity with at least one of the amino acid sequences encoding a CDR3 region of SEQ ID NOs: 58-114; c) amino acid sequences that have 3, 2, or 1 amino acid difference with at least one of the amino acid sequences encoding a CDR3 region of SEQ ID NOs: 58-114; and/or CDR2 is chosen from the group consisting of: d) the amino acid sequences of SEQ ID NOs: 347-352; e) amino acid sequences that have at least 80% amino acid identity with at least one of the amino acid sequences of SEQ ID NOs: 347-352; f) amino acid sequences that have 3, 2, or 1 amino acid difference with at least one of the amino acid sequences of SEQ ID NOs: 347-352; and/or CDR3 is chosen from the group consisting of: g) the amino acid sequences of SEQ ID NOs: 359-364; h) amino acid sequences that have at least 80% amino acid identity with at least one of the amino acid sequences of SEQ ID NOs: 359-364; i) amino acid sequences that have 3, 2, or 1 amino acid difference with at least one of the amino acid sequences of SEQ ID NOs: 359-364.

In one embodiment, the amino acid sequence essentially consists of 4 framework regions (FR1 to FR4, respectively) and 3 complementarity determining regions (CDR1 to CDR3, respectively), in which:—CDR1 is chosen from the group consisting of: a) the amino acid sequences of SEQ ID NOs: 335-340; b) amino acid sequences that have at least 80% amino acid identity with at least one of the amino acid sequences of SEQ ID NOs: 335-340; c) amino acid sequences that have 3, 2, or 1 amino acid difference with at least one of the amino acid sequences of SEQ ID NOs: 335-340; and CDR2 is chosen from the group consisting of: d) the amino acid sequences of SEQ ID NOs: 347-352; e) amino acid sequences that have at least 80% amino acid identity with at least one of the amino acid sequences of SEQ ID NOs: 347-352; 0 amino acid sequences that have 3, 2, or 1 amino acid difference with at least one of the amino acid sequences of SEQ ID NOs: 347-352; and CDR3 is chosen from the group consisting of: g) the amino acid sequences of SEQ ID NOs: 359-364; h) amino acid sequences that have at least 80% amino acid identity with at least one of the amino acid sequences of SEQ ID NOs: 359-364; i) amino acid sequences that have 3, 2, or 1 amino acid difference with at least one of the amino acid sequences of SEQ ID NOs: 359-364.

In one embodiment, the amino acid sequence can specifically bind to an APC target or a T-cell target with a dissociation constant (K D) of 10⁻⁵ to 10⁻¹² moles/litre or less

In one embodiment, the amino acid sequence can specifically bind to an APC target or a T-cell target with a dissociation constant (K D) of 10⁻⁷ to 10⁻¹² moles/litre or less

In one embodiment, the amino acid sequence can specifically bind to an APC target or a T-cell target with a dissociation constant (K D) of 10⁻⁸ to 10⁻² moles/litre.

In one embodiment, the amino acid sequence can specifically bind to an APC target or a T-cell target with a rate of association (kₒₙ-rate) of between 10² M⁻¹ S⁻¹ to about 10⁷ M⁻¹ S⁻¹.

In one embodiment, the amino acid sequence can specifically bind to an APC target or a T-cell target with a rate of association (kₒₙ-rate) of between 10³ M⁻¹ S⁻¹ and 10⁷ M⁻¹ S⁻¹.

In one embodiment, the amino acid sequence can specifically bind to an APC target or a T-cell target with a rate of association (kₒₙ-rate) of between 10⁴ M⁻¹ S⁻¹ and 10⁷ M⁻¹ S⁻¹.

In one embodiment, the amino acid sequence can specifically bind to an APC target or a T-cell target with a rate of association (kₒₙ-rate) of between 10⁵ M⁻¹ S⁻¹ and 10⁷ M⁻¹ S⁻¹.

In one embodiment, the amino acid sequence can specifically bind to an APC target or a T-cell target with a rate of dissociation (k_{off} rate) between 1 s⁻¹ and 10⁻⁶ s⁻¹.

In one embodiment, the amino acid sequence can specifically bind to an APC target or a T-cell target with a rate of dissociation (k_{off} rate) between 10⁻² s⁻¹ and 10⁻⁶ s⁻¹.

In one embodiment, the amino acid sequence can specifically bind to an APC target or a T-cell target with a rate of dissociation (k_{off} rate) between 10⁻³ s⁻¹ and 10⁻⁶ s⁻¹.

In one embodiment, the amino acid sequence can specifically bind to an APC target or a T-cell target with a rate of dissociation (k_{w} rate) between 10⁻⁴ s⁻¹ and 10⁻⁶ S⁻¹.

In one embodiment, the amino acid sequence can specifically bind to an APC target or a T-cell target with an affinity less than 500 nM, preferably less than 200 nM, more preferably less than 10 nM, such as less than 500 pM.

### DETAILED DESCRIPTION

In order to provide improvement in the pharmacokinetics of antibody molecules, the present disclosure provides single domain variable region polypeptides that are linked to polymers which provide increased stability and half-life. In particular, the present disclosure provides improved molecules for the antagonism of CD28 activity including antibody single variable domains of human light chain origin, and including antibody single variable domains of human heavy chain origin. Included herein are compositions and methods for the attachment of polymer molecules (e.g., polyethylene glycol; PEG) to proteins to modulate the pharmacokinetic properties of the modified proteins. For example, PEG modification of proteins has been shown to alter the in vivo circulating half-life, antigenicity, solubility, and resistance to proteolysis of the protein (Abuchowski et al., J. Biol. Chem. 1977, 252:3578; Nucci et al., Adv. Drug Delivery Reviews 1991, 6:133; Francis et al., Pharmaceutical Biotechnology Vol. 3 (Borchardt, R. T. ed.); and Stability of Protein Pharmaceuticals: in vivo Pathways of Degradation and Strategies for Protein Stabilization 1991 pp 235-263, Plenum, NY).

### BRIEF DESCRIPTION OF THE DRAWINGS

FIG. 1A shows a schematic of plasmid pDOM4. FIG. 1B shows a schematic of plasmid pDOM5.

FIG. 2 shows ELISAs of soluble monoclonal antibody single variable domain binding to recombinant human CD28/Fc Chimera and Fc control coated plates.

FIG. 3 shows ELISAs of soluble monoclonal antibody single variable domain binding in the presence or absence of protein A to recombinant human CD28/Fc Chimera and Fc control coated plates.

FIG. 4 Biacore traces of dAb clones binding to a CM5 chip coated with 12500 units CD28-Fc.

FIG. 5 shows the ability of dAb clones to inhibit the activity of CD28 in a cell based in vitro assay.

FIG. 6 shows the ability of dAb clones DOM21-4 and DOM21-28 to inhibit the activity of CD28 in a cell based in vitro assay.

FIG. 7 shows the amino acid and nucleic acid sequences of MSA16 and MSA26.

### 1. DEFINITIONS

In accordance with this detailed description, the following abbreviations and definitions apply. It must be noted that as used herein, the singular forms “a”, “an”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth. Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods. Unless otherwise stated, all ranges described herein are inclusive of the specific endpoints. The following terms are provided below.

As used herein, the term “antagonist of CD28” or “anti-CD28 antagonist” or the like includes an agent (e.g., a molecule, a compound) which binds CD28 and can inhibit a (i.e., one or more) functions of CD28. For example, an antagonist of CD28 can inhibit the binding of CD28 to CD80 and/or CD86 and/or inhibit signal transduction mediated through CD28. Accordingly, CD28-mediated processes and cellular responses (e.g., CD28-induced T cell activation as indicated by cytokine secretion and/or proliferation in a standard assays) can be inhibited with an antagonist of CD28.

As used herein, “peptide” refers to about two to about 50 amino acids that are joined together via peptide bonds.

As used herein, “polypeptide” refers to at least about 50 amino acids that are joined together by peptide bonds. Polypeptides generally comprise tertiary structure and fold into functional domains.

As used herein, “target antigen” refers to an antigen which is specifically or selectively bound by a polypeptide or peptide. For example, when a polypeptide is an antibody single variable domain, a classical antibody, or antigen-binding fragment thereof, the target antigen can be any desired antigen or epitope that specifically binds to the antigen binding site of the antibody single variable domain or antibody. Binding to the target antigen is dependent upon the polypeptide or peptide being functional.

As used herein an antibody refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As used herein, “antibody format” refers to any suitable polypeptide structure in which one or more antibody single variable domains can optionally be incorporated into and still retain its ability to bind its target antigen in a monovalent fashion. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), an antibody single variable domain (e.g., a dAb, V_{H}, V_{HH}, V_{L}), and modified versions of any of the foregoing (e.g., modified by the covalent attachment of polyethylene glycol or other suitable polymer or a humanized V_{HH}).

The phrase “immunoglobulin single variable domain” refers to an antibody variable domain (e.g., V_{H}, V_{HH}, V_{L}) that specifically binds an antigen or epitope independently of other V regions or domains. An antibody single variable domain can be present in a format (e.g., homo- or hetero-multimer) with other variable regions or variable domains where the other regions or domains are not required for antigen binding by the antibody single variable domain (i.e., where the antibody single variable domain binds antigen independently of the additional variable domains). Exemplary formats as herein defined includes any one or more of those selected from the following: an antibody molecule comprising at least (i) the CL (kappa or lambda subclass) domain of an antibody; or (ii) the CH1 or CH2 or CH3 domain of an antibody heavy chain; an antibody molecule comprising the CH1 and CH2, or the CH2 and CH3, or the CH1 and CH3 domains of an antibody heavy chain; an antibody molecule comprising the CH1, CH2 and CH3 domains of an antibody heavy chain; or any of the subset (ii) in conjunction with the CL (kappa or lambda subclass) domain of an antibody. A hinge region domain may also be included. Such combinations of domains may, for example, mimic the format of natural antibodies, such as IgG or IgM, or fragments thereof, such as Fv, scFv, Fab, or F(ab′)₂ molecules. Those skilled in the art will be aware that this list is not intended to be exhaustive.

A “domain antibody”, a “dAb”, an “immunoglobulin single variable domain” a “single immunoglobulin variable domain”, a “single antibody variable domain” or an “antibody single variable domain” are equivalent. An immunoglobulin single variable domain is in one embodiment a human antibody variable domain, but also includes antibody single variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized by methods well known in the art. A V_{HH} domain can also be referred to herein as a “immunoglobulin single variable domain.”

A “immunoglobulin single variable domain” (in particular VHH sequences and partially humanized Nanobodies) can be defined as an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein. In particular, a Immunoglobulin single variable domain can be an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which: i) preferably one or more of the amino acid residues at positions 11, 37, 44, 45, 47, 83, 84, 103, 104 and 108 according to the Kabat numbering are preferably chosen from L and V for position 11, V and F at position 37, G at position 44, L at position 45, W and Y at position 47, R and K at position 83, A, T and D at position 84, W at position 103, G at position 104, and L, M and T at position 108; and in which: ii) said amino acid sequence has at least 80% amino acid identity with at least one of the amino acid sequences of SEQ ID NOs: 58-114, in which for the purposes of determining the degree of amino acid identity, the amino acid residues that form the CDR sequences are disregarded.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “monovalent domain” as used herein represents a domain which specifically binds its cognate ligand or a target antigen independently of an additional domain. A monovalent domain as used herein is broader than an antibody single variable domain in that it is not necessarily comprised of antibody related sequences. An “antibody single variable domain” is a folded polypeptide domain comprising sequences characteristic of a classical antibody variable region. An “antibody single variable domain” therefore in one embodiment is a complete antibody variable region which, unlike a classical antibody variable region, monovalently specifically binds antigen. With regard to an antibody single variable domain, the binding to antigen, e.g., CD28, is mediated by the single immunoglobulin V region, e.g. an antibody single variable domain, without a requirement for a complementary V region.

In another embodiment, an antibody single domain is a modified antibody variable region which monovalently binds antigen, where such a modification includes, for example, a variable region in which one or more of the CDRs have been replaced, or in which the antibody framework region(s) have been truncated and/or replaced or fused with a non antibody framework such as fibronectin and lipocallin described infra.

As used herein, the phrase “specifically binds” refers to the binding of an antigen by an antibody variable domain with a dissociation constant (k_{d}) of 1 pM or lower as measured by surface plasmon resonance analysis using, for example, a Biacore™ surface plasmon resonance system and Biacore™ kinetic evaluation software (e.g., version 2.1). The affinity or K_{d} for a specific binding interaction, in an aspect, is about 500 nM or lower, and in another aspect, about 300 nM or lower. As used herein, the term “binds” is equivalent to the phrase “specifically binds”.

A “universal framework” is a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. The use of a single framework, or a set of such frameworks, which has been found to permit the derivation of virtually any binding specificity though variation in the hypervariable regions alone, is included herein. Thus, libraries and repertoires can use a single framework, or a set of such frameworks, which has been found to permit the derivation of virtually any binding specificity though variation in the hypervariable regions alone.

As used herein, the term “dose” refers to the quantity of ligand administered to a subject all at one time (unit dose), or in two or more administrations over a defined time interval. For example, dose can refer to the quantity of ligand (e.g., ligand comprising an immunoglobulin single variable domain that binds target antigen) administered to a subject over the course of one day (24 hours) (daily dose), two days, one week, two weeks, three weeks or one or more months (e.g., by a single administration, or by two or more administrations). The interval between doses can be any desired amount of time.

As referred to herein, the term “competes” or “crossblocks” means that the binding of a first target to its cognate target binding domain is inhibited in the presence of a second binding domain that is specific for said cognate target. For example, binding may be inhibited sterically, for example by physical blocking of a binding domain or by alteration of the structure or environment of a binding domain such that its affinity or avidity for a target is reduced. Competition between a first and a second binding domain can be analyzed by performing competition ELISA and competition Biacore experiments. See WO2006038027. For example, the following two Biacore protocols can be used to determine if two antibody single variable domains compete with each other for binding to CD28. In the first method, an antigen is immobilized on the sensor surface followed by the pair-wise binding analysis of different antibody single variable domains. In this example a super-shift in binding following saturation of the chip surface by the first antibody single variable domain indicates that the two antibody single variable domains bind non-overlapping epitopes. If the two antibody single variable domains bound overlapping epitopes, no (or a minimal) super-shift in signal would be observed.

The second Biacore protocol involves the capture of one antibody single variable domain on the chip surface. The antigen and then the antibody single variable domain are passed over the chip surface. The level of binding is determined relative to a control consisting of the same antibody single variable domain used for immobilization being passed over the chip surface and competing with itself for binding to antigen. An increase in signal indicates that the two antibody single variable domains do not compete and therefore bind distinct epitopes. No signal increase indicates that the two antibody single variable domains compete and therefore bind overlapping epitopes. Both Biacore protocols are described by Ann-Christin Malmborg and Carl A. K. Borrebaeck, (1995), Biacore as a tool in antibody engineering. Journal of Immunological Methods, 183, 7-13.

ELISA based protocols can also be used to determine if two antibody single variable domains compete with each other in binding antigen. In a first ELISA protocol, VH antibody single variable domains are mixed with VK antibody single variable domains and the level of binding to passively coated antigen (or biotinylated antigen captured by neutavidin coated ELISA plates) determined by detection with protein-L HRP or protein-A HRP as appropriate. In this example, if the VK antibody single variable domain is the detection antibody single variable domain, then if both the VK and VH antibody single variable domains bound to overlapping epitopes of CD28 they would compete for binding and the amount of V_{K} being detected by protein-L HRP would decrease relative to a non-competition V_{K} control at the same molar concentration. Generally the competing antibody single variable domain (the VH in this example) would be added in molar excess to the detection antibody single variable domain. If the two d antibody single variable domains bound non-overlapping epitopes they would not compete and there would be no decrease in ELISA signal relative to the antibody single variable domain control. A similar approach can be used to determine if two VH antibody single variable domains or two VK antibody single variable domains compete. In this case the competing antibody single variable domain needs to be labelled, for example biotinylated or contain a peptide tag such as the FLAG tag.

A second ELISA approach for analyzing the competition between two antibody single variable domains involves the immobilization or passive adsorption onto an ELISA plate of one antibody single variable domain. Antigen is captured by the coated antibody single variable domain and then a second antibody single variable domain is added and the degree of binding is determined as described above. Various ELISA competition methods (direct or bead based) exist in the literature, examples are: described by Ju-Won Kwak and Chang-Soon Yoon, (1996), Journal of Immunological Methods 191, 49-54, and by Xiao-Chi Jia, et al., (2004), Journal of Immunological Methods 288, 91-98.

Flow cytometry protocols can also be used for analyzing the competition between two antibody single variable domains in a similar manner as described for the ELISA method, except the degree of binding of competing/non-competing antibody single variable domains to cells or beads coated with recombinant antigen is determined by flow cytometry. See Reed D S, et al., (2002), Cytometry, 49(1), 1-7, and see Sai A. Patibandla, et al., (1997), The Journal of Clinical Endocrinology & Metabolism, 82(6), 1885-1893.

Crystallography can also be used for analyzing the competition between two antibody single variable domains, for example: forming a CD28 antibody single variable domain plus CD28 co-crystal.

Sequences similar (e.g., having at least about 70% sequence identity) to the sequences disclosed herein are also included herein. In some embodiments, the sequence identity at the amino acid level can be about 50%, 65%, 70%, 75%, 80%, 85%, 90%, 91%, 92%, 93%, 94%, 95%, 96%, 97%, 98%, 99%, or higher. At the nucleic acid level, the sequence identity can be about 50%, 65%, 70%, 75%, 80%, 85%, 90%, 91%, 92%, 93%, 94%, 95%, 96%, 97%, 98%, 99%, or higher. The nucleic acids may be present in whole cells, in a cell lysate, or in a partially purified or substantially pure form.

The term “substantial identity” or “percent identity” means that two peptide sequences, when optimally aligned, such as by the programs GAP or BESTFIT using default gap weights, share at least 50 percent sequence identity, preferably at least 65, 70, 75, 80, 85 or 90 percent sequence identity, more preferably at least 95 percent sequence identity or more (e.g., 99 percent sequence identity). Alternatively, the software AlignX™, a component of Vector NTI Suite 8.0 (InforMax, Inc.) can be used. The alignment is created using the Clustal W algorithm (Nucleic Acid Research, 22 (22): 4673-4680, 1994). The basic multiple alignment algorithm consists of three main stages: 1) all pairs of sequences are aligned separately in order to calculate a distance matrix giving the divergence of each pair of sequences; 2) a guide tree is calculated from the distance matrix; 3) the sequences are progressively aligned according to the branching order in the guide tree. Accordingly, a crude similarity between all pairs of sequences is calculated, called a “Parities alignment”. These scores are then used to calculate a “guide tree” or dendrogram, which tells the multiple alignment stage the order in which to align the sequences for the final multiple alignment. Having calculated the dendrogram, the sequences are aligned in larger and larger groups until the entire sequences are incorporated in the final alignment. Calculations of “identity” between two sequences are performed as follows: the sequences are aligned for optimal comparison purposes (e.g., gaps can be introduced in one or both of a first and a second amino acid or nucleic acid sequence for optimal alignment and non-identical sequences can be disregarded for comparison purposes). In one embodiment calculations of identity of amino acid and nucleic acid sequences are determined using the software AlignX.

Pairwise Alignment settings for AlignX were set as follows:

| Use FAST Algorithm: | OFF |
|---|---|
| K-tuple size: | 1 |
| Number of best diagonals: | 5 |
| Window Size: | 5 |
| Gap penalty: | 3 |
| Gap opening penalty: | 10 |
| Gap extension penalty: | 0.1 |

Multiple Alignment Settings for AlignX were set as follows:

| | |
|---|---|
| Gap opening penalty: | 10 |
| Gap extension penalty: | 0.05 |
| Gap separation penalty range: | 8 |
| No end gap separation penalty: | Unselected |
| % identity for alignment delay: | 40 |
| Residue specific gaps off: | Unselected |
| Hydrophilic residue gap off: | Unselected |
| Transition weighting: | 0 |

In a more general embodiment, the length of a reference sequence aligned for comparison purposes is at least 30%, or at least 40%, or at least 50%, or at least 60%, or at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. Amino acid and nucleotide sequence alignments and identity, as defined herein may also be prepared and determined using the algorithm BLAST 2 Sequences, using default parameters (Tatusova, T. A. et al., FEMS Microbiol Lett, 174:187-188 (1999)).

In one embodiment, a human immunoglobulin single variable domain or an antibody single variable domain has at least 70% amino acid identity (including, for example, 71% 73%, 75%, 77%, 79%, 81%, 83%, 85%, 87%, 90%, 93%, 95%, 97%, 99%, or higher identity) to a naturally-occurring human immunoglobulin variable domain sequence. In an embodiment, a human antibody single variable domain has at least 85% amino acid similarity or identity (including, for example, 71% 73%, 75%, 77%, 79%, 81%, 83%, 85%, 87%, 90%, 93%, 95%, 97%, 99%, or higher identity) to a naturally-occurring human antibody single variable domain sequence, e.g. a naturally occurring human antibody single variable domain sequence disclosed in Kabat (“Sequences of Proteins of Immunological Interest”, U.S. Department of Health and Human Services, 1991).

An antibody single variable domain lacks the antibody constant regions. However, one or more, or all of the antibody constant regions can be fused to an antibody single variable domain. Alternatively, fusion proteins, e.g. derivitaives, comprising an antibody single variable domain and other proteins or polypeptides are encompassed herein.

Parts or fragments of the Immunoglobulin single variable domains of the invention have amino acid sequences in which, compared to the amino acid sequence of the corresponding full length Immunoglobulin single variable domain of the invention, one or more of the amino acid residues at the N-terminal end, one or more amino acid residues at the C-terminal end, one or more contiguous internal amino acid residues, or any combination thereof, have been deleted and/or removed. The parts or fragments of the Immunoglobulin single variable domains described herein bind to the APC target or T-cell target, e.g. CD28, with an affinity (suitably measured and/or expressed as a Ko-value (actual or apparent), a KA-value (actual or apparent), a kₒₙ-rate and/or a k_{off}-rate, or alternatively as an IC50 value, that is as defined herein for the Immunoglobulin single variable domains described herein. Any part or fragment of the Immunoglobulin single variable domains described herein preferably comprises at least 10 contiguous amino acid residues, preferably at least 20 contiguous amino acid residues, preferably at least 30 contiguous amino acid residues, such as at least 40 contiguous amino acid residues, of the amino acid sequence of the corresponding full length Immunoglobulin single variable domain described herein. Also, any part or fragment preferably comprises at least one of a CDR1, CDR2 and/or CDR3 or at least part thereof, and preferably, at least CDR3 or at least part thereof. Preferably, any part or fragment comprises at least one of the CDRs, preferably at least CDR3 or part thereof, and at least one other CDR (e.g., CDR1 or CDR2) or at least part thereof, and is preferably connected by suitable framework sequence(s) or at least part(s) thereof. Preferably, any part or fragment comprises at least one of the CDRs, and preferably at least CDR3 or part thereof, and at least part of the two remaining CDR's, again preferably connected by framework sequence(s) or at least part thereof.

The antibody variable domain of an antibody single variable domain can be a modified. A modified antibody single variable domain, retains a dissociation constant of 500 nM or less, (e.g., 450 nM or less, 400 nM or less, 350 nM or less, 300 nM or less, 250 nM or less, 200 nM or less, 150 nM or less, 100 nM or less) and retains the target antigen specificity of the full-length domain. Where necessary or in case of any doubt, the numbering convention and boundaries set forth by Kabat et al. (Kabat et al., 1991, Sequences of Immunological Interest, 5^{th} ed. U.S. Dept. Health & Human Services, Washington, D.C.) are applicable to immunoglobulin variable and constant domains referred to herein.

An antibody single variable domain is in one embodiment a human antibody variable domain, but also includes an antibody single variable domain from other species such as rodent (for example, as disclosed in WO 00/29004, the contents of which are incorporated by reference in their entirety), nurse shark and camelid V_{HH} dAbs. Camelid V_{HH} are immunoglobulin single variable domain polypeptides that are derived from species including camel, llama, alpacea, dromedary, and guanaco, which produce heavy chain antibodies naturally devoid of light chains. The V_{HH} may be humanized.

As used herein, the term “human” when applied to an antibody single variable domain or to an immunoglobulin single variable domain means that the polypeptide has a sequence derived from a human immunoglobulin sequence. A sequence is “derived from” a human immunoglobulin coding sequence when the sequence is either: a) isolated from a human individual or from cells or a cell line derived from a human individual; b) isolated from a library of human antibody gene sequences (or a library of human antibody V domain sequences); or c) when a cloned human antibody sequence (or a cloned human V region sequence (including, e.g., a germline V gene segment)) was used to generate one or more diversified sequences that were then selected for binding to a desired target antigen.

Camelid antibodies are described in, for example, U.S. Pat. Nos. 5,759,808; 5,800,988; 5,840,526; 5,874,541; 6,005,079; and 6,015,695, the contents of each of which are incorporated herein in their entirety. Camelid V_{HH} antibody single variable domain polypeptides set forth herein include a class of camelid antibody single variable domain polypeptides having human-like sequences, where the class is characterized in that the V_{HH} domains carry an amino acid at position 45 selected from glycine, alanine, valine, leucine, isoleucine, proline, phenylalanine, tyrosine, tryptophan, methionine, serine, threonine, asparagine, or glutamine, e.g., L45, and further comprise a tryptophan at position 103 according to the Kabat numbering (Kabat et al., 1991, Sequences of Immunological Interest, 5^{th} ed. U.S. Dept. Health & Human Services, Washington, D.C.). In one embodiment, the V_{HH} has a glycine position 44 and optionally also an arginine, lysine, cysteine or charged amino acid at position 45. Humanized camelid V_{HH} polypeptides are taught, for example in WO04/041862, the teachings of which are incorporated herein in their entirety. It will be understood by one of skill in the art that naturally occurring camelid antibody single variable domain polypeptides may be modified according to the teachings of WO04/041862 (e.g., amino acid substitutions at positions 45 and 103) to generate humanized camelid V_{HH} polypeptides. Also included herein are antibody single variable domain polypeptides which are nurse shark V_{HH}. Nurse shark dAbs are antibody single variable domain polypeptides derived from the nurse shark that comprise heavy chain antibodies naturally devoid of light chain. Nurse Shark single variable domains are described, for example, in Greenberg et al. (Nature 374:168-173, 1995) and U.S. Publication No. 20050043519.

As used herein, the term “epitope” refers to a unit of structure of an antigen conventionally bound by an immunoglobulin V_{H}/V_{L} pair. Epitopes define the minimum binding site for an antibody, and thus represent the target of specificity of an antibody. Epitopes can be linear or conformational, and can be a small as three amino acids. In the case of an antibody single variable domain, an epitope represents the unit of structure bound by a single variable domain in isolation. That is, an epitope is the binding site is provided by one immunoglobulin single variable domain.

As used herein, the term “extended release”, or the equivalent terms “controlled release” or “slow release”, refer to drug formulations that release active drug, such as a polypeptide drug, over a period of time following administration to an individual. Extended release of polypeptide drugs, which can occur over a range of desired times, e.g., minutes, hours, days, weeks, or longer, depending upon the drug formulation, is in contrast to standard formulations in which substantially the entire dosage unit is available for immediate absorption or immediate distribution via the bloodstream. Preferred extended release formulations result in a level of circulating drug from a single administration that is sustained, for example, for 8 hours or more, 12 hours or more, 24 hours or more, 36 hours or more, 48 hours or more, 60 hours or more, 72 hours or more 84 hours or more, 96 hours or more, or even, for example, for 1 week or 2 weeks or more, for example, 1 month or more.

As used herein, “CD28 activity” is an activity involving or resulting from the binding of CD80 and/or CD86 to CD28, and includes, but is not limited to, activation of CD28-mediated T cell signaling. CD28 activity also includes the induction of T cell proliferation and the induction of cytokine secretion, e.g., interleukin 2 (IL-2), by T cells, which can be assayed by methods well known to one of skill in the art.

As used herein, the term “does not substantially agonize” means that a given agent, e.g., an anti-CD28 antibody single variable domain, does not activate one or more of the CD28 mediated activities as the term “activate” is defined herein. Specifically, an agent that “does not substantially agonize” means that the agent does not activate more than 20% of the activity which is activated by CD80 and/or CD86 binding to CD28, and in an aspect, the agent does not activate more than 10%, 8%, 5%, 3%, or 2%, including zero activation, of the activity which is mediated by CD80 and/or CD86 binding to CD28. By way of a non-limiting example, an anti-CD28 monovalent polypeptide domain as set forth herein, that does not substantially agonize CD28 activity, means that it does not agonize CD28 activity to more than 5% of the CD28 mediated activity obtained upon agonism of CD28 activity by anti-CD28 mAb 9.3 (Gibson, et al. (1996) JBC, 271:7079-7083) under otherwise identical assay conditions.

As used herein, the terms “inhibit,” “inhibits” and “inhibited”, or “prevent” refer to a decrease in or decreased level of a given measurable activity (e.g., binding activity) by at least 10% relative to a reference. Where inhibition is desired, such inhibition is at least 20%, 30%, 40%, 50%, 60%, 70%, 80%, 90%, or more, up to and including 100%, i.e., complete inhibition or absence of the given activity. Inhibition of CD28 binding to CD80 or CD86 can be measured as described in the working examples herein. As used herein, the term “substantially inhibits” refers to a decrease in a given measurable activity (e.g., the binding of CD28 to CD80 or CD86) by at least 50% relative to a reference. For example, “substantially inhibits” refers to a decrease in a given measurable activity of at least 50%, 55%, 60%, 65%, 70%, 75%, 80%, 85%, 90%, 95%, and up to and including 100% relative to a reference. As used herein, “inhibits the binding”, with reference to the binding of a CD28 antibody single variable domain's binding to CD28, CD80's binding to CD28, and/or CD86's binding to CD28, refers to a decrease in binding of CD80 and/or CD86 to CD28 at least 10% in the presence of the CD28 antibody single variable domain, relative to its absence. “Inhibits the binding” refers to a decrease in binding of at least 20%, 30%, 40%, 50%, 60%, 70%, 80%, 90%, or more, up to and including 100% relative to a reference. “Prevents the binding” refers to a decreased level of binding of 20%, 30%, 40%, 50%, 60%, 70%, 80%, 90%, or more, up to and including 100% relative to a reference.

In the context of the present invention, “modulating” or “to modulate” generally means either reducing or inhibiting the activity of, or alternatively increasing the activity of T-cell target expressing CD28 and/or the activity of the CD28 receptor itself, or a cellular pathway linked to CD28, as measured using a suitable in vitro, cellular or in vivo assay, such as those mentioned herein. In particular, “modulating” or “to modulate” may mean either reducing or inhibiting the activity by at least 1%, preferably at least 5%, such as at least 10% or at least 25%, for example by at least 50%, at least 60%, at least 70%, at least 80%, or 90% or more, compared to activity of T-cell target expressing CD28 and/or the activity of the CD28 receptor itself, or a cellular pathway linked to CD28, in the same assay under the same conditions but without the presence of the amino acid sequence, immunoglobulin single variable domain or polypeptide of the invention. As will be clear to the skilled person, “modulating” may also involve effecting a change (which may either be an increase or a decrease) in affinity, avidity, specificity and/or selectivity of CD28, for one or more of its ligands, receptors or substrates; and/or effecting a change (which may either be an increase or a decrease) in the sensitivity of CD28 for one or more of its ligands, receptors or substrates. As will be clear to the skilled person, this may again be determined in any suitable manner and/or using any suitable assay known per se, such as the assays described herein or in the prior art cited herein. “Modulating” may also mean effecting a change (i.e. an activity as an agonist or as an antagonist, respectively) with respect to one or more biological or physiological mechanisms, effects, responses, functions, pathways or activities in which CD28 and/or its substrate(s), ligand(s), receptor(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, such as the assays described herein or in the prior art cited herein. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the amino acid sequence, immunoglobulin single variable domain or polypeptide of the invention.

As used herein, the term “preferentially inhibits” as used in a phrase such as “wherein an antibody single variable domain preferentially inhibits the binding to CD28 by CD86 relative to the binding to CD28 by CD80”, means that the antibody single variable domain effects a higher amount of inhibition of CD86 binding to CD28 as defined above, relative to the amount of inhibition of CD80 binding to CD28 as defined above.

As used herein, the terms “activate”, “activates” and “activated” refer to an increase in a given measurable activity by at least 5% relative to a reference, for example, at least 10%, 25%, 50%, 75%, or even 100% or more.

As used herein, the term “CD28 antagonist” refers to an agent that inhibits at least one activity mediated by CD28, by inhibiting the binding of CD80 and/or CD86 to CD28. A CD28 activity is “antagonized” if the activity is reduced by at least 10%, and in an exemplary embodiment, at least 20%, 30%, 40%, 50%, 60%, 70%, 80%, 90%, 95%, 97%, or even 100% (i.e., no activity) in the presence of an antagonist, relative to the activity in the absence of an antagonist. In an exemplary embodiment, a CD28 antagonist as the term is used herein comprises an antibody single variable domain that binds monovalently to CD28. By way of a non-limiting example, a CD28 antagonist as set forth herein is an agent that inhibits some or all CD28 activity, while at the same time, the agent does not substantially agonize CD28 mediated activity of T cell receptor signaling.

As used herein, the term “CD28 agonist” refers to an agent that activates at least one activity mediated by CD28, either alone or when combined with another co-stimulus, relative to a reference. An activity is “agonized” if the activity is increased by at least 10%, e.g., 50%, in the presence, relative to the absence of an agonist.

As used herein, the term “CTLA4” activity includes, but is not limited to, inhibition of T cell function. Such functions include, among others, T cell receptor mediated signaling, T cell proliferation, and induction of cytokine secretion.

As used herein, “immune disease” refers to any disease which is associated with the development of an immune reaction in an individual, including a cellular and/or a humoral immune reaction. Examples of immune diseases include, but are not limited to, inflammation, allergy, transplantation, arthritis and autoimmune diseases.

As used herein, “autoimmune disease” refers to disease conditions and states wherein the immune response of an individual is directed against the individual's own constituents, resulting in an undesirable and often debilitating condition. As used herein, “autoimmune disease” is intended to further include autoimmune conditions, syndromes, and the like. Autoimmune diseases include, but are not limited to, Addison's disease, autoimmune diseases of the ear, autoimmune diseases of the eye, autoimmune hepatitis, Crohn's disease, diabetes (Type I), epididymitis, glomerulonephritis, Graves' disease, Guillain-Barre syndrome, Hashimoto's disease, hemolytic anemia, systemic lupus erythematosus, male infertility, multiple sclerosis, myasthenia gravis, pemphigus, psoriasis, rheumatic fever, rheumatoid arthritis, sarcoidosis, scleroderma, Sjogren's syndrome, spondyloarthropathies, thyroiditis, and vasculitis. Autoimmune-mediated conditions also include, but are not limited to, conditions in which the tissue affected is the primary target, and in some cases, the secondary target. Such conditions include, but are not limited to, AIDS, atopic allergy, bronchial asthma, eczema, leprosy, schizophrenia, inherited depression, transplantation of tissues and organs, chronic fatigue syndrome, Alzheimer's disease, Parkinson's disease, myocardial infarction, stroke, autism, epilepsy, Arthus's phenomenon, anaphylaxis, and alcohol and drug addiction.

As used herein, the term “monovalent” means that a given antibody single variable polypeptide domain, CD28 binder, CD28 antagonist, CD28 binding polypeptide or CD28 binding domain or moiety can bind only a single molecule of its target. Naturally-occurring antibodies are generally “divalent”, in that they have two functional antigen-binding arms, each arm comprising a VH and a VL domain. Where steric hindrance is not an issue, a divalent antibody can bind two separate molecules of the same antigen. In contrast, a “monovalent” antibody has the capacity to bind only one such antigen molecule. An antibody single variable domain, e.g., a V_{H} or a V_{L} domain, has the capacity to bind an antigen such as CD28, without the need for a corresponding V_{L} or V_{H} domain, respectively. An antibody single variable domain lacks the capacity to cross link molecules of a single antigen.

As used herein, the terms “V_{H} domain” and “V_{L} domain” refer to antibody variable regions, e.g., as generally defined by Kabat et al. (Kabat et al., 1991, Sequences of Immunological Interest, 5^{th} ed. U.S. Dept. Health & Human Services, Washington, D.C.), which is incorporated herein by reference.

Derivatives of the immunoglobulin single variable domains of the invention and/or of one or more of the amino acid residues that form the immunoglobulin single variable domains of the invention as used herein include modification, and in particular by chemical and/or biological (e.g enzymatical) modification, of the immunoglobulin single variable domains of the invention and/or of one or more of the amino acid residues that form the immunoglobulin single variable domains of the invention. The modifications can, for example, function to increase the half life, and/or stability of the immunoglobulin single variable domains of the invention and/or of one or more of the amino acid residues that form the immunoglobulin single variable domains of the invention. Likewise, modifications can, for example, function as detectable labels or signal generating moieties for the immunoglobulin single variable domains of the invention and/or of one or more of the amino acid residues that form the immunoglobulin single variable domains of the invention.

Examples of such modifications, as well as examples of amino acid residues within the immunoglobulin single variable domain sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the immunoglobulin single variable domain of the invention, and in particular of one or more functional groups, residues or moieties that confer one or more desired properties or functionalities to the immunoglobulin single variable domain of the invention. Example of such functional groups will be clear to the skilled person.

For example, such modification may comprise the introduction (e.g. by covalent binding or in any other suitable manner) of one or more functional groups that increase the half-life, the solubility and/or the absorption of the immunoglobulin single variable domain of the invention, that reduce the immunogenicity and/or the toxicity of the immunoglobulin single variable domain of the invention, that eliminate or attenuate any undesirable side effects of the immunoglobulin single variable domain of the invention, and/or that confer other advantageous properties to and/or reduce the undesired properties of the immunoglobulin single variable domain and/or polypeptides of the invention; or any combination of two or more of the foregoing.

As used herein, “linked” refers to the attachment of a polymer moiety, such as PEG to an amino acid residue of an antibody single variable domain. Attachment of a PEG polymer to an amino acid residue of an antibody single variable domain, e.g., an anti-CD28 dAb, is referred to as “PEGylation” and may be achieved using several PEG attachment moieties including, but not limited to N-hydroxylsuccinimide (NHS) active ester, succinimidyl propionate (SPA), maleimide (MAL), vinyl sulfone (VS), or thiol. A PEG polymer, or other polymer, can be linked to an antibody single variable domain at either a predetermined position, or may be randomly linked to the antibody single variable domain molecule. It is preferred, however, that the PEG polymer be linked to an antibody single variable domain at a predetermined position. A PEG polymer may be linked to any residue in the an antibody single variable domain, however, it is preferable that the polymer is linked to either a lysine or cysteine, which is either naturally occurring in the antibody single variable domain, or which has been engineered into the antibody single variable domain, for example, by mutagenesis of a naturally occurring residue in the antibody single variable domain to either a cysteine or lysine. PEG-linkage can also be mediated through a peptide linker attached to an antibody single variable domain. That is, the PEG moiety can be attached to a peptide linker fused to an antibody single variable domain, where the linker provides the site, e.g., a free cysteine or lysine, for PEG attachment. As used herein, “linked” can also refer to the association of two or more antibody single variable domains, e.g., dAb monomers, to form a dimer, trimer, tetramer, or other multimer. Antibody single variable domain monomers can be linked to form a multimer by several methods known in the art, including, but not limited to, expression of the antibody single variable domain monomers as a fusion protein, linkage of two or more monomers via a peptide linker between monomers, or by chemically joining monomers after translation, either to each other directly, or through a linker by disulfide bonds, or by linkage to a di-, tri- or multivalent linking moiety (e.g., a multi-arm PEG). While dAb multimers are specifically contemplated herein, e.g., in the context of dual- or multi-specific antibody single variable domain constructs, it is emphasized that for any given antibody single variable domain construct, the construct should only be able to bind one molecule of CD28, i.e., the constructs can have only one CD28-binding element, and cannot cross link CD28.

As used herein, “polymer” refers to a macromolecule made up of repeating monomeric units, and can refer to a synthetic or naturally occurring polymer such as an optionally substituted straight or branched chain polyalkylene, polyalkenylene, or polyoxyalkylene polymer or a branched or unbranched polysaccharide. A “polymer” as used herein, specifically refers to an optionally substituted or branched chain poly(ethylene glycol), poly(propylene glycol), or poly(vinyl alcohol) and derivatives thereof.

As used herein, “PEG” or “PEG polymer” refers to polyethylene glycol, and more specifically can refer to a derivitized form of PEG, including, but not limited to N-hydroxylsuccinimide (NHS) active esters of PEG such as succinimidyl propionate, benzotriazole active esters, PEG derivatized with maleimide, vinyl sulfones, or thiol groups. For example, PEG formulations can include PEG-O—CH₂CH₂CH₂—CO₂—NHS; PEG-O—CH₂—NHS; PEG-O—CH₂CH₂—CO₂—NHS; PEG-S—CH₂CH₂—CO—NHS; PEG-O2CNH—CH(R)—CO₂—NHS; PEG-NHCO—CH₂CH₂—CO—NHS; and PEG-O—CH₂—CO₂—NHS; where R is (CH₂)₄)NHCO2(mPEG). PEG polymers set forth herein may be linear molecules, or may be branched wherein multiple PEG moieties are present in a single polymer. Some particularly preferred PEG conformations include, but are not limited to the following:

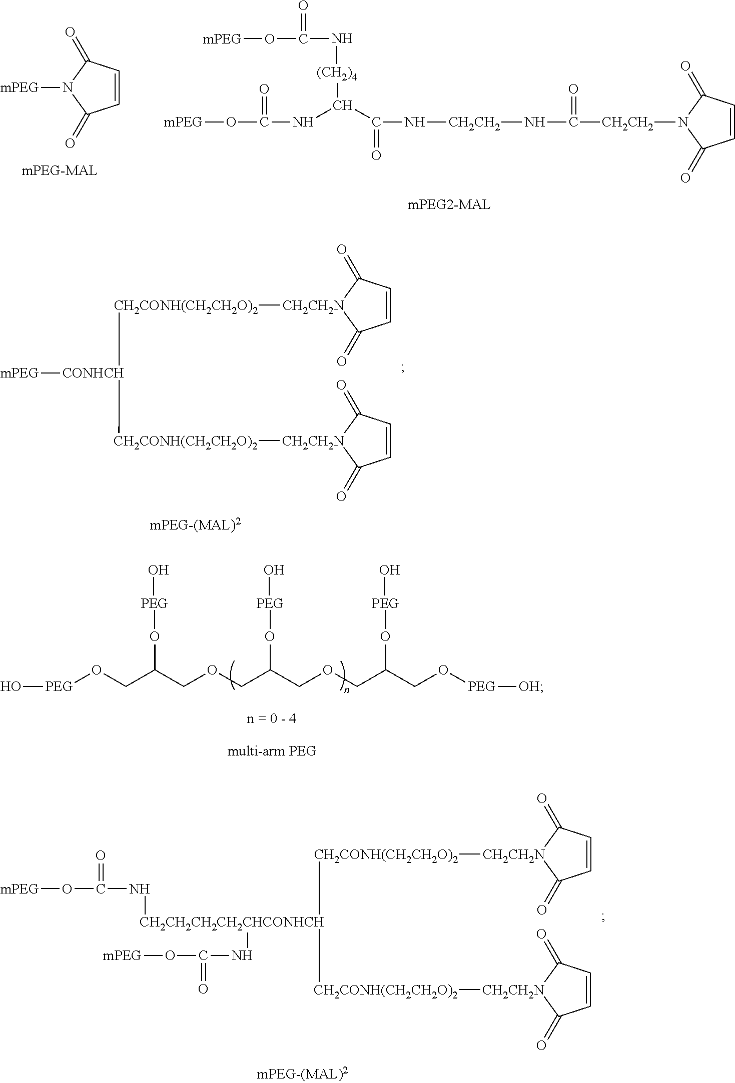

As used herein, a “sulfhydryl-selective reagent” is a reagent which is useful for the attachment of a PEG polymer to a thiol-containing amino acid. Thiol groups on the amino acid residue cysteine are particularly useful for interaction with a sulfhydryl-selective reagent. Sulfhydryl-selective reagents which are useful for such attachment include, but are not limited to maleimide, vinyl sulfone, and thiol. The use of sulfhydryl-selective reagents for coupling to cysteine residues is known in the art and may be adapted as needed (See, e.g., Zalipsky, 1995, Bioconjug. Chem. 6:150; Greenwald et al., 2000, Crit. Rev. Ther. Drug Carrier Syst. 17:101; Herman et al., 1994, Macromol. Chem. Phys. 195:203).

The attachment of PEG or another agent, e.g., human serum albumin (HSA), to an antibody single variable domain or to an immunoglobulin single variable domain polypeptide as described herein in an exemplary embodiment, will not impair the ability of the antibody single variable domain to specifically bind CD28. That is, the PEG-linked antibody single variable domain or immunoglobulin single variable domain polypeptide will retain its binding activity relative to a non-PEG-linked counterpart. As used herein, “retains activity” refers to a level of activity of a PEG-linked antibody single variable domain which is at least 10% of the level of activity of a non-PEG-linked antibody single variable domain, including at least 20%, 30%, 40%, 50%, 60%, 70%, 80% and up to 90%, including up to 95%, 98%, and up to 100% of the activity of a non-PEG-linked antibody single variable domain comprising the same antigen-binding domain or domains. More specifically, the activity of a PEG-linked antibody single variable domain compared to a non-PEG linked antibody variable domain should be determined on an antibody single variable domain molar basis; that is equivalent numbers of moles of each of the PEG-linked and non-PEG-linked antibody single variable domains should be used in each trial. In determining whether a particular PEG-linked antibody single variable domain “retains activity”, it is preferred that the activity of a PEG-linked antibody single variable domain be compared with the activity of the same antibody single variable domain in the absence of PEG.

As used herein, the term “in vivo half-life” refers to the time taken for the serum concentration of a CD 28 antagonist and/or an immunoglobulin single variable domain, or an antibody single variable domain, to be reduced by 50%, in vivo, for example due to degradation of and/or clearance or sequestration by natural mechanisms. The anti-CD28 antibody single variable domains or immunoglobulin single variable domain polypeptides described herein can be stabilized in vivo and their half-life increased by binding to molecules, such as PEG, which resist degradation and/or clearance or sequestration. The half-life of an antibody single variable domain is increased if its functional activity persists, in vivo, for a longer period than a similar antibody single variable domain which is not linked to a PEG polymer. Typically, the half-life of a PEGylated antibody single variable domain is increased by 10%, 20%, 30%, 40%, 50%, or more relative to a non-PEGylated antibody single variable domain. Increases in the range of 2×, 3×, 4×, 5×, 10×, 20×, 30×, 40×, 50×, or more of the half-life are possible. Alternatively, or in addition, increases in the range of up to 30×, 40×, 50×, 60×, 70×, 80×, 90×, 100×, or 150× of the half-life are possible. As set forth herein, a PEG-linked antibody single variable domain has a half-life of between 0.25 and two weeks, including between 1 and 100 hours, further including between 30 and 100 hours, and still further including between 50 and 100 hours, and up to 170, 180, 190, and 200 hours or more.

As used herein, “resistant to degradation” or “resists degradation” with respect to a PEG or other polymer-linked antibody single variable domain monomer or multimer means that the PEG- or other polymer-linked antibody single variable domain monomer or multimer is degraded by no more than 10% when exposed to pepsin at pH 2.0 for 30 minutes and in an aspect, not degraded at all.

As used herein, “hydrodynamic size” refers to the apparent size of a molecule (e.g., a protein molecule) based on the diffusion of the molecule through an aqueous solution. The diffusion, or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation of the protein. Hydrodynamic size is measured, for example, by size exclusion chromatography. The hydrodynamic size of a PEG-linked immunoglobulin single variable domain, e.g., an antibody single variable domain (including antibody variable domain multimers as described herein), or polypeptide comprising said antibody single variable domain, can be in the range of 24 kDa to 500 kDa; 30 to 500 kDa; 40 to 500 kDa; 50 to 500 kDa; 100 to 500 kDa; 150 to 500 kDa; 200 to 500 kDa; 250 to 500 kDa; 300 to 500 kDa; 350 to 500 kDa; 400 to 500 kDa, and 450 to 500 kDa. In an aspect, the hydrodynamic size of a PEGylated antibody single variable domain is 30 to 40 kDa; 70 to 80 kDa, or 200 to 300 kDa. Where an antibody single variable domain polypeptide is desired for use in imaging applications, the polypeptide should have a hydrodynamic size of between 50 and 100 kDa. Alternatively, where an antibody single variable domain polypeptide is desired for therapeutic applications, the polypeptide preparation should have a hydrodynamic size of greater than 200 kDa.

As used herein, the term “IC₅₀” refers to the concentration of an inhibitor necessary to inhibit a given activity by 50%. IC₅₀ is determined by assaying a given activity, e.g., binding of CD28 to CD80 or CD86, in the presence of varying amounts of the inhibitor (e.g., monovalent anti-CD28 antibody single variable domain), and plotting the inhibitor concentration versus the activity being targeted. Binding to CD28 by CD80 or CD86 is measured herein by the method described the working examples. Alternatively, surface plasmon resonance (SPR) can be used.

As used herein, the term “fused to an antibody single variable domain” means that a polypeptide is fused to a given antibody generally through use of recombinant DNA techniques, though fusion may occur chemically at the protein level. Thus, an antibody “fused to” another polypeptide, e.g., to another antibody of different binding specificity, does not exist in nature and is generated through recombinant means. The term “fused to an antibody single variable domain” also encompasses the linkage of a polypeptide to a given antibody single variable domain through, for example, disulfide or other chemical linkages, where the fused polypeptide is not naturally found fused to the antibody single variable domain. Recombinant and chemical methods of fusing a polypeptide to another polypeptide, e.g., to an antibody, are well known in the art.

As used herein, the term “Fc domain” refers to the antibody constant regions and sequences thereof, comprising one or more of the Fc regions, preferably CH2 and CH3 constant regions as delimited according to Kabat et al., supra. The Fc portion of the heavy chain polypeptide has the ability to self-associate, a function which facilitates the formation of divalent antibodies. The term “lacks an Fc domain” means that a given antibody single variable domain lacks at least the portion of an immunoglobulin Fc domain sufficient to mediate the dimerization of Fc-containing monovalent antibody single variable domains. Dimerization of Fc-containing antibody single variable domains is measured, for example, by chromatographic methods or by surface plasmon resonance. An antibody single variable domain lacking an Fc domain avoids Fc-platelet interactions and therefore avoids induction of platelet aggregation.

As used herein “treat”, “reduce”, “prevent”, or “alleviate” as it relates to a symptom of disease refer to a decrease of the a symptom by at least 10% based on a clinically measurable parameter, or by at least one point on a clinically-accepted scale of disease or symptom severity. As used herein, the term “symptom(s) of systemic lupus erythematosus” refers to any of the clinically relevant symptoms of SLE known to those of skill in the art. Non-limiting examples include the accumulation of IgG autoantibodies (e.g., against nuclear antigens such as chromatin, snRNPs (especially U1, Sm, Ro/SSA and La/SSB), phospholipids and cell surface molecules), hemolytic anemia, thrombocytopenia, leukopenia, glomerulonephritis, vasculitis, arthritis, and serositis). A reduction in such a symptom is a reduction by at least 10% in a clinically measurable parameter, or by at least one point on a clinically-accepted scale of disease severity.

As used herein, a “generic ligand” is a ligand that binds a substantial proportion of functional members in a given repertoire, e.g., in a phage display library. Thus, the same generic ligand can bind many members of the repertoire regardless of their target ligand specificities. In general, the presence of a functional generic ligand binding site indicates that the repertoire member is expressed and folded correctly. Thus, binding of the generic ligand to its binding site provides a method for preselecting functional polypeptides from a repertoire of polypeptides. Generic ligands include, for example, Protein A, Protein G and Protein L.

### Overview:

Monovalent polypeptide domains which specifically bind CD28, including antibody single variable domains, are provided that are monovalent for binding to CD28. While not being bound by any particular theory, monovalency for CD28 binding removes the possibility for cross-linking cell surface receptors that occurs with prior art antibodies. With respect to inhibiting CD28-mediated T cell activation, a monovalent binder of CD28 has unique advantages with respect to antagonizing a T cell response mediated through CD28 over an anti-CD28 dimeric antibody which clusters the cell surface CD28 molecules promoting T cell stimulation. Thus, the monovalency of CD28 specific antibody single variable domains can inhibit CD28 activity, dampening an immune response, while avoiding potential undesirable stimulatory effects that can occur with antibodies capable of divalent or multivalent binding of CD28. Thus, in a preferred aspect, the anti-CD28 antibodies disclosed herein not only inhibit or antagonize the binding of CD80 or CD86 to CD28, they do not substantially agonize CD28 activity.

In one aspect, the antibodies monovalent for CD28 binding are human antibody single variable domains. Human antibody single variable domains can be administered to human patients while largely avoiding the anti-antibody immune response often provoked by the administration of antibodies from other species, e.g., mouse.

As described above, antibody single variable domains are distinct from conventional antibodies. The heavy and light polypeptide chains of conventional antibodies comprise variable (V) regions that directly participate in antigen interactions, and constant (C) regions that provide structural support and function in non-antigen-specific interactions with immune effectors. The antigen binding domain of a conventional antibody is formed by the interaction of two separate domains: a heavy chain variable domain (V_{H}) and a light chain variable domain (V_{L}: which can be either V_{κ} or V_{λ}). The antigen binding site of a conventional antibody is formed by six polypeptide loops: three from the V_{H} domain (H1, H2 and H3) and three from the V_{L} domain (L1, L2 and L3). In vivo, a diverse primary repertoire of V genes that encode the V_{H} and V_{L} domains of conventional antibodies is produced by the combinatorial rearrangement of gene segments. C regions include the light chain C regions (referred to as C_{L} regions) and the heavy chain C regions (referred to as C_{H}1, C_{H}2 and C_{H}3 regions). A naturally-occurring, conventional antibody generally comprises two antigen binding domains and is therefore divalent. Unlike antibody single variable domains, each antigen binding domain of a naturally occurring, traditional antibody, comprises a heavy and light polypeptide chain which together cooperatively form an antigen binding domain. In contrast, the antigen binding domain of an antibody single variable domain, requires only one domain, e.g., one peptide or polypeptide chain.

A number of smaller antigen binding fragments of conventional, natural occurring antibodies have been identified following protease digestion. These include, for example, the “Fab fragment” (V_{L}-C_{L}/C_{H}1-V_{H}), “Fab′ fragment” (a Fab with the heavy chain hinge region), and “F(ab′)₂ fragment” (a dimer of Fab′ fragments joined by the heavy chain hinge region). Recombinant methods have been used to generate such fragments and to generate even smaller antigen-binding fragments, e.g., those referred to as “single chain Fv” (variable fragment) or “scFv,” consisting of V_{L} and V_{H} joined by a peptide linker (V_{L}-linker-V_{H}). Fab fragments, Fab′ fragments and scFv fragments are monovalent for antigen binding, as they each comprise only one antigen binding site which comprises 2 variable regions, i.e., a V_{H}/V_{L} dimer.

As described supra, the term “dAb” is synonymous with an antibody single variable domain (V_{H} or V_{L}) polypeptide that specifically binds antigen through a single domain. The preparation of antibody single variable domains is described and exemplified herein below.

Antibody single variable domains, for example, V_{HH}, are the smallest antigen-binding antibody unit known. For use in therapy, human antibodies are preferred, primarily because they are not as likely to provoke an immune response when administered to a patient. As noted above, isolated non-camelid V_{H} domains tend to be relatively insoluble and are often poorly expressed. Comparisons of camelid V_{HH} with the V_{H} domains of human antibodies reveals several key differences in the framework regions of the camelid V_{HH} domain corresponding to the V_{H}/V_{L} interface of the human V_{H} domains. Mutation of these residues of human V_{H}3 to more closely resemble the V_{HH} sequence (specifically Gly 44→Glu, Leu 45→Arg and Trp 47-+Gly) has been performed to produce “camelized” human V_{H} domains that retain antigen binding activity (Davies & Riechmann, 1994, FEBS Lett. 339: 285-290) yet have improved expression and solubility. (Variable domain amino acid numbering used herein is consistent with the Kabat numbering convention (Kabat et al., 1991, Sequences of Immunological Interest, 5^{th} ed. U.S. Dept. Health & Human Services, Washington, D.C.)) WO 03/035694 (Muyldermans) reports that the Trp 103→Arg mutation improves the solubility of non-camelid V_{H} domains. Davies & Riechmann (1995, Biotechnology N.Y. 13: 475-479) also report production of a phage-displayed repertoire of camelized human V_{H} domains and selection of clones that bind hapten with affinities in the range of 100-400 nM, but clones selected for binding to protein antigen had weaker affinities. General Strategy and Methods for Design of monovalent CD28 antagonists comprising a conventional antigen binding site formed by the VH and VL of a conventional antibody.

The means of generating monovalent conventional antibodies specific for CD28 is well known in the art. One means is to amplify and express the V_{H} and V_{L} regions of the heavy chain and light chain gene sequences isolated, for example, from a hybridoma (e.g., a mouse hybridoma) that expresses conventional anti-CD28 monoclonal antibody. The boundaries of V_{H} and V_{L} domains are set out by Kabat et al. (Kabat et al., 1991, Sequences of Immunological Interest, 5^{th} ed. U.S. Dept. Health & Human Services, Washington, D.C.). The information regarding the boundaries of the V_{H} and V_{L} domains of heavy and light chain genes is used to design PCR primers that amplify the V domain from a heavy or light chain coding sequence encoding a conventional antibody known to bind CD28. The amplified V domains are inserted into a suitable expression vector, e.g., pHEN-1 (Hoogenboom et al., 1991, Nucleic Acids Res. 19: 4133-4137) and expressed, e.g., as a fusion of the V_{H} and V_{L} in an scFv or other suitable monovalent format. The resulting polypeptide is then screened for high affinity monovalent binding to CD28 by virtue of an antigen binding site formed by the pairs of V_{H} and V_{L} polypeptides. In conjunction with the methods set forth herein, screening for binding is performed as known in the art or as described herein below.

Alternatively, library screening methods can be used to identify monovalent CD28-specific binding proteins. Phage display technology (see, e.g., Smith, 1985, Science 228: 1315; Scott & Smith, 1990, Science 249: 386; McCafferty et al., 1990, Nature 348: 552) provides an approach for the selection of antibody variable regions which bind a desired target from among large, diverse repertoires of antibody variable regions. These phage-antibody libraries can be grouped into two categories: natural libraries which use rearranged V genes harvested from human B cells (Marks et al., 1991, J. Mol. Biol., 222: 581; Vaughan et al., 1996, Nature Biotech., 14: 309) or synthetic libraries whereby germline V gene segments or other antibody variable region coding sequences are ‘rearranged’ in vitro (Hoogenboom & Winter, 1992, J. Mol. Biol., 227: 381; Nissim et al., 1994, EMBO J., 13: 692; Griffiths et al., 1994, EMBO J., 13: 3245; De Kruif et al., 1995, J. Mol. Biol., 248: 97) or where synthetic CDRs are incorporated into a single rearranged V gene (Barbas et al., 1992. Proc. Natl. Acad. Sci. USA, 89: 4457). Methods involving genetic display packages (e.g., phage display, polysome display) are well-suited for the selection of monovalent CD28-specific antibody constructs because they generally express only monovalent fragments, rather than whole, divalent antibodies, on the display packages. Methods for the preparation of phage display libraries displaying various antibody fragments are described in the preceding references. Such methods are also described, for example, in U.S. Pat. No. 6,696,245, which is incorporated herein by reference. The methods described in the '245 patent generally involve the randomization of selected regions of immunoglobulin gene coding regions, in particular V_{H} and V_{L} coding regions, while leaving other regions non-randomized (see below). The '245 patent also describes the generation of scFv constructs comprising individually randomized V_{H} and V_{L} domains.

Analysis of the structures and sequences of antibodies has shown that five of the six antigen binding loops (H1, H2, L1, L2, L3) possess a limited number of main-chain conformations or canonical structures (Chothia and Lesk (1987) J. Mol. Biol. 196: 901; Chothia et al. (1989) Nature 342: 877). The main-chain conformations are determined by (i) the length of the antigen binding loop, and (ii) particular residues, or types of residue, at certain key positions in the antigen binding loop and the antibody framework. Analysis of the loop lengths and key residues has enabled the prediction of the main-chain conformations of H1, H2, L1, L2 and L3 encoded by the majority of human antibody sequences (Chothia et al. (1992) J. Mol. Biol. 227: 799; Tomlinson et al. (1995) EMBO J. 14: 4628; Williams et al. (1996) J. Mol. Biol. 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol. 263: 800; Shirai et al. (1996) FEBS Letters 399: 1.

While, in one approach, diversity can be added to synthetic repertoires at any site in the CDRs of the various antigen-binding loops, this approach results in a greater proportion of V domains that do not properly fold and therefore contribute to a lower proportion of molecules with the potential to bind antigen. An understanding of the residues contributing to the main chain conformation of the antigen-binding loops permits the identification of specific residues to diversify in a synthetic repertoire of V_{H} or V_{L} domains. That is, diversity is best introduced in residues that are not essential to maintaining the main chain conformation. As an example, for the diversification of loop L2, the conventional approach would be to diversify all the residues in the corresponding CDR (CDR2) as defined by Kabat et al. (Kabat et al., 1991, Sequences of Immunological Interest, 5^{th} ed. U.S. Dept. Health & Human Services, Washington, D.C.), some seven residues. However, for L2, it is known that positions 50 and 53 are diverse in naturally occurring antibodies and are observed to make contact with the antigen. The preferred approach would be to diversify only those two residues in this loop. This represents a significant improvement in terms of the functional diversity required to create a range of antigen binding specificities.

Immunoglobulin polypeptide libraries can advantageously be designed to be based on predetermined variable domain main chain conformation. Such libraries may be constructed as described in International Patent Application WO 99/20749, the contents of which are incorporated herein by reference. Such variable region polypeptides can be used for the production of scFvs or Fabs, e.g., an scFv or Fab comprising (i) an antibody heavy chain variable domain (V_{H}), or antigen binding fragment thereof, which comprises the amino acid sequence of germline V_{H} segment DP-47 and (ii) an antibody light chain variable domain (V_{I}), or antigen binding fragment thereof, which comprises the amino acid sequence of germline V, segment DPK9. Diversification of sequences within the context of the selected heavy and light chain germline gene segments, e.g., DP-47, DPK 9, DP45, DP38, etc. can generate a repertoire of diverse immunoglobulin coding sequences. One approach to diversification is described below in the context of generating a library of diversified antibody single variable domain or scFv sequences. Thus, in one aspect, an antibody single variable domain comprises the amino acid sequence of a given human germline V region gene segment, e.g., V_{H} germline gene segment DP-47, or V_{κ} germline gene segment DPK9. These variable region polypeptides can also be expressed as antibody single variable domains and screened for high affinity binding to CD28. The repertoire can be cloned into or generated in a vector suitable for phage display, e.g., a lambda or filamentous bacteriophage display vector and is then screened for binding to a given target antigen, e.g., CD28.

### Preparation of CD28 Monovalent Binders

An antibody single variable domain is a folded polypeptide domain which comprises sequences characteristic of antibody variable domains and which specifically binds an antigen (e.g., dissociation constant of 500 nM or less), and which binds antigen as a single variable domain; that is, there is one binding site provided by an antibody single variable domain without any complementary variable domain. An antibody single variable domain therefore includes complete antibody variable domains as well as modified variable domains, for example in which one or more loops have been replaced by sequences which are not characteristic of antibody variable domains or antibody variable domains which have been truncated or comprise N- or C-terminal extensions, as well as folded fragments of variable domains which retain a dissociation constant of 500 nM less (e.g., 450 nM or less, 400 nM or less, 350 nM or less, 300 nM or less, 250 nM or less, 200 nM or less, 150 nM or less, 100 nM or less) and the target antigen specificity of the full-length domain. In an exemplary embodiment, an antibody single variable domain useful in the compositions and methods set forth herein is selected from the group of V_{H} and V_{L}, including Vₖₐₚₚₐ and V_{lambda}, and V_{HH}. In an exemplary embodiment, the antibody single variable domains of use herein are “human” as that term is defined herein.

### Format and Structure of Antibody Single Variable Domains

According to one aspect disclosed herein, two or more antibody single variable domains may be linked while still maintaining the ability of each antibody single variable domain to bind its respective antigen independently of each other. Advantageously, the two or more linked antibody single variable domains may be further attached to a skeleton which may, as an alternative, or on addition to a linker described herein, facilitate the formation and/or maintenance of the ability of each antibody single variable domain to bind its respective antigen independently of each other. Alternatively, the monomeric anti-CD28 antibody single variable domain polypeptides disclosed herein may be constructed using scaffold or frameworks as discussed herein. Antibody single variable domain skeletons may be based on antibody chain molecules or may be non-antibody in origin as set forth elsewhere herein.

Each epitope binding domain comprises a protein scaffold and one or more CDRs which are involved in the specific interaction of the domain with one or more epitopes. Advantageously, an epitope binding domain disclosed herein comprises three CDRs. Suitable protein scaffolds, in addition to those based on antibody domains, may also be based on protein scaffolds or skeletons other than immunoglobulin domains which are non-antibody in origin. For example natural bacterial receptors such as Staphylococcus protein A have been used as scaffolds for the grafting of CDRs to generate ligands which bind specifically to one or more epitopes. Details of this procedure are described in U.S. Pat. No. 5,831,012. Other suitable scaffolds include those based on fibronectin and affibodies (Affibody, Bromma, Sweeden). Details of suitable procedures are described in WO 98/58965. Other suitable scaffolds include lipocallin and CTLA4, as described in van den Beuken et al., J. Mol. Biol. (2001) 310, 591-601, and scaffolds such as those described in WO00/69907 (Medical Research Council), which are based for example on the ring structure of bacterial GroEL or other chaperone polypeptides. Other non-immunoglobulin based scaffolds which may be used include those based on the LDL receptor class A, EGF domain monomers and multimers, and scaffolds available from Biorexis (King of Prussia, Pa.) or Avidia (Mountain View, Calif.). Other non-immunoglobulin scaffolds which may be used are described, for example, in WO05/040229, WO04/044011, and US2005/0089932

### Selection of the Antibody Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2, and L3 found in the majority of human antibodies (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length, and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

The antibody single variable domains disclosed herein can be selected and/or assembled from libraries containing variable domains, such as libraries of V_{H} domains and/or libraries of V_{I}, domains. Moreover, the ligands disclosed herein may themselves be provided in the form of libraries. In one aspect disclosed herein, libraries of ligands and/or domains are designed in which certain loop lengths and key residues have been chosen to ensure that the main-chain conformation of the members is known. Advantageously, these are real conformations of immunoglobulin superfamily molecules found in nature, to minimize the chances that they are non-functional, as discussed above. Germline V gene segments serve as one exemplary basic framework for constructing antibody or T cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on ligand sequences and to chose residues for diversification which do not affect the canonical structure. It is known that, in the human V_{κ} region, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} region alone, different canonical structures can combine to create a range of different main-chain conformations. Given that the V_{λ} region encodes a different range of canonical structures for the L1, L2, and L3 loops, and that V_{κ} and V_{λ} regions can pair with any V_{H} region which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure a single naturally occurring conformation can be used as the basis for an entire library. Thus, in a preferred aspect, the antibody single variable domains disclosed herein possess a single known main-chain conformation.

The single main-chain conformation that is chosen is, in an aspect, commonplace among molecules of the immunoglobulin superfamily type in question. A conformation is commonplace when a significant number of naturally occurring molecules are observed to adopt it. Accordingly, in a preferred aspect disclosed herein, the natural occurrence of the different main-chain conformations for each binding loop of an immunoglobulin domain are considered separately and then a naturally occurring variable domain is chosen which possesses the desired combination of main-chain conformations for the different loops. If none is available, the nearest equivalent may be chosen. It is preferable that the desired combination of main-chain conformations for the different loops is created by selecting germline gene segments which encode the desired main-chain conformations. It is more preferable, that the selected germline gene segments are frequently expressed in nature, and most preferable that they are the most frequently expressed of all natural germline gene segments.

In designing ligands or libraries thereof the incidence of the different main-chain conformations for each of the antigen binding loops may be considered separately. For H1, H2, L1, L2, and L3, a given conformation that is adopted by between 20% and 100% of the antigen binding loops of naturally occurring molecules is chosen. Typically, its observed incidence is above 35% (i.e. between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1-CS1 (79% of the expressed repertoire), H2-CS 3 (46%), L1-CS 2 of V_{κ} (39%), L2-CS1 (100%), L3-CS1 of V_{κ} (36%) (calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modeling (2cgr and 1tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}1. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five, or for all six of the antigen binding loops can be determined. Here, it is preferable that the chosen conformation is commonplace in naturally occurring antibodies and most preferable that it observed most frequently in the natural repertoire. Thus, in human antibodies, for example, when natural combinations of the five antigen binding loops, H1, H2, L1, L2, and L3, are considered, the most frequent combination of canonical structures is determined and then combined with the most popular conformation for the H3 loop, as a basis for choosing the single main-chain conformation.

### Preparation of Antibody Single Variable Domains

Antibody single variable domains are prepared in a number of ways. For each of these approaches, well-known methods of preparing (e.g., amplifying, mutating, etc.) and manipulating nucleic acid sequences are applicable.

One means of preparing antibody single variable domains is to amplify and express the V_{H} or V_{L} region of a heavy chain or light chain gene for a cloned antibody or V_{HH} known to bind the desired antigen. That is, the V_{H} or V_{L} region of a known anti-CD28 antibody coding region can be amplified and expressed as a single domain (or as a fusion of a single domain) and evaluated for binding to CD28. The boundaries of V_{H} and V_{L} regions are set out by Kabat et al. (Kabat et al., 1991, Sequences of Immunological Interest, 5^{th} ed. U.S. Dept. Health & Human Services, Washington, D.C.). The information regarding the boundaries of the V_{H} and V_{L} regions of heavy and light chain genes is used to design PCR primers that amplify the V domain from a cloned heavy or light chain coding sequence encoding an antibody known to bind CD28. The amplified V domain is inserted into a suitable expression vector, e.g., pHEN-1 (Hoogenboom et al., 1991, Nucleic Acids Res. 19: 4133-4137) and expressed, either alone or as a fusion with another polypeptide sequence.

The V_{H} gene is produced by the recombination of three gene segments, V_{H}, D and J_{H}. In humans, there are approximately 51 functional V_{H} segments (Cook and Tomlinson (1995) Immunol Today 16: 237), 25 functional D segments (Corbett et al. (1997) J. Mol. Biol. 268: 69) and 6 functional J_{H} segments (Ravetch et al. (1981) Cell 27: 583), depending on the haplotype. The V_{H} segment encodes the region of the polypeptide chain which forms the first and second antigen binding loops of the V_{H} region (H1 and H2), while the V_{H}, D and J_{H} segments combine to form the third antigen binding loop of the V_{H} region (H3).

The V_{L} gene is produced by the recombination of only two gene segments, V_{L} and J_{L}. In humans, there are approximately 40 functional V_{κ} segments (Schäble and Zachau (1993) Biol. Chem. Hoppe-Seyler 374: 1001), 31 functional V_{λ} segments (Williams et al. (1996) J. Mol. Biol. 264: 220; Kawasaki et al. (1997) Genome Res. 7: 250), 5 functional J_{κ} segments (Hieter et al. (1982) J. Biol. Chem. 257: 1516) and 4 functional J_{λ} segments (Vasicek and Leder (1990) J. Exp. Med. 172: 609), depending on the haplotype. The V_{L} segment encodes the region of the polypeptide chain which forms the first and second antigen binding loops of the V_{L} region (L1 and L2), while the V_{L} and J_{L} segments combine to form the third antigen binding loop of the V_{L} region (L3). Antibodies selected from this primary repertoire are believed to be sufficiently diverse to bind almost all antigens with at least moderate affinity. High affinity antibodies are produced in vivo by “affinity maturation” of the rearranged genes, in which point mutations are generated and selected by the immune system on the basis of improved binding.

In a preferred approach, a repertoire of V_{H} or V_{L} domains, in an aspect, human V_{H} or V_{L} domains, is screened by, for example, phage display, panning against the desired antigen. Methods for the construction of bacteriophage display libraries and lambda phage expression libraries are well known in the art, and taught, for example, by McCafferty et al., 1990, Nature 348: 552; Kang et al., 1991, Proc. Natl. Acad. Sci. U.S.A., 88: 4363; Clackson et al., 1991, Nature 352: 624; Lowman et al., 1991, Biochemistry 30: 10832; Burton et al., 1991, Proc. Natl. Acad. Sci. U.S.A. 88: 10134; Hoogenboom et al., 1991, Nucleic Acids Res. 19: 4133; Chang et al., 1991, J. Immunol. 147: 3610; Breitling et al., 1991, Gene 104: 147; Marks et al., 1991, J. Mol. Biol. 222: 581; Barbas et al., 1992, Proc. Natl. Acad. Sci. U.S.A. 89: 4457; Hawkins and Winter (1992) J. Immunol., 22: 867; Marks et al. (1992) J. Biol. Chem., 267: 16007; and Lerner et al. (1992) Science, 258: 1313. Fab phage display libraries are taught, for example, by U.S. Pat. No. 5,922,545. scFv phage libraries are taught, for example, by Huston et al., 1988, Proc. Natl. Acad. Sci. U.S.A. 85: 5879-5883; Chaudhary et al., 1990, Proc. Natl. Acad. Sci. U.S.A. 87: 1066-1070; McCafferty et al., 1990, supra; Clackson et al., 1991, supra; Marks et al., 1991, supra; Chiswell et al., 1992, Trends Biotech. 10: 80; and Marks et al., 1992, supra. Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys, supra).

The repertoire of V_{H} or V_{L} domains can be a naturally-occurring repertoire of immunoglobulin sequences or a synthetic repertoire. A naturally-occurring repertoire is one prepared, for example, from immunoglobulin-expressing cells harvested from one or more individuals. Such repertoires can be “naïve,” i.e., prepared, for example, from human fetal or newborn immunoglobulin-expressing cells, or rearranged, i.e., prepared from, for example, adult human B cells. Natural repertoires are described, for example, by Marks et al., 1991, J. Mol. Biol. 222: 581 and Vaughan et al., 1996, Nature Biotech. 14: 309. If desired, clones identified from a natural repertoire, or any repertoire, for that matter, that bind the target antigen are then subjected to mutagenesis and further screening in order to produce and select variants with improved binding characteristics.

Synthetic repertoires of immunoglobulin single variable domains, including antibody single variable domains, are prepared by artificially introducing diversity into a cloned V domain. Synthetic repertoires are described, for example, by Hoogenboom & Winter, 1992, J. Mol. Biol. 227: 381; Barbas et al., 1992, Proc. Natl. Acad. Sci. U.S.A. 89: 4457; Nissim et al., 1994, EMBO J. 13: 692; Griffiths et al., 1994, EMBO J. 13: 3245; DeKriuf et al., 1995, J. Mol. Biol. 248: 97; and WO 99/20749.

In one aspect, synthetic variable domain repertoires are prepared in V_{H} or V_{κ} backgrounds, based on artificially diversified germline V_{H} or V_{κ} sequences. For example, the V_{H} domain repertoire can be based on cloned germline V_{H} gene segments V3-23/DP47 (Tomlinson et al., 1992, J. Mol. Biol. 227: 7768) and JH4b. The V_{κ} domain repertoire can be based, for example, on germline V_{κ} gene segments O2/O12/DPK9 (Cox et al., 1994, Eur. J. Immunol. 24: 827) and J_{κ}1. Diversity is introduced into these or other gene segments by, for example, PCR mutagenesis. Diversity can be randomly introduced, for example, by error prone PCR (Hawkins, et al., 1992, J. Mol. Biol. 226: 889) or chemical mutagenesis. As discussed above, however it is preferred that the introduction of diversity is targeted to particular residues. It is further preferred that the desired residues are targeted by introduction of the codon NNK using mutagenic primers (using the IUPAC nomenclature, where N=G, A, T or C, and K=G or T), which encodes all amino acids and the TAG stop codon. Other codons which achieve similar ends are also of use, including the NNN codon (which leads to the production of the additional stop codons TGA and TAA), DVT codon ((A/G/T) (A/G/C)T), DVC codon ((A/G/T)(A/G/C)C), and DVY codon ((A/G/T)(A/G/C)(C/T). The DVT codon encodes 22% serine and 11% tyrosine, asparagine, glycine, alanine, aspartate, threonine and cysteine, which most closely mimics the distribution of amino acid residues for the antigen binding sites of natural human antibodies. Repertoires are made using PCR primers having the selected degenerate codon or codons at each site to be diversified. PCR mutagenesis is well known in the art.

In one aspect, diversity is introduced into the sequence of human germline V_{H} gene segments V3-23/DP47 (Tomlinson et al., 1992, J. Mol. Biol. 227: 7768) and JH4b using the NNK codon at sites H30, H31, H33, H35, H50, H52, H52a, H53, H55, H56, H58, H95, H97, and H98, corresponding to diversity in CDRs 1, 2 and 3, with the numbering as used in U.S. Pat. No. 6,696,245.

In another aspect, diversity is also introduced into the sequence of human germline V_{H} gene segments V3-23/DP47 and JH4b, for example, using the NNK codon at sites H30, H31, H33, H35, H50, H52, H52a, H53, H55, H56, H58, H95, H97, H98, H99, H100, H100a, and H100b, corresponding to diversity in CDRs 1, 2 and 3, with the numbering as used in U.S. Pat. No. 6,696,245.

In another aspect, diversity is introduced into the sequence of human germline V_{κ} gene segments O2/O12/DPK9 and J_{κ}1, for example, using the NNK codon at sites L30, L31, L32, L34, L50, L53, L91, L92, L93, L94, and L96, corresponding to diversity in CDRs 1, 2 and 3, with the numbering as used in U.S. Pat. No. 6,696,245.

Diversified repertoires are cloned into phage display vectors as known in the art and as described, for example, in WO 99/20749. In general, the nucleic acid molecules and vector constructs required for the compositions and methods set forth herein are available in the art and are constructed and manipulated as set forth in standard laboratory manuals, such as Sambrook et al. (1989), Molecular Cloning: A Laboratory Manual, Cold Spring Harbor, USA and subsequent editions.

The manipulation of nucleic acids as set forth herein is typically carried out in recombinant vectors. As used herein, “vector” refers to a discrete element that is used to introduce heterologous DNA into cells for the expression and/or replication thereof. Methods by which to select or construct and, subsequently, use such vectors are well known to one of skill in the art. Numerous vectors are publicly available, including bacterial plasmids, bacteriophage, artificial chromosomes and episomal vectors. Such vectors may be used for simple cloning and mutagenesis; alternatively, as is typical of vectors in which repertoire (or pre-repertoire) members herein are carried, a gene expression vector is employed. A vector of use set forth herein is selected to accommodate a polypeptide coding sequence of a desired size, typically from 0.25 kilobase (kb) to 40 kb in length. A suitable host cell is transformed with the vector after in vitro cloning manipulations. Each vector contains various functional components, which generally include a cloning (or “polylinker”) site, an origin of replication and at least one selectable marker gene. If a given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member as set forth herein.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector also contains a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Because the replication of vectors herein is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19. However, other plasmid microorganism combinations can also be reasonably substituted.

Expression vectors usually contain a promoter that is recognized by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Dalgarno sequence operably linked to the coding sequence.

In libraries or repertoires as described herein, the preferred vectors are expression vectors that enable the expression of a nucleotide sequence corresponding to a polypeptide library member. Thus, selection is performed by separate propagation and expression of a single clone expressing the polypeptide library member or by use of any selection display system. As described above, a preferred selection display system uses bacteriophage display. Thus, phage or phagemid vectors can be used. Preferred vectors are phagemid vectors, which have an E. coli origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase or other selectable marker gene to confer selectivity on the phagemid, and a lac promoter upstream of a expression cassette that consists (N to C terminal) of a pelB leader sequence (which directs the expressed polypeptide to the periplasmic space), a multiple cloning site (for cloning the nucleotide version of the library member), optionally, one or more peptide tags (for detection), optionally, one or more TAG stop codons and the phage protein pIII. In one embodiment, the vector encodes, rather than the pelB leader sequence, a eukaryotic GAS1 leader sequence which serves to direct the secretion of the fusion polypeptide to the periplasmic space in E. coli or to the medium in eukaryotic cell systems. Using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only, or produce phage, some of which contain at least one copy of the polypeptide-pIII fusion on their surface.

An example of a preferred vector is the pHEN1 phagemid vector (Hoogenboom et al., 1991, Nucl. Acids Res. 19: 4133-4137; sequence is available, e.g., as SEQ ID NO: 7 in WO 03/031611), in which the production of pIII fusion protein is under the control of the LacZ promoter, which is inhibited in the presence of glucose and induced with IPTG. When grown in suppressor strains of E. coli, e.g., TG1, the gene III fusion protein is produced and packaged into phage, while growth in non-suppressor strains, e.g., HB2151, permits the secretion of soluble fusion protein into the bacterial periplasm and into the culture medium. Because the expression of gene III prevents later infection with helper phage, the bacteria harboring the phagemid vectors are propagated in the presence of glucose before infection with VCSM13 helper phage for phage rescue.

Construction of vectors as set forth herein employs conventional ligation techniques. Isolated vectors or DNA fragments are cleaved, tailored, and re-ligated in the form desired to generate the required vector. If desired, sequence analysis to confirm that the correct sequences are present in the constructed vector is performed using standard methods. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridization, immunocytochemistry or sequence analysis of nucleic acid or protein molecules. Those skilled in the art will readily envisage how these methods may be modified, if desired.

### Screening Antibody Single Variable Domains, for Antigen Binding

Following expression of a repertoire of antibody single variable domains on the surface of phage, selection is performed by contacting the phage repertoire with immobilized target antigen, washing to remove unbound phage, and propagation of the bound phage, the whole process frequently referred to as “panning”. This process is applicable to the screening of antibody single variable domains as well as other antibody fragments that can be expressed on a display library, e.g., scFv, Fab, etc. Alternatively, phage are pre-selected for the expression of properly folded member variants by panning against an immobilized generic ligand (e.g., protein A or protein L) that is only bound by folded members. This has the advantage of reducing the proportion of non-functional members, thereby increasing the proportion of members likely to bind a target antigen. Pre-selection with generic ligands is taught in WO 99/20749, for example. The screening of phage antibody libraries is generally described, for example, by Harrison et al., 1996, Meth. Enzymol. 267: 83-109.

Screening is commonly performed using purified antigen immobilized on a solid support, for example, plastic tubes or wells, or on a chromatography matrix, for example Sepharose™ (Pharmacia). Screening or selection can also be performed on complex antigens, such as the surface of cells (Marks et al., 1993, BioTechnology 11: 1145; de Kruif et al., 1995, Proc. Natl. Acad. Sci. U.S.A. 92: 3938). Another alternative involves selection by binding biotinylated antigen in solution, followed by capture on streptavidin-coated beads.

In a preferred aspect, panning is performed by immobilizing antigen (generic or specific) on tubes or wells in a plate, e.g., Nunc MAXISORP™ immunotube 8 well strips. Wells are coated with 150 μl of antigen (100 μg/ml in PBS) and incubated overnight. The wells are then washed 3 times with PBS and blocked with 400 μl PBS-2% skim milk (2% MPBS) at 37° C. for 2 hr. The wells are rinsed 3 times with PBS and phage are added in 2% MPBS. The mixture is incubated at room temperature for 90 minutes and the liquid, containing unbound phage, is removed. Wells are rinsed 10 times with PBS-0.1% tween 20, and then 10 times with PBS to remove detergent. Bound phage are eluted by adding 200 μl of freshly prepared 100 mM triethylamine, mixing well and incubating for 10 min at room temperature. Eluted phage are transferred to a tube containing 100 μl of 1 M Tris-HCl, pH 7.4 and vortexed to neutralize the triethylamine. Exponentially-growing E. coli host cells (e.g., TG1) are infected with, for example, 150 ml of the eluted phage by incubating for 30 min at 37° C. Infected cells are spun down, resuspended in fresh medium and plated in top agarose. Phage plaques are eluted or picked into fresh cultures of host cells to propagate for analysis or for further rounds of selection. One or more rounds of plaque purification are performed if necessary to ensure pure populations of selected phage. Other screening approaches are described by Harrison et al., 1996, supra.

Following identification of phage expressing an antibody single variable domain that binds a desired target, if a phagemid vector such as pHEN1 has been used, the variable domain fusion proteins are easily produced in soluble form by infecting non-suppressor strains of bacteria, e.g., HB2151 that permit the secretion of soluble gene III fusion protein. If a GAS1 secretion signal peptide is encoded by the vector, the fusion polypeptide can be secreted by eukaryotic (e.g., yeast or mammalian) or prokaryotic (e.g., E. coli) cells. Alternatively, the V domain sequence can be sub-cloned into an appropriate expression vector to produce soluble protein according to methods known in the art.

### Purification and Concentration of Antibody Single Variable Domains

Antibody single variable domain polypeptides or other monovalent antibody single variable domains secreted into the periplasmic space or into the medium of bacteria are harvested and purified according to known methods (Harrison et al., 1996, supra). Skerra & Pluckthun (1988, Science 240: 1038) and Breitling et al. (1991, Gene 104: 147) describe the harvest of antibody single variable domains from the periplasm, and Better et al. (1988, Science 240: 1041) describes harvest from the culture supernatant. For some antibody single variable domains, purification can also be achieved by binding to generic ligands, such as protein A or Protein L. Alternatively, the variable domains can be expressed with a peptide tag, e.g., the Myc, HA or 6×-His tags, which facilitates purification by affinity chromatography.

If necessary, monovalent anti-CD28 antibody single variable domains are concentrated by any of several methods well known in the art, including, for example, ultrafiltration, diafiltration and tangential flow filtration. The process of ultrafiltration uses semi-permeable membranes and pressure to separate molecular species on the basis of size and shape. The pressure is provided by gas pressure or by centrifugation. Commercial ultrafiltration products are widely available, e.g., from Millipore (Bedford, Mass.; examples include the Centricon™ and Microcon™ concentrators) and Vivascience (Hannover, Germany; examples include the Vivaspin™ concentrators). By selection of a molecular weight cutoff smaller than the target polypeptide (usually ⅓ to ⅙ the molecular weight of the target polypeptide, although differences of as little as 10 kD can be used successfully), the polypeptide is retained when solvent and smaller solutes pass through the membrane. Thus, a molecular weight cutoff of about 5 kD is useful for concentration of anti-CD28 antibody single variable domain polypeptides described herein.

Diafiltration, which uses ultrafiltration membranes with a “washing” process, is used where it is desired to remove or exchange the salt or buffer in a polypeptide preparation. The polypeptide is concentrated by the passage of solvent and small solutes through the membrane, and remaining salts or buffer are removed by dilution of the retained polypeptide with a new buffer or salt solution or water, as desired, accompanied by continued ultrafiltration. In continuous diafiltration, new buffer is added at the same rate that filtrate passes through the membrane. A diafiltration volume is the volume of polypeptide solution prior to the start of diafiltration—using continuous diafiltration, greater than 99.5% of a fully permeable solute can be removed by washing through six diafiltration volumes with the new buffer. Alternatively, the process can be performed in a discontinuous manner, wherein the sample is repeatedly diluted and then filtered back to its original volume to remove or exchange salt or buffer and ultimately concentrate the polypeptide. Equipment for diafiltration and detailed methodologies for its use are available, for example, from Pall Life Sciences (Ann Arbor, Mich.) and Sartorius AG/Vivascience (Hannover, Germany).

Tangential flow filtration (TFF), also known as “cross-flow filtration,” also uses ultrafiltration membrane. Fluid containing the target polypeptide is pumped tangentially along the surface of the membrane. The pressure causes a portion of the fluid to pass through the membrane while the target polypeptide is retained above the filter. In contrast to standard ultrafiltration, however, the retained molecules do not accumulate on the surface of the membrane, but are carried along by the tangential flow. The solution that does not pass through the filter (containing the target polypeptide) can be repeatedly circulated across the membrane to achieve the desired degree of concentration. Equipment for TFF and detailed methodologies for its use are available, for example, from Millipore (e.g., the ProFlux M12™ Benchtop TFF system and the Pellicon™ systems), Pall Life Sciences (e.g., the Minim™ Tangential Flow Filtration system).

Protein concentration is measured in a number of ways that are well known in the art. These include, for example, amino acid analysis, absorbance at 280 nm, the “Bradford” and “Lowry” methods, and SDS-PAGE. The most accurate method is total hydrolysis followed by amino acid analysis by HPLC, concentration is then determined then comparison with the known sequence of the antibody single variable domain polypeptide. While this method is the most accurate, it is expensive and time-consuming. Protein determination by measurement of UV absorbance at 280 nm faster and much less expensive, yet relatively accurate and is preferred as a compromise over amino acid analysis. Absorbance at 280 nm was used to determine protein concentrations reported in the Examples described herein.

“Bradford” and “Lowry” protein assays (Bradford, 1976, Anal. Biochem. 72: 248-254; Lowry et al., 1951, J. Biol. Chem. 193: 265-275) compare sample protein concentration to a standard curve most often based on bovine serum albumin (BSA). These methods are less accurate, tending to underestimate the concentration of antibody single variable domains. Their accuracy could be improved, however, by using a V_{H} or V_{κ} single domain polypeptide as a standard.

An additional protein assay method that can be utilized is the bicinchoninic acid assay described in U.S. Pat. No. 4,839,295 (incorporated herein by reference) and marketed by Pierce Biotechnology (Rockford, Ill.) as the “BCA Protein Assay” (e.g., Pierce Catalog No. 23227).

The SDS-PAGE method uses gel electrophoresis and Coomassie Blue staining in comparison to known concentration standards, e.g., known amounts of an antibody single variable domain polypeptide. Quantitation can be done by eye or by densitometry.

Human antibody single variable domain polypeptides described herein retain solubility at high concentration (e.g., at least 4.8 mg (˜400 μM) in aqueous solution (e.g., PBS), and in an aspect, at least 5 mg/ml (˜417 μM), 10 mg/ml (˜833 μM), 20 mg/ml (˜1.7 mM), 25 mg/ml (˜2.1 mM), 30 mg/ml (˜2.5 mM), 35 mg/ml (˜2.9 mM), 40 mg/ml (˜3.3 mM), 45 mg/ml (˜3.75 mM), 50 mg/ml (˜4.2 mM), 55 mg/ml (˜4.6 mM), 60 mg/ml (˜5.0 mM), 65 mg/ml (˜5.4 mM), 70 mg/ml (˜5.8 mM), 75 mg/ml (˜6.3 mM), 100 mg/ml (˜8.33 mM), 150 mg/ml (˜12.5 mM), 200 mg/ml (˜16.7 mM), 240 mg/ml (˜20 mM) or higher). One structural feature that promotes high solubility is the relatively small size of the antibody single variable domain polypeptides. A full length conventional four chain antibody, e.g., IgG is about 150 kD in size. In contrast, antibody single variable domains, which all have a general structure comprising 4 framework (FW) regions and 3 CDRs, have a size of approximately 12 kD, or less than 1/10 the size of a conventional antibody. Similarly, antibody single variable domains are approximately half the size of a scFv molecule (˜26 kD), and approximately one-fifth the size of a Fab molecule (˜60 kD). It is preferred that the size of an antibody single variable domain-containing structure disclosed herein is 100 kD or less, including structures of, for example, about 90 kD or less, 80 kD or less, 70 kD or less, 60 kD or less, 50 kD or less, 40 kD or less, 30 kD or less, 20 kD or less, down to and including about 12 kD, or an antibody single variable domain in isolation.

The solubility of a polypeptide is primarily determined by the interactions of the amino acid side chains with the surrounding solvent. Hydrophobic side chains tend to be localized internally as a polypeptide folds, away from the solvent-interacting surfaces of the polypeptide. Conversely, hydrophilic residues tend to be localized at the solvent-interacting surfaces of a polypeptide. Generally, polypeptides having a primary sequence that permits the molecule to fold to expose more hydrophilic residues to the aqueous environment are more soluble than one that folds to expose fewer hydrophilic residues to the surface. Thus, the arrangement and number of hydrophobic and hydrophilic residues is an important determinant of solubility. Other parameters that determine polypeptide solubility include solvent pH, temperature, and ionic strength. In a common practice, the solubility of polypeptides can be maintained or enhanced by the addition of glycerol (e.g., ˜10% v/v) to the solution.

As discussed above, specific amino acid residues have been identified in conserved residues of human V_{H} domains that vary in the V_{H} domains of camelid species, which are generally more soluble than human V_{H} domains. These include, for example, Gly 44 (Glu in camelids), Leu 45 (Arg in camelids) and Trp 47 (Gly in camelids). Amino acid residue 103 of V_{H} is also implicated in solubility, with mutation from Trp to Arg tending to confer increased V_{H} solubility.

In preferred aspects as set forth herein, antibody single variable domain polypeptides are based on the DP47 germline V_{H} gene segment or the DPK9 germline V_{κ} gene segment. Thus, these germline gene segments are capable, particularly when diversified at selected structural locations described herein, of producing specific binding antibody single variable domain polypeptides that are highly soluble. In particular, the four framework regions, which are, in an aspect, not diversified, can contribute to the high solubility of the resulting proteins.

It is expected that a single human antibody variable domain that is highly homologous to one having a known high solubility will also tend to be highly soluble. Thus, as one means of prediction or recognition that a given antibody single variable domain would have the high solubility recited herein, one can compare the sequence of an antibody single variable domain polypeptide to one or more antibody single variable domain polypeptides having known solubility. Thus, when an antibody single variable domain polypeptide is identified that has high binding affinity but unknown solubility, comparison of its amino acid sequence with that of one or more (in an aspect, more) human antibody single variable domain polypeptides known to have high solubility (e.g., a dAb sequence disclosed herein) can permit prediction of its solubility. While it is not an absolute predictor, where there is a high degree of i to a known highly soluble sequence, e.g., 90-95% or greater similarity, and particularly where there is a high degree of similarity with respect to hydrophilic amino acid residues, or residues likely to be exposed at the solvent interface, it is more likely that a newly identified binding polypeptide will have solubility similar to that of the known highly soluble sequence.

Molecular modeling software can also be used to predict the solubility of a polypeptide sequence relative to that of a polypeptide of known solubility. For example, the substitution or addition of a hydrophobic residue at the solvent-exposed surface, relative to a molecule of known solubility that has a less hydrophobic or even hydrophilic residue exposed in that position is expected to decrease the relative solubility of the polypeptide. Similarly, the substitution or addition of a more hydrophilic residue at such a location is expected to increase the relative solubility. That is, a change in the net number of hydrophilic or hydrophobic residues located at the surface of the molecule (or the overall hydrophobic or hydrophilic nature of the surface-exposed residues) relative to an antibody single variable domain polypeptide structure with known solubility can predict the relative solubility of an antibody single variable domain polypeptide.

Alternatively, or in conjunction with such prediction, one can determine limits of an antibody single variable domain polypeptide's solubility by simply concentrating the polypeptide.

### Affinity Determination

Isolated antibody single variable domain polypeptide-containing polypeptides as described herein, in an aspect, have affinities (dissociation constant, K_{d},=K_{off}/Kₒₙ) of at least 500 nM or less, and in an aspect, at least 400 nM-50 pM, 300 nM-50 pM, 200 nM-50 pM, and in a further aspect, at least 100 nM-50 pM, 75 nM-50 pM, 50 nM-50 pM, 25 nM-50 pM, 10 nM-50 pM, 5 nM-50 pM, 1 nM-50 pM, 950 pM-50 pM, 900 pM-50 pM, 850 pM-50 pM, 800 pM-50 pM, 750 pM-50 pM, 700 pM-50 pM, 650 pM-50 pM, 600 pM-50 pM, 550 pM-50 pM, 500 pM-50 pM, 450 pM-50 pM, 400 pM-50 pM, 350 pM-50 pM, 300 pM-50 pM, 250 pM-50 pM, 200 pM-50 pM, 150 pM-50 pM, 100 pM-50 pM, 90 pM-50 pM, 80 pM-50 pM, 70 pM-50 pM, 60 pM-50 pM, or even as low as 50 pM.

The antigen-binding affinity of an antibody single variable domain, e.g., an antibody single variable domain polypeptide or other monovalent antibody single variable domain, can be conveniently measured by Surface Plasmon Resonance (SPR) using the Biacore system (Pharmacia Biosensor, Piscataway, N.J.). In this method, antigen is coupled to the Biacore chip at known concentrations, and variable domain polypeptides are introduced. Specific binding between the single variable domain polypeptide and the immobilized antigen results in increased protein concentration on the chip matrix and a change in the SPR signal. Changes in SPR signal are recorded as resonance units (RU) and displayed with respect to time along the Y axis of a sensorgram. Baseline signal is taken with solvent alone (e.g., PBS) passing over the chip. The net difference between baseline signal and signal after completion of antibody single variable domain injection represents the binding value of a given sample. To determine the off rate (K_{off}), on rate (Kₒₙ) and dissociation rate (k_{d}) constants, Biacore kinetic evaluation software (e.g., version 2.1) is used.

Thus, SPR can be used to monitor antagonism of CD28 binding to CD80 or CD86 by a monovalent anti-CD28 antibody preparation by measuring the displacement or inhibition of binding of CD28 to CD80 or CD86 caused the monovalent antibody preparation. SPR can also be used to monitor the dimerization, or in an aspect, the lack of dimerization, occurring via Fc region in antibody preparations as described herein.

High affinity is dependent upon the complementarity between a surface of the antigen and the CDRs of the antibody or antibody fragment. Complementarity is determined by the type and strength of the molecular interactions possible between portions of the target and the CDR, for example, the potential ionic interactions, van der Waals attractions, hydrogen bonding or other interactions that can occur. CDR3 tends to contribute more to antigen binding interactions than CDRs 1 and 2, probably due to its generally larger size, which provides more opportunity for favorable surface interactions. (See, e.g., Padlan et al., 1994, Mol. Immunol. 31: 169-217; Chothia & Lesk, 1987, J. Mol. Biol. 196: 904-917; and Chothia et al., 1985, J. Mol. Biol. 186: 651-663.) High affinity indicates antibody single variable domain/antigen pairings that have a high degree of complementarity, which is directly related to the structures of the variable domain and the target.

In one aspect, a monovalent anti-CD28 antibody single variable domain, e.g., an antibody single variable domain polypeptide, is linked to another antibody single variable domain to form a heterodimer in which each individual antibody single variable domain is capable of binding a different cognate antigen. Fusing antibody single variable domains, such as antibody single variable domains, as heterodimers, wherein each monomer binds a different target antigen, can produce a dual-specific ligand capable, for example, of bridging the respective target antigens. Such dual specific ligands may be used to target cytokines and other molecules which cooperate synergistically in therapeutic situations in the body of an organism. Thus, there is provided a method for synergizing the activity of two or more cytokines, comprising administering a dual specific antibody heterodimer capable of binding to the two or more cytokines.

Anti-CD28 antibody single variable domains set forth herein include CD28-binding antibody single variable domain clones, and clones with substantial sequence identity that also bind target antigen with high affinity.

An additional measure of identity is the ability to hybridize under highly stringent hybridization conditions. Thus, a first sequence encoding an antibody single variable domain polypeptide is substantially similar to a second coding sequence if the first sequence hybridizes to the second sequence (or its complement) under highly stringent hybridization conditions (such as those described by Sambrook et al., Molecular Cloning, Laboratory Manuel, Cold Spring, Harbor Laboratory press, New York). “Highly stringent hybridization conditions” refer to hybridization in 6×SSC at about 45° C., followed by one or more washes in 0.2×SSC, 0.1% SDS at 65° C. “Very highly stringent hybridization conditions” refer to hybridization in 0.5M sodium phosphate, 7% SDS at 65° C., followed by one or more washes at 0.2×SSC, 1% SDS at 65° C.

### 4.0 Assays for CD28 Activities

In an exemplary embodiment, a monovalent anti-CD28 antibody single variable domain as described herein binds to CD28 yet does not substantially agonize CD28 signaling. Activation of the CD28 pathway manifests a number of different outcomes that can be measured in order to assess the effect of a given monovalent anti-CD28 antibody single variable domain on the activity of the pathway. However, for the assessment of the antagonist or agonist function of monovalent anti-CD28 antibody single variable domains described herein, at least one of the following CD28 assays can be used.

In an embodiment, activation of T cells is measured. In the assay, human CD4 positive T-cells are stimulated with anti-CD3 plus transfected CHO cells expressing either CD80 or CD86. This results in proliferation of the T cells and is CD28 dependent as anti-CD28 mAbs block the proliferation response.

In another embodiment, induction of T cell proliferation and induction of cytokine secretion is measured. The assay comprises stimulation of human CD4 positive T cells with anti-CD28 mAb 9.3 (Gibson, et al. (1996) JBC, 271:7079-7083). This results in upregulation of T cell receptor-mediated signaling and secretion of cytokines and is CD28-dependent, as mAb 9.3 blocks the proliferation response. Secreted cytokines that may be measured include, but are not limited to, IL-2, IL-6, IL-10, IL-13, TNF-α and IFN-γ. One or more of such cytokines may be detected and/or measured according to the disclosure set forth herein.

It will be understood, based on the disclosure herein, that anti-CD28 antibody single variable domains set forth herein can possess multiple functions and activities, and therefore, may be assayed by multiple distinct assays. As set forth in detail elsewhere herein, anti-CD28 antibody single variable domains have multiple defining characteristics (e.g., CD28 binding affinity, CDR domain identity, and amino acid sequence, among others), and therefore, each distinct polypeptide can be characterized in multiple ways and through multiple parameters. The characterization of each such polypeptide, alone or in conjunction with the activity and/or CD28 binding properties of the polypeptide, can therefore provide unique identifying characteristics for the polypeptide.

### 5.0 PEGylation of CD28 Monovalent Binders

Also provided herein are PEGylated monovalent anti-CD28 antibody single variable domains which have increased half-life and in an aspect, also resistance to degradation without a loss in activity (e.g., binding affinity) relative to non-PEGylated antibody single variable domains.

Both site-specific and random PEGylation of protein molecules is known in the art (See, for example, Zalipsky and Lee, Poly(ethylene glycol) Chemistry: Biotechnical and Biomedical Applications 1992, pp 347-370, Plenum, NY; Goodson and Katre, 1990, Bio/Technology, 8:343; Hershfield et al., 1991, PNAS 88:7185). More specifically, random PEGylation of antibody molecules has been described at lysine residues and thiolated derivatives (Ling and Mattiasson, 1983, Immunol. Methods 59: 327; Wilkinson et al., 1987, Immunol. Letters, 15: 17; Kitamura et al., 1991, Cancer Res. 51:4310; Delgado et al., 1996 Br. J. Cancer, 73: 175; Pedley et al., 1994, Br. J. Cancer, 70:1126)

Accordingly, monovalent anti-CD28 antibody single variable domains according to this aspect can be coupled, using methods known in the art to polymer molecules (in an aspect, PEG) useful for achieving the increased half-life and degradation resistance properties encompassed herein. Polymer moieties which can be utilized can be synthetic or naturally occurring and include, but are not limited to straight or branched chain polyalkylene, polyalkenylene or polyoxyalkylene polymers, or a branched or unbranched polysaccharide such as a homo- or heteropolysaccharide. Preferred examples of synthetic polymers which may be used include straight or branched chain poly(ethylene glycol) (PEG), polypropylene glycol), or poly(vinyl alcohol) and derivatives or substituted forms thereof. Particularly preferred substituted polymers useful include substituted PEG, including methoxy(polyethylene glycol). Naturally occurring polymer moieties which may be used herein in addition to or in place of PEG include lactose, amylose, dextran, or glycogen, as well as derivatives thereof which would be recognized by one of skill in the art. Derivatized forms of polymer molecules as set forth herein include, for example, derivatives which have additional moieties or reactive groups present therein to permit interaction with amino acid residues of the dAb polypeptides described herein. Such derivatives include N-hydroxylsuccinimide (NHS) active esters, succinimidyl propionate polymers, and sulfhydryl-selective reactive agents such as maleimide, vinyl sulfone, and thiol. Particularly preferred derivatized polymers include, but are not limited to PEG polymers having the formulae: PEG-O—CH₂CH₂CH₂—CO₂—NHS; PEG-O—CH₂—NHS; PEG-O—CH₂CH₂—CO₂—NHS; PEG-S—CH₂CH₂—CO—NHS; PEG-O2CNH—CH(R)—CO₂—NHS; PEG-NHCO—CH₂CH₂—CO—NHS; and PEG-O—CH₂—CO₂—NHS; where R is (CH₂)₄)NHCO2(mPEG). PEG polymers useful as set forth herein may be linear molecules, or may be branched wherein multiple PEG moieties are present in a single polymer. Some particularly preferred PEG derivatives which are useful as set forth herein include, but are not limited to mPEG-MAL, mPEG2-MAL, mPEG-(MAL)², multi-arm PEG, mPEG-SPA, mPEG2-NHS, and mPEG2-(MAL)², illustrated below:

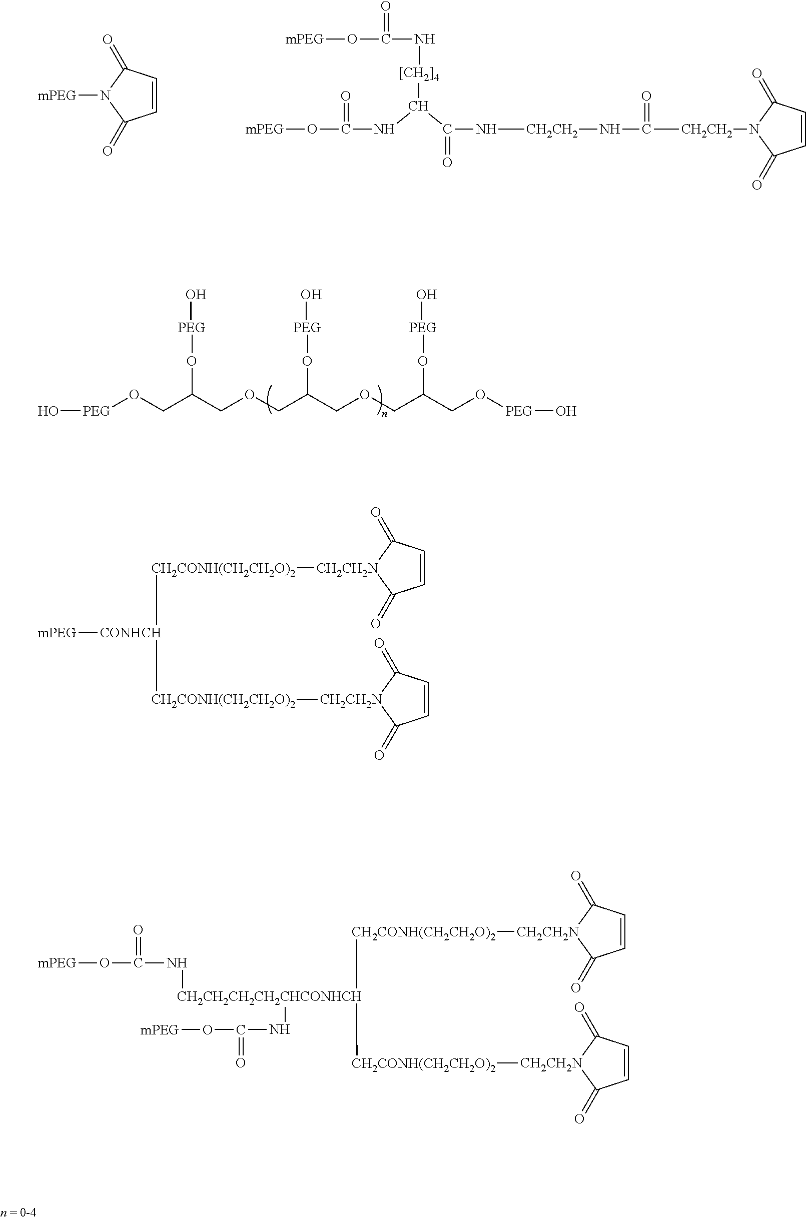

The reactive group (e.g., MAL, NHS, SPA, VS, or Thiol) may be attached directly to the PEG polymer or may be attached to PEG via a linker molecule.

The size of polymers useful as set forth herein can be in the range of between 500 Da to 60 kDa, for example, between 1000 Da and 60 kDa, 10 kDa and 60 kDa, 20 kDa and 60 kDa, 30 kDa and 60 kDa, 40 kDa and 60 kDa, and up to between 50 kDa and 60 kDa. The polymers used herein, particularly PEG, can be straight chain polymers or can possess a branched conformation. Depending on the combination of molecular weight and conformation, the polymer molecules useful as set forth herein, when attached to a monovalent anti-CD28 antibody single variable domain, will yield a molecule having an average hydrodynamic size of between 24 and 500 kDa. The hydrodynamic size of a polymer molecule used herein refers to the apparent size of a molecule (e.g., a protein molecule) based on the diffusion of the molecule through an aqueous solution. The diffusion, or motion of a protein through solution can be processed to derive an apparent size of the protein, where the size is given by the Stokes radius or hydrodynamic radius of the protein particle. The “hydrodynamic size” of a protein depends on both mass and shape (conformation), such that two proteins having the same molecular mass may have differing hydrodynamic sizes based on the overall conformation of the protein. The hydrodynamic size of a PEG-linked monovalent anti-CD28 antibody single variable domain, e.g., an anti-CD28 antibody single variable domain as described herein, can be in the range of 24 kDa to 500 kDa; 30 to 500 kDa; 40 to 500 kDa; 50 to 500 kDa; 100 to 500 kDa; 150 to 500 kDa; 200 to 500 kDa; 250 to 500 kDa; 300 to 500 kDa; 350 to 500 kDa; 400 to 500 kDa, and 450 to 500 kDa. In an exemplary embodiment, the hydrodynamic size of a PEGylated antibody single variable domain as described herein is 30 to 40 kDa; 70 to 80 kDa or 200 to 300 kDa. The size of a polymer molecule attached to a monovalent anti-CD28 antibody single variable domain may thus be varied depending upon the desired application. For example, where the PEGylated antibody single variable domain is intended to leave the circulation and enter into peripheral tissues, it is desirable to keep the size of the attached polymer low to facilitate extravazation from the blood stream. Alternatively, where it is desired to have the PEGylated antibody single variable domain remain in the circulation for a longer period of time, a higher molecular weight polymer can be used (e.g., a 30 to 60 kDa polymer).

The polymer (PEG) molecules useful as set forth herein can be attached to antibody single variable domains using methods that are well known in the art. The first step in the attachment of PEG or other polymer moieties to an antibody single variable domain is the substitution of the hydroxyl end-groups of the PEG polymer by electrophile-containing functional groups. Particularly, PEG polymers are attached to either cysteine or lysine residues present in the antibody single variable domain. The cysteine and lysine residues can be naturally occurring, or can be engineered into the antibody single variable domain molecule. For example, cysteine residues can be recombinantly engineered at the C-terminus of antibody single variable domains, or residues at specific solvent accessible locations in the antibody single variable domain can be substituted with cysteine or lysine. In a preferred embodiment, a PEG moiety is attached to a cysteine residue which is present in the hinge region at the C-terminus of an antibody single variable domain.

In a further preferred embodiment a PEG moiety or other polymer is attached to a cysteine or lysine residue which is either naturally occurring at or engineered into the N-terminus of antibody single variable domain polypeptide as set forth herein. In a still further embodiment, a PEG moiety or other polymer is attached to an antibody single variable domain as set forth herein at a cysteine or lysine residue (either naturally occurring or engineered) which is at least 2 residues away from (e.g., internal to) the C- and/or N-terminus of the antibody single variable domain polypeptide.

In one embodiment, the PEG polymer(s) is attached to one or more cysteine or lysine residues present in a framework region (FR or FWs) and one or more heterologous CDRs of an antibody single variable domain. CDRs and framework regions (e.g., CDR1-CDR3 and FW1-FW4) are those regions of an antibody variable domain as defined in the Kabat database of Sequences of Proteins of Immunological Interest (Kabat et al., 1991, Sequences of Immunological Interest, 5^{th} ed. U.S. Dept. Health & Human Services, Washington, D.C.). In a preferred embodiment, a PEG polymer is linked to a cysteine or lysine residue in the V_{H} framework segment DP47, or the Vₖ framework segment DPK9. Cysteine and/or lysine residues of DP47 which may be linked to PEG disclosed herein include the cysteine at positions 22, or 96 and the lysine at positions 43, 65, 76, or 98 of SEQ ID NO: 1. Cysteine and/or lysine residues of DPK9 which may be linked to PEG disclosed herein include the cysteine residues at positions 23, or 88 and the lysine residues at positions 39, 42, 45, 103, or 107 of SEQ ID NO: 3. In addition, specific cysteine or lysine residues may be linked to PEG in the V_{H} canonical framework region DP38, or DP45.

In addition, specific solvent accessible sites in the antibody molecule which are not naturally occurring cysteine or lysine residues may be mutated to a cysteine or lysine for attachment of a PEG polymer. Solvent accessible residues in any given antibody, e.g., a dAb, can be determined using methods known in the art such as analysis of the crystal structure of the antibody single variable domain. For example, using the solved crystal structure of the V_{H} dAb HEL4 (SEQ ID NO: 327; a dAb that binds hen egg lysozyme), the residues Gln-13, Pro-14, Gly-15, Pro-41, Gly-42, Lys-43, Asp-62, Lys-65, Arg-87, Ala-88, Glu-89, Gln-112, Leu-115, Thr-117, Ser-119, and Ser-120 have been identified as being solvent accessible, and disclosed herein would be attractive candidates for mutation to cysteine or lysine residues for the attachment of a PEG polymer. In addition, using the solved crystal structure of the Vₖ dummy dAb (SEQ ID NO: 328), the residues Val-15, Pro-40, Gly-41, Ser-56, Gly-57, Ser-60, Pro-80, Glu-81, Gln-100, Lys-107, and Arg-108 have been identified as being solvent accessible, and disclosed herein would be attractive candidates for mutation to cysteine or lysine residues for the attachment of a PEG polymer. In one embodiment as disclosed herein, a PEG polymer is linked to multiple solvent accessible cysteine or lysine residues, or to solvent accessible residues which have been mutated to a cysteine or lysine residue. Alternatively, only one solvent accessible residue is linked to PEG, either where the particular antibody single variable domain only possesses one solvent accessible cysteine or lysine (or residue modified to a cysteine or lysine) or where a particular solvent accessible residue is selected from among several such residues for PEGylation.

| Primary amino acid sequence of HEL4. | |
|---|---|
| (SEQ ID NO: 327) | |
| 1 | EVQLLESGGG LVQPGGSLRL SCAASGFRIS DEDMGWVRQA PGKGLEWVSS |
| 51 | IYGPSGSTYY ADSVKGRFTI SRDNSKNTLY LQMNSLRAED TAVYYCASAL |
| 101 | EPLSEPLGFW GQGTLVTVSS |
| Primary amino acid sequence of Vₖ dummy. | |
| (SEQ ID NO: 328) | |
| 1 | DIQMTQSPSS LSASVGDRVT ITCRASQSIS SYLNWYQQKP GKAPKLLIYA |
| 51 | ASSLQSGVPS RFSGSGSGTD FTLTISSLQP EDFATYYCQQ SYSTPNTFGQ |
| 101 | GTKVEIKR |

Several PEG attachment schemes disclosed herein are provided by the company Nektar (SanCarlos, Calif.). For example, where attachment of PEG or other polymer to a lysine residue is desired, active esters of PEG polymers which have been derivatized with N-hydroxylsuccinimide, such as succinimidyl propionate may be used. Where attachment to a cysteine residue is intended, PEG polymers which have been derivatized with sulfhydryl-selective reagents such as maleimide, vinyl sulfone, or thiols may be used. Other examples of specific embodiments of PEG derivatives which may be used as disclosed herein to generate PEGylated antibodies can be found in the Nektar Catalog (available on the world wide web at nektar.com). In addition, several derivitized forms of PEG may be used as disclosed herein to facilitate attachment of the PEG polymer to an antibody single variable domain. PEG derivatives disclosed herein include, but are not limited to PEG-succinimidyl succinate, urethane linked PEG, PEG phenylcarbonate, PEG succinimidyl carbonate, PEG-carboxymethyl azide, dimethylmaleic anhydride PEG, PEG dithiocarbonate derivatives, PEG-tresylates (2,2,2-trifluoroethanesolfonates), mPEG imidoesters, and other as described in Zalipsky and Lee, (1992) (“Use of functionalized poly(ethylene glycol)s for modification of peptides” in Poly(Ethylene Glycol) Chemistry: Biotechnical and Biomedical Applications, J. Milton Harris, Ed., Plenum Press, NY).

In one embodiment disclosed herein, an anti-CD28 antibody single variable domain composition comprising an antibody single variable domain and PEG polymer wherein the ratio of PEG polymer to antibody single variable domain is a molar ratio of at least 0.25:1. In a further embodiment, the molar ratio of PEG polymer to antibody single variable domain is 0.33:1 or greater. In a still further embodiment the molar ratio of PEG polymer to antibody single variable domain is 0.5:1 or greater.

6.0 Modification of CD28 monovalent binders 6.1.0 Diversification of the Canonical Sequence

Having selected several known main-chain conformations or, in an aspect, a single known main-chain conformation, ligands disclosed herein or libraries for use herein can be constructed by varying the binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or are in an aspect, selected. The variation can then be achieved either by randomization, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomized to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomization has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

In a preferred embodiment, only those residues which are directly involved in creating or modifying the desired function of the molecule are diversified. For many molecules, the function will be to bind a target and therefore diversity should be concentrated in the target binding site, while avoiding changing residues which are crucial to the overall packing of the molecule or to maintaining the chosen main-chain conformation.

### 6.1.1 Diversification of the Canonical Sequence as it Applies to Antibody Domains

In the case of the ligands disclosed herein, the binding site for the target is most often the antigen binding site. Thus, in a highly preferred aspect, libraries of or for the assembly of antibody ligands in which only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (Kabat et al., 1991, Sequences of Immunological Interest, 5^{th} ed. U.S. Dept. Health & Human Services, Washington, D.C.), some seven residues compared to the two diversified in the library for use as disclosed herein. This represents a significant improvement in terms of the functional diversity required to create a range of antigen binding specificities.

In nature, antibody diversity is the result of two processes: somatic recombination of germline V, D, and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) J. Mol. Biol., 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire is created where some, but not all, of the residues in the antigen binding site are diversified. As used herein in this context, the term “naive” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

Disclosed herein are two different naive repertoires of binding domains for the construction of ligands, or a naïve library of ligands, in which some or all of the residues in the antigen binding site are varied. The “primary” library mimics the natural primary repertoire, with diversity restricted to residues at the centre of the antigen binding site that are diverse in the germline V gene segments (germline diversity) or diversified during the recombination process (junctional diversity). Those residues which are diversified include, but are not limited to, HS0, H52, H52a, H53, H55, H56, H58, H95, H96, H97, H98, L50, L53, L91, L92, L93, L94, and L96. In the “somatic” library, diversity is restricted to residues that are diversified during the recombination process (junctional diversity) or are highly somatically mutated). Those residues which are diversified include, but are not limited to: H31, H33, H35, H95, H96, H97, H98, L30, L31, L32, L34, and L96. All the residues listed above as suitable for diversification in these libraries are known to make contacts in one or more antibody-antigen complexes. Since in both libraries, not all of the residues in the antigen binding site are varied, additional diversity is incorporated during selection by varying the remaining residues, if it is desired to do so. It shall be apparent to one skilled in the art that any subset of any of these residues (or additional residues which comprise the antigen binding site) can be used for the initial and/or subsequent diversification of the antigen binding site.

In the construction of libraries for use as disclosed herein, diversification of chosen positions is typically achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is, in an aspect, used in order to introduce the required diversity. Other codons which achieve the same ends are also of use, including the NNN codon, which leads to the production of the additional stop codons TGA and TAA.

A feature of side-chain diversity in the antigen binding site of human antibodies is a pronounced bias which favors certain amino acid residues. If the amino acid composition of the ten most diverse positions in each of the V_{H}, V_{K}, and Vₓ regions are summed, more than 76% of the side-chain diversity comes from only seven different residues, these being, serine (24%), tyrosine (14%), asparagine (11%), glycine (9%), alanine (7%), aspartate (6%), and threonine (6%). This bias towards hydrophilic residues and small residues which can provide main-chain flexibility probably reflects the evolution of surfaces which are predisposed to binding a wide range of antigens or epitopes and may help to explain the required promiscuity of antibodies in the primary repertoire.

Since it is preferable to mimic this distribution of amino acids, the distribution of amino acids at the positions to be varied, in an aspect, mimics that seen in the antigen binding site of antibodies. Such bias in the substitution of amino acids that permits selection of certain polypeptides (not just antibody single variable domains) against a range of target antigens is easily applied to any polypeptide repertoire. There are various methods for biasing the amino acid distribution at the position to be varied (including the use of tri-nucleotide mutagenesis, see WO 97/08320), of which the preferred method, due to ease of synthesis, is the use of conventional degenerate codons. By comparing the amino acid profile encoded by all combinations of degenerate codons (with single, double, triple, and quadruple degeneracy in equal ratios at each position) with the natural amino acid use it is possible to calculate the most representative codon. The codons (AGT)(AGC)T, (AGT)(AGC)C, and (AGT)(AGC)(CT) are those closest to the desired amino acid profile: they encode 22% serine and 11% tyrosine, asparagine, glycine, alanine, aspartate, threonine, and cysteine, and in an aspect, these codons are used in the construction of a library.

6.2.0 Dual-Specific Polypeptides comprising multiple antibody single variable domains

Also provided herein are dual-specific polypeptides comprising antibody single variable domains which each have respective specificities; that is, the first and the second epitopes bound by the dual-specific ligand are in an aspect, different or are two copies of the same epitopes being bound by a respective variable domain. As used herein a “dual-specific polypeptide” refers to a polypeptide comprising a first antibody single variable domain and a second antibody single variable domain as herein defined, wherein each of the variable domains is capable of binding one of two different antigens or one of two epitopes on the same antigen. For example, the two epitopes may be on the same antigen. The dual specific polypeptides disclosed herein are composed of antibody single variable domains which have different specificities, and do not contain mutually complementary variable domain pairs which have the same specificity. Dual-specific polypeptides may be, or be part of, polypeptides, proteins, or nucleic acids, which may be naturally occurring or synthetic. In this respect, the antibody single variable domains disclosed herein may bind an epitope or antigen and act as an antagonist or agonist (e.g., CD28 antagonist). The epitope binding domains of the dual specific polypeptides in one embodiment have the same epitope specificity, and may for example simultaneously bind their epitope when multiple copies of the epitope are present on the same antigen. In another embodiment, these epitopes are provided on different antigens such that the ligand can bind the epitopes and bridge the antigens. One skilled in the art will appreciate that the choice of epitopes and antigens is large and varied. They may be for instance human or animal proteins, cell surface proteins, cytokines, cytokine receptors, enzymes co-factors for enzymes or DNA binding proteins.

In one embodiment of the dual specific polypeptides disclosed herein, the variable domains are derived from an antibody directed against the first and/or second antigen or epitope. In a preferred embodiment the variable domains are derived from a repertoire of single variable antibody domains. In one example, the repertoire is a repertoire that is not created in an animal or a synthetic repertoire. In another example, the single variable domains are not isolated (at least in part) by animal immunization. Thus, the antibody single variable domains can be isolated from a naïve library.

In another aspect, disclosed herein is a multi-specific polypeptide comprising a first epitope binding antibody single variable domain having a first epitope binding specificity and a non-complementary second epitope binding antibody single variable domain having a second epitope binding specificity. The first and second binding specificities may be the same or different.

In a further aspect, disclosed herein a closed conformation multi-specific polypeptide comprising a first epitope antibody single variable domain having a first epitope binding specificity and a non-complementary second epitope antibody single variable domain having a second epitope binding specificity wherein the first and second binding specificities are capable of competing for epitope binding such that the closed conformation multi-specific polypeptide cannot bind both epitopes simultaneously.

In a still further aspect, disclosed herein re open conformation polypeptides comprising non-complementary antibody single variable domains, wherein the domains are specific for a different epitope on the same target. Such polypeptides bind to targets with increased avidity. Similarly, disclosed herein are multivalent polypeptides comprising non-complementary antibody single variable domains specific for the same epitope and directed to targets which comprise multiple copies of said epitope.

In a similar aspect, polypeptides comprising non-complementary antibody single variable domains disclosed herein can be configured to bind individual epitopes with low affinity, such that binding to individual epitopes is not therapeutically significant; but the increased avidity resulting from binding to two epitopes provides a therapeutic benefit. In a particular example, epitopes may be targeted which are present individually on normal cell types, but present together only on abnormal or diseased cells, such as tumor cells. In such a situation, only the abnormal or diseased cells are effectively targeted by the bispecific polypeptides comprising non-complementary antibody single variable domains disclosed herein.

Polypeptides comprising non-complementary antibody single variable domains specific for multiple copies of the same epitope, or adjacent epitopes, on the same target (known as chelating dAbs) may also be trimeric or polymeric (tetrameric or more), that is comprising three, four or more non-complementary antibody single variable domains. For example, ligands may be constructed comprising three or four V_{H} domains or V_{L} domains.

Moreover, polypeptides comprising non-complementary antibody single variable domains are provided which bind to multisubunit targets, wherein each binding domain is specific for a subunit of said target. The ligand may be dimeric, trimeric, or polymeric.

Also disclosed herein are dual specific polypeptides comprising a first antibody single variable domain having a binding specificity to a first antigen and a second antibody single variable domain having a binding specificity to a second antigen, wherein the first antigen is CD28 and the second single variable domain is an antigen presenting cell surface antigen or a T cell surface antigen, e.g., T cell receptor. The antigen presenting cell (APC) surface antigen can be selected from one of the group consisting of dendritic cell surface antigens, activated macrophage surface antigens, activated B cell surface antigens, co-stimulatory signal pathway surface antigens, and MHC, such as MHC II alpha or beta.

In an exemplary embodiment, the multi-specific polypeptides according to the above aspects disclosed herein are obtainable by the method comprising the steps of:
a) selecting a first epitope binding single variable domain by its ability to bind to a first epitope,
b) selecting a second epitope binding single variable domain by its ability to bind to a second epitope,
c) combining the epitope binding single variable domains; and
d) selecting the closed conformation multispecific polypeptide by its ability to bind to said first second epitope and said second epitope.

Advantageously the first epitope binding single variable domain and the second epitope binding single variable domains are non-complementary antibody variable domains, where each single variable domain binds its antigen or cognate ligand independently of the second single variable domain as herein defined. For example, such a multi-specific polypeptide may have the following nonlimiting formats: V_{H}-V_{H} or V_{L}-V_{L} single variable domains.

Chelating polypeptides comprising or consisting of antibody single variable domains in particular may be prepared according to a preferred aspect disclosed herein, namely the use of anchor antibody single variable domains, in which a library of dimeric, trimeric, or multimeric antibody single variable domains is constructed using a vector which comprises a constant antibody single variable domain upstream or downstream of a linker sequence, with a repertoire of second, third, and further antibody single variable domains being inserted on the other side of the linker. In alternative methodologies, the use of linkers may be avoided, for example by the use of non-covalent bonding or natural affinity between binding domains such as V_{H} and V_{K}. Accordingly, a method is provided for preparing a multimeric polypeptide comprising antibody single variable domains comprising the steps of:
(a) providing a vector comprising a nucleic acid sequence encoding an antibody single variable domain specific for a first epitope on a target;
(b) providing a vector encoding a repertoire comprising second an antibody single variable domain specific for a second epitope on said target, which epitope can be the same or different to the first epitope, said second epitope being adjacent to said first epitope; and
(c) expressing said first and second antibody single variable domains; and
(d) isolating those combinations of first and second an antibody single variable domains which combine together to produce a target-binding dimer.

The first and second epitopes are adjacent such that such a multimeric polypeptide is capable of binding to both epitopes simultaneously, providing the advantages of increased avidity of binding. Where the epitopes are the same, the increased avidity is obtained by the presence of multiple copies of the epitope on the target, allowing at least two copies to be simultaneously bound in order to obtain the increased avidity effect.

In an alternative embodiment of the above aspect of the second configuration disclosed herein, at least one epitope binding domain comprises a non-immunoglobulin ‘protein scaffold’ or ‘protein skeleton’ as herein defined. Suitable non-immunoglobulin protein scaffolds include but are not limited to any of those selected from the group consisting of: SpA, fibronectin, GroEL and other chaperones, lipocallin, CTLA4, and affibodies, as set forth above.

According to the above aspect of the second configuration disclosed herein, advantageously, the epitope binding domains are attached to a ‘protein skeleton’. Advantageously, a protein skeleton disclosed herein is an immunoglobulin skeleton.

As used herein, the term ‘immunoglobulin skeleton’ refers to a protein which comprises at least one immunoglobulin fold and which acts as a nucleus for one or more epitope binding domains, as defined herein.

Examples of immunoglobulin skeletons as herein defined includes any one or more of those selected from the following: an immunoglobulin molecule comprising at least (i) the CL (kappa or lambda subclass) domain of an antibody; or (ii) the CH1 or the CH2 or the CH3 domain of an antibody heavy chain; an immunoglobulin molecule comprising the CH1 and CH2 domains of the CH2 and CH3 domains, or the CH1 and CH3 domains of an antibody heavy chain; an immunoglobulin molecule comprising the CH1, CH2 and CH3 domains of an antibody heavy chain; or any of the subset (ii) in conjunction with the CL (kappa or lambda subclass) domain of an antibody. A hinge region domain may also be included. Such combinations of domains may, for example, mimic natural antibodies, such as IgG or IgM, or fragments thereof, such as Fv, scFv, Fab, or F(ab′)₂ molecules. Those skilled in the art will be aware that this list is not intended to be exhaustive.

Linking of the skeleton to the epitope binding domains, as herein defined may be achieved at the polypeptide level, that is after expression of the nucleic acid encoding the skeleton and/or the epitope binding domains. Alternatively, the linking step may be performed at the nucleic acid level. Methods of linking a protein skeleton disclosed herein, to the one or more epitope binding domains include the use of protein chemistry and/or molecular biology techniques which will be familiar to those skilled in the art and are described herein.

Advantageously, the dual- or multispecific polypeptides comprising multiple antibody single variable domains, may comprise a first domain capable of binding a target molecule, e.g., CD28, and a second domain capable of binding a molecule or group which extends the half-life of the ligand. For example, the molecule or group may be a bulky agent, such as human serum albumin (HSA) or a cell matrix protein. As used herein, the phrase “molecule or group which extends the half-life of an antibody single variable domain or polypeptide comprising an antibody single variable domain” refers to a molecule or chemical group which, when bound by a dual-specific or multi-specific polypeptide comprising multiple single variable domains as described herein, increases the in vivo half-life of such a dual specific polypeptide when administered to an animal, relative to a respective dual or multi specific polypeptide that does not bind that molecule or group. Examples of molecules or groups that extend the half-life of a dual specific polypeptide are described herein below. In a preferred embodiment, the closed conformation multispecific or dual specific polypeptide may be capable of binding the target molecule only on displacement of the half-life enhancing molecule or group. Thus, for example, a closed conformation multispecific polypeptide comprising multiple antibody single variable domains is maintained in circulation in the bloodstream of a subject by a bulky molecule such as HSA. When a target molecule is encountered, competition between the binding domains of the closed conformation multispecific polypeptide comprising multiple antibody single variable domains results in displacement of the HSA and binding of the target. Molecules which increase half-life are discussed in further detail above.

Antibody single variable domains and/or polypeptides comprising antibody single variable domains according to any aspect disclosed herein, as well as antibody single variable domain monomers useful in constructing such polypeptides, may advantageously dissociate from their cognate target(s) with a K_{d} of 300 nM to 1 pM or 5 pM (i.e., 3×10″⁷ to 5×10⁻¹² M), in an aspect, 50 nM to 20 pM, or 5 nM to 200 pM or 1 nM to 100 pM, 1×10⁻⁷ M or less, 1×10⁻⁸ M or less, 1×10⁻⁹ M or less, 1×10⁻¹⁰ M or less, 1×10⁻¹¹ M or less; and/or a K_{off} rate constant of 5×10⁻¹ to 1×10⁻⁷S⁻¹, in an aspect, 1×10⁻² to 1×10⁻⁶ S⁻¹, or 5×10⁻³ to 1×10⁻⁵ S⁻¹, or 5×10⁻¹S⁻¹ or less, or 1×10⁻²S⁻¹ or less, or 1×10⁻³S⁻¹ or less, or 1×10⁻⁴S⁻¹ or less, or 1×10⁻⁵S⁻¹ or less, or 1×10⁻⁶S⁻¹ or less as determined by surface plasmon resonance. The K_{d} rate constant is defined as K_{off}/Kₒₙ.

The invention in one embodiment provides a polypeptide or antagonist (e.g., dual specific polypeptide comprising an anti-CD28 antibody single variable domain (a first antibody single variable domain)) that binds to CD28 and a second antibody single variable domain that binds serum albumin (SA), the second dAb binding SA with a K_{D} as determined by surface plasmon resonance of 1 nM to 1, 2, 3, 4, 5, 10, 20, 30, 40, 50, 60, 70, 100, 200, 300, 400 or 500 μM (i.e., ×10⁻⁹ to 5×10⁻⁴), or 100 nM to 10 μM, or 1 to 5 μM or 3 to 70 nM or 10 nM to 1, 2, 3, 4 or 5μM. For example 30 to 70 nM as determined by surface plasmon resonance. In one embodiment, the first antibody single variable domain (or a dAb monomer) binds SA (e.g., Human Serum Albumin) with a K_{D} as determined by surface plasmon resonance of approximately 1, 50, 70, 100, 150, 200, 300 nM or 1, 2 or 3 μM. In one embodiment, for a dual specific ligand comprising a first anti-SA dAb and a second dAb to CD28, the affinity (e.g. K_{D} and/or K_{off} as measured by surface plasmon resonance, e.g. using Biacore) of the second dAb for its target is from 1 to 100000 times (e.g., 100 to 100000, or 1000 to 100000, or 10000 to 100000 times) the affinity of the first antibody single variable domain for SA. In one embodiment, the serum albumin is human serum albumin (HSA). For example, the first antibody single variable domain binds SA with an affinity of approximately 10 pM, while the second antibody single variable domain binds its target with an affinity of 100 pM. In one embodiment, the serum albumin is human serum albumin (HSA). In one embodiment, the first antibody single variable domain binds SA (e.g., HSA) with a K_{D} of approximately 50, for example 70, 100, 150 or 200 nM. Details of dual specific polypeptides comprising antibody single variable domains are found in WO03002609, WO04003019 and WO04058821, which are incorporated by reference herein in their entirety.

Also provided are dimers, trimers and polymers of the aforementioned antibody single variable domain monomers, in accordance with the foregoing aspect.

Antibody single variable domain monomers, dimers and trimers, disclosed herein, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides. Alternatively, ligands disclosed herein may be free of an Fc domain.

In a further aspect is provided one or more nucleic acid molecules encoding at least a dual- or multi-specific polypeptide comprising an antibody single variable domain as herein defined. In one embodiment, the dual- or multi-specific polypeptide is a closed conformation ligand. In another embodiment, it is an open conformation ligand. The dual- or multi-specific polypeptide may be encoded by a single nucleic acid molecule; alternatively, each dual- or multi-specific polypeptide may be encoded by a separate nucleic acid molecule. Where the dual- or multi-specific polypeptide is encoded by a single nucleic acid molecule, the domains may be expressed as a fusion polypeptide, or may be separately expressed and subsequently linked together, for example using chemical linking agents. Dual- or multi-specific polypeptides expressed from separate nucleic acids will be linked together by appropriate means.

The nucleic acid may further encode a signal sequence for export of the polypeptides from a host cell upon expression and may be fused with a surface component of a filamentous bacteriophage particle (or other component of a selection display system) upon expression. Leader sequences, which may be used in bacterial expression and/or phage or phagemid display, include pelB, stII, ompA, phoA, bla, ompT and pelA.

In a further aspect of the second configuration disclosed herein includes a vector comprising nucleic acid.

In a yet further aspect is provided a host cell transfected with a vector.

Expression from such a vector may be configured to produce, for example on the surface of a bacteriophage particle, epitope binding domains for selection. This allows selection of displayed domains and thus selection of ‘multispecific ligands’ using the method disclosed herein.

### 6.2.1 Structure of ‘Dual-Specific or Multi-Specific Polypeptides

As described above, a conventional antibody is herein defined as an antibody or fragment (Fab, Fv, disulfide linked Fv, scFv, diabody) which comprises at least one heavy and a light chain variable domain, at least two heavy chain variable domains or at least two light chain variable domains. It may be at least partly derived from any species naturally producing an antibody, or created by recombinant DNA technology; whether isolated from serum, B-cells, hybridomas, transfectomas, yeast or bacteria).

In one embodiment of a dual-specific or multi-specific polypeptide comprising antibody single variable domains, the multi-specific polypeptide comprises at least one heavy chain antibody single variable domain and one light chain antibody single variable domain, or two heavy or light chain antibody single variable domains. For example, the multi-specific polypeptide may comprise a V_{H}/V_{L} pair, a pair of V_{H} domains or a pair of V_{L} domains.

The first and the second variable domains of such a the multi-specific polypeptide may be on the same polypeptide chain. Alternatively they may be on separate polypeptide chains. In the case that they are on the same polypeptide chain they may be linked by a linker, which is preferentially a peptide sequence, as described above.

The first and second variable domains may be covalently or non-covalently associated. In the case that they are covalently associated, the covalent bonds may be disulphide bonds.

In the case that the variable domains are selected from V-gene repertoires selected for instance using phage display technology as herein described, then these variable domains comprise a universal framework region, such that is they may be recognized by a specific generic ligand as herein defined. The use of universal frameworks, generic ligands, and the like is described in WO 99/20749.

Where V-gene repertoires are used variation in polypeptide sequence is, in an aspect, located within the structural loops of the variable domains. The polypeptide sequences of either antibody single variable domain may be altered by DNA shuffling or by mutation. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

In one embodiment, the ‘dual-specific polypeptide’ comprising antibody single variable domains is in the format of a single chain Fv fragment. In an alternative embodiment, the ‘dual-specific ligand’ is in the format of a Fab format.

A further aspect disclosed herein provides nucleic acid encoding at least a ‘dual-specific polypeptide’ as herein defined.

One skilled in the art will appreciate that, depending on the aspect, both antigens or epitopes may bind simultaneously to the same antibody molecule. Alternatively, they may compete for binding to the same antibody molecule. For example, where both epitopes are bound simultaneously, both antibody single variable domains of a dual specific polypeptide are able to independently bind their target epitopes. Where the antibody single variable domains compete, the one antibody single variable domain is capable of binding its target, but not at the same time as the other antibody single variable domain binds its cognate target; or the first antibody single variable domain is capable of binding its target, but not at the same time as the second antibody single variable domain binds its cognate target.

The antibody single variable domains may be derived from variable regions of antibodies directed against target antigens or epitopes, e.g., CD28. Alternatively they may be derived from a repertoire of single antibody domains such as those expressed on the surface of filamentous bacteriophage. Selection may be performed as described below.

In general, the nucleic acid molecules and vector constructs required for the performance as disclosed herein may be constructed and manipulated as set forth in standard laboratory manuals, such as Sambrook et al. (1989) Molecular Cloning: A Laboratory Manual, Cold Spring Harbor, USA.

The manipulation of nucleic acids useful as disclosed herein is typically carried out in recombinant vectors.

Thus, a further aspect disclosed herein provides a vector comprising nucleic acid encoding at least a ‘dual-specific polypeptide’ comprising multiple antibody single variable domains as herein defined.

As used herein, vector refers to a discrete element that is used to introduce heterologous DNA into cells for the expression and/or replication thereof. Methods by which to select or construct and, subsequently, use such vectors are well known to one of ordinary skill in the art. Numerous vectors are publicly available, including bacterial plasmids, bacteriophage, artificial chromosomes, and episomal vectors. Such vectors may be used for simple cloning and mutagenesis; alternatively gene expression vector is employed. A vector of use as disclosed herein may be selected to accommodate a polypeptide coding sequence of a desired size, typically from 0.25 kilobase (kb) to 40 kb or more in length. A suitable host cell is transformed with the vector after in vitro cloning manipulations. Each vector contains various functional components, which generally include a cloning (or “polylinker”) site, an origin of replication, and at least one selectable marker gene. If given vector is an expression vector, it additionally possesses one or more of the following: enhancer element, promoter, transcription termination, and signal sequences, each positioned in the vicinity of the cloning site, such that they are operatively linked to the gene encoding a ligand as disclosed herein.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

### 6.2.2 Combining Single Variable Domains

Domains useful as disclosed herein, once selected using methods exemplified above, may be combined by a variety of methods known in the art, including covalent and non-covalent methods.

Preferred methods include the use of polypeptide linkers, as described, for example, in connection with scFv molecules (Bird et al., (1988) Science 242:423-426). Discussion of suitable linkers is provided in Bird et al. Science 242, 423-426; Hudson et al, Journal Immunol Methods 231 (1999) 177-189; Hudson et al, Proc Nat Acad Sci USA 85, 5879-5883. Linkers are in an aspect, flexible, allowing the two single domains to interact. One linker example is a (Gly₄ Ser)ₙ linker, where n=1 to 8, e.g., 2, 3, 4, 5, or 7. The linkers used in diabodies, which are less flexible, may also be employed (Holliger et al., (1993) PNAS (USA) 90: 6444-6448).

In one embodiment, the linker employed is not an immunoglobulin hinge region.

Variable domains may be combined using methods other than linkers. For example, the use of disulphide bridges, provided through naturally-occurring or engineered cysteine residues, may be exploited to stabilize V_{H}-V_{H}, V_{L}-V_{L} or V_{H}-V_{L} dimers (Reiter et al., (1994) Protein Eng. 7: 697-704) or by remodeling the interface between the variable domains to improve the “fit” and thus the stability of interaction (Ridgeway et al., (1996) Protein Eng. 7:617-621; Zhu et al., (1997) Protein Science 6: 781-788).

Other techniques for joining or stabilizing variable domains of immunoglobulins, and in particular antibody V_{H} domains, may be employed as appropriate.

As disclosed herein, dual specific polypeptides can be in “closed” conformations in solution. A “closed” configuration is that in which the two domains (for example V_{H} and V_{L}) are present in associated form, such as that of an associated V_{H}-V_{L} pair which forms an antibody binding site. For example, scFv may be in a closed conformation, depending on the arrangement of the linker used to link the V_{H} and V_{L} domains. If this is sufficiently flexible to allow the domains to associate, or rigidly holds them in the associated position, it is likely that the domains will adopt a closed conformation.

Similarly, V_{H} domain pairs and V_{L} domain pairs may exist in a closed conformation. Generally, this will be a function of close association of the domains, such as by a rigid linker, in the ligand molecule. Polypeptides comprising multiple antibody single variable domains in a closed conformation will be unable to bind both the molecule which increases the half-life of the polypeptide comprising multiple antibody single variable domains and a second target molecule. Thus, the polypeptide comprising multiple antibody single variable domains will typically only bind the second target molecule on dissociation from the molecule which increases the half-life of the ligand.

Moreover, the construction of V_{H}/V_{H}, V_{L}/V_{L} or V_{H}/V_{L} dimers comprising multiple antibody single variable domains without linkers provides for competition between the domains.

Polypeptides comprising multiple antibody single variable domains disclosed herein may moreover be in an open conformation. In such a conformation, the ligands will be able to simultaneously bind both the molecule which increases the half-life of the ligand and the second target molecule. Typically, the domains in a polypeptide comprising multiple antibody single variable domains in an open configuration are (in the case of V_{H}-V_{L} pairs) held far enough apart for the domains not to interact and form an antibody binding site and not to compete for binding to their respective epitopes. In the case of dimers in the V_{H}/V_{H} or V_{L}/V_{L} format, the domains are not forced together by rigid linkers. Naturally, such domain pairings will not compete for antigen binding nor will such pairing produce a single antibody binding site formed by the two paired domains together.

Polypeptides comprising multiple antibody single variable domains disclosed herein, presented in a format of Fab fragments and whole antibodies, will exist primarily in the closed conformation, although it will be appreciated that open and closed dual-specific polypeptides are likely to exist in a variety of equilibria under different circumstances. Binding of one domain to a target is likely to shift the balance of the equilibrium towards the open configuration. Thus, certain dual-specific polypeptides disclosed herein can exist in two conformations in solution, one of which (the open form) can bind two antigens or epitopes independently, while the alternative conformation (the closed form) can only bind one antigen or epitope; antigens or epitopes thus compete for binding to the ligand in this closed conformation.

Although the open form of the dual specific polypeptides comprising multiple antibody single variable domains disclosed herein may thus exist in equilibrium with the closed form in solution, it is envisaged that the equilibrium will favor the closed form; moreover, the open form can be sequestered by target binding into a closed conformation. In an exemplary embodiment, therefore, certain dual specific polypeptides comprising multiple antibody single variable domains disclosed herein are present in an equilibrium between two (open and closed) conformations.

Dual specific polypeptides comprising multiple antibody single variable domains disclosed herein may be modified in order to favor an open or closed conformation. For example, stabilization of V_{H}-V_{L} interactions with disulphide bonds stabilizes the closed conformation. Moreover, linkers used to join the domains, including V_{H} domain and V_{L} domain pairs, may be constructed such that the open from is favored; for example, the linkers may sterically hinder the association of the domains, such as by incorporation of large amino acid residues in opportune locations, or the designing of a suitable rigid structure which will keep the domains physically spaced apart.

### 6.2.3 Characterization of the Dual-Specific Polypeptide Comprising Multiple Antibody Single Variable Domains

The binding of the dual-specific polypeptide comprising multiple antibody single variable domains disclosed herein to its specific antigens or epitopes can be tested by methods which will be familiar to those skilled in the art and include ELISA. In a preferred embodiment, binding is tested using monoclonal phage ELISA.

Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. (1994) Ann. Rev. Immunology 12, 433-55 and references cited therein.

The diversity of the selected phage monoclonal antibodies may also be assessed by gel electrophoresis of PCR products (Marks et al. 1991, supra; Nissim et al. 1994 supra), probing (Tomlinson et al., 1992) J. Mol. Biol. 227, 776) or by sequencing of the vector DNA.

### 7.0 Increasing Polypeptide Stability

### 7.1 Increasing Half-Life

In vivo, the PEGylated monovalent anti-CD28 antibody single variable domains as described herein confer a distinct advantage over non-PEGylated antibody single variable domains, in that the PEGylated antibody single variable domains molecules will have a greatly prolonged in vivo half-life. It will be understood, in the context of the present disclosure, that a particular half-life of any composition may be either increased or decreased by the route of administration of the composition to a patient.

Nonetheless, without being bound to one particular theory, it is believed that the increased half-life of the molecules described herein is conferred by the increased hydrodynamic size of the antibody single variable domain resulting from the attachment of PEG polymer(s). More specifically, it is believed that two parameters play an important role in determining the serum half-life of PEGylated antibody single variable domains. The first criterion is the nature and size of the PEG attachment, i.e., if the polymer used is simply a linear chain or a branched/forked chain, wherein the branched/forked chain gives rise to a longer half-life. The second is the location of the PEG moiety or moieties on the antibody single variable domain in the final format and how many “free” unmodified PEG arms the molecule has. The resulting hydrodynamic size of the PEGylated antibody single variable domain, as estimated, for example, by size exclusion chromatography, reflects the serum half-life of the molecule. Accordingly, the larger the hydrodynamic size of the PEGylated molecule, the greater the serum half-life.

Increased half-life is useful in vivo applications of immunoglobulins, especially antibodies and most especially antibody fragments of small size. Such fragments (Fvs, Fabs, scFvs, dAbs) suffer from rapid clearance from the body; thus, while they are able to reach most parts of the body rapidly, and are quick to produce and easier to handle, their in vivo applications have been limited by their only brief persistence in vivo.

In one aspect, a monovalent anti-CD28 antibody single variable domain as described herein is stabilized in vivo by fusion with a moiety, such as PEG, that increases the hydrodynamic size of the antibody single variable domain. Methods for pharmacokinetic analysis and determination of half-life will be familiar to those skilled in the art. Details may be found in Kenneth et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t-α and t-β half lives and area under the curve (AUC).

Typically, the half-life of a PEGylated antibody single variable domain as described herein is increased by 10%, 20%, 30%, 40%, 50%, or more relative to a non-PEGylated dAb (wherein the antibody single variable domain of the PEGylated antibody single variable domain and non-PEGylated antibody single variable domain are the same). Increases in the range of 2×, 3×, 4×, 5×, 7×, 10×, 20×, 30×, 40×, and up to 50× or more of the half-life are possible. Alternatively, or in addition, increases in the range of up to 30×, 40×, 50×, 60×, 70×, 80×, 90×, 100×, or 150× of the half-life are possible.

Half lives (t1/2-α and t1/2-β) and AUC can be determined from a curve of serum concentration of ligand against time. The WinNonlin analysis package (available from Pharsight Corp., Mountain View, Calif. 94040, USA) can be used, for example, to model the curve. In a first phase (the alpha phase) the antibody single variable domain is undergoing mainly distribution in the patient, with some elimination. A second phase (beta phase) is the terminal phase when the antibody single variable domain has been distributed and the serum concentration is decreasing as the antibody single variable domain is cleared from the patient. The “tα half-life” is the half-life of the first phase and the “tβ half-life” is the half-life of the second phase. “Half-life” as used herein, unless otherwise noted, refers to the overall half-life of an antibody single variable domain disclosed herein determined by non-compartment modeling (as contrasted with biphasic modeling, for example). Beta half-life is a measurement of the time it takes for the amount of antibody single variable domain monomer or multimer to be cleared from the mammal to which it is administered. Thus, advantageously, an antibody single variable domain-containing composition, e.g., a antibody single variable domain-effector group composition is contemplated having a tα half-life in the range of 0.25 hours to 6 hours or more. In one embodiment, the lower end of the range is 30 minutes, 45 minutes, 1 hour, 1.3 hours, 2 hours, 3 hours, 4 hours, 5 hours, 6 hours, 7 hours, 10 hours, 11 hours, or 12 hours. In addition or alternatively, an antibody single variable domain containing composition will have a tα half-life in the range of up to and including 12 hours. In one embodiment, the upper end of the range is 11, 10, 9, 8, 7, 6, or 5 hours. An example of a suitable range is 1.3 to 6 hours, 2 to 5 hours, or 3 to 4 hours.

Advantageously, a composition containing an antibody single variable domain has a tf3 half-life in the range of 1-170 hours or more. In one embodiment, the lower end of the range is 2.5, 3, 4, 5, 6, 7, 8, 9, 10, 11, 12, 13, 14, 15, 20, 24, 36, 48, or 72 hours and the upper end of the range is up to 24 hours, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, and up to 31 days. In one embodiment an antibody single variable domain composition has a tβ half-life in the range of at least 12 hours up to 24 hours, 14 days, 28 days, 4 weeks, and up to one month. Advantageously, a dAb containing composition disclosed herein will have a tβ half-life in the range 2-100 hours, 4-80 hours, and 10-40 hours. In a further embodiment, it will be in the range 12-48 hours. In a further embodiment still, it will be in the range 12-26 hours. Disclosed herein is a composition containing an antibody single variable domain having a half-life in the range of 1-170 hours or more. In one embodiment, the lower end of the range is 1.3, 2.5, 3, 4, 5, 6, 7, 8, 9, 10, 11, 12, 13, 14, 15, 20, 24, 36, 48, or 72 hours. In addition, or alternatively, a composition containing an antibody single variable domain has a half-life in the range of up to and including 21 days. In one embodiment, the upper end of the range is up to 12 hours, 24 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, and up to 31 days.

In addition, or alternatively to the above criteria, a composition containing an antibody single variable domain has an AUC value (area under the curve) in the range of 1 mg.min/ml or more. In one embodiment, the lower end of the range is 5, 10, 15, 20, 30, 100, 200, or 300 mg.min/ml. In addition, or alternatively, a composition containing an antibody single variable domain disclosed herein has an AUC in the range of up to 600 mg.min/ml. In one embodiment, the upper end of the range is 500, 400, 300, 200, 150, 100, 75, or 50 mg.min/ml. Exemplary compositions containing an antibody single variable domain disclosed herein will have an AUC in the range selected from the group consisting of the following: 15 to 150 mg.min/ml, 15 to 100 mg.min/ml, 15 to 75 mg.min/ml, and 15 to 50 mg.min/ml.

The polypeptides containing one or more antibody single variable domains disclosed herein, including, mono-, dual- and multi-specific polypeptides, in one configuration thereof, are capable of binding to one or more molecules which can increase the half-life of the ligand in vivo. Typically, such molecules are polypeptides which occur naturally in vivo and which resist degradation or removal by endogenous mechanisms which remove unwanted material from the organism.

For example, the molecule which increases the half-life in the organism may be selected from the following:
Proteins from the extracellular matrix; for example collagen, laminins, integrins, and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs, or type II collagen found in cartilage, invertebral disc, notochord, vitreous humour of the eye.
Proteins found in blood, including: Plasma proteins such as fibrin, α-2 macroglobulin, serum albumin, fibrinogen A, fibrinogen B, serum amyloid protein A, heptaglobin, profilin, ubiquitin, uteroglobulin, and β-2-microglobulin;
Enzymes and inhibitors such as plasminogen, lysozyme, cystatin C, alpha-1-antitrypsin, and pancreatic trypsin inhibitor. Plasminogen is the inactive precursor of the trypsin-like serine protease plasmin. It is normally found circulating through the blood stream. When plasminogen becomes activated and is converted to plasmin, it unfolds a potent enzymatic domain that dissolves the fibrinogen fibers that entangle the blood cells in a blood clot. This is called fibrinolysis.
Immune system proteins, such as IgE, IgG, and IgM.
Transport proteins such as retinol binding protein, α-1 microglobulin.
Defensins such as beta-defensin 1, neutrophil defensins 1, 2, and 3.
Proteins found at the blood brain barrier or in neural tissues, such as melanocortin receptor, myelin, ascorbate transporter.
Transferrin receptor specific ligand-neuropharmaceutical agent fusion proteins (see U.S. Pat. No. 5,977,307); brain capillary endothelial cell receptor, transferrin, transferrin receptor, insulin, insulin-like growth factor 1 (IGF 1) receptor, insulin-like growth factor 2 (IGF 2) receptor, insulin receptor.
Proteins localised to the kidney, such as polycystin, type IV collagen, organic anion transporter K1, Heymann's antigen.
Proteins localised to the liver, for example alcohol dehydrogenase, G250.
Blood coagulation factor X
α1 antitrypsin
HNF 1α
Proteins localised to the lung, such as secretory component (binds IgA).
Proteins localised to the heart, for example HSP 27. This is associated with dilated cardiomyopathy.
Proteins localised to the skin, for example keratin.
Bone specific proteins, such as bone morphogenic proteins (BMPs), which are a subset of the transforming growth factor β superfamily that demonstrate osteogenic activity. Examples include BMP-2, -4, -5, -6, -7 (also referred to as osteogenic protein (OP-1) and -8 (OP-2).
Tumour specific proteins, including human trophoblast antigen, herceptin receptor, oestrogen receptor, cathepsins, e.g., cathepsin B (found in liver and spleen).
Disease-specific proteins, such as antigens expressed only on activated T-cells: including LAG-3 (lymphocyte activation gene), osteoprotegerin ligand (OPGL) (see Nature 402, 304-309; 1999); OX40 (a member of the TNF receptor family, expressed on activated T cells and the only costimulatory T cell molecule known to be specifically up-regulated in human T cell leukemia virus type-I (HTLV-I)-producing cells.) (see J. Immunol. 2000 Jul. 1; 165(1):263-70); metalloproteases (associated with arthritis/cancers), including CG6512 Drosophila, human paraplegin, human FtsH, human AFG3L2, murine ftsH; angiogenic growth factors, including acidic fibroblast growth factor (FGF-1), basic fibroblast growth factor (FGF-2), vascular endothelial growth factor/vascular permeability factor (VEGF/VPF), transforming growth factor-a (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), fractalkine.
Stress proteins (heat shock proteins). HSPs are normally found intracellularly. When they are found extracellularly, it is an indicator that a cell has died and spilled out its contents. This unprogrammed cell death (necrosis) only occurs when as a result of trauma, disease, or injury, and therefore in vivo, extracellular HSPs trigger a response from the immune system that will fight infection and disease. A dual specific which binds to extracellular HSP can be localized to a disease site.
Proteins involved in Fc transport such as:
   The Brambell receptor (also known as FcRB). This Fc receptor has two functions, both of which are potentially useful for delivery. The functions include the transport of IgG from mother to child across the placenta and the protection of IgG from degradation thereby prolonging its serum half-life of IgG. It is thought that the receptor recycles IgG from endosome (See Holliger et al, Nat. Biotechnol. 1997 July; 15(7): 632-6).
   Other proteins involved in Fc transport include the neonatal Fc receptor (FcRn) described in Gastinel et al., 1992, PNAS 89:638; and Roopenian et al., 2003 J. Immunol. 170:3528.
      Antibody single variable domains disclosed herein may designed to be specific for the above targets without requiring any increase in or increasing half-life in vivo. For example, antibody single variable domains disclosed herein can be specific for targets selected from the foregoing which are tissue-specific, thereby enabling tissue-specific targeting of the dual specific polypeptide comprising multiple antibody single variable domains, or of the antibody single variable domain monomer that binds a tissue-specific therapeutically relevant target, irrespective of any increase in half-life, although this may result. Moreover, where the dual specific polypeptide comprising multiple antibody single variable domains or the antibody single variable domain monomer targets kidney or liver, this may redirect the dual specific polypeptide or antibody single variable domain monomer to an alternative clearance pathway in vivo (for example, the antibody single variable domain monomer may be directed away from liver clearance to kidney clearance).

### 7.2 Increasing Resistance to Protease Degradation

Also disclosed herein is that the PEGylated antibody single variable domain monomer and antibody single variable domain multimers described herein possess increased stability to the action of proteases. In the presence of pepsin many antibody single variable domains are totally degraded at pH 2 because the protein is unfolded under the acid conditions, thus making the protein more accessible to the protease enzyme. Provided herein are PEGylated antibody single variable domain monomer and multimers, wherein it is believed that the PEG polymer provides protection of the polypeptide backbone due the physical coverage of the backbone by the PEG polymer, thereby preventing the protease from gaining access to the antibody single variable domain polypeptide backbone and cleaving it. In a preferred embodiment a PEGylated antibody single variable domain having a higher hydrodynamic size (e.g., 200 to 500 kDa) is generated as disclosed herein, because the larger hydrodynamic size will confirm a greater level of protection from protease degradation than a PEGylated dAb having a lower hydrodynamic size. In one embodiment, a PEG- or other polymer-linked antibody single variable domain monomer or multimer is degraded by no more than 10% when exposed to one or more of pepsin, trypsin, elastase, chymotrypsin, or carboxypeptidase, wherein if the protease is pepsin then exposure is carried out at pH 2.0 for 30 minutes, and if the protease is one or more of trypsin, elastase, chymotrypsin, or carboxypeptidase, then exposure is carried out at pH 8.0 for 30 minutes. In a preferred embodiment, a PEG- or other polymer-linked antibody single variable domain monomer or multimer is degraded by no more than 10% when exposed to pepsin at pH 2.0 for 30 minutes, in an aspect, no more than 5%, and in another aspect, not degraded at all. In a further preferred embodiment, a PEG- or other polymer-linked antibody single variable domain multimer (e.g., hetero- or homodimer, trimer, tetramer, octamer, etc.) disclosed herein is degraded by less than 5%, and is, in an aspect, not degraded at all in the presence of pepsin at pH 2.0 for 30 minutes. In an exemplary embodiment, a PEG- or other polymer-linked antibody single variable domain monomer or multimer is degraded by no more than 10% when exposed to trypsin, elastase, chymotrypsin, or carboxypeptidase at pH 8.0 for 30 minutes, in an aspect, no more than 5%, and, in a further aspect, not degraded at all. In a further exemplary embodiment, a PEG- or other polymer-linked antibody single variable domain multimer (e.g., hetero- or homodimer, trimer, tetramer, octamer, etc.) disclosed herein is degraded by less than 5%, and is, in an aspect, not degraded at all in the presence of trypsin, elastase, chymotrypsin, or carboxypeptidase at pH 8.0 for 30 minutes.

The relative ratios of protease:PEG-antibody single variable domain polypeptide may be altered as disclosed herein to achieve the desired level of degradation as described above. For example the ratio or protease to PEG-antibody single variable domain may be from about 1:30, to about 10:40, to about 20:50, to about 30:50, about 40:50, about 50:50, about 50:40, about 50:30, about 50:20, about 50:10, about 50:1, about 40:1, and about 30:1.

Accordingly, disclosed herein is a method for decreasing the degradation of an antibody single variable domain comprising linking an antibody single variable domain monomer or multimer to a PEG polymer according to any of the embodiments described herein. As disclosed herein, the antibody single variable domain is degraded by no more than 10% in the presence of pepsin at pH 2.0 for 30 minutes. In particular, a PEG-linked antibody single variable domain multimer is degraded by no more than 5%, and, in an aspect, not degraded at all in the presence of pepsin at pH 2.0 for 30 minutes. In an alternate embodiment, the antibody single variable domain is degraded by no more than 10% when exposed to trypsin, elastase, chymotrypsin, or carboxypeptidase at pH 8.0 for 30 minutes, in an aspect, no more than 5%, and, in another aspect, not degraded at all.

Degradation of PEG-linked antibody single variable domain monomers and multimers as set forth herein may be measured using methods which are well known to those of skill in the art. For example, following incubation of a PEG-linked antibody single variable domain with pepsin at pH 2.0 for 30 minutes, or with trypsin, elastase, chymotrypsin, or carboxypeptidase at pH 8.0 for 30 minutes, the antibody single variable domain samples may be analyzed by gel filtration, wherein degradation of the antibody single variable domain monomer or multimer is evidenced by a gel band of a smaller molecular weight than an un-degraded (i.e., control antibody single variable domain not treated with pepsin, trypsin, chymotrypsin, elastase, or carboxypeptidase) dAb. Molecular weight of the antibody single variable domain bands on the gel may be determined by comparing the migration of the band with the migration of a molecular weight ladder. Other methods of measuring protein degradation are known in the art and may be adapted to evaluate the PEG-linked antibody single variable domain monomers and multimers as disclosed herein.

### 8.0 Uses of Monovalent Anti-CD28 Antibody Single Variable Domains

Anti-CD28 antibody single variable domains as described herein are useful for antagonizing the activity of a CD28. Therefore, anti-CD28 antibody single variable domains as described herein can be used to treat a patient having a condition, disease or disorder mediated in whole or in part by CD28 activity.

Anti-CD28 antibody single variable domains as described herein are useful for the treatment or prevention of diseases or disorders in which inappropriate activation of a CD28-mediated pathway is involved. Anti-CD28 antibody single variable domains as described herein are also useful for the treatment, prevention, or alleviation of symptoms of diseases or disorders in which inappropriate activation of a CD28-mediated pathway is involved.

In an aspect, autoimmune diseases frequently involve inappropriate regulation or activity of CD28 pathways. Administration of an anti-CD28 antibody single variable domain as described herein to an individual suffering from such a disease, including an autoimmune disease, can reduce one or more symptoms of the disease. Non-limiting examples of diseases for which the antibody single variable domains described herein can be therapeutically useful include, but are not limited to, Addison's disease, allergy, ankylosing spondylitis, asthma, atherosclerosis, autoimmune diseases of the ear, autoimmune diseases of the eye, autoimmune hepatitis, autoimmune parotitis, colitis, coronary heart disease, Crohn's disease, diabetes (Type I), diabetes, including Type 1 and/or Type 2 diabetes, epididymitis, glomerulonephritis, Graves' disease, Guillain-Barre syndrome, Hashimoto's disease, hemolytic anemia, idiopathic thrombocytopenic purpura, inflammatory bowel disease (IBD), immune response to recombinant drug products, e.g., factor VII in hemophilia, systemic lupus erythematosus, male infertility, multiple sclerosis, myasthenia gravis, pemphigus, psoriasis, rheumatic fever, rheumatoid arthritis, sarcoidosis, scleroderma, Sjogren's syndrome, spondyloarthropathies, thyroiditis, transplant rejection, and vasculitis. Autoimmune-mediated conditions also include, but are not limited to, conditions in which the tissue affected is the primary target, and in some cases, the secondary target. Such conditions include, but are not limited to, AIDS, atopic allergy, bronchial asthma, eczema, leprosy, schizophrenia, inherited depression, transplantation of tissues and organs, chronic fatigue syndrome, Alzheimer's disease, Parkinson's disease, myocardial infarction, stroke, autism, epilepsy, Arthus's phenomenon, anaphylaxis, and alcohol and drug addiction.

The anti-CD28 antibody single variable domains described herein are additionally useful in the way that generally any antibody preparation is useful, e.g., for in vivo imaging or diagnostic uses, in vitro diagnostic uses, etc.

For these and other uses it may be desirable to label the anti-CD28 antibody single variable domains, e.g., with a fluorescent, colorimetric, enzymatic or radioactive label. Methods of labeling antibody single variable domains are well known in the art.

### 9.0 Pharmaceutical Compositions, Dosage, and Administration

The antibody single variable domains set forth herein can be incorporated into pharmaceutical compositions suitable for administration to a subject. Typically, the pharmaceutical composition comprises a monovalent anti-CD28 antibody single variable domain and a pharmaceutically acceptable carrier. As used herein, “pharmaceutically acceptable carrier” includes any and all solvents, dispersion media, coatings, antibacterial, and antifungal agents, isotonic and absorption delaying agents, and the like that are physiologically compatible. The term “pharmaceutically acceptable carrier” excludes tissue culture medium comprising bovine or horse serum. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances include minor amounts of auxiliary substances such as wetting or emulsifying agents, preservatives or buffers, which enhance the shelf life or effectiveness of the antibody single variable domain.

The compositions as described herein may be in a variety of forms. These include, for example, liquid, semi-solid, and solid dosage forms, such as liquid solutions (e.g., injectable and infusible solutions), dispersions or suspensions, powders, liposomes, and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular).

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants.

The antibody single variable domains described herein can be administered by a variety of methods known in the art, although for many therapeutic applications, the preferred route/mode of administration is intravenous injection or infusion. The polypeptide can also be administered by intramuscular or subcutaneous injection.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Antibody single variable domains and other relatively small monovalent antibody single variable domains are well suited for formulation as extended release preparations due, in part, to their small size, the number of moles per dose can be significantly higher than the dosage of, for example, full sized antibodies. Biodegradable, biocompatible polymers can be used, such as ethylene vinyl acetate, polyanhydrides, polyglycolic acid, collagen, polyorthoesters, and polylactic acid. Prolonged absorption of injectable compositions can be brought about by including in the composition an agent that delays absorption, for example, monostearate salts and gelatin. Many methods for the preparation of such formulations are patented or generally known to those skilled in the art. See, e.g., SUSTAINED AND CONTROLLED RELEASE DRUG DELIVERY SYSTEMS, J. R. Robinson, ed., Marcel Dekker, Inc., New York, 1978. Additional methods applicable to the controlled or extended release of polypeptide agents such as the monovalent antibody single variable domains disclosed herein are described, for example, in U.S. Pat. Nos. 6,306,406 and 6,346,274, as well as, for example, in U.S. Patent Publication Nos. US20020182254 and US20020051808, all of which are incorporated herein by reference.

Additional active compounds can also be incorporated into the compositions. In certain embodiments, a monovalent anti-CD28 antibody single variable domain is coformulated with and/or coadministered with one or more additional therapeutic agents. For example, a monovalent anti-CD28 antibody single variable domain can be coformulated and/or coadministered with one or more additional antibodies that bind other targets (e.g., antibodies that bind other cytokines or that bind cell surface molecules), or, for example, one or more cytokines. Such combination therapies may utilize lower dosages of the administered therapeutic agents, thus avoiding possible toxicities or complications associated with the various monotherapies.

The pharmaceutical compositions disclosed herein can include a “therapeutically effective amount” or a “prophylactically effective amount” of a monovalent anti-CD28 antibody single variable domain. A “therapeutically effective amount” refers to an amount effective, at dosages and for periods of time necessary, to achieve the desired therapeutic result. A therapeutically effective amount of the antibody single variable domain can vary according to factors such as the disease state, age, sex, and weight of the individual, and the ability of the monovalent anti-CD28 antibody single variable domain to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, because a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

A non-limiting range for a therapeutically or prophylactically effective amount of a monovalent anti-CD28 antibody single variable domain is 0.1-20 mg/kg, and in an aspect, 1-10 mg/kg. It is to be noted that dosage values can vary with the type and severity of the condition to be alleviated. It is to be further understood that for any particular subject, specific dosage regimens should be adjusted over time according to the individual need and the professional judgment of the administering clinician.

The efficacy of treatment with a monovalent anti-CD28 antibody single variable domain as described herein is judged by the skilled clinician on the basis of improvement in one or more symptoms or indicators of the disease state or disorder being treated. An improvement of at least 10% (increase or decrease, depending upon the indicator being measured) in one or more clinical indicators is considered “effective treatment,” although greater improvements are preferred, such as 20%, 30%, 40%, 50%, 75%, 90%, or even 100%, or, depending upon the indicator being measured, more than 100% (e.g., two-fold, three-fold, ten-fold, etc., up to and including attainment of a disease-free state. Indicators can be physical measurements, e.g., enzyme, cytokine, growth factor or metabolite levels, rate of cell growth or cell death, or the presence or amount of abnormal cells. One can also measure, for example, differences in the amount of time between flare-ups of symptoms of the disease or disorder (e.g., for remitting/relapsing diseases, such as multiple sclerosis). Alternatively, non-physical measurements, such as a reported reduction in pain or discomfort or other indicator of disease status can be relied upon to gauge the effectiveness of treatment. Where non-physical measurements are made, various clinically acceptable scales or indices can be used, for example, the Crohn's Disease Activity Index, or CDAI (Best et al., 1976, Gastroenterology 70: 439), which combines both physical indicators, such as hematocrit and the number of liquid or very soft stools, among others, with patient-reported factors such as the severity of abdominal pain or cramping and general well-being, to assign a disease score.

Psoriasis—Salford Psoriasis Index (SPI) or Psoriasis Area Severity Index (PASI). The PASI is most commonly used method to assess psoriasis disease severity in clinical trials, although it can be exceedingly cumbersome for use in daily clinical practice. The method involves the body being divided into four sections (Legs, which have 40% of a person's skin; the Body (trunk area: stomach, chest, back, etc.) with 30%; the Arms (20%); and the Head (10%)). Each of these areas is scored by itself, and then the four scores are combined into the final PASI. For each section, the percent of area of skin involved, is estimated and then transformed into a grade from 0 to 6:

0% of involved area, grade: 0

<10% of involved area, grade: 1

10-29% of involved area, grade: 2

30-49% of involved area, grade: 3

50-69% of involved area, grade: 4

70-89% of involved area, grade: 5

90-100% of involved area, grade: 6

The severity is estimated by four different parameters: Itching, Erythema (redness), Scaling and Thickness (psoriatic skin is thicker than normal skin). Severity parameters are measured on a scale of 0 to 4, from none to maximum.

The sum of all four severity parameters is than calculated for each section of skin, multiplied by the area score for that area and multiplied by weight of respective section (0.1 for head, 0.2 for arms, 0.3 for body and 0.4 for legs). Example: (Ihead+Ehead+Shead+Thead)×Ahead×0.1=Totalhead

At the end the total PASI is calculated as a sum of PASIs for all four skin sections. Computer-aided measurement of psoriatic lesion area was found to improve the power of the clinical trial, compared to the standard approach. The physician's estimations of the psoriatic lesion area tend to overestimate. The adapted PASI index, where the psoriatic area was not converted into an area grade, but was maintained as a continuous variable, also improved the power of the clinical trial. The modified PASI which involves computer aided area measurement as a continuous variable is named: Computer aided psoriasis continuous area and severity score cPcASI]

In one embodiment described herein, psoriasis is ameliorated by administering to an individual having psoriasis, an antagonist anti-CD28 dAb, or composition thereof, described herein.

### Monitoring and Manifestation of Transplant Rejection

The survival rates of organ transplant patients (currently around 70-85% for 5 years for all transplanted organs) has improved as a result of advances in organ preservation and immunosuppressive treatments, (Chien et al. (2004) Current Pharmaceutical Biotechnology, 5: 551-566). According to Chien et al., organ rejection, especially the acute rejection that occurs in the first few weeks following surgery, as well as chronic graft rejection, is still one of the major causes of functional failure in organ transplantation. Current diagnosis or confirmation of graft rejection following solid organ transplantation requires biopsy of the tissue in order to detect the infiltration of immune cells (e.g., T-cells, macrophages, etc.) into the graft and other pathological changes. Tissue biopsy is not only invasive, it is associated with increased health risk to the patent and is prone to sampling errors that can lead to false negative results. Alternative non-invasive methods are being developed, such as magnetic resonance imaging (MRI) which can be used to monitor the accumulation of immune cells at the rejected organ.

Acute rejection occurs within a week to approximately 4 months after transplantation according to a Medscape article published on the web at medscape.com/viewarticle/436533_{—}12. Acute rejection is a cellular immune response involving mononuclear, cytotoxic and Th cells, monokines, and lymphokines. Quiescent, nonactivated Th cells encounter specific class II antigens displayed on the donor organ, become activated, and synthesize receptors for lymphokines that are simultaneously released from monocytes. Activated monocytes release the lymphokine IL-1, which causes clonal expansion of activated Th cells. Monocytes also release the lymphokine IL-2, which activates and causes the clonal expansion of CTLs. Thus, one embodiment of the instant invention encompasses blocking the activation and/or proliferation of T cells using a CD28 dAb described herein, that functions as an antagonist to CD28 mediated T cell signaling.

Chronic rejection is a prolonged process of declining allograft function. Chronic rejection probably begins at the time of transplantation, but may take months or years to manifest clinically according to a Medscape article published on the web at medscape.com/viewarticle/436533_{—}12. While the clinical and biochemical signs are organ-specific, the result of chronic rejection is the same for all solid organ allografts. Slowly deteriorating graft function (DGF) caused by fibrosis of the graft parenchyma and widespread arteriopathy are the hallmarks of chronic rejection that lead to loss of function and eventual graft loss. T cells and B cells contribute to the damage characteristic of chronic rejection, Demetris A J, Duquesnor R J, Fung J J, et al. Pathophysiology of chronic allograft rejection. Medscape Transplantation. 2000, at the URL: http://transplantation.medscape.com/Medscape/transplantation/ClinicalMgmt/CM.v02/public/ind ex.CM.v02.html. Overproduction of cytokines, including TGF-beta and platelet-derived growth factor, contribute to fibrosis. Continuous production of alloantibody by B cells under the influence of T cells contributes to the arteriopathy, and recipient immune reactivity against the allograft also contributes to the development of DGF.

In one embodiment described herein, symptoms originating from the rejection of an organ transplant are ameliorated by administering an antagonist anti-CD28 dAb described herein, or a composition comprising said anti-CD28 dAb. An anti-CD28 dAb can be administered in conjunction with additional medicants as part of a therapeutic regimen, including pravastatin, an HMG CoA reductase inhibitor with relatively low lipophilicity, has been associated with enhanced heart allograft survival and a reduced incidence of acute rejection among recipients of kidney allografts.

As the term is used herein, “prophylaxis” performed using a composition as described herein is “effective” if the onset or severity of one or more symptoms is delayed or reduced by at least 10%, or abolished, relative to such symptoms in a similar individual (human or animal model) not treated with the composition.

Whereas the monovalent anti-CD28 antibody single variable domains described herein must bind human CD28, where one is to evaluate its effect in an animal model system, the polypeptide must cross-react with one or more antigens in the animal model system, in an aspect, at high affinity. One of skill in the art can readily determine if this condition is satisfied for a given animal model system and a given monovalent anti-CD28 antibody single variable domain. If this condition is satisfied, the efficacy of the monovalent anti-CD28 antibody single variable domain can be examined by administering it to an animal model under conditions which mimic a disease state and monitoring one or more indicators of that disease state for at least a 10% improvement.

10.0 Animal Models

Monovalent anti-CD28 antibody single variable domains as described herein are useful for the treatment of autoimmune disorders in which CD28 signaling is inappropriately active. There are several animal models in which the therapeutic efficacy of a given monovalent anti-CD28 antibody single variable domain can be assessed, as discussed below.

### 10.1 Mouse Model for Chronic Inflammatory Bowel Disease (IBD)

De Winter et al. describe inflammatory bowel disease (IBD) as a multifactorial immune disorder of uncertain etiology, and the use a mouse model of mucosal inflammation that resembles IBD to gather insight into the mechanisms governing both normal and pathological mucosal immune function, De Winter H, Cheroutre H, Kronenberg M., Mucosal immunity and inflammation. II. The yin and yang of T cells in intestinal inflammation: pathogenic and protective roles in a mouse colitis model, Am. J. Physiol. (1999 June), 276 (6 Pt 1): G1317-21. In this widely used adoptive transfer model, the injection into immunodeficient mice of a subset of CD4(+) T lymphocytes, the CD4(+)CD45RBhigh cells, leads to inflammation of the intestine. Pathogenesis is due in part to the secretion of proinflammatory cytokines. The induction of colitis can be prevented by cotransfer of another CD4(+) subpopulation, the CD4(+)CD45RBlow T cells. This population behaves analogously to the CD4(+)CD45RBhigh population in terms of the acquisition of activation markers and homing to the host intestine. However, their lymphokine profile when activated is different, and anti-inflammatory cytokines secreted and/or induced by CD4(+)CD45RBlow T cells prevent colitis.

In one embodiment described herein, chronic inflammation generated by administering CD4(+)CD45RBhigh cells in the above mouse model is ameliorated by administering an antagonist anti-CD28 dAb described herein. In another embodiment chronic inflammation generated by administering CD4(+)CD45RBhigh cells in the above mouse model is prevented by administering an antagonist anti-CD28 dAb described herein before or simultaneously with the administration of CD4(+)CD45RBhigh cells in the above mouse model.

### 10.2 Mouse Model for Rheumatoid Arthritis (RA)

Kouskoff et al. describe Rheumatoid arthritis (RA) as a chronic joint disease characterized by leukocyte invasion and synoviocyte activation followed by cartilage and bone destruction, and the use of a mouse model of spontaneous arthritis to gather insight into the etiology and pathogenesis of RA, Kouskoff V. et al., Cell. (1996 November), 29; 87(5): 811-22. According to Kouskoff et al. this mouse model is generated by crossing a T cell receptor (TCR) transgenic line with the NOD strain. All offspring develop a joint disease highly reminiscent of RA in man. The trigger for the murine disorder is chance recognition of a NOD-derived major histocompatibility complex (MHC) class II molecule by the transgenic TCR; progression to arthritis involves CD4+ T, B, and probably myeloid cells. Thus, Kouskoff et al. propose that a joint-specific disease need not arise from response to a joint-specific antigen but can be precipitated by a breakdown in general mechanisms of self-tolerance resulting in systemic self-reactivity, and suggests that human RA develops by an analogous mechanism.

In one embodiment described herein, RA which spontaneously develops in the above mouse model is ameliorated by administering an antagonist anti-CD28 dAb described herein. In another embodiment described herein, the spontaneous development of RA in the above mouse model is prevented by administering an antagonist anti-CD28 dAb described herein before or simultaneously with the first appearance of symptoms of RA.

### 10.3 Mouse Model for Collagen-Induced Arthritis (CIA)

Brand et al. describe an animal model for arthritis in which an experimental autoimmune disease, Collagen-induced arthritis (CIA), is elicited in susceptible strains of rodents (rat and mouse) and nonhuman primates by immunization with type II collagen (CII), the major constituent protein of articular cartilage, Brand D D et al. Methods Mol. Med. (2004), 102:295-312. Following immunization, these animals develop an autoimmune polyarthritis that shares several clinical and histological features with rheumatoid arthritis. According to Brandt et al., susceptibility to CIA in rodents is linked to the class II molecules of the major histocompatibility complex (MHC), and the immune response to CII is characterized by both the stimulation of collagen-specific T cells and the production of high titers of antibody specific for both the immunogen (heterologous CII) and the autoantigen (mouse CII). Histologically, murine CIA is characterized by an intense synovitis that corresponds precisely with the clinical onset of arthritis. Because of the pathological similarities between CIA and rheumatoid arthritis, the CIA model has been the subject of extensive investigation.

In one embodiment described herein, collagen-induced arthritis elicited in the above mouse model can be ameliorated by administering an antagonist anti-CD28 dAb described herein. In another embodiment described herein, collagen-induced arthritis elicited in the above mouse model is prevented by administering an antagonist anti-CD28 dAb described herein before or simultaneously with the first appearance of an intense synovitis that corresponds precisely with the clinical onset of arthritis. Alternatively, collagen-induced arthritis elicited in the above mouse model is prevented by administering an antagonist anti-CD28 dAb described herein before or simultaneously with the administration of CII.

### 10.4 Acute Mouse Model for Antigen Induced Autoimmunity

Pape et al. describe the use of adoptive transfer of T-cell-antigen-receptor-transgenic T cell into mice for the study of T-cell activation in vivo, in Pape et al. Immunol Rev. (1997 April), 156:67-78. Pape et al. have used this system to show that naive T cells are initially activated within the T-cell zones of secondary lymphoid tissue to proliferate in a B7-dependent manner. If adjuvants or inflammatory cytokines are present during this period, enhanced numbers of T cells accumulate, migrate into B-cell-rich follicles, and acquire the capacity to produce IFN-gamma and help B cells produce IgG2a. If inflammation is absent, most of the initially activated antigen-specific T cells disappear without entering the follicles, and the survivors are poor producers of IL-2 and IFN-gamma. Based on their results, Pape et al. indicate that inflammatory mediators play a key role in regulating the anatomic location, clonal expansion, survival and lymphokine production potential of antigen-stimulated T cells in vivo.

Accordingly, one aspect of the instant invention is to ameliorate an autoimmune diseases generated by administration of TCR-transgenic T cells uniformly expressing an identifiable TCR of known peptide/MHC specificity to a specific autoantigen, by administering an antagonist anti-CD28 dAb described herein. In a preferred embodiment the anti-CD28 dab is divalent, binding to both CD28 and to said TCR.

Accordingly, one aspect of the instant invention is to prevent the occurrence of an autoimmune diseases generated by administration of TCR-transgenic T cells uniformly expressing an identifiable TCR of known peptide/MHC specificity to a specific autoantigen, by administering an antagonist anti-CD28 dAb described herein before or concomitantly with the administration of the transgenic TCR T cells. In a preferred embodiment the anti-CD28 dab is divalent, binding to both CD28 and to said TCR.

### EXAMPLES

### Example 1

### Selection of CD28 Specific Antibody Single Variable Domains

Antibody single variable domains with CD28 binding properties were selected from a library of VH and VK polypeptides. Selections were carried out with recombinant human CD28/Fc Chimera (R&D Systems, Abingdon, UK).

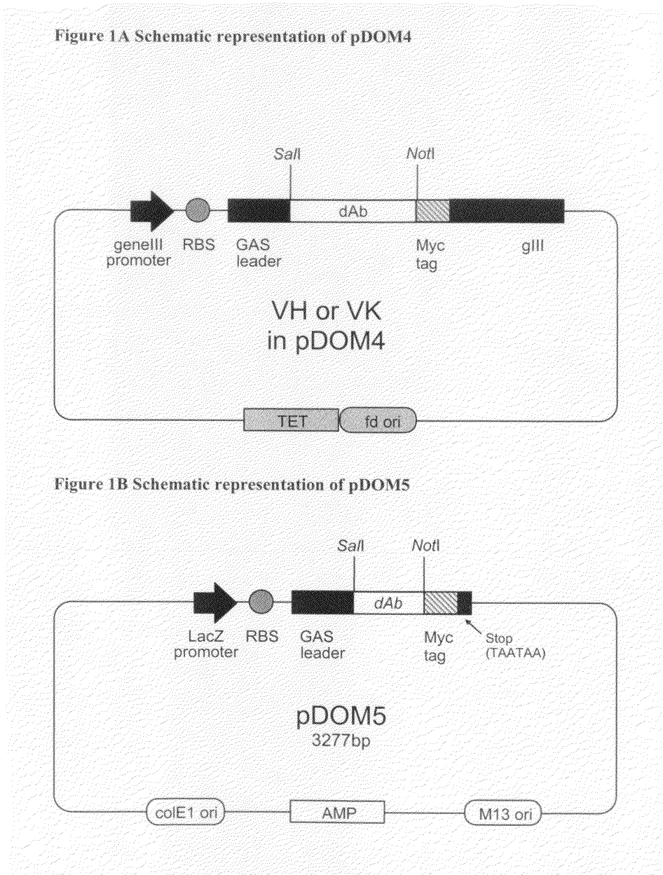

The dAb library used for selections was based on a single human VH framework (V3-23 [locus] DP47 [V Base Entry] and JH4b) and a single human VL framework (012/02 [locus] DPκ9 [V Base Entry] and Jκ1). The dAb genes were genetically linked to the fd phage gene III protein under the control of the GAS1 leader sequence in the pDOM4 vector (FIG. 1A) which contained all the fd genes necessary to generate infective phage particles. The first round of phage selection was performed by premixing phage library (4 pools for the VH libraries [VH11-13, VH14-15, VH16-17, VH18-19] and a single pool for the VK library) with 2% MPBS (Phosphate Buffered Saline supplemented with 2% Marvel dried skim milk powder) and adding CD28-Fc (R&D Systems, UK) to a final concentration of 100 nM. The mixture was incubated for at least 1 hour at room temperature with mixing end-over-end then the antigen-phage complexes captured using protein G Dynabeads (Dynal, Sweden) and washed 8 times with 1 ml PBST (PBS supplemented with 0.1% Tween 20) followed by a singe wash in 1 ml PBS. The washed phage were eluted from the antigen/bead complex by incubating with 0.5 ml of 1 mg/ml trypsin Type XIII from Bovine Pancreas (Sigma Aldrich, UK) in PBS (supplemented with 5 mM Tris-HCl pH 7.4, 0.1 mM CaCl2). Eluted phage were used to infect E. coli and the output phage titres were determined to be between 1×10⁴ to 1×10⁵ t.u./ml.

A second round of selection was performed using enriched phage recovered from the previous round of selection with a final concentration of 50 nM CD28-Fc followed by capture using protein G beads as described above. Output titres were in the range 1×10⁶ to 1×10⁹ t.u./ml.

A third round of selection using 10 nM CD28-Fc followed by capture using protein G beads was performed. The eluted phage titres were in the range of 2×10⁹ to 8×10⁹ t.u./ml.

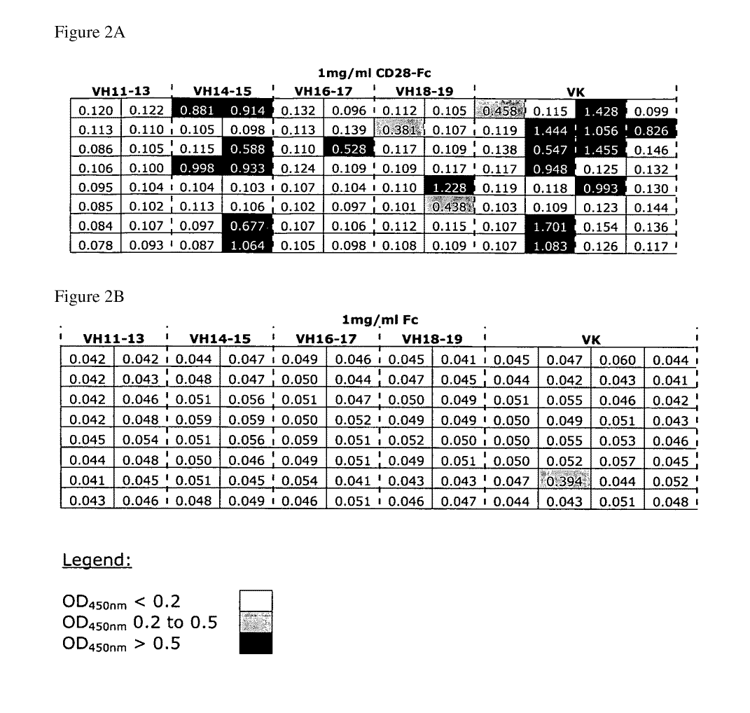

Monoclonal phage ELISAs were carried out following selection rounds 2 and 3. All washes were performed using 3 washes of 250 μl PBST followed by 3 washes of 250 μl PBS. Plates were coated overnight at 4° C. with 1 mg/ml and 0.6 mg/ml CD28-Fc in PBS respectively. Plates were washed, then blocked with 2% MPBS for 1 h at room temperature. The plates were washed and phage supernatants added to an equal volume of 2% MPBS and incubated for 1 h at room temperature. The plates were washed and bound phage detected with anti-M13-HRP conjugate (GE Healthcare, UK) diluted 1:5000 in 2% MPBS and incubated for 1 hour at room temperature. The plates were washed and the ELISA developed using SureBlue 1-Component TMB MicroWell Peroxidase solution (KPL Inc, USA). Specific phage were identified by comparison with a plate coated with 1 mg/ml Fc (Sigma Aldrich, UK). After round 2, specific phage were mainly identified in library pools VH14-15, VH18-19 and VK, whereas by round 3, few specific phage remained. All round 2 pools were subcloned into pDOM5 (FIG. 1B) and screened as soluble phage. The phage ELISA is shown in FIG. 2.

### Example 2

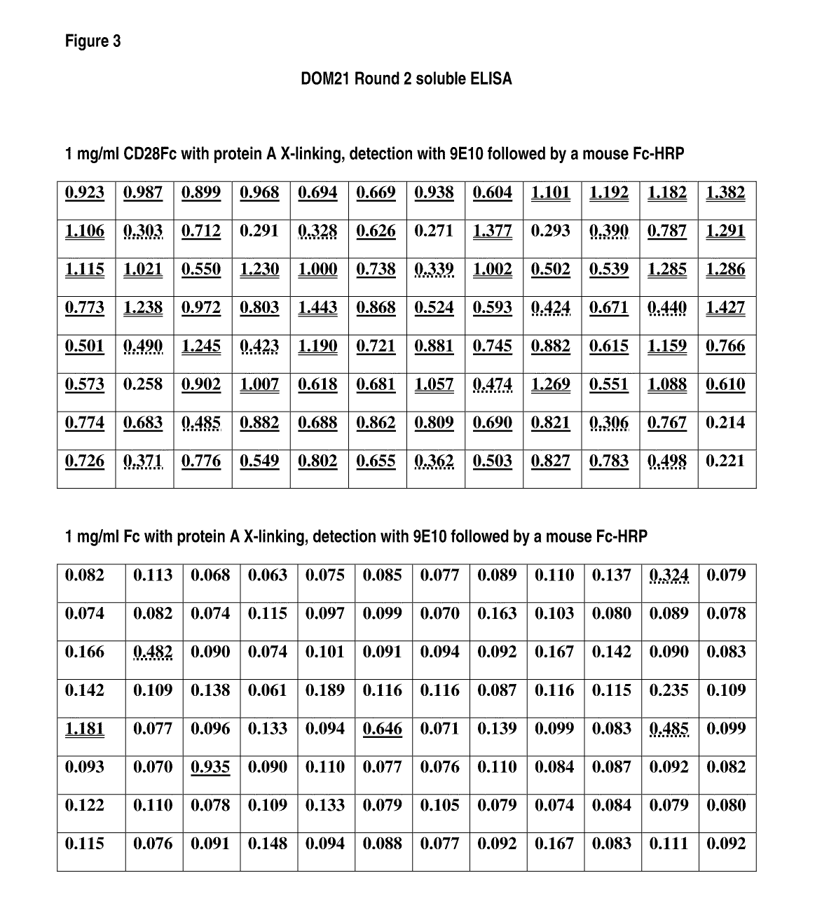

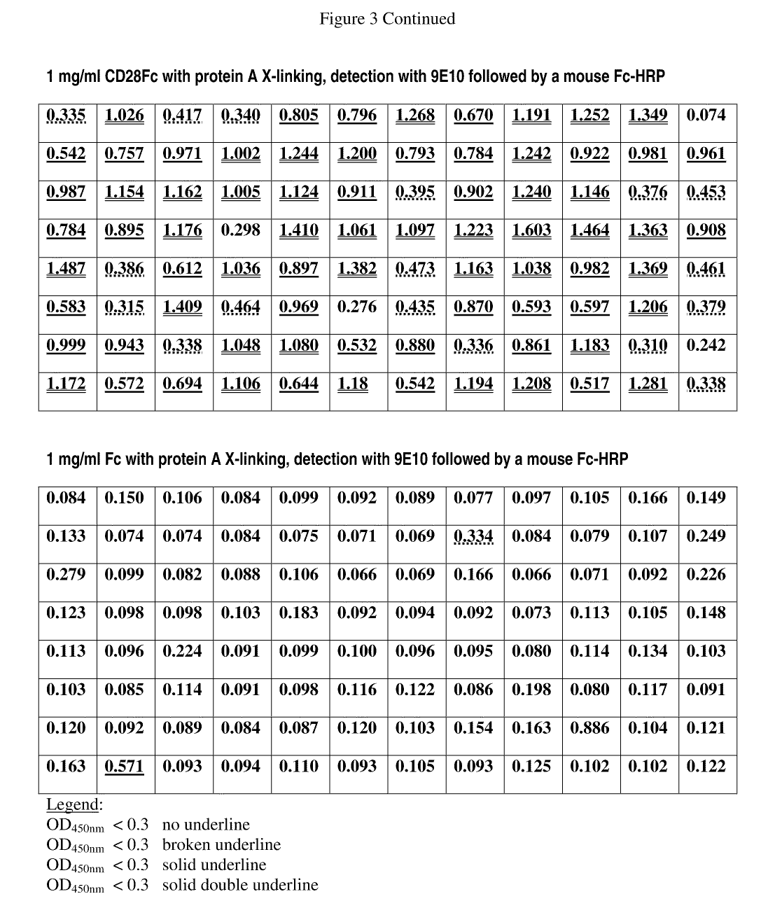

96 individual colonies (pDOM5) were picked from each of the VH14-15, VH18-19 and VK outputs and expressed in 200 μL Terrific Broth containing OnEx Autoinduction media (Novagen, UK) overnight at 37° C. with shaking at 250 rpm in Costar 96 Well Cell Culture Clusters (Corning Incorporated, USA). The cultures were centrifuged to pellet the cells and the supernatants assayed by antigen binding ELISA for CD28 binding dAbs. Maxisorp 96 well immunoplates (Nunc, USA) were coated overnight at 4° C. with 1 mg/ml CD28-Fc in PBS, and then washed. All washes were as described for the phage ELISA. The plates were blocked for 1 h at room temperature with 200 μl of PBS containing 1% Tween 20 and then washed. The clarified antibody single variable domain containing culture supernatant was added to the ELISA plate in the presence of either protein A for VH (Sigma, UK) or protein L for VK (Sigma, UK) to increase the ELISA signal strength by cross-linking the VH or VK antibody single variable domains, respectively. The plates were incubated for 1 h at room temperature and then washed. Bound antibody single variable domain was detected using a two step process, first 9E10 (anti-myc IgG, Sigma-Aldrich, UK) diluted 1:2000 in PBST was added for 1 h at room temperature and then washed, followed by anti-mouse Fc-HRP diluted 1:2000 in PBST for 1 h at room temperature. The plates were washed and the ELISA developed using SureBlue 1-Component TMB MicroWell Peroxidase solution (KPL Inc, USA) and the color was allowed to develop. The colorimetric reaction was stopped by the addition of an equal volume of 1M HCL and the ELISA plate was read at 450 nm. CD28 specific clones were identified by comparison to a control plate coated with Fc alone (see FIG. 3 for example of soluble ELISA). All specific clones were DNA sequenced and initially 28 unique clones were identified (see below for sequences). An additional two plates of antibody single variable domain supernatants were screened for binding to CD28-Fc by BIAcore analysis (GE Healthcare, UK). From this screening, an additional 30 unique sequences were identified (see below for sequences).

### Example 3

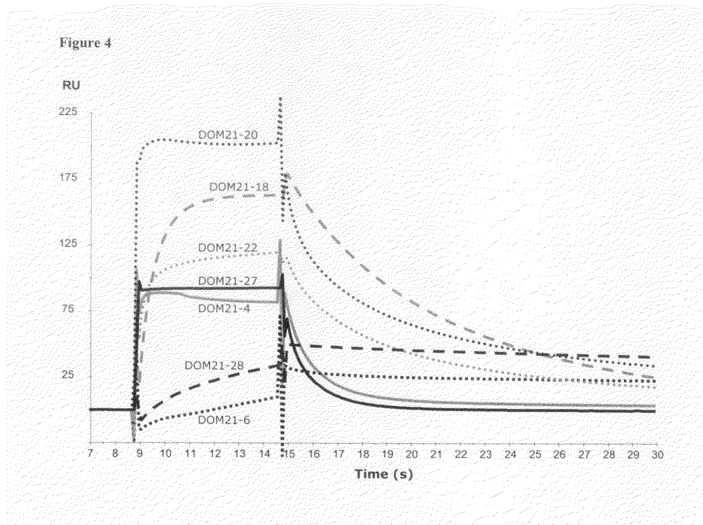

All 58 clones were expressed and purified and tested on the BIAcore against a CM5 chip coated with 12500 RU (response units) of CD28-Fc. A total of nine clones showed binding DOM21-4, DOM21-6, DOM21-18, DOM21-20, DOM21-22, DOM21-27 and DOM21-28 (see FIG. 4 for BIAcore traces) and DOM21-38 and DOM21-44. The protein concentrations used for BIAcore analysis were:

### DOM21-4 42.3 μM

### DOM21-6 68.1 μM

### DOM21-18 13.8 μM

### DOM21-20 57.5 μM

### DOM21-22 19.4 μM

### DOM21-27 14.7 μM

### DOM21-28 16.6 μM

### Example 4

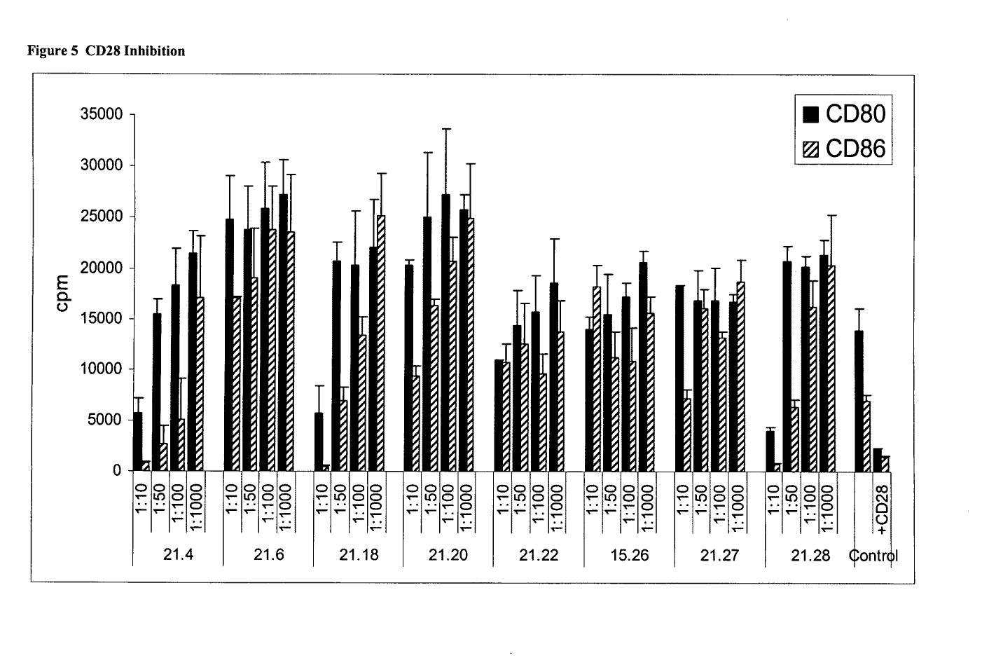

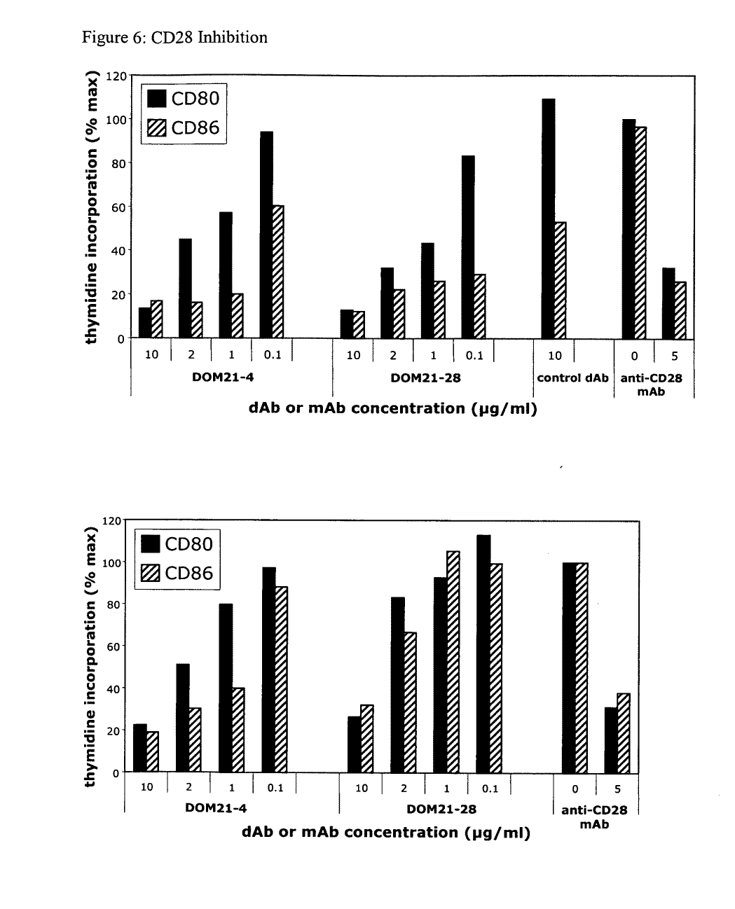

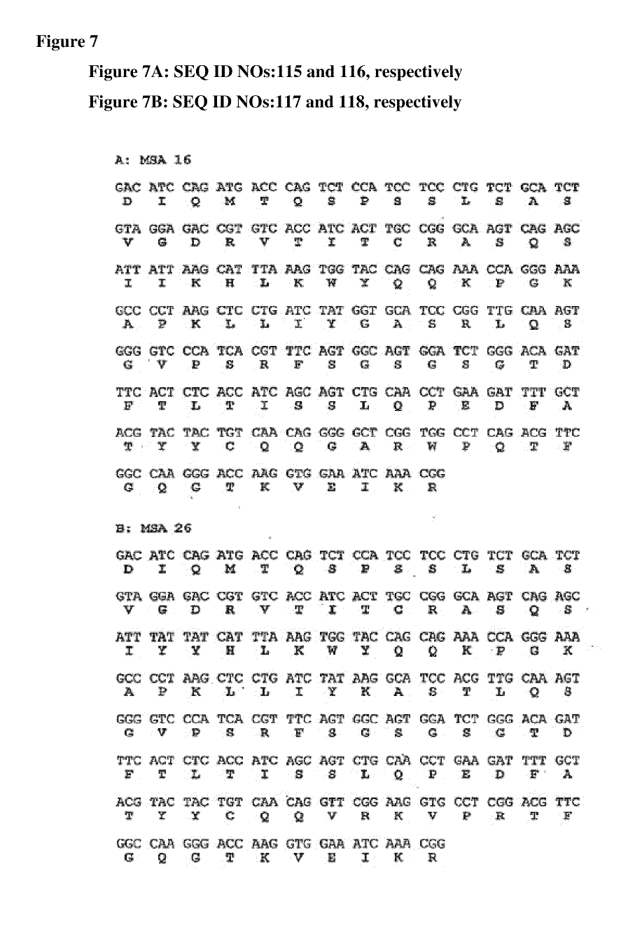

Seven of the dAbs (DOM21-4, DOM21-6, DOM21-18, DOM21-20, DOM21-22, DOM21-27 and DOM21-28) were expressed and purified on a larger scale. Endotoxin depleted dAbs samples at a stock concentration of 100 pM were used to determine whether the dAbs could inhibit the activity of CD28 in a cell based in vitro assay similar to that described by Boulougouris G, J. Immunol. 1998, 161(8): 3919-3924. Proliferation assays were performed in triplicate in 96-well plates in a final volume of 200 μl per well using RPMI 1640 medium containing 10% FCS and antibiotics. Human CD4 positive T-cells (5×10⁴) were cultured in the presence of 1 μg/ml anti-CD3 antibody (OKT3) plus transfected CHO cells expressing either CD80 or CD86 and dAb or control antibody at a range of concentrations. Assays were incubated at 37° C. for between 18 h to 72 h in the presence of 1 μCi [³H]thymidine per well. Cells were harvested onto 96-well filter plates using a Packard (Meriden, Conn.) 96-well harvester, and [³H]thymidine uptake was determined via liquid scintillation counting. Four dAbs, DOM21-4, DOM21-18, DOM21-20 and DOM21-28 showed inhibitory activity (FIG. 5; in FIG. 5, “21.4” refers to DOM21-4; “21.6” refers to DOM21-6; “21.18” refers to DOM21-18; etc.) with DOM21-4 and DOM21-28 showing the greatest degree of inhibition (FIG. 6)

All patents, patent applications, and published references cited herein are hereby incorporated by reference in their entirety. The disclosure set forth herein has been particularly shown and described with references to preferred embodiments thereof, it will be understood by those skilled in the art that various changes in form and details may be made therein without departing from the scope encompassed by the appended claims.

APPENDIX I
DOM21-1;
SEQ ID NO: 1
GAGGTGCAGCTGTTGGAGTCTGGGGGAGGCTTGGTACAGCCTGGGGGG
TCCCTGCGTCTCTCCTGTGCAGCCTCCGGATTCACCTTTGATGCGTAT
TCGATGATTTGGGTCCGCCAGGCTCCAGGGAAGGGTCTAGAGTGGGTC
TCAACTATTACTCCGCAGGGTGATAGGACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGCAAGCTGGTTGGAGTTTTGACTACTGGGGTCAGGGAACCCTGGTC
ACCGTCTCGAGC
DOM21-2;
SEQ ID NO: 2
GAGGTGCAGCTGTTGGAGTCTGGGGGAGGCTTGGTACAGCCTGGGGGG
TCCCTGCGTCTCTCCTGTGCAGCCTCCGGATTCACCTTTGTGGATTAT
GAGATGGCTTGGGTCCGCCAGGCTCCAGGGAAGGGTCTAGAGTGGGTC
TCAACTATTTCGAATGATGGCGCTGCTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGATGATGCTGCTTTTGACTACTGGGGTCAGGGAGCCCTGGTC
ACCGTCTCGAGCG
DOM21-3;
SEQ ID NO: 3
GAGGTGCAGCTGTTGGAGTCTGGGGGAGGCTTGGTACAGCCTGGGGGG
TCCCTGCGTCTCTCCTGTGCAGCCTCCGGATTCACCTTTGGTGCGTAT
TCTATGGGGTGGGCCCGCCAGGCTCCAGGGAAGGGTCTAGAGTGGGTC
TCATGGATTACGGGGAATGGTGGTTCTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGCGGAGGAGCCGTTTGACTACTGGGGTCAGGGAACCCTGGTC
ACCGTCTCGAGC
DOM21-4;
SEQ ID NO: 4
GAGGTGCAGCTGTTGGAGTCTGGGGGAGGCTTGGTACAGCCTGGGGGG
TCCCTGCGTCTCTCCTGTGCAGCCTCCGGATTCACCTTTAGTAGGTAT
CATATGGCGTGGGTCCGCCAGGCTCCAGGGAAGGGTCTAGAGTGGGTC
TCAGTGATTGATTCTCTTGGTCTTCAGACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGGAATATGGTGGTGCGTTTGACTACTGGGGTCAGGGAACCCTGGTC
ACCGTCTCGAGCG
DOM21-5;
SEQ ID NO: 5
GAGGTGCAGCTGTTGGAGTCTGGGGGAGGCTTGGTACAGCCTGGGGGG
TCCCTGCGTCTCTCCTGTGCAGCCTCCGGATTCACCTTTACTCATTAT
TCTATGGGGTGGGTCCGCCAGGCTCCAGGGAAGGGTCTAGAGTGGGTC
TCACATATTACTCCGGATGGTCTTATTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAAGGTAGGTTGGTTGATTTTGACTACTGGGGTCAGGGAACCCTG
GTCACCGTCTCGAGCG
DOM21-6;
SEQ ID NO: 6
GAGGTGCAGCTGTTGGAGTCTGGGGGAGGCTTGGTACAGCCTGGGGGG
TCCCTGCGTCTCTCCTGTGCAGCCTCCGGATTCACCTTTGAGAATTAT
GGTATGGCTTGGGTCCGCCAGGCTCCAGGGAAGGGTCTAGAGTGGGTC
TCAAATATTGGTCGGGCTGGTAGTGTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGTTCAGTCGTGGAGGACTTTTGACTACTGGGGTCAGGGAACC
CTGGTCACCGTCTCGAGC
DOM21-7;
SEQ ID NO: 7
GAGGTGCAGCTGTTGGAGTCTGGGGGAGGCTTGGTACAGCCTGGGGGG
TCCCTGCGTCTCTCCTGTGCAGCCTCCGGATTCACCTTTCCTGCGTAT
TCTATGGGGTGGGTCCGCCAGGCTCCAGAGAAGGGTCTAGAGTGGGTC
TCATATATTGATGGGCGTGGTGCTGAGACATACTACGCAGACTCCGTG
AAGGGCCGGTTCACCATCTCCCGCGACAATTCCAAGAACACGCTGTAT
CTGCAAATGAACAGCCTGCGCGCCGAGGATACCGCGGTATATTACTGT
GCGAAAATTGATACTCTGATTTCTGAGTTTGACTACTGGGGTCAGGGA
ACCCTGGTCACCGTCTCGAGC
DOM21-8;
SEQ ID NO: 8
GAGGTGCAGCTGTTGGAGTCTGGGGGAGGCTTGGTACAGCCTGGGGGG
TCCCTGCGTCTCTCCTGTGCAGCCTCCGGATTCACCTTTCCTAATTAT
ACGATGTGGTGGGTCCGCCAGGCTCCAGGGAAGGGTCTAGAGTGGGTC
TCATCTATTAGTGGTACTGGTCATACTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAATTTGGGCCTAATAATCCTATGTTTGACTACTGGGGTCAGGGA
ACCCTGGTCACCGTCTCGAGC
DOM21-9;
SEQ ID NO: 9
GAGGTGCAGCTGTTGGAGTCTGGGGGAGGCTTGGTACAGCCTGGGGGG
TCCCTGCGTCTCTCCTGTGCAGCCTCCGGATTCACCTTTGCGAGTTAT
GATATGGGTTGGGTCCGCCAGGCTCCAGGGAAGGGTCTAGAGTGGGTC
TCAGCGATTTCGGCGGATGGTACGTTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAATCTTCTTTTGATAAGTATAATTTTGACTACTGGGGTCAGGGA
ACCCTGGTCACCGTCTCGAGC
DOM21-10;
SEQ ID NO: 10
GAGGTGCAGCTGTTGGAGTCTGGGGGAGGCTTGGTACAGCCTGGGGGG
TCCCTGCGTCTCTCCTGTGCAGCCTCCGGATTCACCTTTGCTAAGTAT
ACGATGTGGTGGGTCCGCCAGGCTCCAGGGAAGGGTCTAGAGTGGGTC
TCAAGTATTGATCCTGTTGGTAATTTGACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAAGGGGGCCGACGTCGTCTAATTTTGACTACTGGGGTCAGGGA
ACCCTGGTCACCGTCTCGAGC
DOM21-11;
SEQ ID NO: 11
GAGGTGCAGCTGTTGGAGTCTGGGGGAGGCTTGGTACAGCCTGGGGGG
TCCCTGCGTCTCTCCTGTGCAGCCTCCGGATTCACCTTTAGTGAGTAT
GGTATGAAGTGGGTCCGCCAGGCTCCAGGGAAGGGTCTAGAGTGGGTC
TCAACGATTGATAATGTTGGTTCGGTGACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAACTACGCCTGTTTTGCTGCCGCTTTTTGACTACTGGGGTCAG
GGAACCCTGGTCACCGTCTCGAGC
DOM21-12;
SEQ ID NO: 12
GAGGTGCAGCTGTTGGAGTCTGGGGGAGGCTTGGTACAGCCTGGGGGG
TCCCTGCGTCTCTCCTGTGCAGCCTCCGGATTCACCTTTGATTCTTAT
AATATGGGTTGGGTCCGCCAGGCTCCAGGGAAGGGTCTTGAGTGGGTC
TCAGCTATTGCGGCTAATGGTCGTGTGACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAATGACGAATATGGCGTATGGTAGTTTTGACTACTGGGGTCAG
GGAACCCTGGTCACCGTCTCGAGC
DOM21-13;
SEQ ID NO: 13
GAGGTGCAGCTGTTGGAGTCTGGGGGAGGCTTGGTACAGCCTGGGGGG
TCCCTGCGTCTCTCCTGTGCAGCCTCCGGATTCACCTTTGATCTGTAT
TCGATGGCTTGGGTCCGCCAGGCTCCAGGGAAGGGTCTGGAGTGGGTC
TCACATATTGATAGGGCTGGTATGATTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGTTTCTAATGCTGTTAATATGCAGTTTGACTACTGGGGTCAG
GGAACCCTGGTCACCGTCTCGAGC
DOM21-14;
SEQ ID NO: 14
GAGGTGCAGCTGTTGGAGTCTGGGGGAGGCTTGGTACAGCCTGGGGGG
TCCCTGCGTCTCTCCTGTGCAGCCTCCGGATTCACCTTTGCTAAGTAT
ACGATGTGGTGGGTCCGCCAGGCTCCAGGGAAGGGTCTAGAGTGGGTC
TCAAGTATTGATCCTGTTGGTAATTTGACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACGTCATAGGCCTTCGACGCAGGATTTTGACTACTGGGGTCAG
GGAACCCTGGNCACCGTCTCGAGC
DOM21-15;
SEQ ID NO: 15
GAGGTGCAGCTGTTGGAGTCTGGGGGAGGCTTGGTACAGCCTGGGGGG
TCCCTGCGTCTCTCCTGTGCAGCCTCCGGATTCACCTTTCCTGATTAT
AAGATGGGTTGGGTCCGCCAGGCTCCAGGGAAGGGTCTAGAGTGGGTC
TCATGGATTGATAAGGGTGGTATTATTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAAATGTTTCCTAAGTTTCGGCCGGCTTTTGACTACTGGGGTCAG
GGAACCCTGGTCACCGTCTCGAGCG
DOM21-16;
SEQ ID NO: 16
GAGGTGCAGCTGTTGGAGTCTGGGGGAGGCTTGGTACAGCCTGGGGGG
TCCCTGCGTCTCTCCTGTGCAGCCTCCGGATTCACCTTTGAGGATTAT
GGGATGGGGTGGGTCCGCCAGGCTCCAGGGAAGGGTCTAGAGTGGGTC
TCACATATTAATCGTTCTGGTCTGGTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGTTCTGAATGCTCCTAATTTTAAGTTTGACTACTGGGGTCAG
GGAACCCTGGTCACCGTCTCGAGCG
DOM21-17;
SEQ ID NO: 17
GAGGTGCAGCTGTTGGAGTCTGGGGGAGGCTTGGTACAGCCTGGGGGG
TCCCTGCGTCTCTCCTGTGCAGCCTCCGGATTCACCTTTAATCGTTAT
GCGATGGGTTGGGTCCGCCAGGCTCCAGGGAAGGGTCTAGAGTGGGTC
TCATGGATTGATGGTAATGGTCTGGTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACGGACTAGGTCTCATTCTGATTCGGGTTGGGCTTTTGACTAC
TGGGGTCAGGGAACCCTGGTCACCGTCTCGAGC
DOM21-18;
SEQ ID NO: 18
GACATCCAGATGACCCAGTCTCCATCCTCCCTGTCTGCATCTGTAGGA
GACCGTGTCACCATCACTTGCCGGGCAAGTCAGTATATTGGTACTTCG
TTAAATTGGTACCAGCAGAAACCAGGGAAAGCCCCTAAGCTCCTGACC
TATCAGGCTTCCTTGTTGCAAAGTGGGGTCCCATCACGTTTCAGTGGC
AGTGGATCTGGGACAGATTTCACTCTCACCATCAGCAGTCTGCAACCT
GAAGATTTTGCTACGTACTACTGTCAACAGTTGGCGCTGCGTCCTATG
ACGTTCGGCCAAGGGACCAAGGTGGAAATCAAACGGG
DOM21-19;
SEQ ID NO: 19
GAGGTGCAGCTGTTGGAGTCTGGGGGAGGCTTGGTACAGCCTGGGGGG
TCCCTGCGTCTCTCCTGTGCAGCCTCCGGATTCACCTTTGGTAATTAT
AATATGGGGTGGGTCCGCCAGGCTCCAGGGAAGGGTCTAGAGTGGGTC
TCAGGTATTACGAAGGGTGGTCGGGTGACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAATTGGGTCCGTCGAGGATGCTTAATGAGCCGCTGTTTGACTAC
TGGGGTCAGGGAACCCTGGTCACCGTCTCGAGCG
DOM21-20;
SEQ ID NO: 20
GAGGTGCAGCTGTTGGAGTCTGGGGGAGGCTTGGTACAGCCTGGGGGG
TCCCTGCGTCTCTCCTGTGCAGCCTCCGGATTCACCTTTCCGGCGTAT
TCGATGATTTGGGTCCGCCAGGCTCCAGGGAAGGGTCTAGAGTGGGTC
TCAACGATTTCGCCGCTGGGTTATTCGACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGGAACAGACGGCTTATTTGAATCGTGCTACGGAGCATTTTGACTAC
TGGGGTCAGGGAACCCTGGTCACCGTCTCGAGCG
DOM21-21;
SEQ ID NO: 21
GAGGTGCAGCTGTTGGAGTCTGGGGGAGGCTTGGTACAGCCTGGGGGG
TCCCTGCGTCTCTCCTGTGCAGCCTCCGGATTCACCTTTTCGAAGTAT
GATATGGCTTGGGTCCGCCAGGCTCCAGGGAAGGGTCTGGAGTGGGTC
TCATCGATTTATGCTATTGGTGGTAATACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAATTGAAGTCGGGGATGCAGACTCGGTTGAATTCTTTTGACTAC
TGGGGTCAGGGAACCCTGGTCACCGTCTCGAGCG
DOM21-22;
SEQ ID NO: 22
GAGGTGCAGCTGTTGGAGTCTGGGGGAGGCTTGGTACAGCCTGGGGGG
TCCCTGCGTCTCTCCTGTGCAGCCTCCGGATTCACCTTTGAGCTGTAT
CAGATGGGTTGGGTCCGCCAGGCTCCAGGGAAGGGTCTAGAGTGGGTC
TCAACTATTATGCCTAGTGGTAATCTTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAAATGTGGTCGTTGAATTTGGGGTTTCATGCGGCTTTTGACTAC
TGGGGTCAGGGAACCCTGGTCACCGTCTCGAGC
DOM21-23;
SEQ ID NO: 23
GAGGTGCAGCTGTTGGAGTCTGGGGGAGGCTTGGTACAGCCTGGGGGG
TCCCTGCGTCTCTCCTGTGCAGCCTCCGGATTCACCTTTGGGCAGTAT
GGTATGGGTTGGGTCCGCCAGGCTCCAGGGAAGGGTCTAGAGTGGGTC
TCAGGGATTAGTCCTTCTGGTAATTATACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAAGGGAATGGGTCTCTTCCGCCTCGTGGGTCTATTTTTGACTAC
TGGGGTCAGGGAACCCTGGTCACCGTCTCGAGCG
DOM21-24;
SEQ ID NO: 24
GAGGTGCAGCTGTTGGAGTCTGGGGGAGGCTTGGTACAGCCTGGGGGG
TCCCTGCGTCTCTCCTGTGCAGCCTCCGGATTCACCTTTGGTAATTAT
AATATGGGGTGGGTCCGCCAGGCTCCAGGGAAGGGTCTAGAGTGGGTC
TCAGGTATTACGAAGGGTGGTCGGGTGACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAATTGGGTCCGTCGAGGATGCTTAATGAGCCGCTGTTTGACTAC
TGGGGTCAGGGAACCCTGGTCACCGTCTCGAGCG
DOM21-25;
SEQ ID NO: 25
GAGGTGCAGCTGTTGGAGTCTGGGGGAGGCTTGGTACAGCCTGGGGGG
TCCCTGCGTCTCTCCTGTGCAGCCTCCGGATTCACCTTTGGGACGTAT
TATATGGGGTGGGCCCGCCAGGCTCCAGGGAAGGGTCTAGAGTGGGTC
TCATCTATTGGGGCTAATGGTGCTCCTACATATTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAAATTCGTTCGCTTAATAGGTGGGCGGAGCCTGTGTTTGACTAC
TGGGGTCAGGGAACCCTGGTCACCGTCTCGAGC
DOM21-26;
SEQ ID NO: 26
GAGGTGCAGCTGTTGGAGTCTGGGGGAGGCTTGGTACAGCCTGGGGGG
TCCCTGCGTCTCTCCTGTGCAGCCTCCGGATTCACCTTTGCTGATTAT
TCTATGTATTGGGTCCGCCAGGCTCCAGGGAAGGGTCTGGAGTGGGTC
TCACAGATTAGTCCGGCGGGTTCTTTTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAAGATTCTAAGTCTTTTGACTACTGGGGTCAGGGAACCCTGGTC
ACCGTCTCGAGCG
DOM21-27;
SEQ ID NO: 27
GACATCCAGATGACCCAGTCTCCATCCTCCCTGTCTGCATCTGTAGGA
GACCGTGTCACCATCACTTGCCGGGCAAGTCAGAGTATTGGGACGGGT
TTACGGTGGTACCAGCAGAAACCAGGGAAAGCCCCTATGCTCCTGATC
TATCGGGCGTCCATTTTGCAAAGTGGGGTCCCATCACGTTTTAGTGGC
AGTGGATCTGGGACAGATTTCACTCTCACCATCAGCAGTCTGCAACCT
GAAGATTTTGCTACGTACTACTGTCAACAGACGACTCTTCAGCCTTTT
ACGTTCAGCCAAGGGACTAAGGTGGAAATCAAACGGG
DOM21-28;
SEQ ID NO: 28
GACATCCAGATGACCCAGTCTCCATCCTCCCTGTCTGCATCTGTAGGA
GACCGTGTCACCATCACTTGCCGGGCAAGTCAGTCTATTAGTCATTCG
TTAGTTTGGTACCAGCAGAAACCAGGGAAAGCCCCTAAGCTCCTGATC
TATTGGGCTTCCCTTTTGCAAAGTGGGGTCCCATCACGTTTCAGTGGC
AGTGGATCTGGGACAGATTTCACTCTCACCATCAGCAGTCTGCAACCT
GAAGATTTTGCTACGTACTACTGTCAACAGGGTATGACTACGCCTTTT
ACGTTCGGCCAAGGGACCAAGGTGGAAATCAAACGGG
DOM21-30;
SEQ ID NO: 29
GAGGTGCAGCTGTTGGAGTCTGGGGGAGGCTTGGTACAGCCTGGGGGG
TCCCTGCGTCTCTCCTGTGCAGCCTCCGGATTCACCTTTGATAGTTAT
GATATGAATTGGGTCCGCCAGGCTCCAGGGAAGGGTCTAGAGTGGGTC
TCACAGATTTCTGCTGATGGTCATTTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAATCGCGGAGTAGTTTTGACTACTGGGGTCAGGGAACCCTGGTC
ACCGTCTCGAGC
DOM21-31;
SEQ ID NO: 30
GAGGTGCAGCTGTTGGAGTCTGGGGGAGGCTTGGTACAGCCTGGGGGG
TCCCTGCGTCTCTCCTGTGCAGCCTCCGGATTCACCTTTAGGGATTAT
ATGATGGGGTGGGTCCGCCAGGCTCCAGGGAAGGGTCTAGAGTGGGTC
TCACGTATTGATTCTCATGGTAATCGTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACATATGACGGGTTTTGACTACTGGGGTCAGGGAACCCTGGTC
ACCGTCTCGAGC
DOM21-32;
SEQ ID NO: 31
GAGGTGCAGCTGTTGGAGTCTGGGGGAGGCTTGGTACAGCCTGGGGGG
TCCCTGCGTCTCTCCTGTGCAGCCTCCGGATTCACCTTTAGGGAGTAT
ATGATGGGGTGGGTCCGCCAGGCTCCAGGGAAGGGTCTAGAGTGGGTC
TCACGTATTAATGGTGTGGGTAATTCTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACATCAGGTGGGTTTTGACTACTGGGGTCAGGGAACCCTGGTC
ACCGTCTCGAGC
DOM21-33;
SEQ ID NO: 32
GAGGTGCAGCTGTTGGAGTCTGGGGGAGGCTTGGTACAGCCTGGGGGG
TCCCTGCGTCTCTCCTGTGCAGCCTCCGGATTCACCTTTAGTGATTAT
ATGATGGGTTGGGTCCGCCAGGCTCCAGGGAAGGGTCTAGAGTGGGTC
TCACGTATTACGTCTGAGGGTTCGCATACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACATACGTCTGGTTTTGACTACTGGGGTCAGGGAACCCTGGTC
ACCGTCTCGAGC
DOM21-34;
SEQ ID NO: 33
GAGGTGCAGCTGTTGGAGTCTGGGGGAGGCTTGGTACAGCCTGGGGGG
TCCCTGCGTCTCTCCTGTGCAGCCTCCGGATTCACCTTTGGGAGGTAT
ATGATGGGGTGGGTCCGCCAGGCTCCAGGGAAGGGTCTGGAGTGGGTC
TCACGGATTTCTGGTCCTGGTACGGTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACATGATACGGGGTTTGACTACTGGGGTCAGGGAACCCTGGTC
ACCGTCTCGAGC
DOM21-35;
SEQ ID NO: 34
GAGGTGCAGCTGTTGGAGTCTGGGGGAGGCTTGGTACAGCCTGGGGGG
TCCCTGCGTCTCTCCTGTGCAGCCTCCGGATTCACCTTTTCTTCTTAT
GCTATGATTTGGGTCCGCCAGGCTCCAGGGAAGGGTCTAGAGTGGGTC
TCAGAGATTTCTCCTTATGGTAATCATACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACCTGATCGGCGTTTTGACTACTGGGGTCAGGGAACCCTGGTC
ACCGTCTCGAGC
DOM21-36;
SEQ ID NO: 35
GAGGTGCAGCTGTTGGAGTCTGGGGGAGGCTTGGTACAGCCTGGGGGG
TCCCTGCGTCTCTCCTGTGCAGCCTCCGGATTCACCTTTACTTCGTAT
GGGATGCAGTGGGTCCGCCAGGCTCCAGGGAAGGGTCTAGAGTGGGTC
TCATCGATTTCTACTGATGGTATGGTTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAACTTGGGGTTAATTTTGACTACTGGGGTCAGGGAACCCTGGTC
ACCGTCTCGAGC
DOM21-37;
SEQ ID NO: 36
GAGGTGCAGCTGTTGGAGTCTGGGGGAGGCTTGGTACAGCCTGGGGGG
TCCCTGCGTCTCTCCTGTGCAGCCTCCGGATTCACCTTTGGTGATTAT
ATGATGGGGTGGGTCCGCCAGGCTCCAGGGAAGGGTCTGGAGTGGGTC
TCAATTATTCGTGTGCCTGGTTCGACTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACAGAAGGGTGATGAGTTTGACTACTGGGGTCAGGGAACCCTG
GTCACCGTCTCGAGC
DOM21-38;
SEQ ID NO: 37
GAGGTGCAGCTGTTGGAGTCTGGGGGAGGCTTGGTACAGCCTGGGGGG
TCCCTGCGTCTCTCCTGTGCAGCCTCCGGATTCACCTTTATTCTGTAT
GATATGCAGTGGGTCCGCCAGGCTCCAGGGAAGGGTCTAGAGTGGGTC
TCACGTATTTCTGCTAATGGTCATGATACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGGTCCGCATTATTTGTTTGACTACTGGGGTCAGGGAACCCTG
GTCACCGTCTCGAGC
DOM21-39;
SEQ ID NO: 38
GAGGTGCAGCTGTTGGAGTCTGGGGGAGGCTTGGTACAGCCTGGGGGG
TCCCTGCGTCTCTCCTGCGCAGCCTCCGGATTCACCTTTACTAAGTAT
TTTATGGGTTGGGTCCGCCAGGCTCCAGGGAAGGGTCTAGAGTGGGTC
TCACTGATTGATCCGCGTGGTCCTCATACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACAGTTGGGTGAGGAGTTTGACTACTGGGGTCAGGGAACCCTG
GTCACCGTCTCGAGC
DOM21-40;
SEQ ID NO: 39
GAGGTGCAGCTGTTGGAGTCTGGGGGAGGCTTGGTACAGCCTGGGGGG
TCCCTGCGTCTCTCCTGTGCAGCCTCCGGATTCACCTTTAAGACTTAT
ACGATGAGGTGGGTCCGCCAGGCTCCAGGGAAGGGTCTAGAGTGGGTC
TCAACTATTAATTCGAGTGGTACTTTGACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAATCTAGTTCTTATACGTTTGACTACTGGGGTCAGGGAACCCTG
GTCACCGTCTCGAGC
DOM21-41;
SEQ ID NO: 40
GAGGTGCAGCTGTTGGAGTCTGGGGGAGGCTTGGTACAGCCTGGGGGG
TCCCTGCGTCTCTCCTGTGCAGCCTCCGGATTCACCTTTGCGATGTAT
AGTATGAAGTGGGTCCGCCAGGCTCCAGGGAAGGGTCTAGAGTGGGTC
TCATCGATTTCGAATGCTGGTGATATTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGGAATCGTTTAGGTCTCGTTATTTTGACTACTGGGGTCAGGGAACC
CTGGTCACCGTCTCGAGC
DOM21-42;
SEQ ID NO: 41
GAGGTGCAGCTGTTGGAGTCTGGGGGAGGCTTGGTACAGCCTGGGGGG
TCCCTGCGTCTCTCCTGTGCAGCCTCCGGATTCACCTTTGATGATTAT
CTTATGGGGTGGGTCCGCCAGGCTCCAGGGAAGGGTCTGGAGTGGGTC
TCACTGATTCGTATGAGGGGTTCTGTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACATTCTCTTACTACTAATCTTTTTGACTACTGGGGTCAGGGA
ACCCTGGTCACCGTCTCGAGC
DOM21-43;
SEQ ID NO: 42
GAGGTGCAGCTGTTGGAGTCTGGGGGAGGCTTGGTACAGCCTGGGGGG
TCCCTGCGTCTCTCCTGTGCAGCCTCCGGATTCACCTTTACTGATTAT
ATGATGGCTTGGGCCCGCCAGGCTCCAGGGAAGGGTCTAGAGTGGGTC
TCAATTATTGGGACTACTGGTACGTGGACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAACTAATGCGTATGAGAGTGAGTTTGACTACTGGGGTCAGGGA
ACCCTGGTCACCGTCTCGAGC
DOM21-44;
SEQ ID NO: 43
GAGGTGCAGCTGTTGGAGTCTGGGGGAGGCTTGGTACAGCCTGGGGGG
TCCCTGCGTCTCTCCTGTGCAGCCTCCGGATTCACCTTTGCGCGGTAT
ACTATGGTGTGGGTCCGCCAGGCTCCAGGGAAGGGTCTAGAGTGGGTC
TCAGCTATTCATTTTGATGGTCGGACTACATACTACGCAGACTCCGTG
AAGGGCCGGTTCACCATCTCCCGCGACAATTCCAAGAACACGCTGTAT
CTGCAAATGAATAGCCTGCGTGCCGAGGACACCGCGGTATATTACTGT
GCGAAAAATGAGTGGGCGTCTCTTAAGCATTTTGACTACTGGGGTCAG
GGAACCCTGGTCACCGTCTCGAGC
DOM21-45;
SEQ ID NO: 44
GAGGTGCAGCTGTTGGAGTCTGGGGGAGGCTTGGTACAGCCTGGGGGG
TCCCTGCGTCTCTCCTGTGCAGCCTCCGGATTCACCTTTGAGGATTAT
ATGATGGGTTGGGTCCGCCAGGCTCCAGGGAAGGGTCTAGAGTGGGTC
TCATTTATTAATCTGCCTGGTGGTCGTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACAGACTCATGGGCTGACTGGTTATTTTGACTACTGGGGTCAG
GGAACCCTGGTCACCGTCTCGAGC
DOM21-46;
SEQ ID NO: 45
GAGGTGCAGCTGTTGGAGTCTGGGGGAGGCTTGGTACAGCCTGGGGGG
TCCCTGCGTCTCTCCTGTGCAGCCTCCGGATTCACCTTTGGTTTGTAT
GGTATGGCTTGGGCCCGCCAGGCTCCAGGGAAGGGTCTAGAGTGGGTC
TCATCGATTGGGATGCATGGTGATACTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGTTTGTGGGGCTACGTATTGTAATTTTGACTACTGGGGTCAG
GGAACCCTGGTCACCGTCTCGAGC
DOM21-47;
SEQ ID NO: 46
GAGGTGCAGCTGTTGGAGTCTGGGGGAGGCTTGGTACAGCCTGGGGGG
TCCCTGCGTCTCTCCTGTGCAGCCTCCGGATTCACCTTTGGTAAGTAT
GTTATGGCTTGGGTCCGCCAGGCTCCAGGGAAGGGTCTAGAGTGGGTC
TCAATTATTGATTCCTTGGGTTCTACTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGGGGGTTTGTTGGTTCATTATGATTTTGACTACTGGGGTCAG
GGAACCCTGGTCACCGTCTCGAGC
DOM21-48;
SEQ ID NO: 47
GAGGTGCAGCTGTTGGAGTCTGGGGGAGGCTTGGTACAGCCTGGGGGG
TCCCTGCGTCTCTCCTGTGCAGCCTCCGGATTCACCTTTGAGGTGTAT
GGTATGTCTTGGGCCCGCCAGGCTCCAGGGAAGGGTCTAGAGTGGGTC
TCATTGATTGATGCGGGTGGTCGGAATACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAATCGACGACGCGTGCTTATAGTGATTATTTTGACTACTGGGGT
CAGGGAACCCTGGTCACCGTCTCGAGC
DOM21-49;
SEQ ID NO: 48
GAGGTGCAGCTGTTGGAGTCTGGGGGAGGCTTGGTACAGCCTGGGGGG
TCCCTGCGTCTCTCCTGTGCAGCCTCCGGATTCACCTTTGAGAATTAT
GATATGCATTGGGTCCGCCAGGCTCCAGGGAAGGGTCTAGAGTGGGTC
TCAGGGATTACTACGCATGGTAGGCGTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAAAGTGATAATTTGAATATGAATGIGGATTTTGACTACTGGGGT
CAGGGAACCCTGGTCACCGTCTCGAGC
DOM21-50;
SEQ ID NO: 49
GAGGTGCAGCTGTTGGAGTCTGGGGGAGGCTTGGTACAGCCTGGGGGG
TCCCTGCGTCTCTCCTGTGCAGCCTCCGGATTCACCTTTATTAAGTAT
GATATGTGTTGGGCCCGCCAGGCTCCAGGGAAGGGTCTAGAGTGGGTC
TCATGTATTGAGTCTAGTGGTCAGAATACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAATGTCTGAATGATAGTTGTAATGTTCATTTTGACTACTGGGGT
CAGGGAACCCTGGTCACCGTCTCGAGC
DOM21-51;
SEQ ID NO: 50
GAGGTGCAGCTGTTGGAGTCTGGGGGAGGCTTGGTACAGCCTGGGGGG
TCCCTGCGTCTCTCCTGTGCAGCCTCCGGATTCACCTTTGGTAATTAT
AATATGGGGTGGGTCCGCCAGGCTCCAGGGAAGGGTCTAGAGTGGGTC
TCAGATATTGGTCGTTATGGTAGGGTTACATACTACGCAGACTCCGTG
AAGGGCCGGTTCACCATCTCCCGCGACAATTCCAAGAACACGCTGTAT
CTGCAAATGAACAGCCTGCGTGCCGAGGATACCGCGGTATATTACTGT
GCGAAAACTCAGCGTATGGTTAATCCGTCGCCTTTTGACTACTGGGGT
CAGGGAACCCTGGTCACCGTCTCGAGC
DOM21-52;
SEQ ID NO: 51
GAGGTGCAGCTGTTGGAGTCTGGGGGAGGCTTGGTACAGCCTGGGGGG
TCCCTGCGTCTCTCCTGTGCAGCTTCCGGATTCACCTTTGTTAGTTAT
AGTATGGGTTGGGTCCGCCAGGCTCCAGGGAAGGGTCTAGAGTGGGTC
TCAATTATTTCGGGGCAGGGTACTGTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAATCGCCGATGGTTTTTGCTTTGGATGGGAGGTCTTTTGACTAC
TGGGGTCAGGGAACCCTGGTCACCGTCTCGAGC
DOM21-53;
SEQ ID NO: 52
GAGGTGCAGCTGTTGGAGTCTGGGGGAGGCTTGGTACAGCCTGGGGGG
TCCCTGCGTCTCTCCTGTACAGCCTCCGGATTCACCTTTTCTGAGTAT
AGTATGGGGTGGGTCCGCCAGGCTCCAGGGAAGGGTCTAGAGTGGGTC
TCAAGTATTACGCCTGTTGGTGTTTTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGGGAGGCCTGGGCCGCATGGTTGGTCTTTTCGGTTTGACTAC
TGGGGTCAGGGAACCCTGGTCACCGTCTCGAGC
DOM21-54;
SEQ ID NO: 53
GAGGTGCAGCTGTTGGAGTCTGGGGGAGGCTTGGTACAGCCTGGGGGG
TCCCTGCGTCTCTCCTGTGCAGCCTCCGGATTCACCTTTGGGCAGTAT
ATGATGGGTTGGGTCCGCCAGGCTCCAGGGAAGGGTCTAGAGTGGGTC
TCAACTATTGATAAGTCGGGTTATAGTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAAGTGGGATTGATTCGCGGGGTCTGATGACTAAGTTTGACTAC
TGGGGTCAGGGAACCCTGGTCACCGTCTCGAGC
DOM21-55;
SEQ ID NO: 54
GAGGTGCAGCTGTTGGAGTCTGGGGGAGGCTTGGTACAGCCTGGGGGG
TCCCTGCGTCTCTCCTGTGCAGCCTCCGGATTCACCTTTGCTCGTTAT
CGTATGGCGTGGGTCCGCCAGGCTCCAGGGAAGGGTCTAGAGTGGGTC
TCATCTATTCTGAGTGATGGTGCGGTTACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACCTGGGGGGAATGCGTGGTCTACTCGGGTTACTTTTGACTAC
TGGGGTCAGGGAACCCTGGTCACCGTCTCGAGC
DOM21-56;
SEQ ID NO: 55
GAGGTGCAGCTGTTGGAGTCTGGGGGAGGCTTGGTACAGCCTGGGGGG
TCCCTGCGTCTCTCCTGTGCAGCCTCCGGATTCACCTTTTTTACGTAT
ACNATGGCTTGGGTCCGCCAGGCTCCAGGGAAGGGTCTAGAGTGGGTC
TCATCTATTACGCCGCTTGGTTATAATACATACTACGCAGACTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAACCGTCGGATGTGAAGGTGTCTCCGCTGCCGAGTTTTGACTAC
TGGGGTCGGGGAACCCTGGTCACCGTCTCGAGC
DOM21-57;
SEQ ID NO: 56
GAGGTGCAGCTGTTGGAGTCTGGGGGAGGCTTGGTACAGCCTGGGGGG
TCCCTGCGTCTCTCCTGTGCAGCCTCCGGATTCACCTTTACTATGTAT
GGTATGCATTGGGTCCGCCAGGCTCCAGGGAAGGGTCTAGAGTGGGTC
TCATCGATTTCTCAGTATGGTCTTTCTACATACTACGCAGATTCCGTG
AAGGGCCGGTTCACCATCTCCCGCGACAATTCCAAGAACACGCTGTAT
CTGCAAATGAACAGCCTGCGTGCCGAGGACACCGCGGTATATTACTGT
GCGAAAGGGTCTATGAGGCGGGTGTTTAGTAGTTCGGATACTTTTGAC
TACTGGGGTCAGGGAACCCTGGTCACCGTCTCGAGC
DOM21-58;
SEQ ID NO: 57
GACATCCAGATGACCCAGTCTCCATCCTCCCTGTCTGCATCTGTAGGA
GACCGTGTCACCATCACTTGCCGGGCAAGTCAGAATATAGGTGATCGG
TTACATTGGTACCAGCAGAAACCAGGGAAAGCCCCTAAGCTCCTGATC
TATCGTATTTCCCGTTTGCAAAGTGGGGTCCCATCACGTTTCAGTGGC
AGTGGATCTGGGACAGATTTCACTCTCACCATCAGCAGTCTGCAACCT
GAAGATTTTGCTACGTACTACTGTCAACAGTTTGGGCTGTATCCTACT
ACGTTCGGCCAAGGGACCAAGGTGGAAATCAAACGG
DOM21-1;
SEQ ID NO: 58
EVQLLESGGGLVQPGGSLRLSCAASGFTFDAYSMIWVRQAPGKGLEWV
STITPQGDRTYYADSVKGRFTISRDNSKNTLYLQMNSLRAEDTAVYYC
AQAGWSFDYWGQGTLVTVSS
DOM21-2;
SEQ ID NO: 59
EVQLLESGGGLVQPGGSLRLSCAASGFTFVDYEMAWVRQAPGKGLEWV
STISNDGAATYYADSVKGRFTISRDNSKNTLYLQMNSLRAEDTAVYYC
AKDDAAFDYWGQGALVTVSS
DOM21-3;
SEQ ID NO: 60
EVQLLESGGGLVQPGGSLRLSCAASGFTFGAYSMGWARQAPGKGLEWV
SWITGNGGSTYYADSVKGRFTISRDNSKNTLYLQMNSLRAEDTAVYYC
AKAEEPFDYWGQGTLVTVSS
DOM21-4;
SEQ ID NO: 61
EVQLLESGGGLVQPGGSLRLSCAASGFTFSRYHMAWVRQAPGKGLEWV
SVIDSLGLQTYYADSVKGRFTISRDNSKNTLYLQMNSLRAEDTAVYYC
AEYGGAFDYWGQGTLVTVSS
DOM21-5;
SEQ ID NO: 62
EVQLLESGGGLVQPGGSLRLSCAASGFTFTHYSMGWVRQAPGKGLEWV
SHITPDGLITYYADSVKGRFTISRDNSKNTLYLQMNSLRAEDTAVYYC
AKGRLVDFDYWGQGTLVTVSS
DOM21-6;
SEQ ID NO: 63
EVQLLESGGGLVQPGGSLRLSCAASGFTFENYGMAWVRQAPGKGLEWV
SNIGRAGSVTYYADSVKGRFTISRDNSKNTLYLQMNSLRAEDTAVYYC
AKVQSWRTFDYVVGQGTLVTVSS
DOM21-7;
SEQ ID NO: 64
EVQLLESGGGLVQPGGSLRLSCAASGFTFPAYSMGWVRQAPEKGLEWV
SYIDGRGAETYYADSVKGRFTISRDNSKNTLYLQMNSLRAEDTAVYYC
AKIDTLISEFDYWGQGTLVTVSS
DOM21-8;
SEQ ID NO: 65
EVQLLESGGGLVQPGGSLRLSCAASGFTFPNYTMWWVRQAPGKGLEWV
SSISGTGHTTYYADSVKGRFTISRDNSKNTLYLQMNSLRAEDTAVYYC
AKFGPNNPMFDYWGQGTLVTVSS
DOM21-9;
SEQ ID NO: 66
EVQLLESGGGLVQPGGSLRLSCAASGFTFASYDMGWVRQAPGKGLEWV
SAISADGTFTYYADSVKGRFTISRDNSKNTLYLQMNSLRAEDTAVYYC
AKSSFDKYNFDYWGQGTLVTVSS
DOM21-10;
SEQ ID NO: 67
EVQLLESGGGLVQPGGSLRLSCAASGFTFAKYTMWWVRQAPGKGLEWV
SSIDPVGNLTYYADSVKGRFTISRDNSKNTLYLQMNSLRAEDTAVYYC
AKRGPTSSNFDYWGQGTLVTVSS
DOM21-11;
SEQ ID NO: 68
EVQLLESGGGLVQPGGSLRLSCAASGFTFSEYGMKWVRQAPGKGLEWV
STIDNVGSVTYYADSVKGRFTISRDNSKNTLYLQMNSLRAEDTAVYYC
AKTTPVLLPLFDYVVGQGTLVTVSS
DOM21-12;
SEQ ID NO: 69
EVQLLESGGGLVQPGGSLRLSCAASGFTFDSYNMGWVRQAPGKGLEWV
SAIAANGRVTYYADSVKGRFTISRDNSKNTLYLQMNSLRAEDTAVYYC
AKMTNMAYGSFDYWGQGTLVTVSS
DOM21-13;
SEQ ID NO: 70
EVQLLESGGGLVQPGGSLRLSCAASGFTFDLYSMAWVRQAPGKGLEWV
SHIDRAGMITYYADSVKGRFTISRDNSKNTLYLQMNSLRAEDTAVYYC
AKVSNAVNMQFDYVVGQGTLVTVSS
DOM21-14;
SEQ ID NO: 71
EVQLLESGGGLVQPGGSLRLSCAASGFTFAKYTMWWVRQAPGKGLEWV
SSIDPVGNLTYYADSVKGRFTISRDNSKNTLYLQMNSLRAEDTAVYYC
AKRHRPSTQDFDYWGQGTLXTVSS
DOM21-15;
SEQ ID NO: 72
EVQLLESGGGLVQPGGSLRLSCAASGFTFPDYKMGWVRQAPGKGLEWV
SWIDKGGIITYYADSVKGRFTISRDNSKNTLYLQMNSLRAEDTAVYYC
AKMFPKFRPAFDYWGQGTLVTVSS
DOM21-16;
SEQ ID NO: 73
EVQLLESGGGLVQPGGSLRLSCAASGFTFEDYGMGWVRQAPGKGLEWV
SHINRSGLVTYYADSVKGRFTISRDNSKNTLYLQMNSLRAEDTAVYYC
AKVLNAPNFKFDYWGQGTLVTVSS
DOM21-17;
SEQ ID NO: 74
EVQLLESGGGLVQPGGSLRLSCAASGFTFNRYAMGWVRQAPGKGLEWV
SWIDGNGLVTYYADSVKGRFTISRDNSKNTLYLQMNSLRAEDTAVYYC
AKRTRSHSDSGWAFDYWGQGTLVTVSS
DOM21-18;
SEQ ID NO: 75
DIQMTQSPSSLSASVGDRVTITCRASQYIGTSLNWYQQKPGKAPKLLT
YQASLLQSGVPSRFSGSGSGTDFTLTISSLQPEDFATYYCQQLALRPM
TFGQGTKVEIKR
DOM21-19;
SEQ ID NO: 76
EVQLLESGGGLVQPGGSLRLSCAASGFTFGNYNMGWVRQAPGKGLEWV
SGITKGGRVTYYADSVKGRFTISRDNSKNTLYLQMNSLRAEDTAVYYC
AKLGPSRMLNEPLFDWGQGTLVTVSS
DOM21-20;
SEQ ID NO: 77
EVQLLESGGGLVQPGGSLRLSCAASGFTFPAYSMIWVRQAPGKGLEWV
STISPLGYSTYYADSVKGRFTISRDNSKNTLYLQMNSLRAEDTAVYYC
AEQTAYLNRATEHFDYVVGQGTLVTVSS
DOM21-21;
SEQ ID NO: 78
EVQLLESGGGLVQPGGSLRLSCAASGFTFSKYDMAWVRQAPGKGLEWV
SSIYAIGGNTYYADSVKGRFTISRDNSKNTLYLQMNSLRAEDTAVYYC
AKLKSGMQTRLNSFDYWGQGTLVTVSS
DOM21-22;
SEQ ID NO: 79
EVQLLESGGGLVQPGGSLRLSCAASGFTFELYQMGWVRQAPGKGLEWV
STIMPSGNLTYYADSVKGRFTISRDNSKNTLYLQMNSLRAEDTAVYYC
AKMWSLNLGFHAAFDYWGQGTLVTVSS
DOM21-23;
SEQ ID NO: 80
EVQLLESGGGLVQPGGSLRLSCAASGFTFGQYGMGWVRQAPGKGLEWV
SGISPSGNYTYYADSVKGRFTISRDNSKNTLYLQMNSLRAEDTAVYYC
AKGNGSLPPRGSIFDYVVGQGTLVTVSS
DOM21-24;
SEQ ID NO: 81
EVQLLESGGGLVQPGGSLRLSCAASGFTFGNYNMGWVRQAPGKGLEWV
SGITKGGRVTYYADSVKGRFTISRDNSKNTLYLQMNSLRAEDTAVYYC
AKLGPSRMLNEPLFDWGQGTLVTVSS
DOM21-25;
SEQ ID NO: 82
EVQLLESGGGLVQPGGSLRLSCAASGFTFGTYYMGWARQAPGKGLEWV
SSIGANGAPTYYADSVKGRFTISRDNSKNTLYLQMNSLRAEDTAVYYC
AKIRSLNRWAEPVFDYWGQGTLVTVSS
DOM21-26;
SEQ ID NO: 83
EVQLLESGGGLVQPGGSLRLSCAASGFTFADYSMYWVRQAPGKGLEWV
SQISPAGSFTYYADSVKGRFTISRDNSKNTLYLQMNSLRAEDTAVYYC
AKDSKSFDYWGQGTLVTVSS
DOM21-27;
SEQ ID NO: 84
DIQMTQSPSSLSASVGDRVTITCRASQSIGTGLRWYQQKPGKAPMLLI
YRASILQSGVPSRFSGSGSGTDFTLTISSLQPEDFATYYCQQTTLQPF
TFSQGTKVEIKR
DOM21-28;
SEQ ID NO: 85
DIQMTQSPSSLSASVGDRVTITCRASQSISHSLVWYQQKPGKAPKLLI
YWASLLQSGVPSRFSGSGSGTDFTLTISSLQPEDFATYYCQQGMTTPF
TFGQGTKVEIKR
DOM21-30;
SEQ ID NO: 86
EVQLLESGGGLVQPGGSLRLSCAASGFTFDSYDMNWVRQAPGKGLEWV
SQISADGHFTYYADSVKGRFTISRDNSKNTLYLQMNSLRAEDTAVYYC
AKSRSSFDYWGQGTLVTVSS
DOM21-31;
SEQ ID NO: 87
EVQLLESGGGLVQPGGSLRLSCAASGFTFRDYMMGWVRQAPGKGLEWV
SRIDSHGNRTYYADSVKGRFTISRDNSKNTLYLQMNSLRAEDTAVYYC
AKHMTGFDYWGQGTLVTVSS
DOM21-32;
SEQ ID NO: 88
EVQLLESGGGLVQPGGSLRLSCAASGFTFREYMMGWVRQAPGKGLEWV
SRINGVGNSTYYADSVKGRFTISRDNSKNTLYLQMNSLRAEDTAVYYC
AKHQVGFDYWGQGTLVTVSS
DOM21-33;
SEQ ID NO: 89
EVQLLESGGGLVQPGGSLRLSCAASGFTFSDYMMGWVRQAPGKGLEWV
SRITSEGSHTYYADSVKGRFTISRDNSKNTLYLQMNSLRAEDTAVYYC
AKHTSGFDYWGQGTLVTVSS
DOM21-34;
SEQ ID NO: 90
EVQLLESGGGLVQPGGSLRLSCAASGFTFGRYMMGWVRQAPGKGLEWV
SRISGPGTVTYYADSVKGRFTISRDNSKNTLYLQMNSLRAEDTAVYYC
AKHDTGFDYWGQGTLVTVSS
DOM21-35;
SEQ ID NO: 91
EVQLLESGGGLVQPGGSLRLSCAASGFTFSSYAMIWVRQAPGKGLEWV
SEISPYGNHTYYADSVKGRFTISRDNSKNTLYLQMNSLRAEDTAVYYC
AKPDRRFDYWGQGTLVTVSS
DOM21-36;
SEQ ID NO: 92
EVQLLESGGGLVQPGGSLRLSCAASGFTFTSYGMQWVRQAPGKGLEWV
SSISTDGMVTYYADSVKGRFTISRDNSKNTLYLQMNSLRAEDTAVYYC
AKLGVNFDYWGQGTLVTVSS
DOM21-37;
SEQ ID NO: 93
EVQLLESGGGLVQPGGSLRLSCAASGFTFGDYMMGWVRQAPGKGLEWV
SIIRVPGSTTYYADSVKGRFTISRDNSKNTLYLQMNSLRAEDTAVYYC
AKQKGDEFDYWGQGTLVTVSS
DOM21-38;
SEQ ID NO: 94
EVQLLESGGGLVQPGGSLRLSCAASGFTFILYDMQWVRQAPGKGLEWV
SRISANGHDTYYADSVKGRFTISRDNSKNTLYLQMNSLRAEDTAVYYC
AKGPHYLFDYWGQGTLVTVSS
DOM21-39;
SEQ ID NO: 95
EVQLLESGGGLVQPGGSLRLSCAASGFTFTKYFMGWVRQAPGKGLEWV
SLIDPRGPHTYYADSVKGRFTISRDNSKNTLYLQMNSLRAEDTAVYYC
AKQLGEEFDYWGQGTLVTVSS
DOM21-40;
SEQ ID NO: 96
EVQLLESGGGLVQPGGSLRLSCAASGFTFKTYTMRWVRQAPGKGLEWV
STINSSGTLTYYADSVKGRFTISRDNSKNTLYLQMNSLRAEDTAVYYC
AKSSSYTFDYWGQGTLVTVSS
DOM21-41;
SEQ ID NO: 97
EVQLLESGGGLVQPGGSLRLSCAASGFTFAMYSMKWVRQAPGKGLEWV
SSISNAGDITYYADSVKGRFTISRDNSKNTLYLQMNSLRAEDTAVYYC
AESFRSRYFDYWGQGTLVTVSS
DOM21-42;
SEQ ID NO: 98
EVQLLESGGGLVQPGGSLRLSCAASGFTFDDYLMGWVRQAPGKGLEWV
SLIRMRGSVTYYADSVKGRFTISRDNSKNTLYLQMNSLRAEDTAVYYC
AKHSLTTNLFDYWGQGTLVTVSS
DOM21-43;
SEQ ID NO: 99
EVQLLESGGGLVQPGGSLRLSCAASGFTFTDYMMAWARQAPGKGLEWV
SIIGTTGTWTYYADSVKGRFTISRDNSKNTLYLQMNSLRAEDTAVYYC
AKTNAYESEFDYWGQGTLVTVSS
DOM21-44;
SEQ ID NO: 100
EVQLLESGGGLVQPGGSLRLSCAASGFTFARYTMVWVRQAPGKGLEWV
SAIHFDGRTTYYADSVKGRFTISRDNSKNTLYLQMNSLRAEDTAVYYC
AKNEWASLKHFDYWGQGTLVTVSS
DOM21-45;
SEQ ID NO: 101
EVQLLESGGGLVQPGGSLRLSCAASGFTFEDYMMGWVRQAPGKGLEWV
SFINLPGGRTYYADSVKGRFTISRDNSKNTLYLQMNSLRAEDTAVYYC
AKQTHGLTGYFDYWGQGTLVTVSS
DOM21-46;
SEQ ID NO: 102
EVQLLESGGGLVQPGGSLRLSCAASGFTFGLYGMAWARQAPGKGLEWV
SSIGMHGDTTYYADSVKGRFTISRDNSKNTLYLQMNSLRAEDTAVYYC
AKVCGATYCNFDYWGQGTLVTVSS
DOM21-47;
SEQ ID NO: 103
EVQLLESGGGLVQPGGSLRLSCAASGFTFGKYVMAWVRQAPGKGLEWV
SIIDSLGSTTYYADSVKGRFTISRDNSKNTLYLQMNSLRAEDTAVYYC
AKGGLLVHYDFDYWGQGTLVTVSS
DOM21-48;
SEQ ID NO: 104
EVQLLESGGGLVQPGGSLRLSCAASGFTFEVYGMSWARQAPGKGLEWV
SLIDAGGRNTYYADSVKGRFTISRDNSKNTLYLQMNSLRAEDTAVYYC
AKSTTRAYSDYFDYWGQGTLVTVSS
DOM21-49;
SEQ ID NO: 105
EVQLLESGGGLVQPGGSLRLSCAASGFTFENYDMHWVRQAPGKGLEWV
SGITTHGRRTYYADSVKGRFTISRDNSKNTLYLQMNSLRAEDTAVYYC
AKSDNLNMNVDFDYWGQGTLVTVSS
DOM21-50;
SEQ ID NO: 106
EVQLLESGGGLVQPGGSLRLSCAASGFTFIKYDMCWARQAPGKGLEWV
SCIESSGQNTYYADSVKGRFTISRDNSKNTLYLQMNSLRAEDTAVYYC
AKCLNDSCNVHFDYWGQGTLVTVSS
DOM21-51;
SEQ ID NO: 107
EVQLLESGGGLVQPGGSLRLSCAASGFTFGNYNMGWVRQAPGKGLEWV
SDIGRYGRVTYYADSVKGRFTISRDNSKNTLYLQMNSLRAEDTAVYYC
AKTQRMVNPSPFDYWGQGTLVTVSS
DOM21-52;
SEQ ID NO: 108
EVQLLESGGGLVQPGGSLRLSCAASGFTFVSYSMGWVRQAPGKGLEWV
SIISGQGTVTYYADSVKGRFTISRDNSKNTLYLQMNSLRAEDTAVYYC
AKSPMVFALDGRSFDYWGQGTLVTVSS
DOM21-53;
SEQ ID NO: 109
EVQLLESGGGLVQPGGSLRLSCTASGFTFSEYSMGWVRQAPGKGLEWV
SSITPVGVFTYYADSVKGRFTISRDNSKNTLYLQMNSLRAEDTAVYYC
AKGRPGPHGWSFRFDYWGQGTLVTVSS
DOM21-54;
SEQ ID NO: 110
EVQLLESGGGLVQPGGSLRLSCAASGFTFGQYMMGWVRQAPGKGLEWV
STIDKSGYSTYYADSVKGRFTISRDNSKNTLYLQMNSLRAEDTAVYYC
AKSGIDSRGLMTKFDYWGQGTLVTVSS
DOM21-55;
SEQ ID NO: 111
EVQLLESGGGLVQPGGSLRLSCAASGFTFARYRMAWVRQAPGKGLEWV
SSILSDGAVTYYADSVKGRFTISRDNSKNTLYLQMNSLRAEDTAVYYC
AKPGGNAWSTRVTFDYWGQGTLVTVSS
DOM21-56;
SEQ ID NO: 112
EVQLLESGGGLVQPGGSLRLSCAASGFTFFTYXMAWVRQAPGKGLEWV
SSITPLGYNTYYADSVKGRFTISRDNSKNTLYLQMNSLRAEDTAVYYC
AKPSDVKVSPLPSFDYWGRGTLVTVSS
DOM21-57;
SEQ ID NO: 113
EVQLLESGGGLVQPGGSLRLSCAASGFTFTMYGMHWVRQAPGKGLEWV
SSISQYGLSTYYADSVKGRFTISRDNSKNTLYLQMNSLRAEDTAVYYC
AKGSMRRVFSSSDTFDYWGQGTLVTVSS
DOM21-58;
SEQ ID NO: 114
DIQMTQSPSSLSASVGDRVTITCRASQNIGDRLHWYQQKPGKAPKLLI
YRISRLQSGVPSRFSGSGSGTDFTLTISSLQPEDFATYYCQQFGLYPT
TFGQGTKVEIKR

1. Amino acid sequence that modulates, inhibits, prevents or blocks the interaction between (a target on an antigen presenting cell (APC) and CD28.

2-25. (canceled)

26. Amino acid sequence according to claim 1, that can specifically bind to CD28 with a dissociation constant (K_{D}) of 10⁻⁵ to 10⁻¹² moles/litre or less, and more preferably 10⁻⁷ to 10⁻¹² moles/litre or less and more preferably 10⁻⁸ to 10¹² moles/litre.

27. Amino acid sequence according to claim 1, that can specifically bind to CD28 with a rate of association (kₒₙ-rate) of between 10² M⁻¹ s⁻¹ to about 10⁷ M⁻¹s⁻¹, preferably between 10³ M⁻¹s⁻¹ and 10⁷ M⁻¹ s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹.

28. Amino acid sequence according to claim 1, that can specifically bind to CD28 with a rate of dissociation (k_{off} rate) between 1 s⁻¹ and 10⁻⁶ s⁻¹, preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

29. Amino acid sequence according to claim 1, that can specifically bind to CD28 with an affinity less than 500 nM, or less than 200 nM, or less than 10 nM, or less than 500 pM.

30. (canceled)

31. Amino acid sequence according to claim 1, that comprises an immunoglobulin fold or that under suitable conditions is capable of forming an immunoglobulin fold.

32-35. (canceled)

36. Amino acid sequence according to claim 1, that essentially consists of a light chain variable domain sequence (V_{L}); or of a heavy chain variable domain sequence (V_{H} or V_{HH}).

37. Amino acid sequence according to claim 1, that essentially consists of a heavy chain variable domain sequence that is derived from a conventional four-chain antibody or that essentially consist of a heavy chain variable domain sequence that is derived from heavy chain antibody.

38. Amino acid sequence according to claim 1, that essentially consists of a domain antibody, or an amino acid sequence that is suitable for use as a domain antibody, of a single domain antibody or an amino acid sequence that is suitable for use as a single domain antibody, of a dAb or an amino acid sequence that is suitable for use as a dAb, or of a immunoglobulin single variable domain or a immunoglobulin single variable domain having a V_{HH} sequence.

39. (canceled)

40. Amino acid sequence according to claim 1, that essentially consists of an immunoglobulin single variable domain that i) has at least 80% amino acid identity with at least one of the amino acid sequences of SEQ ID NOs: 58-114, which for the purposes of determining the degree of amino acid identity, the amino acid residues that form the CDR sequences are disregarded; and in which: ii) preferably one or more of the amino acid residues at positions 11, 37, 44, 45, 47, 83, 84, 103, 104 and 108 according to the Kabat numbering are chosen from L and V for position 11, V and F at position 37, G at position 44, L at position 45, W and Y at position 47, R and K at position 83, A, T and D at position 84, W at position 103, G at position 104, and L, M and T at position 108.

41. Amino acid sequence according to claim 1, that essentially consists of a humanized immunoglobulin single variable domain.

42. (canceled)

43. Amino acid sequence according to claim 1, that comprises one or more stretches of amino acid residues selected from the group consisting of: a) the amino acid sequences encoding a CDR1 region of SEQ ID NOs: 58-114; b) amino acid sequences that have at least 80% amino acid identity with at least one of the amino acid sequences encoding a CDR1 region of SEQ ID NOs: 58-114; c) amino acid sequences that have 3, 2, or 1 amino acid difference with at least one of the amino acid sequences encoding a CDR1 region of SEQ ID NOs: 58-114; d) the amino acid sequences encoding a CDR2 region of SEQ ID NOs: 58-114; e) amino acid sequences that have at least 80% amino acid identity with at least one of the amino acid sequences encoding a CDR2 region of SEQ ID NOs: 58-114: f) amino acid sequences that have 3, 2, or 1 amino acid difference with at least one of the amino acid sequences encoding a CDR2 region of SEQ ID NOs: 58-114; g) the amino acid sequences encoding a CDR3 region of SEQ ID NOs: 58-114; h) amino acid sequences that have at least 80% amino acid identity with at least one of the amino acid sequences encoding a CDR3 region of SEQ ID NOs: 58-114; i) amino acid sequences that have 3, 2, or 1 amino acid difference with at least one of the amino acid sequences encoding a CDR3 region of SEQ ID NOs: 58-114; or any combination thereof.

44. Amino acid sequence according to claim 43, in which at least one of said stretches of amino acid residues forms part of the antigen binding site for binding against CD28.

45-48. (canceled)

49. Amino acid sequence according to claim 43, in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NOs: 58-114.

50.-52. (canceled)

53. Amino acid sequence that essentially consists of 4 framework regions (FR1 to FR4, respectively) and 3 complementarity determining regions (CDR1 to CDR3, respectively), in which:
CDR1 is chosen from the group consisting of: a) the amino acid sequences encoding the CDR1 region of SEQ ID NOs: 58-114; b) amino acid sequences that have at least 80% amino acid identity with at least one of the amino acid sequences encoding the CDR1 region of SEQ ID NOs: 58-114; c) amino acid sequences that have 3, 2, or 1 amino acid difference with at least one of the amino acid sequences encoding the CDR1 region of SEQ ID NOs: 58-114; and/or
CDR2 is chosen from the group consisting of: d) the amino acid sequences encoding the CDR2 region of SEQ ID NOs: 58-114; e) amino acid sequences that have at least 80% amino acid identity with at least one of the amino acid sequences encoding the CDR2 region of SEQ ID NOs: 58-114; f) amino acid sequences that have 3, 2, or 1 amino acid difference with at least one of the amino acid sequences encoding the CDR2 region of SEQ ID NOs: 58-114; and/or
CDR3 is chosen from the group consisting of: g) the amino acid sequences encoding the CDR3 region of SEQ ID NOs: 58-114; h) amino acid sequences that have at least 80% amino acid identity with at least one of the amino acid sequences encoding the CDR3 region of SEQ ID NOs: 58-114; i) amino acid sequences that have 3, 2, or 1 amino acid difference with at least one of the amino acid sequences encoding the CDR3 region of SEQ ID NOs: 58-114.

54. (canceled)

55. Amino acid sequence directed against CD28 that inhibits the binding of at least one of the amino acid sequences according to claim 53 to CD28.

56. Amino acid sequence directed against CD28 that is inhibited from binding to CD28 by at least one of the amino acid sequences according to claim 53.

57.-99. (canceled)

100. Compound or construct, that comprises or essentially consists of one or more amino acid sequences according to claim 1 and optionally further comprises one or more other groups, residues, moieties or binding units, optionally linked via one or more linkers.

101.-103. (canceled)

104. Compound or construct according to claim 100, in which said one or more other groups, residues, moieties or binding units are chosen from the group consisting of an immunoglobulin single variable domain, domain antibodies, amino acid sequences that are suitable for use as a domain antibody, single domain antibodies, amino acid sequences that are suitable for use as a single domain antibody, dAbs, amino acid sequences that are suitable for use as a dAb or a immunoglobulin single variable domain.

105. (canceled)

106. Compound or construct according to claim 100, in which said one or more amino acid sequences of the invention are chosen from the group consisting of an immunoglobulin single variable domain, domain antibodies, amino acid sequences that are suitable for use as a domain antibody, single domain antibodies, amino acid sequences that are suitable for use as a single domain antibody, dAbs, amino acid sequences that are suitable for use as a dAb, or Immunoglobulin single variable domains.

107. (canceled)

108. Compound or construct according to claim 100, which is a multivalent construct.

109. Compound or construct according to claim 100, which is a multispecific construct.

110. Compound or construct according to claim 100, which has an increased half-life, compared to the corresponding amino acid sequence.

111. Compound or construct according to claim 110, in which said one or more other groups, residues, moieties or binding units provide the compound or construct with increased half-life, compared to the corresponding amino acid sequence according to claim 1.

112. Compound or construct according to claim 111, in which said one or more other groups, residues, moieties or binding units that provide the compound or construct with increased half-life is chosen from the group consisting of serum proteins or fragments thereof, binding units that can bind to serum proteins, an Fc portion, and small proteins or peptides that can bind to serum proteins.

113. Compound or construct according to claim 111, in which said one or more other groups, residues, moieties or binding units that provide the compound or construct with increased half-life is chosen from the group consisting of human serum albumin or fragments thereof.

114. Compound or construct according to claim 112, in which said one or more other groups, residues, moieties or binding units that provides the compound or construct with increased half-life are chosen from the group consisting of binding units that can bind to serum albumin (such as human serum albumin) or a serum immunoglobulin (such as IgG).

115. Compound or construct according to claim 111, in which said one or more other groups, residues, moieties or binding units that provides the compound or construct with increased half-life are chosen from the group consisting of domain antibodies, amino acid sequences that are suitable for use as a domain antibody, single domain antibodies, amino acid sequences that are suitable for use as a single domain antibody, dAbs, amino acid sequences that are suitable for use as a dAb, or immunoglobulin single variable domains that can bind to serum albumin (such as human serum albumin) or a serum immunoglobulin (such as IgG).

116. Compound or construct according to claim 111, in which said one or more other groups, residues, moieties or binding units that provides the compound or construct with increased half-life is an immunoglobulin single variable domains that can bind to serum albumin (such as human serum albumin) or a serum immunoglobulin (such as IgG).

117. Compound or construct according to claim 110, that has a serum half-life that is at least 1.5 times, preferably at least 2 times, such as at least 5 times, for example at least 10 times or more than 20 times, greater than the half-life of the corresponding amino acid sequence.

118. Compound or construct according to claim 110, that has a serum half-life that is increased with more than 1 hours, preferably more than 2 hours, more preferably more than 6 hours, such as more than 12 hours, or even more than 24, 48 or 72 hours, compared to the corresponding amino acid sequence.

119. Compound or construct according to claim 110, that has a serum half-life in human of at least about 12 hours, preferably at least 24 hours, more preferably at least 48 hours, even more preferably at least 72 hours or more; for example, of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

120. Monovalent construct, comprising or essentially consisting of one amino acid sequence according to any of claim 1.

121.-131. (canceled)

132. Nucleic acid or nucleotide sequence, that encodes an amino acid sequence according to claim 1, immunoglobulin single variable domain according to claim 57 or 73, a compound or construct according claim 100, or a monovalent construct according to claim 120.

133. Nucleic acid or nucleotide sequence according to claim 132, that is in the form of a genetic construct.

134. Host or host cell that expresses, or that under suitable circumstances is capable of expressing, an amino acid sequence according to claim 1.

135. Method for producing an amino acid sequence, said method at least comprising the steps of: a) expressing, in a suitable host cell or host organism or in another suitable expression system, a nucleic acid or nucleotide sequence according to claim 132, optionally followed by: b) isolating and/or purifying the amino acid sequence thus obtained.

136.-137. (canceled)

138. Composition, comprising at least one amino acid sequence according to claim 1.

139. (canceled)

140. Composition according to claim 138, which is a pharmaceutical composition, that further comprises at least one pharmaceutically acceptable carrier, diluent or excipient and/or adjuvant, and that optionally comprises one or more further pharmaceutically active polypeptides and/or compounds.

141. Method for the prevention and/or treatment of at least one autoimmune disease, allergy, asthma, transplant rejection (acute and chronic), cancer, tumor, effector cell exhaustion, or infection, said method comprising administering, to a subject in need thereof, a pharmaceutically active amount of at least one amino acid sequence according to claim 1.

142. Method for the prevention and/or treatment of at least one disease or disorder that is associated with CD28, with its biological or pharmacological activity, and/or with the biological pathways or signaling in which an APC target or a T-cell target is involved, said method comprising administering, to a subject in need thereof, a pharmaceutically active amount of at least one amino acid sequence according to claim 1.

143. (canceled)

144. Method for immunotherapy, said method comprising administering, to a subject in need thereof, a pharmaceutically active amount of at least one an amino acid sequence according to claim 1.

145.-234. (canceled)

235. Amino acid according to claim 1, that is a pegylated derivative.

236.-242. (canceled)

243. An antagonist of CD28 comprising a-monovalent polypeptide domain which specifically binds CD28 and which competes for binding to CD28 with an antibody single variable domain selected from the group consisting of DOM21-4, DOM21-18, DOM21-28, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-27, DOM21-29, DOM21-30, DOM2′-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-58, DOM21-59, DOM21-60, DOM21-61, DOM21-62, DOM21-63, DOM21-64, DOM21-65, DOM21-66, DOM21-67, and DOM21-68.

244. (canceled)

245. An antagonist of CD28 comprising a monovalent polypeptide domain which specifically binds CD28 and which comprises an amino sequence which is at least 70% identical to an amino acid sequence of an antibody single variable domain selected from the group consisting of: DOM21-4, DOM21-18, DOM21-28, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-27, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM2′-46, DOM21-47, DOM21-48, DOM21-49, DOM21-50, DOM21-51, DOM21-52, DOM21-53, DOM21-54, DOM21-55, DOM21-56, DOM21-57, DOM21-58, DOM21-59, DOM21-60, DOM21-61, DOM21-62, DOM21-63, DOM21-64, DOM21-65, DOM21-66, DOM21-67, and DOM21-68.

246. (canceled)

247. The antagonist of claim 245, wherein said monovalent polypeptide domain which specifically binds CD28 is an antibody single variable domain.

248. (canceled)

249. (canceled)

250. (canceled)

251. (canceled)

252. (canceled)

253. (canceled)

254. (canceled)

255. The antagonist of claim 245, wherein said monovalent polypeptide domain which specifically binds CD28 has a CDR1 amino acid sequence that is at least 50% identical to the CDR1 amino acid sequence of a single variable domain selected from the group consisting of: DOM21-4, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-59, DOM21-60, DOM21-61, DOM21-62, DOM21-63, DOM21-64, DOM21-65, DOM21-66, DOM21-67, and DOM21-68.

256. The antagonist of claim 245, wherein said monovalent polypeptide domain which specifically binds CD28 has a CDR2 amino acid sequence that is at least 50% identical to the amino acid sequence of a CDR2 of a single variable domain selected from the group consisting of: DOM21-18, DOM21-27, DOM21-28 and DOM21-58.

257. (canceled)

258. (canceled)

259. (canceled)

260. The antagonist of claim 245, wherein said monovalent polypeptide domain which specifically binds CD28 has a CDR2 amino acid sequence that is at least 50% identical to the CDR2 amino acid sequence of a single variable domain selected from the group consisting of: DOM21-4, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-59, DOM21-60, DOM21-61, DOM21-62, DOM21-63, DOM21-64, DOM21-65, DOM21-66, DOM21-67, and DOM21-68.

261. The antagonist of claim 245, wherein said monovalent polypeptide domain which specifically binds CD28 comprises a CDR2 amino acid sequence selected from the group consisting of: YYADSVKG, wherein YY is at Kabat position 58 and 59, respectively.

262. (canceled)

263. (canceled)

264. (canceled)

265. (canceled)

266. (canceled)

267. (canceled)

268. (canceled)

269. (canceled)

270. (canceled)

271. The antagonist of claim 245, wherein said monovalent polypeptide domain which specifically binds CD28 has a CDR3 amino acid sequence that is at least 50% identical to the amino acid sequence of a CDR3 of a single variable domain selected from the group consisting of: DOM21-4, DOM21-1, DOM21-2, DOM21-3, DOM21-5, DOM21-6, DOM21-7, DOM21-8, DOM21-9, DOM21-10, DOM21-11, DOM21-12, DOM21-13, DOM21-14, DOM21-15, DOM21-16, DOM21-17, DOM21-19, DOM21-20, DOM21-21, DOM21-22, DOM21-23, DOM21-24 DOM21-25, DOM21-26, DOM21-29, DOM21-30, DOM21-31, DOM21-32, DOM21-33, DOM21-34, DOM21-35, DOM21-36, DOM21-37, DOM21-38, DOM21-39, DOM21-40, DOM21-41, DOM21-42, DOM21-43, DOM21-44, DOM21-45, DOM21-46, DOM21-47, DOM21-48, DOM21-49, DOM21-50, DOM21-51, DOM21-52, DOM21-53, DOM21-54, DOM21-55, DOM21-56, DOM21-57, DOM21-59, DOM21-60, DOM21-61, DOM21-62, DOM21-63, DOM21-64, DOM21-65, DOM21-66, DOM21-67, and DOM21-68.

272. (canceled)

273. (canceled)

274. (canceled)

275. (canceled)

276. (canceled)

277. (canceled)

278. (canceled)

279. (canceled)

280. (canceled)

281. (canceled)

282. (canceled)

283. (canceled)

284. (canceled)

285. (canceled)

286. (canceled)

287. The antagonist of claim 245, wherein said monovalent polypeptide domain which specifically binds CD28 has a FR2 with a V or an A at position 37.

288. (canceled)

289. (canceled)

290. (canceled)

291. (canceled)

292. (canceled)

293. (canceled)

294. (canceled)

295. (canceled)

296. (canceled)

297. (canceled)

298. (canceled)

299. (canceled)

300. (canceled)

301. (canceled)

302. (canceled)

303. (canceled)

304. The antagonist of claim 245, wherein said monovalent polypeptide domain which specifically binds CD28 is fused to a heterologous protein or molecule.

305. The antagonist of claim 245, wherein said monovalent polypeptide domain which specifically binds CD28, inhibits binding to CD28 by CD80 and/or CD86.

306. (canceled)

307. The antagonist of claim 305, wherein said monovalent polypeptide domain which specifically binds CD28, preferentially inhibits the binding to CD28 by CD86 relative to the binding to CD28 by CD80.

308. The antagonist of claim 245, wherein said monovalent polypeptide domain which specifically binds CD28, inhibits the binding to CD28 by CD80 and/or CD86 with an IC₅₀ in the range of 50 pM up to and including 3.0 μM.

309. The antagonist of claim 245, wherein said monovalent polypeptide domain which specifically binds CD28, inhibits the binding to CD28 by CD80 and/or CD86 with an IC₅₀ selected from the group consisting of: about 5 nM, about 4 nM, about 3 nM, about 2.5 nM, about 2.0 nM, about 1.9 nM, about 1.8 nM, about 1.7 nM, about 1.6 nM, about 1.5 nM, about 1.4 nM, about 1.3 nM, about 1.2, nM, about 1.1 nM, about 1.0 nM, about 0.9 nM, about 0.8 nM, about 0.7 nM, about 0.6 nM, about 0.5 nM, about 0.4 nM, about 0.3 nM, about 0.2 nM, about 0.1 nM and about 0.05 nM.

310. The antagonist of claim 245, wherein said monovalent polypeptide domain which specifically binds CD28, does not cross react with CTLA4.

311. The antagonist of claim 245, wherein said monovalent polypeptide domain which specifically binds CD28 is encoded by a nucleic acid molecule comprising a sequence selected from the group consisting of SEQ ID NOs: 1-57.

312. The antagonist of claim 245, wherein said antagonist further comprises an additional antibody single variable domain which specifically binds an antigen other than CD28.

313. (canceled)

314. (canceled)

315. (canceled)

316. (canceled)

317. (canceled)

318. (canceled)

319. (canceled)

320. (canceled)

321. The antagonist of claim 245, further comprising PEG.

322. (canceled)

323. (canceled)

324. An isolated composition comprising the antagonist of claim 245, and a carrier.

325. (canceled)

326. An isolated pharmaceutical composition comprising the antagonist of claim 245, and a pharmaceutically acceptable carrier.

327. (canceled)

328. (canceled)

329. (canceled)

330. A method of treating or preventing an autoimmune disease in an individual, the method comprising administering the antagonist of claim 245 or composition thereof, to said individual.

331. (canceled)

332. A method of treating or preventing a transplantation disease in an individual, the method comprising administering the antagonist of claim 245, composition thereof, to said individual.

333. (canceled)

334. (canceled)

335. (canceled)

336. (canceled)

337. The antagonist of claim 245, wherein binding of said monovalent polypeptide domain to CD28 does not substantially agonize CD28 activity in combination with T cell receptor signaling.

338. The antagonist of claim 245, wherein said monovalent polypeptide domain which specifically binds CD28 has a FW2 sequence encoded by a germline gene segment.

339. The antagonist of claim 338, wherein said germline gene segment is selected from the group consisting of DP47 and DPK9.

340. An immunoglobulin single variable domain antibody that binds to CD28, the antibody having at least three characteristics selected from the group consisting of:
i. prevents CD80 and CD86 binding to CD28;
ii. does not cross-react with CTLA4;
iii. has a tα half-life of about 15 seconds to about 12 hours; and
iv. has a tβ half-life of about 12 hours to about 96 hours.

341. The antagonist of claim 245, wherein said agonist has a serum tβ half-life in the range of about 12 hours to about 31 days.

342. (canceled)

343. (canceled)

344. (canceled)

345. (canceled)


SEQUENCE LISTING (180 sequences)

SEQ ID NO: 1 - DNA, 348 bp, Artificial Sequence
  Compositions monovalent for CD28 binding and methods of use
gaggtgcagctgttggagtctgggggaggcttggtacagcctggggggtccctgcgtctctcctgtgcagcctccggattcacctttgatgcgtattcgatgatttgggtccgccaggctccagggaagggtctagagtgggtctcaactattactccgcagggtgataggacatactacgcagactccgtgaagggccggttcaccatctcccgcgacaattccaagaacacgctgtatctgcaaatgaacagcctgcgtgccgaggacaccgcggtatattactgtgcgcaagctggttggagttttgactactggggtcagggaaccctggtcaccgtctcgagc

SEQ ID NO: 2 - DNA, 349 bp, Artificial Sequence
  DOM21-2 CD28 antibody single variable domain
gaggtgcagctgttggagtctgggggaggcttggtacagcctggggggtccctgcgtctctcctgtgcagcctccggattcacctttgtggattatgagatggcttgggtccgccaggctccagggaagggtctagagtgggtctcaactatttcgaatgatggcgctgctacatactacgcagactccgtgaagggccggttcaccatctcccgcgacaattccaagaacacgctgtatctgcaaatgaacagcctgcgtgccgaggacaccgcggtatattactgtgcgaaagatgatgctgcttttgactactggggtcagggagccctggtcaccgtctcgagcg

SEQ ID NO: 3 - DNA, 348 bp, Artificial Sequence
  DOM21-3 CD28 antibody single variable domain
gaggtgcagctgttggagtctgggggaggcttggtacagcctggggggtccctgcgtctctcctgtgcagcctccggattcacctttggtgcgtattctatggggtgggcccgccaggctccagggaagggtctagagtgggtctcatggattacggggaatggtggttctacatactacgcagactccgtgaagggccggttcaccatctcccgcgacaattccaagaacacgctgtatctgcaaatgaacagcctgcgtgccgaggacaccgcggtatattactgtgcgaaagcggaggagccgtttgactactggggtcagggaaccctggtcaccgtctcgagc

SEQ ID NO: 4 - DNA, 349 bp, Artificial Sequence
  DOM21-4 CD28 antibody single variable domain
gaggtgcagctgttggagtctgggggaggcttggtacagcctggggggtccctgcgtctctcctgtgcagcctccggattcacctttagtaggtatcatatggcgtgggtccgccaggctccagggaagggtctagagtgggtctcagtgattgattctcttggtcttcagacatactacgcagactccgtgaagggccggttcaccatctcccgcgacaattccaagaacacgctgtatctgcaaatgaacagcctgcgtgccgaggacaccgcggtatattactgtgcggaatatggtggtgcgtttgactactggggtcagggaaccctggtcaccgtctcgagcg

SEQ ID NO: 5 - DNA, 352 bp, Artificial Sequence
  DOM21-5 CD28 antibody single variable domain
gaggtgcagctgttggagtctgggggaggcttggtacagcctggggggtccctgcgtctctcctgtgcagcctccggattcacctttactcattattctatggggtgggtccgccaggctccagggaagggtctagagtgggtctcacatattactccggatggtcttattacatactacgcagactccgtgaagggccggttcaccatctcccgcgacaattccaagaacacgctgtatctgcaaatgaacagcctgcgtgccgaggataccgcggtatattactgtgcgaaaggtaggttggttgattttgactactggggtcagggaaccctggtcaccgtctcgagcg

SEQ ID NO: 6 - DNA, 354 bp, Artificial Sequence
  DOM21-6 CD28 antibody single variable domain
gaggtgcagctgttggagtctgggggaggcttggtacagcctggggggtccctgcgtctctcctgtgcagcctccggattcacctttgagaattatggtatggcttgggtccgccaggctccagggaagggtctagagtgggtctcaaatattggtcgggctggtagtgttacatactacgcagactccgtgaagggccggttcaccatctcccgcgacaattccaagaacacgctgtatctgcaaatgaacagcctgcgtgccgaggacaccgcggtatattactgtgcgaaagttcagtcgtggaggacttttgactactggggtcagggaaccctggtcaccgtctcgagc

SEQ ID NO: 7 - DNA, 357 bp, Artificial Sequence
  DOM21-7 CD28 antibody single variable domain
gaggtgcagctgttggagtctgggggaggcttggtacagcctggggggtccctgcgtctctcctgtgcagcctccggattcacctttcctgcgtattctatggggtgggtccgccaggctccagagaagggtctagagtgggtctcatatattgatgggcgtggtgctgagacatactacgcagactccgtgaagggccggttcaccatctcccgcgacaattccaagaacacgctgtatctgcaaatgaacagcctgcgcgccgaggataccgcggtatattactgtgcgaaaattgatactctgatttctgagtttgactactggggtcagggaaccctggtcaccgtctcgagc

SEQ ID NO: 8 - DNA, 357 bp, Artificial Sequence
  DOM21-8 CD28 antibody single variable domain
gaggtgcagctgttggagtctgggggaggcttggtacagcctggggggtccctgcgtctctcctgtgcagcctccggattcacctttcctaattatacgatgtggtgggtccgccaggctccagggaagggtctagagtgggtctcatctattagtggtactggtcatactacatactacgcagactccgtgaagggccggttcaccatctcccgcgacaattccaagaacacgctgtatctgcaaatgaacagcctgcgtgccgaggataccgcggtatattactgtgcgaaatttgggcctaataatcctatgtttgactactggggtcagggaaccctggtcaccgtctcgagc

SEQ ID NO: 9 - DNA, 357 bp, Artificial Sequence
  DOM21-9 CD28 antibody single variable domain
gaggtgcagctgttggagtctgggggaggcttggtacagcctggggggtccctgcgtctctcctgtgcagcctccggattcacctttgcgagttatgatatgggttgggtccgccaggctccagggaagggtctagagtgggtctcagcgatttcggcggatggtacgtttacatactacgcagactccgtgaagggccggttcaccatctcccgcgacaattccaagaacacgctgtatctgcaaatgaacagcctgcgtgccgaggacaccgcggtatattactgtgcgaaatcttcttttgataagtataattttgactactggggtcagggaaccctggtcaccgtctcgagc

SEQ ID NO: 10 - DNA, 357 bp, Artificial Sequence
  DOM21-10 CD28 antibody single variable domain
gaggtgcagctgttggagtctgggggaggcttggtacagcctggggggtccctgcgtctctcctgtgcagcctccggattcacctttgctaagtatacgatgtggtgggtccgccaggctccagggaagggtctagagtgggtctcaagtattgatcctgttggtaatttgacatactacgcagactccgtgaagggccggttcaccatctcccgcgacaattccaagaacacgctgtatctgcaaatgaacagcctgcgtgccgaggacaccgcggtatattactgtgcgaaaagggggccgacgtcgtctaattttgactactggggtcagggaaccctggtcaccgtctcgagc

SEQ ID NO: 11 - DNA, 360 bp, Artificial Sequence
  DOM21-11 CD28 antibody single variable domain
gaggtgcagctgttggagtctgggggaggcttggtacagcctggggggtccctgcgtctctcctgtgcagcctccggattcacctttagtgagtatggtatgaagtgggtccgccaggctccagggaagggtctagagtgggtctcaacgattgataatgttggttcggtgacatactacgcagactccgtgaagggccggttcaccatctcccgcgacaattccaagaacacgctgtatctgcaaatgaacagcctgcgtgccgaggacaccgcggtatattactgtgcgaaaactacgcctgttttgctgccgctttttgactactggggtcagggaaccctggtcaccgtctcgagc

SEQ ID NO: 12 - DNA, 360 bp, Artificial Sequence
  DOM21-12 CD28 antibody single variable domain
gaggtgcagctgttggagtctgggggaggcttggtacagcctggggggtccctgcgtctctcctgtgcagcctccggattcacctttgattcttataatatgggttgggtccgccaggctccagggaagggtcttgagtgggtctcagctattgcggctaatggtcgtgtgacatactacgcagactccgtgaagggccggttcaccatctcccgcgacaattccaagaacacgctgtatctgcaaatgaacagcctgcgtgccgaggacaccgcggtatattactgtgcgaaaatgacgaatatggcgtatggtagttttgactactggggtcagggaaccctggtcaccgtctcgagc

SEQ ID NO: 13 - DNA, 360 bp, Artificial Sequence
  DOM21-13 CD28 antibody single variable domain
gaggtgcagctgttggagtctgggggaggcttggtacagcctggggggtccctgcgtctctcctgtgcagcctccggattcacctttgatctgtattcgatggcttgggtccgccaggctccagggaagggtctggagtgggtctcacatattgatagggctggtatgattacatactacgcagactccgtgaagggccggttcaccatctcccgcgacaattccaagaacacgctgtatctgcaaatgaacagcctgcgtgccgaggacaccgcggtatattactgtgcgaaagtttctaatgctgttaatatgcagtttgactactggggtcagggaaccctggtcaccgtctcgagc

SEQ ID NO: 14 - DNA, 360 bp, Artificial Sequence
  DOM21-14 CD28 antibody single variable domain
gaggtgcagctgttggagtctgggggaggcttggtacagcctggggggtccctgcgtctctcctgtgcagcctccggattcacctttgctaagtatacgatgtggtgggtccgccaggctccagggaagggtctagagtgggtctcaagtattgatcctgttggtaatttgacatactacgcagactccgtgaagggccggttcaccatctcccgcgacaattccaagaacacgctgtatctgcaaatgaacagcctgcgtgccgaggacaccgcggtatattactgtgcgaaacgtcataggccttcgacgcaggattttgactactggggtcagggaaccctggncaccgtctcgagc

SEQ ID NO: 15 - DNA, 361 bp, Artificial Sequence
  DOM21-15 CD28 antibody single variable domain
gaggtgcagctgttggagtctgggggaggcttggtacagcctggggggtccctgcgtctctcctgtgcagcctccggattcacctttcctgattataagatgggttgggtccgccaggctccagggaagggtctagagtgggtctcatggattgataagggtggtattattacatactacgcagactccgtgaagggccggttcaccatctcccgcgacaattccaagaacacgctgtatctgcaaatgaacagcctgcgtgccgaggataccgcggtatattactgtgcgaaaatgtttcctaagtttcggccggcttttgactactggggtcagggaaccctggtcaccgtctcgagcg

SEQ ID NO: 16 - DNA, 361 bp, Artificial Sequence
  DOM21-16 CD28 antibody single variable domain
gaggtgcagctgttggagtctgggggaggcttggtacagcctggggggtccctgcgtctctcctgtgcagcctccggattcacctttgaggattatgggatggggtgggtccgccaggctccagggaagggtctagagtgggtctcacatattaatcgttctggtctggttacatactacgcagactccgtgaagggccggttcaccatctcccgcgacaattccaagaacacgctgtatctgcaaatgaacagcctgcgtgccgaggacaccgcggtatattactgtgcgaaagttctgaatgctcctaattttaagtttgactactggggtcagggaaccctggtcaccgtctcgagcg

SEQ ID NO: 17 - DNA, 369 bp, Artificial Sequence
  DOM21-17 CD28 antibody single variable domain
gaggtgcagctgttggagtctgggggaggcttggtacagcctggggggtccctgcgtctctcctgtgcagcctccggattcacctttaatcgttatgcgatgggttgggtccgccaggctccagggaagggtctagagtgggtctcatggattgatggtaatggtctggttacatactacgcagactccgtgaagggccggttcaccatctcccgcgacaattccaagaacacgctgtatctgcaaatgaacagcctgcgtgccgaggacaccgcggtatattactgtgcgaaacggactaggtctcattctgattcgggttgggcttttgactactggggtcagggaaccctggtcaccgtctcgagc

SEQ ID NO: 18 - DNA, 325 bp, Artificial Sequence
  DOM21-18 CD28 antibody single variable domain
gacatccagatgacccagtctccatcctccctgtctgcatctgtaggagaccgtgtcaccatcacttgccgggcaagtcagtatattggtacttcgttaaattggtaccagcagaaaccagggaaagcccctaagctcctgacctatcaggcttccttgttgcaaagtggggtcccatcacgtttcagtggcagtggatctgggacagatttcactctcaccatcagcagtctgcaacctgaagattttgctacgtactactgtcaacagttggcgctgcgtcctatgacgttcggccaagggaccaaggtggaaatcaaacggg

SEQ ID NO: 19 - DNA, 370 bp, Artificial Sequence
  DOM21-19 CD28 antibody single variable domain
gaggtgcagctgttggagtctgggggaggcttggtacagcctggggggtccctgcgtctctcctgtgcagcctccggattcacctttggtaattataatatggggtgggtccgccaggctccagggaagggtctagagtgggtctcaggtattacgaagggtggtcgggtgacatactacgcagactccgtgaagggccggttcaccatctcccgcgacaattccaagaacacgctgtatctgcaaatgaacagcctgcgtgccgaggataccgcggtatattactgtgcgaaattgggtccgtcgaggatgcttaatgagccgctgtttgactactggggtcagggaaccctggtcaccgtctcgagcg

SEQ ID NO: 20 - DNA, 370 bp, Artificial Sequence
  DOM21-20 CD28 antibody single variable domain
gaggtgcagctgttggagtctgggggaggcttggtacagcctggggggtccctgcgtctctcctgtgcagcctccggattcacctttccggcgtattcgatgatttgggtccgccaggctccagggaagggtctagagtgggtctcaacgatttcgccgctgggttattcgacatactacgcagactccgtgaagggccggttcaccatctcccgcgacaattccaagaacacgctgtatctgcaaatgaacagcctgcgtgccgaggacaccgcggtatattactgtgcggaacagacggcttatttgaatcgtgctacggagcattttgactactggggtcagggaaccctggtcaccgtctcgagcg

SEQ ID NO: 21 - DNA, 370 bp, Artificial Sequence
  DOM21-21 CD28 antibody single variable domain
gaggtgcagctgttggagtctgggggaggcttggtacagcctggggggtccctgcgtctctcctgtgcagcctccggattcaccttttcgaagtatgatatggcttgggtccgccaggctccagggaagggtctggagtgggtctcatcgatttatgctattggtggtaatacatactacgcagactccgtgaagggccggttcaccatctcccgcgacaattccaagaacacgctgtatctgcaaatgaacagcctgcgtgccgaggacaccgcggtatattactgtgcgaaattgaagtcggggatgcagactcggttgaattcttttgactactggggtcagggaaccctggtcaccgtctcgagcg

SEQ ID NO: 22 - DNA, 369 bp, Artificial Sequence
  DOM21-22 CD28 antibody single variable domain
gaggtgcagctgttggagtctgggggaggcttggtacagcctggggggtccctgcgtctctcctgtgcagcctccggattcacctttgagctgtatcagatgggttgggtccgccaggctccagggaagggtctagagtgggtctcaactattatgcctagtggtaatcttacatactacgcagactccgtgaagggccggttcaccatctcccgcgacaattccaagaacacgctgtatctgcaaatgaacagcctgcgtgccgaggataccgcggtatattactgtgcgaaaatgtggtcgttgaatttggggtttcatgcggcttttgactactggggtcagggaaccctggtcaccgtctcgagc

SEQ ID NO: 23 - DNA, 370 bp, Artificial Sequence
  DOM21-23 CD28 antibody single variable domain
gaggtgcagctgttggagtctgggggaggcttggtacagcctggggggtccctgcgtctctcctgtgcagcctccggattcacctttgggcagtatggtatgggttgggtccgccaggctccagggaagggtctagagtgggtctcagggattagtccttctggtaattatacatactacgcagactccgtgaagggccggttcaccatctcccgcgacaattccaagaacacgctgtatctgcaaatgaacagcctgcgtgccgaggataccgcggtatattactgtgcgaaagggaatgggtctcttccgcctcgtgggtctatttttgactactggggtcagggaaccctggtcaccgtctcgagcg

SEQ ID NO: 24 - DNA, 370 bp, Artificial Sequence; identical to SEQ ID NO: 19
  DOM21-24 CD28 antibody single variable domain

SEQ ID NO: 25 - DNA, 369 bp, Artificial Sequence
  DOM21-25 CD28 antibody single variable domain
gaggtgcagctgttggagtctgggggaggcttggtacagcctggggggtccctgcgtctctcctgtgcagcctccggattcacctttgggacgtattatatggggtgggcccgccaggctccagggaagggtctagagtgggtctcatctattggggctaatggtgctcctacatattacgcagactccgtgaagggccggttcaccatctcccgcgacaattccaagaacacgctgtatctgcaaatgaacagcctgcgtgccgaggataccgcggtatattactgtgcgaaaattcgttcgcttaataggtgggcggagcctgtgtttgactactggggtcagggaaccctggtcaccgtctcgagc

SEQ ID NO: 26 - DNA, 349 bp, Artificial Sequence
  DOM21-26 CD28 antibody single variable domain
gaggtgcagctgttggagtctgggggaggcttggtacagcctggggggtccctgcgtctctcctgtgcagcctccggattcacctttgctgattattctatgtattgggtccgccaggctccagggaagggtctggagtgggtctcacagattagtccggcgggttcttttacatactacgcagactccgtgaagggccggttcaccatctcccgcgacaattccaagaacacgctgtatctgcaaatgaacagcctgcgtgccgaggataccgcggtatattactgtgcgaaagattctaagtcttttgactactggggtcagggaaccctggtcaccgtctcgagcg

SEQ ID NO: 27 - DNA, 325 bp, Artificial Sequence
  DOM21-27 CD28 antibody single variable domain
gacatccagatgacccagtctccatcctccctgtctgcatctgtaggagaccgtgtcaccatcacttgccgggcaagtcagagtattgggacgggtttacggtggtaccagcagaaaccagggaaagcccctatgctcctgatctatcgggcgtccattttgcaaagtggggtcccatcacgttttagtggcagtggatctgggacagatttcactctcaccatcagcagtctgcaacctgaagattttgctacgtactactgtcaacagacgactcttcagccttttacgttcagccaagggactaaggtggaaatcaaacggg

SEQ ID NO: 28 - DNA, 325 bp, Artificial Sequence
  DOM21-28 CD28 antibody single variable domain
gacatccagatgacccagtctccatcctccctgtctgcatctgtaggagaccgtgtcaccatcacttgccgggcaagtcagtctattagtcattcgttagtttggtaccagcagaaaccagggaaagcccctaagctcctgatctattgggcttcccttttgcaaagtggggtcccatcacgtttcagtggcagtggatctgggacagatttcactctcaccatcagcagtctgcaacctgaagattttgctacgtactactgtcaacagggtatgactacgccttttacgttcggccaagggaccaaggtggaaatcaaacggg

SEQ ID NO: 29 - DNA, 348 bp, Artificial Sequence
  DOM21-30 CD28 antibody single variable domain
gaggtgcagctgttggagtctgggggaggcttggtacagcctggggggtccctgcgtctctcctgtgcagcctccggattcacctttgatagttatgatatgaattgggtccgccaggctccagggaagggtctagagtgggtctcacagatttctgctgatggtcattttacatactacgcagactccgtgaagggccggttcaccatctcccgcgacaattccaagaacacgctgtatctgcaaatgaacagcctgcgtgccgaggacaccgcggtatattactgtgcgaaatcgcggagtagttttgactactggggtcagggaaccctggtcaccgtctcgagc

SEQ ID NO: 30 - DNA, 348 bp, Artificial Sequence
  DOM21-31 CD28 antibody single variable domain
gaggtgcagctgttggagtctgggggaggcttggtacagcctggggggtccctgcgtctctcctgtgcagcctccggattcacctttagggattatatgatggggtgggtccgccaggctccagggaagggtctagagtgggtctcacgtattgattctcatggtaatcgtacatactacgcagactccgtgaagggccggttcaccatctcccgcgacaattccaagaacacgctgtatctgcaaatgaacagcctgcgtgccgaggacaccgcggtatattactgtgcgaaacatatgacgggttttgactactggggtcagggaaccctggtcaccgtctcgagc

SEQ ID NO: 31 - DNA, 348 bp, Artificial Sequence
  DOM21-32 CD28 antibody single variable domain
gaggtgcagctgttggagtctgggggaggcttggtacagcctggggggtccctgcgtctctcctgtgcagcctccggattcacctttagggagtatatgatggggtgggtccgccaggctccagggaagggtctagagtgggtctcacgtattaatggtgtgggtaattctacatactacgcagactccgtgaagggccggttcaccatctcccgcgacaattccaagaacacgctgtatctgcaaatgaacagcctgcgtgccgaggacaccgcggtatattactgtgcgaaacatcaggtgggttttgactactggggtcagggaaccctggtcaccgtctcgagc

SEQ ID NO: 32 - DNA, 348 bp, Artificial Sequence
  DOM21-33 CD28 antibody single variable domain
gaggtgcagctgttggagtctgggggaggcttggtacagcctggggggtccctgcgtctctcctgtgcagcctccggattcacctttagtgattatatgatgggttgggtccgccaggctccagggaagggtctagagtgggtctcacgtattacgtctgagggttcgcatacatactacgcagactccgtgaagggccggttcaccatctcccgcgacaattccaagaacacgctgtatctgcaaatgaacagcctgcgtgccgaggacaccgcggtatattactgtgcgaaacatacgtctggttttgactactggggtcagggaaccctggtcaccgtctcgagc

SEQ ID NO: 33 - DNA, 348 bp, Artificial Sequence
  DOM21-34 CD28 antibody single variable domain
gaggtgcagctgttggagtctgggggaggcttggtacagcctggggggtccctgcgtctctcctgtgcagcctccggattcacctttgggaggtatatgatggggtgggtccgccaggctccagggaagggtctggagtgggtctcacggatttctggtcctggtacggttacatactacgcagactccgtgaagggccggttcaccatctcccgcgacaattccaagaacacgctgtatctgcaaatgaacagcctgcgtgccgaggacaccgcggtatattactgtgcgaaacatgatacggggtttgactactggggtcagggaaccctggtcaccgtctcgagc

SEQ ID NO: 34 - DNA, 348 bp, Artificial Sequence
  DOM21-35 CD28 antibody single variable domain
gaggtgcagctgttggagtctgggggaggcttggtacagcctggggggtccctgcgtctctcctgtgcagcctccggattcaccttttcttcttatgctatgatttgggtccgccaggctccagggaagggtctagagtgggtctcagagatttctccttatggtaatcatacatactacgcagactccgtgaagggccggttcaccatctcccgcgacaattccaagaacacgctgtatctgcaaatgaacagcctgcgtgccgaggacaccgcggtatattactgtgcgaaacctgatcggcgttttgactactggggtcagggaaccctggtcaccgtctcgagc

SEQ ID NO: 35 - DNA, 348 bp, Artificial Sequence
  DOM21-36 CD28 antibody single variable domain
gaggtgcagctgttggagtctgggggaggcttggtacagcctggggggtccctgcgtctctcctgtgcagcctccggattcacctttacttcgtatgggatgcagtgggtccgccaggctccagggaagggtctagagtgggtctcatcgatttctactgatggtatggttacatactacgcagactccgtgaagggccggttcaccatctcccgcgacaattccaagaacacgctgtatctgcaaatgaacagcctgcgtgccgaggataccgcggtatattactgtgcgaaacttggggttaattttgactactggggtcagggaaccctggtcaccgtctcgagc

SEQ ID NO: 36 - DNA, 351 bp, Artificial Sequence
  DOM21-37 CD28 antibody single variable domain
gaggtgcagctgttggagtctgggggaggcttggtacagcctggggggtccctgcgtctctcctgtgcagcctccggattcacctttggtgattatatgatggggtgggtccgccaggctccagggaagggtctggagtgggtctcaattattcgtgtgcctggttcgactacatactacgcagactccgtgaagggccggttcaccatctcccgcgacaattccaagaacacgctgtatctgcaaatgaacagcctgcgtgccgaggacaccgcggtatattactgtgcgaaacagaagggtgatgagtttgactactggggtcagggaaccctggtcaccgtctcgagc

SEQ ID NO: 37 - DNA, 351 bp, Artificial Sequence
  DOM21-38 CD28 antibody single variable domain
gaggtgcagctgttggagtctgggggaggcttggtacagcctggggggtccctgcgtctctcctgtgcagcctccggattcacctttattctgtatgatatgcagtgggtccgccaggctccagggaagggtctagagtgggtctcacgtatttctgctaatggtcatgatacatactacgcagactccgtgaagggccggttcaccatctcccgcgacaattccaagaacacgctgtatctgcaaatgaacagcctgcgtgccgaggacaccgcggtatattactgtgcgaaaggtccgcattatttgtttgactactggggtcagggaaccctggtcaccgtctcgagc

SEQ ID NO: 38 - DNA, 351 bp, Artificial Sequence
  DOM21-39 CD28 antibody single variable domain
gaggtgcagctgttggagtctgggggaggcttggtacagcctggggggtccctgcgtctctcctgcgcagcctccggattcacctttactaagtattttatgggttgggtccgccaggctccagggaagggtctagagtgggtctcactgattgatccgcgtggtcctcatacatactacgcagactccgtgaagggccggttcaccatctcccgcgacaattccaagaacacgctgtatctgcaaatgaacagcctgcgtgccgaggacaccgcggtatattactgtgcgaaacagttgggtgaggagtttgactactggggtcagggaaccctggtcaccgtctcgagc

SEQ ID NO: 39 - DNA, 351 bp, Artificial Sequence
  DOM21-40 CD28 antibody single variable domain
gaggtgcagctgttggagtctgggggaggcttggtacagcctggggggtccctgcgtctctcctgtgcagcctccggattcacctttaagacttatacgatgaggtgggtccgccaggctccagggaagggtctagagtgggtctcaactattaattcgagtggtactttgacatactacgcagactccgtgaagggccggttcaccatctcccgcgacaattccaagaacacgctgtatctgcaaatgaacagcctgcgtgccgaggataccgcggtatattactgtgcgaaatctagttcttatacgtttgactactggggtcagggaaccctggtcaccgtctcgagc

SEQ ID NO: 40 - DNA, 354 bp, Artificial Sequence
  DOM21-41 CD28 antibody single variable domain
gaggtgcagctgttggagtctgggggaggcttggtacagcctggggggtccctgcgtctctcctgtgcagcctccggattcacctttgcgatgtatagtatgaagtgggtccgccaggctccagggaagggtctagagtgggtctcatcgatttcgaatgctggtgatattacatactacgcagactccgtgaagggccggttcaccatctcccgcgacaattccaagaacacgctgtatctgcaaatgaacagcctgcgtgccgaggataccgcggtatattactgtgcggaatcgtttaggtctcgttattttgactactggggtcagggaaccctggtcaccgtctcgagc

SEQ ID NO: 41 - DNA, 357 bp, Artificial Sequence
  DOM21-42 CD28 antibody single variable domain
gaggtgcagctgttggagtctgggggaggcttggtacagcctggggggtccctgcgtctctcctgtgcagcctccggattcacctttgatgattatcttatggggtgggtccgccaggctccagggaagggtctggagtgggtctcactgattcgtatgaggggttctgttacatactacgcagactccgtgaagggccggttcaccatctcccgcgacaattccaagaacacgctgtatctgcaaatgaacagcctgcgtgccgaggacaccgcggtatattactgtgcgaaacattctcttactactaatctttttgactactggggtcagggaaccctggtcaccgtctcgagc

SEQ ID NO: 42 - DNA, 357 bp, Artificial Sequence
  DOM21-43 CD28 antibody single variable domain
gaggtgcagctgttggagtctgggggaggcttggtacagcctggggggtccctgcgtctctcctgtgcagcctccggattcacctttactgattatatgatggcttgggcccgccaggctccagggaagggtctagagtgggtctcaattattgggactactggtacgtggacatactacgcagactccgtgaagggccggttcaccatctcccgcgacaattccaagaacacgctgtatctgcaaatgaacagcctgcgtgccgaggacaccgcggtatattactgtgcgaaaactaatgcgtatgagagtgagtttgactactggggtcagggaaccctggtcaccgtctcgagc

SEQ ID NO: 43 - DNA, 360 bp, Artificial Sequence
  DOM21-44 CD28 antibody single variable domain
gaggtgcagctgttggagtctgggggaggcttggtacagcctggggggtccctgcgtctctcctgtgcagcctccggattcacctttgcgcggtatactatggtgtgggtccgccaggctccagggaagggtctagagtgggtctcagctattcattttgatggtcggactacatactacgcagactccgtgaagggccggttcaccatctcccgcgacaattccaagaacacgctgtatctgcaaatgaatagcctgcgtgccgaggacaccgcggtatattactgtgcgaaaaatgagtgggcgtctcttaagcattttgactactggggtcagggaaccctggtcaccgtctcgagc

SEQ ID NO: 44 - DNA, 360 bp, Artificial Sequence
  DOM21-45 CD28 antibody single variable domain
gaggtgcagctgttggagtctgggggaggcttggtacagcctggggggtccctgcgtctctcctgtgcagcctccggattcacctttgaggattatatgatgggttgggtccgccaggctccagggaagggtctagagtgggtctcatttattaatctgcctggtggtcgtacatactacgcagactccgtgaagggccggttcaccatctcccgcgacaattccaagaacacgctgtatctgcaaatgaacagcctgcgtgccgaggacaccgcggtatattactgtgcgaaacagactcatgggctgactggttattttgactactggggtcagggaaccctggtcaccgtctcgagc

SEQ ID NO: 45 - DNA, 360 bp, Artificial Sequence
  DOM21-46 CD28 antibody single variable domain
gaggtgcagctgttggagtctgggggaggcttggtacagcctggggggtccctgcgtctctcctgtgcagcctccggattcacctttggtttgtatggtatggcttgggcccgccaggctccagggaagggtctagagtgggtctcatcgattgggatgcatggtgatactacatactacgcagactccgtgaagggccggttcaccatctcccgcgacaattccaagaacacgctgtatctgcaaatgaacagcctgcgtgccgaggacaccgcggtatattactgtgcgaaagtttgtggggctacgtattgtaattttgactactggggtcagggaaccctggtcaccgtctcgagc

SEQ ID NO: 46 - DNA, 360 bp, Artificial Sequence
  DOM21-47 CD28 antibody single variable domain
gaggtgcagctgttggagtctgggggaggcttggtacagcctggggggtccctgcgtctctcctgtgcagcctccggattcacctttggtaagtatgttatggcttgggtccgccaggctccagggaagggtctagagtgggtctcaattattgattccttgggttctactacatactacgcagactccgtgaagggccggttcaccatctcccgcgacaattccaagaacacgctgtatctgcaaatgaacagcctgcgtgccgaggacaccgcggtatattactgtgcgaaagggggtttgttggttcattatgattttgactactggggtcagggaaccctggtcaccgtctcgagc

SEQ ID NO: 47 - DNA, 363 bp, Artificial Sequence
  DOM21-48 CD28 antibody single variable domain
gaggtgcagctgttggagtctgggggaggcttggtacagcctggggggtccctgcgtctctcctgtgcagcctccggattcacctttgaggtgtatggtatgtcttgggcccgccaggctccagggaagggtctagagtgggtctcattgattgatgcgggtggtcggaatacatactacgcagactccgtgaagggccggttcaccatctcccgcgacaattccaagaacacgctgtatctgcaaatgaacagcctgcgtgccgaggataccgcggtatattactgtgcgaaatcgacgacgcgtgcttatagtgattattttgactactggggtcagggaaccctggtcaccgtctcgagc

SEQ ID NO: 48 - DNA, 363 bp, Artificial Sequence
  DOM21-49 CD28 antibody single variable domain
gaggtgcagctgttggagtctgggggaggcttggtacagcctggggggtccctgcgtctctcctgtgcagcctccggattcacctttgagaattatgatatgcattgggtccgccaggctccagggaagggtctagagtgggtctcagggattactacgcatggtaggcgtacatactacgcagactccgtgaagggccggttcaccatctcccgcgacaattccaagaacacgctgtatctgcaaatgaacagcctgcgtgccgaggataccgcggtatattactgtgcgaaaagtgataatttgaatatgaatgtggattttgactactggggtcagggaaccctggtcaccgtctcgagc

SEQ ID NO: 49 - DNA, 363 bp, Artificial Sequence
  DOM21-50 CD28 antibody single variable domain
gaggtgcagctgttggagtctgggggaggcttggtacagcctggggggtccctgcgtctctcctgtgcagcctccggattcacctttattaagtatgatatgtgttgggcccgccaggctccagggaagggtctagagtgggtctcatgtattgagtctagtggtcagaatacatactacgcagactccgtgaagggccggttcaccatctcccgcgacaattccaagaacacgctgtatctgcaaatgaacagcctgcgtgccgaggacaccgcggtatattactgtgcgaaatgtctgaatgatagttgtaatgttcattttgactactggggtcagggaaccctggtcaccgtctcgagc

SEQ ID NO: 50 - DNA, 363 bp, Artificial Sequence
  DOM21-51 CD28 antibody single variable domain
gaggtgcagctgttggagtctgggggaggcttggtacagcctggggggtccctgcgtctctcctgtgcagcctccggattcacctttggtaattataatatggggtgggtccgccaggctccagggaagggtctagagtgggtctcagatattggtcgttatggtagggttacatactacgcagactccgtgaagggccggttcaccatctcccgcgacaattccaagaacacgctgtatctgcaaatgaacagcctgcgtgccgaggataccgcggtatattactgtgcgaaaactcagcgtatggttaatccgtcgccttttgactactggggtcagggaaccctggtcaccgtctcgagc

SEQ ID NO: 51 - DNA, 369 bp, Artificial Sequence
  DOM21-52 CD28 antibody single variable domain
gaggtgcagctgttggagtctgggggaggcttggtacagcctggggggtccctgcgtctctcctgtgcagcttccggattcacctttgttagttatagtatgggttgggtccgccaggctccagggaagggtctagagtgggtctcaattatttcggggcagggtactgttacatactacgcagactccgtgaagggccggttcaccatctcccgcgacaattccaagaacacgctgtatctgcaaatgaacagcctgcgtgccgaggacaccgcggtatattactgtgcgaaatcgccgatggtttttgctttggatgggaggtcttttgactactggggtcagggaaccctggtcaccgtctcgagc

SEQ ID NO: 52 - DNA, 369 bp, Artificial Sequence
  DOM21-53 CD28 antibody single variable domain
gaggtgcagctgttggagtctgggggaggcttggtacagcctggggggtccctgcgtctctcctgtacagcctccggattcaccttttctgagtatagtatggggtgggtccgccaggctccagggaagggtctagagtgggtctcaagtattacgcctgttggtgtttttacatactacgcagactccgtgaagggccggttcaccatctcccgcgacaattccaagaacacgctgtatctgcaaatgaacagcctgcgtgccgaggacaccgcggtatattactgtgcgaaagggaggcctgggccgcatggttggtcttttcggtttgactactggggtcagggaaccctggtcaccgtctcgagc

SEQ ID NO: 53 - DNA, 369 bp, Artificial Sequence
  DOM21-54 CD28 antibody single variable domain
gaggtgcagctgttggagtctgggggaggcttggtacagcctggggggtccctgcgtctctcctgtgcagcctccggattcacctttgggcagtatatgatgggttgggtccgccaggctccagggaagggtctagagtgggtctcaactattgataagtcgggttatagtacatactacgcagactccgtgaagggccggttcaccatctcccgcgacaattccaagaacacgctgtatctgcaaatgaacagcctgcgtgccgaggacaccgcggtatattactgtgcgaaaagtgggattgattcgcggggtctgatgactaagtttgactactggggtcagggaaccctggtcaccgtctcgagc

SEQ ID NO: 54 - DNA, 369 bp, Artificial Sequence
  DOM21-55 CD28 antibody single variable domain
gaggtgcagctgttggagtctgggggaggcttggtacagcctggggggtccctgcgtctctcctgtgcagcctccggattcacctttgctcgttatcgtatggcgtgggtccgccaggctccagggaagggtctagagtgggtctcatctattctgagtgatggtgcggttacatactacgcagactccgtgaagggccggttcaccatctcccgcgacaattccaagaacacgctgtatctgcaaatgaacagcctgcgtgccgaggacaccgcggtatattactgtgcgaaacctggggggaatgcgtggtctactcgggttacttttgactactggggtcagggaaccctggtcaccgtctcgagc

SEQ ID NO: 55 - DNA, 369 bp, Artificial Sequence
  DOM21-56 CD28 antibody single variable domain
gaggtgcagctgttggagtctgggggaggcttggtacagcctggggggtccctgcgtctctcctgtgcagcctccggattcaccttttttacgtatacnatggcttgggtccgccaggctccagggaagggtctagagtgggtctcatctattacgccgcttggttataatacatactacgcagactccgtgaagggccggttcaccatctcccgcgacaattccaagaacacgctgtatctgcaaatgaacagcctgcgtgccgaggacaccgcggtatattactgtgcgaaaccgtcggatgtgaaggtgtctccgctgccgagttttgactactggggtcggggaaccctggtcaccgtctcgagc

SEQ ID NO: 56 - DNA, 372 bp, Artificial Sequence
  DOM21-57 CD28 antibody single variable domain
gaggtgcagctgttggagtctgggggaggcttggtacagcctggggggtccctgcgtctctcctgtgcagcctccggattcacctttactatgtatggtatgcattgggtccgccaggctccagggaagggtctagagtgggtctcatcgatttctcagtatggtctttctacatactacgcagattccgtgaagggccggttcaccatctcccgcgacaattccaagaacacgctgtatctgcaaatgaacagcctgcgtgccgaggacaccgcggtatattactgtgcgaaagggtctatgaggcgggtgtttagtagttcggatacttttgactactggggtcagggaaccctggtcaccgtctcgagc

SEQ ID NO: 57 - DNA, 324 bp, Artificial Sequence
  DOM21-58 CD28 antibody single variable domain
gacatccagatgacccagtctccatcctccctgtctgcatctgtaggagaccgtgtcaccatcacttgccgggcaagtcagaatataggtgatcggttacattggtaccagcagaaaccagggaaagcccctaagctcctgatctatcgtatttcccgtttgcaaagtggggtcccatcacgtttcagtggcagtggatctgggacagatttcactctcaccatcagcagtctgcaacctgaagattttgctacgtactactgtcaacagtttgggctgtatcctactacgttcggccaagggaccaaggtggaaatcaaacgg

SEQ ID NO: 58 - protein, 116 aa, Artificial Sequence
  DOM21-1 CD28 antibody single variable domain
EVQLLESGGGLVQPGGSLRLSCAASGFTFDAYSMIWVRQAPGKGLEWVSTITPQGDRTYYADSVKGRFTISRDNSKNTLYLQMNSLRAEDTAVYYCAQAGWSFDYWGQGTLVTVSS

SEQ ID NO: 59 - protein, 116 aa, Artificial Sequence
  DOM21-2 CD28 antibody single variable domain
EVQLLESGGGLVQPGGSLRLSCAASGFTFVDYEMAWVRQAPGKGLEWVSTISNDGAATYYADSVKGRFTISRDNSKNTLYLQMNSLRAEDTAVYYCAKDDAAFDYWGQGALVTVSS

SEQ ID NO: 60 - protein, 116 aa, Artificial Sequence
  DOM21-3 CD28 antibody single variable domain
EVQLLESGGGLVQPGGSLRLSCAASGFTFGAYSMGWARQAPGKGLEWVSWITGNGGSTYYADSVKGRFTISRDNSKNTLYLQMNSLRAEDTAVYYCAKAEEPFDYWGQGTLVTVSS

SEQ ID NO: 61 - protein, 116 aa, Artificial Sequence
  DOM21-4 CD28 antibody single variable domain
EVQLLESGGGLVQPGGSLRLSCAASGFTFSRYHMAWVRQAPGKGLEWVSVIDSLGLQTYYADSVKGRFTISRDNSKNTLYLQMNSLRAEDTAVYYCAEYGGAFDYWGQGTLVTVSS

SEQ ID NO: 62 - protein, 117 aa, Artificial Sequence
  DOM21-5 CD28 antibody single variable domain
EVQLLESGGGLVQPGGSLRLSCAASGFTFTHYSMGWVRQAPGKGLEWVSHITPDGLITYYADSVKGRFTISRDNSKNTLYLQMNSLRAEDTAVYYCAKGRLVDFDYWGQGTLVTVSS

SEQ ID NO: 63 - protein, 118 aa, Artificial Sequence
  DOM21-6 CD28 antibody single variable domain
EVQLLESGGGLVQPGGSLRLSCAASGFTFENYGMAWVRQAPGKGLEWVSNIGRAGSVTYYADSVKGRFTISRDNSKNTLYLQMNSLRAEDTAVYYCAKVQSWRTFDYWGQGTLVTVSS

SEQ ID NO: 64 - protein, 119 aa, Artificial Sequence
  DOM21-7 CD28 antibody single variable domain
EVQLLESGGGLVQPGGSLRLSCAASGFTFPAYSMGWVRQAPEKGLEWVSYIDGRGAETYYADSVKGRFTISRDNSKNTLYLQMNSLRAEDTAVYYCAKIDTLISEFDYWGQGTLVTVSS

SEQ ID NO: 65 - protein, 119 aa, Artificial Sequence
  DOM21-8 CD28 antibody single variable domain
EVQLLESGGGLVQPGGSLRLSCAASGFTFPNYTMWWVRQAPGKGLEWVSSISGTGHTTYYADSVKGRFTISRDNSKNTLYLQMNSLRAEDTAVYYCAKFGPNNPMFDYWGQGTLVTVSS

SEQ ID NO: 66 - protein, 119 aa, Artificial Sequence
  DOM21-9 CD28 antibody single variable domain
EVQLLESGGGLVQPGGSLRLSCAASGFTFASYDMGWVRQAPGKGLEWVSAISADGTFTYYADSVKGRFTISRDNSKNTLYLQMNSLRAEDTAVYYCAKSSFDKYNFDYWGQGTLVTVSS

SEQ ID NO: 67 - protein, 119 aa, Artificial Sequence
  DOM21-10 CD28 antibody single variable domain
EVQLLESGGGLVQPGGSLRLSCAASGFTFAKYTMWWVRQAPGKGLEWVSSIDPVGNLTYYADSVKGRFTISRDNSKNTLYLQMNSLRAEDTAVYYCAKRGPTSSNFDYWGQGTLVTVSS

SEQ ID NO: 68 - protein, 120 aa, Artificial Sequence
  DOM21-11 CD28 antibody single variable domain
EVQLLESGGGLVQPGGSLRLSCAASGFTFSEYGMKWVRQAPGKGLEWVSTIDNVGSVTYYADSVKGRFTISRDNSKNTLYLQMNSLRAEDTAVYYCAKTTPVLLPLFDYWGQGTLVTVSS

SEQ ID NO: 69 - protein, 120 aa, Artificial Sequence
  DOM21-12 CD28 antibody single variable domain
EVQLLESGGGLVQPGGSLRLSCAASGFTFDSYNMGWVRQAPGKGLEWVSAIAANGRVTYYADSVKGRFTISRDNSKNTLYLQMNSLRAEDTAVYYCAKMTNMAYGSFDYWGQGTLVTVSS

SEQ ID NO: 70 - protein, 120 aa, Artificial Sequence
  DOM21-13 CD28 antibody single variable domain
EVQLLESGGGLVQPGGSLRLSCAASGFTFDLYSMAWVRQAPGKGLEWVSHIDRAGMITYYADSVKGRFTISRDNSKNTLYLQMNSLRAEDTAVYYCAKVSNAVNMQFDYWGQGTLVTVSS

SEQ ID NO: 71 - protein, 120 aa, Artificial Sequence
  DOM21-14 CD28 antibody single variable domain
EVQLLESGGGLVQPGGSLRLSCAASGFTFAKYTMWWVRQAPGKGLEWVSSIDPVGNLTYYADSVKGRFTISRDNSKNTLYLQMNSLRAEDTAVYYCAKRHRPSTQDFDYWGQGTLXTVSS

SEQ ID NO: 72 - protein, 120 aa, Artificial Sequence
  DOM21-15 CD28 antibody single variable domain
EVQLLESGGGLVQPGGSLRLSCAASGFTFPDYKMGWVRQAPGKGLEWVSWIDKGGIITYYADSVKGRFTISRDNSKNTLYLQMNSLRAEDTAVYYCAKMFPKFRPAFDYWGQGTLVTVSS

SEQ ID NO: 73 - protein, 120 aa, Artificial Sequence
  DOM21-16 CD28 antibody single variable domain
EVQLLESGGGLVQPGGSLRLSCAASGFTFEDYGMGWVRQAPGKGLEWVSHINRSGLVTYYADSVKGRFTISRDNSKNTLYLQMNSLRAEDTAVYYCAKVLNAPNFKFDYWGQGTLVTVSS

SEQ ID NO: 74 - protein, 123 aa, Artificial Sequence
  DOM21-17 CD28 antibody single variable domain
EVQLLESGGGLVQPGGSLRLSCAASGFTFNRYAMGWVRQAPGKGLEWVSWIDGNGLVTYYADSVKGRFTISRDNSKNTLYLQMNSLRAEDTAVYYCAKRTRSHSDSGWAFDYWGQGTLVTVSS

SEQ ID NO: 75 - protein, 108 aa, Artificial Sequence
  DOM21-18 CD28 antibody single variable domain
DIQMTQSPSSLSASVGDRVTITCRASQYIGTSLNWYQQKPGKAPKLLTYQASLLQSGVPSRFSGSGSGTDFTLTISSLQPEDFATYYCQQLALRPMTFGQGTKVEIKR

SEQ ID NO: 76 - protein, 123 aa, Artificial Sequence
  DOM21-19 CD28 antibody single variable domain
EVQLLESGGGLVQPGGSLRLSCAASGFTFGNYNMGWVRQAPGKGLEWVSGITKGGRVTYYADSVKGRFTISRDNSKNTLYLQMNSLRAEDTAVYYCAKLGPSRMLNEPLFDYWGQGTLVTVSS

SEQ ID NO: 77 - protein, 123 aa, Artificial Sequence
  DOM21-20 CD28 antibody single variable domain
EVQLLESGGGLVQPGGSLRLSCAASGFTFPAYSMIWVRQAPGKGLEWVSTISPLGYSTYYADSVKGRFTISRDNSKNTLYLQMNSLRAEDTAVYYCAEQTAYLNRATEHFDYWGQGTLVTVSS

SEQ ID NO: 78 - protein, 123 aa, Artificial Sequence
  DOM21-21 CD28 antibody single variable domain
EVQLLESGGGLVQPGGSLRLSCAASGFTFSKYDMAWVRQAPGKGLEWVSSIYAIGGNTYYADSVKGRFTISRDNSKNTLYLQMNSLRAEDTAVYYCAKLKSGMQTRLNSFDYWGQGTLVTVSS

SEQ ID NO: 79 - protein, 123 aa, Artificial Sequence
  DOM21-22 CD28 antibody single variable domain
EVQLLESGGGLVQPGGSLRLSCAASGFTFELYQMGWVRQAPGKGLEWVSTIMPSGNLTYYADSVKGRFTISRDNSKNTLYLQMNSLRAEDTAVYYCAKMWSLNLGFHAAFDYWGQGTLVTVSS

SEQ ID NO: 80 - protein, 123 aa, Artificial Sequence
  DOM21-23 CD28 antibody single variable domain
EVQLLESGGGLVQPGGSLRLSCAASGFTFGQYGMGWVRQAPGKGLEWVSGISPSGNYTYYADSVKGRFTISRDNSKNTLYLQMNSLRAEDTAVYYCAKGNGSLPPRGSIFDYWGQGTLVTVSS

SEQ ID NO: 81 - protein, 123 aa, Artificial Sequence; identical to SEQ ID NO: 76
  DOM21-24 CD28 antibody single variable domain

SEQ ID NO: 82 - protein, 123 aa, Artificial Sequence
  DOM21-25 CD28 antibody single variable domain
EVQLLESGGGLVQPGGSLRLSCAASGFTFGTYYMGWARQAPGKGLEWVSSIGANGAPTYYADSVKGRFTISRDNSKNTLYLQMNSLRAEDTAVYYCAKIRSLNRWAEPVFDYWGQGTLVTVSS

SEQ ID NO: 83 - protein, 116 aa, Artificial Sequence
  DOM21-26 CD28 antibody single variable domain
EVQLLESGGGLVQPGGSLRLSCAASGFTFADYSMYWVRQAPGKGLEWVSQISPAGSFTYYADSVKGRFTISRDNSKNTLYLQMNSLRAEDTAVYYCAKDSKSFDYWGQGTLVTVSS

SEQ ID NO: 84 - protein, 108 aa, Artificial Sequence
  DOM21-27 CD28 antibody single variable domain
DIQMTQSPSSLSASVGDRVTITCRASQSIGTGLRWYQQKPGKAPMLLIYRASILQSGVPSRFSGSGSGTDFTLTISSLQPEDFATYYCQQTTLQPFTFSQGTKVEIKR

SEQ ID NO: 85 - protein, 108 aa, Artificial Sequence
  DOM21-28 CD28 antibody single variable domain
DIQMTQSPSSLSASVGDRVTITCRASQSISHSLVWYQQKPGKAPKLLIYWASLLQSGVPSRFSGSGSGTDFTLTISSLQPEDFATYYCQQGMTTPFTFGQGTKVEIKR

SEQ ID NO: 86 - protein, 116 aa, Artificial Sequence
  DOM21-30 CD28 antibody single variable domain
EVQLLESGGGLVQPGGSLRLSCAASGFTFDSYDMNWVRQAPGKGLEWVSQISADGHFTYYADSVKGRFTISRDNSKNTLYLQMNSLRAEDTAVYYCAKSRSSFDYWGQGTLVTVSS

SEQ ID NO: 87 - protein, 116 aa, Artificial Sequence
  DOM21-31 CD28 antibody single variable domain
EVQLLESGGGLVQPGGSLRLSCAASGFTFRDYMMGWVRQAPGKGLEWVSRIDSHGNRTYYADSVKGRFTISRDNSKNTLYLQMNSLRAEDTAVYYCAKHMTGFDYWGQGTLVTVSS

SEQ ID NO: 88 - protein, 116 aa, Artificial Sequence
  DOM21-32 CD28 antibody single variable domain
EVQLLESGGGLVQPGGSLRLSCAASGFTFREYMMGWVRQAPGKGLEWVSRINGVGNSTYYADSVKGRFTISRDNSKNTLYLQMNSLRAEDTAVYYCAKHQVGFDYWGQGTLVTVSS

SEQ ID NO: 89 - protein, 116 aa, Artificial Sequence
  DOM21-33 CD28 antibody single variable domain
EVQLLESGGGLVQPGGSLRLSCAASGFTFSDYMMGWVRQAPGKGLEWVSRITSEGSHTYYADSVKGRFTISRDNSKNTLYLQMNSLRAEDTAVYYCAKHTSGFDYWGQGTLVTVSS

SEQ ID NO: 90 - protein, 116 aa, Artificial Sequence
  DOM21-34 CD28 antibody single variable domain
EVQLLESGGGLVQPGGSLRLSCAASGFTFGRYMMGWVRQAPGKGLEWVSRISGPGTVTYYADSVKGRFTISRDNSKNTLYLQMNSLRAEDTAVYYCAKHDTGFDYWGQGTLVTVSS

SEQ ID NO: 91 - protein, 116 aa, Artificial Sequence
  DOM21-35 CD28 antibody single variable domain
EVQLLESGGGLVQPGGSLRLSCAASGFTFSSYAMIWVRQAPGKGLEWVSEISPYGNHTYYADSVKGRFTISRDNSKNTLYLQMNSLRAEDTAVYYCAKPDRRFDYWGQGTLVTVSS

SEQ ID NO: 92 - protein, 116 aa, Artificial Sequence
  DOM21-36 CD28 antibody single variable domain
EVQLLESGGGLVQPGGSLRLSCAASGFTFTSYGMQWVRQAPGKGLEWVSSISTDGMVTYYADSVKGRFTISRDNSKNTLYLQMNSLRAEDTAVYYCAKLGVNFDYWGQGTLVTVSS

SEQ ID NO: 93 - protein, 117 aa, Artificial Sequence
  DOM21-37 CD28 antibody single variable domain
EVQLLESGGGLVQPGGSLRLSCAASGFTFGDYMMGWVRQAPGKGLEWVSIIRVPGSTTYYADSVKGRFTISRDNSKNTLYLQMNSLRAEDTAVYYCAKQKGDEFDYWGQGTLVTVSS

SEQ ID NO: 94 - protein, 117 aa, Artificial Sequence
  DOM21-38 CD28 antibody single variable domain
EVQLLESGGGLVQPGGSLRLSCAASGFTFILYDMQWVRQAPGKGLEWVSRISANGHDTYYADSVKGRFTISRDNSKNTLYLQMNSLRAEDTAVYYCAKGPHYLFDYWGQGTLVTVSS

SEQ ID NO: 95 - protein, 117 aa, Artificial Sequence
  DOM21-39 CD28 antibody single variable domain
EVQLLESGGGLVQPGGSLRLSCAASGFTFTKYFMGWVRQAPGKGLEWVSLIDPRGPHTYYADSVKGRFTISRDNSKNTLYLQMNSLRAEDTAVYYCAKQLGEEFDYWGQGTLVTVSS

SEQ ID NO: 96 - protein, 117 aa, Artificial Sequence
  DOM21-40 CD28 antibody single variable domain
EVQLLESGGGLVQPGGSLRLSCAASGFTFKTYTMRWVRQAPGKGLEWVSTINSSGTLTYYADSVKGRFTISRDNSKNTLYLQMNSLRAEDTAVYYCAKSSSYTFDYWGQGTLVTVSS

SEQ ID NO: 97 - protein, 118 aa, Artificial Sequence
  DOM21-41 CD28 antibody single variable domain
EVQLLESGGGLVQPGGSLRLSCAASGFTFAMYSMKWVRQAPGKGLEWVSSISNAGDITYYADSVKGRFTISRDNSKNTLYLQMNSLRAEDTAVYYCAESFRSRYFDYWGQGTLVTVSS

SEQ ID NO: 98 - protein, 119 aa, Artificial Sequence
  DOM21-42 CD28 antibody single variable domain
EVQLLESGGGLVQPGGSLRLSCAASGFTFDDYLMGWVRQAPGKGLEWVSLIRMRGSVTYYADSVKGRFTISRDNSKNTLYLQMNSLRAEDTAVYYCAKHSLTTNLFDYWGQGTLVTVSS

SEQ ID NO: 99 - protein, 119 aa, Artificial Sequence
  DOM21-43 CD28 antibody single variable domain
EVQLLESGGGLVQPGGSLRLSCAASGFTFTDYMMAWARQAPGKGLEWVSIIGTTGTWTYYADSVKGRFTISRDNSKNTLYLQMNSLRAEDTAVYYCAKTNAYESEFDYWGQGTLVTVSS

SEQ ID NO: 100 - protein, 120 aa, Artificial Sequence
  DOM21-44 CD28 antibody single variable domain
EVQLLESGGGLVQPGGSLRLSCAASGFTFARYTMVWVRQAPGKGLEWVSAIHFDGRTTYYADSVKGRFTISRDNSKNTLYLQMNSLRAEDTAVYYCAKNEWASLKHFDYWGQGTLVTVSS

SEQ ID NO: 101 - protein, 120 aa, Artificial Sequence
  DOM21-45 CD28 antibody single variable domain
EVQLLESGGGLVQPGGSLRLSCAASGFTFEDYMMGWVRQAPGKGLEWVSFINLPGGRTYYADSVKGRFTISRDNSKNTLYLQMNSLRAEDTAVYYCAKQTHGLTGYFDYWGQGTLVTVSS

SEQ ID NO: 102 - protein, 120 aa, Artificial Sequence
  DOM21-46 CD28 antibody single variable domain
EVQLLESGGGLVQPGGSLRLSCAASGFTFGLYGMAWARQAPGKGLEWVSSIGMHGDTTYYADSVKGRFTISRDNSKNTLYLQMNSLRAEDTAVYYCAKVCGATYCNFDYWGQGTLVTVSS

SEQ ID NO: 103 - protein, 120 aa, Artificial Sequence
  DOM21-47 CD28 antibody single variable domain
EVQLLESGGGLVQPGGSLRLSCAASGFTFGKYVMAWVRQAPGKGLEWVSIIDSLGSTTYYADSVKGRFTISRDNSKNTLYLQMNSLRAEDTAVYYCAKGGLLVHYDFDYWGQGTLVTVSS

SEQ ID NO: 104 - protein, 121 aa, Artificial Sequence
  DOM21-48 CD28 antibody single variable domain
EVQLLESGGGLVQPGGSLRLSCAASGFTFEVYGMSWARQAPGKGLEWVSLIDAGGRNTYYADSVKGRFTISRDNSKNTLYLQMNSLRAEDTAVYYCAKSTTRAYSDYFDYWGQGTLVTVSS

SEQ ID NO: 105 - protein, 121 aa, Artificial Sequence
  DOM21-49 CD28 antibody single variable domain
EVQLLESGGGLVQPGGSLRLSCAASGFTFENYDMHWVRQAPGKGLEWVSGITTHGRRTYYADSVKGRFTISRDNSKNTLYLQMNSLRAEDTAVYYCAKSDNLNMNVDFDYWGQGTLVTVSS

SEQ ID NO: 106 - protein, 121 aa, Artificial Sequence
  DOM21-50 CD28 antibody single variable domain
EVQLLESGGGLVQPGGSLRLSCAASGFTFIKYDMCWARQAPGKGLEWVSCIESSGQNTYYADSVKGRFTISRDNSKNTLYLQMNSLRAEDTAVYYCAKCLNDSCNVHFDYWGQGTLVTVSS

SEQ ID NO: 107 - protein, 121 aa, Artificial Sequence
  DOM21-51 CD28 antibody single variable domain
EVQLLESGGGLVQPGGSLRLSCAASGFTFGNYNMGWVRQAPGKGLEWVSDIGRYGRVTYYADSVKGRFTISRDNSKNTLYLQMNSLRAEDTAVYYCAKTQRMVNPSPFDYWGQGTLVTVSS

SEQ ID NO: 108 - protein, 123 aa, Artificial Sequence
  DOM21-52 CD28 antibody single variable domain
EVQLLESGGGLVQPGGSLRLSCAASGFTFVSYSMGWVRQAPGKGLEWVSIISGQGTVTYYADSVKGRFTISRDNSKNTLYLQMNSLRAEDTAVYYCAKSPMVFALDGRSFDYWGQGTLVTVSS

SEQ ID NO: 109 - protein, 123 aa, Artificial Sequence
  DOM21-53 CD28 antibody single variable domain
EVQLLESGGGLVQPGGSLRLSCTASGFTFSEYSMGWVRQAPGKGLEWVSSITPVGVFTYYADSVKGRFTISRDNSKNTLYLQMNSLRAEDTAVYYCAKGRPGPHGWSFRFDYWGQGTLVTVSS

SEQ ID NO: 110 - protein, 123 aa, Artificial Sequence
  DOM21-54 CD28 antibody single variable domain
EVQLLESGGGLVQPGGSLRLSCAASGFTFGQYMMGWVRQAPGKGLEWVSTIDKSGYSTYYADSVKGRFTISRDNSKNTLYLQMNSLRAEDTAVYYCAKSGIDSRGLMTKFDYWGQGTLVTVSS

SEQ ID NO: 111 - protein, 123 aa, Artificial Sequence
  DOM21-55 CD28 antibody single variable domain
EVQLLESGGGLVQPGGSLRLSCAASGFTFARYRMAWVRQAPGKGLEWVSSILSDGAVTYYADSVKGRFTISRDNSKNTLYLQMNSLRAEDTAVYYCAKPGGNAWSTRVTFDYWGQGTLVTVSS

SEQ ID NO: 112 - protein, 123 aa, Artificial Sequence
  DOM21-56 CD28 antibody single variable domain
EVQLLESGGGLVQPGGSLRLSCAASGFTFFTYXMAWVRQAPGKGLEWVSSITPLGYNTYYADSVKGRFTISRDNSKNTLYLQMNSLRAEDTAVYYCAKPSDVKVSPLPSFDYWGRGTLVTVSS

SEQ ID NO: 113 - protein, 124 aa, Artificial Sequence
  DOM21-57 CD28 antibody single variable domain
EVQLLESGGGLVQPGGSLRLSCAASGFTFTMYGMHWVRQAPGKGLEWVSSISQYGLSTYYADSVKGRFTISRDNSKNTLYLQMNSLRAEDTAVYYCAKGSMRRVFSSSDTFDYWGQGTLVTVSS

SEQ ID NO: 114 - protein, 108 aa, Artificial Sequence
  DOM21-58 CD28 antibody single variable domain
DIQMTQSPSSLSASVGDRVTITCRASQNIGDRLHWYQQKPGKAPKLLIYRISRLQSGVPSRFSGSGSGTDFTLTISSLQPEDFATYYCQQFGLYPTTFGQGTKVEIKR

SEQ ID NO: 115 - protein, 108 aa, Artificial Sequence
  MSA16 serum albumin
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 116 - DNA, 324 bp, Artificial Sequence
  MSA16 serum albumin
gacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgg

SEQ ID NO: 117 - protein, 108 aa, Artificial Sequence
  MSA26 serum albumin
DIQMTQSPSSLSASVGDRVTITCRASQSIYYHLKWYQQKPGKAPKLLIYKASTLQSGVPSRFSGSGSGTDFTLTISSLQPEDFATYYCQQVRKVPRTFGQGTKVEIKR

SEQ ID NO: 118 - DNA, 324 bp, Artificial Sequence
  MSA26 serum albumin
gacatccagatgacccagtctccatcctccctgtctgcatctgtaggagaccgtgtcaccatcacttgccgggcaagtcagagcatttattatcatttaaagtggtaccagcagaaaccagggaaagcccctaagctcctgatctataaggcatccacgttgcaaagtggggtcccatcacgtttcagtggcagtggatctgggacagatttcactctcaccatcagcagtctgcaacctgaagattttgctacgtactactgtcaacaggttcggaaggtgcctcggacgttcggccaagggaccaaggtggaaatcaaacgg

SEQ ID NO: 119 - protein, 120 aa, Artificial Sequence
  HEL4 a VH dAb that binds hen egg lysozyme
EVQLLESGGGLVQPGGSLALSCAASGFRISDEDMGWVRQAPGKGLEWVSSIYGPSGSTYYADSVKGRFTISRDNSKNWLYLQMNSLRAEDTAVYYCASALEPLSEPLGFWGQGTLVTVSS

SEQ ID NO: 120 - protein, 108 aa, Artificial Sequence
  Vkappa dummy dAb
DIQMTQSPSSLSASVGDRVWITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 121 - protein, 4 aa, Artificial Sequence
  fragment of CDR1 of CD28 VL dAbs
RASQ

SEQ ID NO: 122 - protein, 6 aa, Artificial Sequence
  fragment of CDR1 of CD28 VL dAbs
RASQXI

SEQ ID NO: 123 - protein, 7 aa, Artificial Sequence
  fragment of CDR1 of CD28 VL dAbs
RASQXIG

SEQ ID NO: 124 - protein, 10 aa, Artificial Sequence
  fragment of CDR1 of CD28 VL dAbs
RASQXIGXXL

SEQ ID NO: 125 - protein, 6 aa, Artificial Sequence
  fragment of CDR1 of CD28 VL dAbs
XSXLQS

SEQ ID NO: 126 - protein, 7 aa, Artificial Sequence
  fragment of CDR1 of CD28 VL dAbs
QASLLQS

SEQ ID NO: 127 - protein, 7 aa, Artificial Sequence
  fragment of CDR1 of CD28 VL dAbs
RASILQS

SEQ ID NO: 128 - protein, 7 aa, Artificial Sequence
  fragment of CDR1 of CD28 VL dAbs
WASLLQS

SEQ ID NO: 129 - protein, 7 aa, Artificial Sequence
  fragment of CDR1 of CD28 VL dAbs
RISRLQS

SEQ ID NO: 130 - protein, 8 aa, Artificial Sequence
  fragment of CDR2 of CD28 VL dAbs
YYADSVKG

SEQ ID NO: 131 - protein, 5 aa, Artificial Sequence
  fragment of CDR2 of CD28 VL dAbs
TYYAD

SEQ ID NO: 132 - protein, 4 aa, Artificial Sequence
  fragment of CDR2 of CD28 VL dAbs
TYYA

SEQ ID NO: 133 - protein, 6 aa, Artificial Sequence
  fragment of CDR2 of CD28 VL dAbs
TYYADS

SEQ ID NO: 134 - protein, 7 aa, Artificial Sequence
  fragment of CDR2 of CD28 VL dAbs
TYYADSV

SEQ ID NO: 135 - protein, 8 aa, Artificial Sequence
  fragment of CDR2 of CD28 VL dAbs
TYYADSVK

SEQ ID NO: 136 - protein, 9 aa, Artificial Sequence
  fragment of CDR2 of CD28 VL dAbs
TYYADSVKG

SEQ ID NO: 137 - protein, 9 aa, Artificial Sequence
  fragment of CDR3 of CD28 VL dAbs
QQXXXXPXT

SEQ ID NO: 138 - protein, 11 aa, Artificial Sequence
  sequence comprised by CDR1 of CD28 VL dAbs
RASQYIGTSLN

SEQ ID NO: 139 - protein, 11 aa, Artificial Sequence
  sequence comprised by CDR1 of CD28 VL dAbs
RASQSIGTGLR

SEQ ID NO: 140 - protein, 11 aa, Artificial Sequence
  sequence comprised by CDR1 of CD28 VL dAbs
RASQSISHSLV

SEQ ID NO: 141 - protein, 11 aa, Artificial Sequence
  sequence comprised by CDR1 of CD28 VL dAbs
RASQNIGDRLH

SEQ ID NO: 142 - protein, 7 aa, Artificial Sequence
  sequence comprised by CDR2 of CD28 VL dAbs
QASLLQS

SEQ ID NO: 143 - protein, 7 aa, Artificial Sequence
  sequence comprised by CDR2 of CD28 VL dAbs
RASILQS

SEQ ID NO: 144 - protein, 7 aa, Artificial Sequence
  sequence comprised by CDR2 of CD28 VL dAbs
WASLLQS

SEQ ID NO: 145 - protein, 7 aa, Artificial Sequence
  sequence comprised by CDR2 of CD28 VL dAbs
RISRLQS

SEQ ID NO: 146 - protein, 9 aa, Artificial Sequence
  sequence comprised by CDR3 of CD28 VL dAbs
QQLALRPMT

SEQ ID NO: 147 - protein, 9 aa, Artificial Sequence
  sequence comprised by CDR3 of CD28 VL dAbs
QQTTLQPFT

SEQ ID NO: 148 - protein, 9 aa, Artificial Sequence
  sequence comprised by CDR3 of CD28 VL dAbs
QQGMTTPFT

SEQ ID NO: 149 - protein, 9 aa, Artificial Sequence
  sequence comprised by CDR3 of CD28 VL dAbs
QQFGLYPTT

SEQ ID NO: 150 - protein, 5 aa, Artificial Sequence
  sequence comprised by CDR1 of CD28 VH dAbs
RYHMA

SEQ ID NO: 151 - protein, 17 aa, Artificial Sequence
  sequence comprised by CDR2 of CD28 Vh dAbs
VIDSLGLQTYYADSVKG

SEQ ID NO: 152 - protein, 8 aa, Artificial Sequence
  sequence comprised by CDR3 of CD28 VH dAbs
EYGGAFDY

SEQ ID NO: 153 - protein, 23 aa, Artificial Sequence
  sequence comprised by Framework region 1 of CD28 VL dAbs
DIQMTQSPSSLSASVGDRVTITC

SEQ ID NO: 154 - protein, 15 aa, Artificial Sequence
  sequence comprised by Framework region 2 of CD28 VL dAbs
WYQQKPGKAPXLLXY

SEQ ID NO: 155 - protein, 32 aa, Artificial Sequence
  sequence comprised by Framework region 3 of CD28 VL dAbs
GVPSRFSGSGSGTDFTLTISSLQPEDFATYYC

SEQ ID NO: 156 - protein, 11 aa, Artificial Sequence
  sequence comprised by Framework region 4 of CD28 VL dAbs
FGQGTKVEIKR

SEQ ID NO: 157 - protein, 5 aa, Artificial Sequence
  sequence comprised by Framework region 1 of CD28 VH dAbs
VXLLE

SEQ ID NO: 158 - protein, 6 aa, Artificial Sequence
  sequence comprised by Framework region 1 of CD28 VH dAbs
EVXLLE

SEQ ID NO: 159 - protein, 7 aa, Artificial Sequence
  sequence comprised by Framework region 1 of CD28 VH dAbs
EVXLLES

SEQ ID NO: 160 - protein, 4 aa, Artificial Sequence
  sequence comprised by Framework region 1 of CD28 VH dAbs
LLES

SEQ ID NO: 161 - protein, 5 aa, Artificial Sequence
  sequence comprised by Framework region 1 of CD28 VH dAbs
LLESG

SEQ ID NO: 162 - protein, 6 aa, Artificial Sequence
  sequence comprised by Framework region 1 of CD28 VH dAbs
LLESGG

SEQ ID NO: 163 - protein, 29 aa, Artificial Sequence
  sequence comprised by Framework region 1 of CD28 VH dAbs
EVQLLESGGGLVQPGGSLRLSCAASGFTF

SEQ ID NO: 164 - protein, 30 aa, Artificial Sequence
  sequence comprised by Framework region 1 of CD28 VH dAbs
EVQLLESGGGLVQPGGSLRLSCAASGFTFS

SEQ ID NO: 165 - protein, 4 aa, Artificial Sequence
  sequence comprised by Framework region 2 of CD28 VH dAbs
GLEW

SEQ ID NO: 166 - protein, 11 aa, Artificial Sequence
  sequence comprised by Framework region 2 of CD28 VH dAbs
VXXXXXXGLEW

SEQ ID NO: 167 - protein, 11 aa, Artificial Sequence
  sequence comprised by Framework region 2 of CD28 VH dAbs
AXXXXXXGLEW

SEQ ID NO: 168 - protein, 11 aa, Artificial Sequence
  sequence comprised by Framework region 2 of CD28 VH dAbs
VRQAPGKGLEW

SEQ ID NO: 169 - protein, 12 aa, Artificial Sequence
  sequence comprised by Framework region 2 of CD28 VH dAbs
AVRQAPGKGLEW

SEQ ID NO: 170 - protein, 14 aa, Artificial Sequence
  sequence comprised by Framework region 2 of CD28 VH dAbs
AVRQAPGKGLEWVS

SEQ ID NO: 171 - protein, 6 aa, Artificial Sequence
  sequence comprised by Framework region 3 of CD28 VH dAbs
SKNTLY

SEQ ID NO: 172 - protein, 14 aa, Artificial Sequence
  sequence comprised by Framework region 3 of CD28 VH dAbs
SKNTLYXXXXXXXA

SEQ ID NO: 173 - protein, 23 aa, Artificial Sequence
  sequence comprised by Framework region 3 of CD28 VH dAbs
SKNTLYLQMNSLRAEDTAVYYCA

SEQ ID NO: 174 - protein, 31 aa, Artificial Sequence
  sequence comprised by Framework region 3 of CD28 VH dAbs
RFTISRDNSKNTLYLQMNSLRAEDTAVYYCA

SEQ ID NO: 175 - protein, 4 aa, Artificial Sequence
  sequence comprised by Framework region 4 of CD28 VH dAbs
GLTV

SEQ ID NO: 176 - protein, 4 aa, Artificial Sequence
  sequence comprised by Framework region 4 of CD28 VH dAbs
LTVS

SEQ ID NO: 177 - protein, 5 aa, Artificial Sequence
  sequence comprised by Framework region 4 of CD28 VH dAbs
QGLTV

SEQ ID NO: 178 - protein, 5 aa, Artificial Sequence
  sequence comprised by Framework region 4 of CD28 VH dAbs
LTVSS

SEQ ID NO: 179 - protein, 11 aa, Artificial Sequence
  sequence comprised by Framework region 4 of CD28 VH dAbs
TGQGTLVTVSS

SEQ ID NO: 180 - protein, 6 aa, Artificial Sequence
  sequence comprised by extracellular portion of both CTLA4 and CD28
MYPPPY